(19) United States
(12) Patent Application Publication
(10) Pub. No.: US 2025/0302625 A1
(43) Pub. Date: Oct. 2, 2025

(54) SYSTEMS AND METHODS FOR HEART VALVE LEAFLET REPAIR

(71) Applicant: EDWARDS LIFESCIENCES CORPORATION, Irvine, CA (US)

(72) Inventors: Bhumica A. Amin, Modesto, CA (US); Jean-Pierre Michel Rabbah, Tustin, CA (US); Alana Tyler Stein, Irvine, CA (US); Travis Zenyo Oba, Yorba Linda, CA (US); Andrew Charles May, Irvine, CA (US); Sam Shafigh, Mission Viejo, CA (US); Cristobal R. Hernandez, Santa Ana, CA (US); Shrojalkumar M. Desai, Tustin, CA (US); Jeffrey Michael Koslosky, Coto De Caza, CA (US); David M. Taylor, Lake Forest, CA (US); Hsingching C. Hsu, Santa Ana, CA (US); Sirous Darabian, Rancho Santa Margarita, CA (US); Mark Chau, Laguna Hills, CA (US); Arnold Cruz Tuason, Claremont, CA (US); Paul Kaye, Modiin (IL); Nicolas Schleiger, Huntington Beach, CA (US)

(21) Appl. No.: 19/235,960

(22) Filed: Jun. 12, 2025

Related U.S. Application Data
(63) Continuation of application No. PCT/US2023/083744, filed on Dec. 13, 2023.
(60) Provisional application No. 63/387,498, filed on Dec. 14, 2022.
(60) Provisional application No. 63/497,194, filed on Apr. 19, 2023.
(60) Provisional application No. 63/507,068, filed on Jun. 8, 2023.

(51) Int. Cl. A61F 2/24

(52) U.S. Cl. CPC A61F 2/2466; A61F 2/2454; A61F 2/2457; A61F 2220/0016

(57) ABSTRACT

A system comprises an anchor, having an anchor head that defines a forward-torque face and a reverse-torque face. A delivery tool comprises a transluminally advanceable driver, comprising a driveshaft, and a distal drive head. The drive head is shaped to engage the anchor head such that while the driveshaft presses the drive head distally into engagement with the anchor head, forward rotation of the drive head screws the anchor into the tissue. Reverse rotation of the drive head hooks the drive head onto the anchor head. While the drive head remains hooked onto the anchor head, reverse rotation of the drive head, and pulling the anchor head by tensioning of the driveshaft, unscrews the anchor from the tissue. Tensioning the driveshaft while the drive head is not hooked onto the anchor head pulls the drive head away from the anchor head. Other embodiments are also disclosed.

### CROSS-REFERENCES TO RELATED APPLICATIONS

This application is a continuation of International Patent Application No. PCT/US2023/083744, filed Dec. 13, 2023, which claims the benefit of: U.S. Patent Application No. 63/387,498, filed Dec. 14, 2022; U.S. Patent Application No. 63/497,194, filed Apr. 19, 2023; and U.S. Patent Application No. 63/507,068, filed Jun. 8, 2023, the entire disclosures all of which are incorporated by reference for all purposes.

### BACKGROUND

The native heart valves (e.g., the aortic, pulmonary, tricuspid, and mitral valves) serve critical functions in assuring the forward flow of an adequate supply of blood through the cardiovascular system. These heart valves can be rendered less effective by congenital malformations, inflammatory processes, infectious conditions, or disease. Such damage to the valves can result in serious cardiovascular compromise or death. Treatment for such disorders can be done with the surgical repair or replacement of the valve during open heart surgery or with transcatheter transvascular techniques for introducing and implanting prosthetic devices in a manner that is much less invasive than open heart surgery.

A healthy heart has a generally conical shape that tapers to a lower apex. The heart has four chambers: the left atrium, right atrium, left ventricle, and right ventricle. The left and right sides of the heart are separated by a wall generally referred to as the septum. The native mitral valve of the human heart connects the left atrium to the left ventricle. The mitral valve includes an annulus portion, which is an annular portion of the native valve tissue surrounding the mitral valve orifice, and a pair leaflets (as referred to as cusps) that extend downward from the annulus into the left ventricle. The mitral valve annulus can form a “D” shaped, oval, or otherwise out-of-round cross-sectional shape having major and minor axes. The anterior leaflet can be larger than the posterior leaflet, forming a generally “C” shaped boundary between the abutting free edges of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the muscles of the left ventricle relax, the oxygenated blood that is collected in the left atrium flows into the left ventricle. When the muscles of the left atrium relax and the muscles of the left ventricle contract, the increased blood pressure in the left ventricle urges the two leaflets together, thereby closing the one-way mitral valve so that blood cannot flow back to the left atrium and is instead expelled out of the left ventricle through the aortic valve. To prevent the two leaflets from prolapsing or flailing under pressure and folding back through the mitral annulus toward the left atrium, a plurality of fibrous cords called chordae tendineae tether the leaflets to papillary muscles in the left ventricle.

Valve regurgitation occurs when the native valve fails to close properly and blood flows into the left atrium from the left ventricle during the systole phase of heart contraction. Valve regurgitation (especially mitral valve regurgitation) is the most common form of valvular heart disease. Mitral regurgitation has different causes, including leaflet prolapse or flail, restricted leaflet motion (e.g., due to leaflet rigidity/leaflet calcification), and/or dysfunctional papillary muscles stretching.

There is a continuing need for effective devices and methods for treating valve issues, including leaflet flail, prolapse, and restricted leaflet motion.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure can be included in the examples summarized here.

In some implementations, a system is provided for use with a real or simulated heart valve, such as a mitral valve or a tricuspid valve. In some implementations, a delivery tool comprising a shaft is transluminally advanced through a catheter while the shaft is engaged with an interface, e.g., via a ripcord that extends through both the shaft and a portion of the interface. In some implementations, the shaft can be used to deploy the implant so that the implant expands out of the catheter.

In some such implementations, implant is self-expandable and can expand into an expanded state when the implant is advanced out of the catheter.

In some such implementations, implant is mechanically expandable and can be actuated to expand into an expanded state when or after the implant is advanced out of the catheter.

In some implementations, the system comprises an implant that includes a wing and an interface (e.g., an anchor receiver, etc.). In some implementations, the delivery tool is reversibly engaged to the implant via the interface. In some implementations, the shaft defines a latch that reversibly engages the shaft to the interface.

In some implementations, the delivery tool includes a driver that is used to anchor an anchor through the interface and to the tissue site. In some implementations, the driver is extended through a distal end portion of the shaft, to the interface.

In some implementations, the anchor includes a helical tissue-engaging element that extends from an anchor head. Alternatively or in addition, the anchor includes a shape-memory material that changes shape and/or defines barbs that expand upon the anchor being released from compression.

In some implementations, the implant includes an anchor receiver that is separate from the interface. In some implementations, the driver is extended through a lateral opening of the shaft to the anchor receiver, e.g., while the delivery tool is coupled to the implant's interface. In some implementations, the anchor is advanced through the interface and the tissue's surface, along a curved path within the tissue such that a distal part of the anchor exits the tissue and is received by the anchor receiver.

In some implementations, the interface is at a root portion of the wing, from which the wing extends to a tip portion of the wing.

In some implementations, an expansion element aids in expanding and/or maintaining expansion of the wing.

In some implementations, the shaft is used to position the implant with the interface at a tissue site, such that the wing extends over a leaflet of the valve, toward an opposing leaflet of the valve.

In some implementations, the wing includes a frame that is covered by a flexible sheet. For example, the sheet can define lateral flaps that extend laterally beyond the frame, such that the lateral flaps enter the commissures of the native valve.

In some implementations, the interface is a ratcheting interface that facilitates abutment of the implant to tissue site by allowing the user to pull the driver and the anchor proximally with respect to the interface.

In some implementations, the shaft bifurcates into two branches, and a respective driver extends through each branch to a respective interface. In some implementations, each of the respective drivers are operable, e.g., via a controller, either simultaneously or individually.

In some implementations, each interface extends obliquely from the wing, which can facilitate interaction between a pair of anchors with the pair of interfaces as the anchors are screwed (e.g., along nonparallel axes) into respective tissue sites. For example, each shaft branch can be skewed aside, in order to reduce a risk of “shadowing” artifact caused by the shaft when visualizing the implant, e.g., using imaging devices that face the wing orthogonally.

In some implementations, the implant can be configured to facilitate ingrowth of tissue at the anchor receiver, and to inhibit ingrowth of tissue at the wing's tip portion, which can facilitate upstream and downstream deflection of the wing in response to the cardiac cycle.

In some implementations, while the interface is anchored to the site, the wing provides resistance to upstream deflection of the leaflet.

In some implementations, the wing provides greater resistance to upstream deflection of a root portion of the leaflet, while allowing a lip portion of the leaflet to behave more flexibly. For example, the wing's root portion can include stiffer material and/or be more densely populated with supportive members, than the wing's tip portion.

In some implementations, the wing includes a flex element that facilitates movement of the wing's tip portion with respect to the wing's root portion during the cardiac cycle. In some implementations, the flex element biases the tip portion to be deflected into the ventricle and away from the opposing leaflet of the valve. For example, the flex element can transition away from a relaxed state (e.g., can become strained) as the heart cycles into systole, and toward the relaxed state as the heart cycles into diastole.

In some implementations, the flex element is a hinge that facilitates articulation of the wing's tip portion with respect to the wing's root portion. In this way, the wing's root portion being anchored directly to the site (and optionally, being stiffer than the tip portion) can provide greater support to a portion of the leaflet experiencing prolapse, while articulation of the tip portion with respect to the root portion can improve coaptation of a flailing portion of the leaflet.

In some implementations, the wing provides dynamic support to the leaflet during the cardiac cycle. In some implementations, the wing provides greater resistance to upward deflection of the leaflet when the leaflet reaches and/or passes an upstream deflection-limit of the leaflet.

In some implementations, a limb or extension coupled to the wing extends away from the wing such that the limb/extension contacts tissue of the heart adjacent a root of an opposing leaflet when the root portion of the wing is placed against the annulus. By contacting the tissue, the limb/extension moderates upstream deflection of the wing. In some implementations, the limb/extension is a leg that contacts ventricular tissue, such as an underside of the valve, e.g., adjacent a commissure and/or a subannular groove of the valve.

In some implementations, a pair of arms are coupled to the wing. In some implementations, each arm arcs divergently away from the wing to an anchor point of the arm, such that when the anchor point is anchored to the annulus, each arm arcs from the anchor point, along the annulus and to the wing, e.g., such that the arms define an annular support.

In some such implementations, the arms are connected to the wing by a hinged coupling at which the wing articulates with respect to the annular support while the wing is compressed, and expansion of the wing inhibits the articulation by restraining the hinged coupling.

In some implementations, a hinge couples each arm to the wing, and articulation of the hinge in response to deflection of the wing helps to keep the wing's root portion in contact with the annulus during the cardiac cycle.

In some implementations, a pair arms extend laterally away from the wing's tip portion to define a lateral portion of each arm.

In some implementations, the implant is implanted such that the arms' lateral portions each press in an upstream direction against a respective lateral site on a downstream side of the leaflet. In some implementations, the arms' lateral portions press the wing against a medial site of the leaflet's upstream side, thereby pinching the leaflet between the wing and the arms' lateral portions.

In some implementations, the implant includes a limiter that limits upstream deflection of the wing and leaflet, e.g., by limiting range of motion of a hinge that couples the tip portion to the root portion. In some implementations, the limiter can contact the implant (e.g., the interface and/or a portion of the wing) when the wing reaches the deflection-limit.

In some implementations, the limiter includes a backstop portion that extends away from the wing and the interface. For example, the backstop portion can be wider than the wing, and/or can be anchored to tissue of the heart, for greater stability of the limiter.

In some implementations, the wing's deflection-limit is adjustable by adjusting the limiter, e.g., by pressing the backstop portion of the limiter against annular tissue. In some implementations, the limiter is adjusted by adjusting a depth to which the anchor is anchored within the tissue, and/or by adjusting an angle of the limiter with respect to the interface.

In some implementations, the interface itself is adjustable, and adjusting the interface, e.g., by changing an angle between the interface and the wing's root portion, adjusts the wing's deflection-limit.

In some implementations, the limiter comprises a tether that becomes tensioned as the wing reaches the deflection-limit. In some implementations, the deflection-limit is adjustable by adjusting a length of the tether.

In some implementations, the wing can limit its own deflection. In some implementations, the wing's frame can provide greater resistance to upstream deflection of the wing (e.g., past the deflection-limit) than to downstream deflection of the wing. For example, the frame can define notches that widen while the wing deflects downstream, and that close while the wing deflects upstream, inhibiting upstream deflection of the wing beyond the deflection-limit.

In some implementations, the implant is adjustable in size. In some implementations, a bulking element is actuated to change a bulkiness of at least a portion of the implant (e.g., the wing's tip portion, a mid portion, an end portion, etc.).

In some implementations, the wing is adjustable in size. In some implementations, a shape-memory member is coupled to the wing, and heating (e.g., electrically heating) the shape-memory member changes its shape, which resizes the wing.

In some implementations, the wing is adjustable in shape. In some implementations, a beam is connected to the wing, and a line is coupled to the beam such that tensioning the line strains the beam, thereby reshaping the wing.

In some implementations, the implant includes a lock that locks the wing such that the wing retains the new size, even after shape-memory member is no longer heated.

In some implementations, the delivery tool includes a lock having a plurality of units, the lock being unlockable such that that the units are separated and translatable away from each other. In some such implementations, a tether connects the lock's units while the lock is unlocked, e.g., and tensioning the tether relocks the lock.

In some implementations, the wing is slidable (e.g., intracardially slidable using an adjustment rod) and/or pivotable with respect to the anchor. In some implementations, the anchor receiver defines an oblong opening, and the wing is slidable along a major axis of the opening. In some implementations, after sliding and/or allowing the wing to pivot with respect to the anchor, the implant is locked to the anchor, e.g., by sandwiching the anchor receiver between a first collar and a second collar of the interface.

In accordance with some implementations, a system and/or an apparatus (which can be used with a valve of a heart, e.g., of a living subject or of a simulation, the valve having an annulus, a first leaflet and an opposing leaflet opposing the first leaflet) includes an anchor, an implant, and/or a delivery tool. The implant can include, among other components, a wing, and/or an interface. The wing can be configured to define a first face (e.g., a contact face), and a second face (e.g., an opposing face, a face opposite to the first face, etc.).

In some implementations, the anchor comprises an anchor head, and a tissue-engaging element that extends from the anchor head. In some implementations, an outer diameter of the anchor head is greater than an outer diameter of the tissue-engaging element.

In some implementations, the wing can have a root portion and a tip portion, as well as a flex element that couples the tip portion to the root portion.

In some implementations, the interface can be coupled to the root portion of the wing, can be configured to receive the anchor, and/or can be configured to be anchored by the anchor.

In some implementations, the delivery tool includes, among other components, a catheter, a shaft, and a driver. The catheter is transluminally advanceable to the chamber.

In some implementations, the shaft can be engaged with the interface, and/or configured, via the engagement with the interface, to (i) deploy the implant out of the catheter, and/or (ii) position the implant in a position.

In some implementations, in the position, the interface can be at a site upstream of the valve, and/or the wing can extend over the first leaflet toward the opposing leaflet, with the first face or contact face facing the first leaflet.

In some implementations, the driver can be engaged with the anchor, and/or can be configured to secure the implant in the position by using the anchor to anchor the interface to tissue of the heart at the site.

In some implementations, the implant is sterile. In some implementations, the anchor is sterile. In some implementations, the delivery tool is sterile.

In some implementations, the flex element protrudes from the first face or contact face of the wing.

In some implementations, the flex element protrudes from the second face or opposing face of the wing.

In some implementations, the flex element is a flexure.

In some implementations, the flex element is a hinge.

In some implementations, the flex element is a living hinge.

In some implementations, the flex element includes a pair of interlocking loops, a first one of the loops defined by the root portion, and a second one of the loops defined by the tip portion.

In some implementations, the flex element includes a plurality of coiled wires connecting the tip portion to the root portion.

In some implementations, the flex element includes a plurality of rings connecting the tip portion to the root portion.

In some implementations, the flex element includes a plurality of sutures connecting the tip portion to the root portion.

In some implementations, the flex element includes a tube through which respective portions of the tip portion and the root portion extend alongside each other, such that the tip portion and root portion can articulate in relation to each other.

In some implementations, the root portion is stiffer than the tip portion.

In some implementations, the implant is configured such that, while the implant is secured in the position, flexing of the flex element facilitates deflection of the tip portion with respect to the root portion in response to a cardiac cycle of the heart.

In some implementations, the flex element is protrusive, and the implant is configured such that, while the implant is secured in the position, the flex element abuts a hinge-point between a leaflet of the valve and an annulus of the valve.

In some implementations, the flex element is protrusive so as to abut a hinge-point between a leaflet of the valve and an annulus of the valve and is positioned within the implant such that abutment of the flex element against the hinge-point positions the interface at the site.

In some implementations, the flex element is protrusive, and the shaft is configured to position the interface at the site by abutting the flex element against a hinge-point between a leaflet of the valve and an annulus of the valve.

In some implementations, the wing includes a frame, and a flexible sheet disposed over the frame, and/or the frame defines the flex element.

In some implementations, the flex element is a torsion spring.

In some implementations, the flex element is a hinge.

In some implementations, the flex element is a ball-and-socket hinge.

In some implementations, adjusting flexibility of the delivery tool's shaft can moderate deflectability of the wing while the implant is anchored to the tissue. In some implementations, the shaft can influence the valve's function by supporting the tissue via the interface.

In some implementations, increasing the shaft's flexibility can compensate for support the shaft provides, facilitating assessment of the implant's influence upon the valve's function while the shaft remains engaged to the interface.

In some implementations, the anchor and the interface are configured to prevent a gap from opening between the tissue and the interface. In some implementations, a portion of the torque that the driver transfers to the anchor head is translated into a distal pushing force upon anchor receiver.

In some implementations, when the anchor head is fully seated within the interface, further rotation of the anchor moves the anchor proximally away from the interface.

In some implementations, the anchor and the interface are configured to prevent a gap from opening between the tissue and the interface by inhibiting non-helical advancement of the anchor distally through the interface.

In some implementations, the anchor head is rotatably coupled to the interface, while the anchor head remains longitudinally fixed with respect to the interface. In some implementations, the interface facilitates non-helical withdrawal of the anchor proximally through the interface.

In some implementations, the interface comprises a stopper that is configured for the anchor's tissue-engaging element to be screwed through the stopper, and for the anchor's helical advancement to halt when the anchor head reaches the stopper.

In accordance with some implementations, a system and/or an apparatus (which can be used with a valve of a heart, e.g., of a living subject or of a simulation, the valve having an annulus, and a first leaflet and an opposing leaflet opposing the first leaflet) includes an anchor, an implant, and/or a delivery tool. In some implementations, the implant can include, among other components, an interface, a first wing and second wing.

In some implementations, the first wing can extend from a first root portion of the first wing to a first tip portion of the first wing, and can define a first face (e.g., a contact face), and a second face (e.g., an opposing face, a face opposite to the first face, etc.).

In some implementations, the second wing can extend, over the second face or opposing face of the first wing, from a second root portion of the second wing to a second tip portion of the second wing, such that the first wing is deflectable toward and away from the second wing.

In some implementations, the interface is coupled to the first root portion and to the second root portion.

In some implementations, the interface is configured to receive the anchor, and/or is configured to be anchored to a site in the chamber.

In some implementations, the delivery tool includes, among other components, a catheter, a shaft, and a driver. The catheter is transluminally advanceable to the chamber.

In some implementations, the shaft can be engaged with the interface, and/or configured, via the engagement with the interface, to (i) deploy the implant out of the catheter, and/or (ii) position the implant in a position.

In some implementations, in the position, the interface can be at a site upstream of the valve, the first wing can extend over the first leaflet toward the opposing leaflet, with the first face/contact face facing the first leaflet, and/or the second wing can extend over the second face/opposing face of the first wing.

In some implementations, the driver can be engaged with the anchor, and/or can be configured to secure the implant in the position by using the anchor to anchor the interface to tissue of the heart at the site.

In some implementations, the implant is sterile. In some implementations, the anchor is sterile. In some implementations, the delivery tool is sterile.

In some implementations, the implant is configured such that, while the implant remains secured in the position, during ventricular diastole the first wing deflects away from the second wing.

In some implementations, the implant further includes a third wing, the third wing having a third root portion that is coupled to the interface, and extending, over the second wing, from the third root portion to a third tip portion of the third wing, the second wing being deflectable toward and away from the third wing.

In some implementations, the third wing is shorter than the second wing.

In some implementations, the second wing is more flexible than the third wing.

In some implementations, the first wing is more flexible than the second wing.

In some implementations, the implant is configured such that, while the implant remains secured in the position, during ventricular diastole the second wing deflects away from the third wing.

In some implementations, the implant is configured such that, while the implant remains secured in the position, during ventricular diastole the first wing deflects away from the second wing.

In some implementations, the implant is configured such that, while the implant remains secured in the position, during ventricular systole the first wing deflects into contact with the second wing.

In some implementations, the implant is configured such that, while the implant remains secured in the position, during ventricular systole the second wing deflects into contact with the third wing.

In some implementations, the first wing defines multiple holes therethrough.

In some implementations, the implant is configured such that, while the implant is secured in the position, during ventricular systole the first wing deflects into contact with the second wing in a manner that obstructs blood flow through the holes.

In some implementations, the second wing defines multiple holes therethrough, the holes of the first wing being positioned such that, while the first wing is in contact with the second wing, the holes of the first wing are offset with respect to the holes of the second wing.

In some implementations, the second wing is stiffer than the first wing.

In some implementations, the second wing is shorter than the first wing.

In some implementations, the implant further includes a flexible pouch, the first and second wings being disposed within the pouch.

In some implementations, the pouch is configured to expand during ventricular diastole, and to contract during ventricular systole.

In some implementations, the pouch is coupled to the interface.

In some implementations, on a first side of the pouch, the pouch defines multiple first-pouch-side holes that provide fluid communication between inside and outside of the pouch.

In some implementations, on a second side of the pouch, opposite the first side, the pouch defines multiple second-pouch-side holes that provide fluid communication between inside and outside of the pouch.

In some implementations, the pouch is configured such that, while the implant is secured in the position, when the first wing deflects toward the second wing during ventricular systole the first side of the pouch moves toward the second side of the pouch in a manner that inhibits blood flow through the first-pouch-side holes and the second-pouch-side holes.

In some implementations, the first wing and the second wing are each stiffer than the pouch.

In some implementations, the first-pouch-side holes and the second-pouch-side holes are positioned such that, while the implant is secured in the position and the first wing deflects toward the second wing, the first-pouch-side holes are offset with respect to the second-pouch-side holes.

In accordance with some implementations, a system and/or an apparatus (which can be used with a valve of a heart, e.g., of a living subject or of a simulation, the valve having an annulus, a first leaflet and an opposing leaflet opposing the first leaflet) includes an implant. In some implementations, the implant can include, among other components, a wing, an interface and/or a limiter.

In some implementations, the wing can have a root portion and/or a tip portion, and can define a first face (e.g., a contact face), and a second face (e.g., an opposing face, a face opposite to the first face, etc.).

In some implementations, the interface is coupled to the root portion of the wing. In some implementations, the interface can be configured to receive the anchor.

In some implementations, the interface can be configured to be anchored to a site in the chamber such that the implant is secured in a position in which the wing extends over the first leaflet toward the opposing leaflet, and responsively to a cardiac cycle of the heart, the wing deflects, in a reciprocating manner, in an upstream direction and in a downstream direction.

In some implementations, the limiter is configured to define a deflection-limit of the wing, and to inhibit deflection of the wing in the upstream direction beyond the deflection-limit by providing an opposing force upon the wing reaching the deflection-limit.

In some implementations, the implant is sterile.

In some implementations, the implant is configured such that, upon the wing reaching the deflection-limit, the wing contacts the limiter.

In some implementations, the limiter includes a tether, the tether being configured to become tensioned as the wing reaches the deflection-limit.

In some implementations, the limiter is coupled to the interface, such that the interface is deflectable toward and away from the limiter.

In some implementations, the implant is configured such that, upon the wing reaching the deflection-limit, the interface contacts the limiter.

In some implementations, the limiter is coupled to the interface, and extends, away from the interface and over the wing, such that the wing is deflectable toward and away from the limiter.

In some implementations, the limiter is stiffer than the wing.

In some implementations, the deflection-limit is defined by a relative position between the limiter and the wing.

In some implementations, the limiter extends away from the interface and over the second face or opposing face of the wing, the wing is deflectable toward the limiter such that the wing contacts the limiter upon reaching the deflection-limit.

In some implementations, the limiter is shaped such that the wing contacts the limiter at a contact-portion of the wing that is between the root portion of the wing and the tip portion of the wing.

In some implementations, the limiter is shaped to define a cross-brace that, upon the wing reaching the deflection-limit, lies in contact with the wing, widthways across the wing.

In some implementations, the wing is a first wing, and the limiter includes a second wing.

In some implementations, the second wing is shorter than the first wing.

In some implementations, the second wing is narrower than the first wing.

In some implementations, the limiter has a backstop portion that is shaped to press against tissue of the chamber upon anchoring of the interface to the site.

In some implementations, the system/apparatus further includes an anchor, and/or the backstop portion defines an anchor receiver that is configured to receive the anchor in a manner that anchors the anchor receiver to tissue of the chamber.

In some implementations, the backstop portion is wider than the wing.

In some implementations, the backstop portion extends from the interface away from the wing.

In some implementations, the limiter is shaped to define a cross-brace, along a width of the limiter, that can be configured to press against tissue of the chamber upon anchoring of the interface to the site.

In some implementations, the limiter includes a frame that includes a first portion that comprises or is formed from sheet metal, and/or a second portion, coupled to the first portion that comprises or is formed from wire.

In some implementations, the first portion is shaped to define a plurality of adjoining cells.

In some implementations, the wire comprises or is formed from a shape-memory alloy.

In some implementations, the second portion is more flexible than the first portion.

In some implementations, the implant is configured such that, as the wing approaches the deflection-limit, the interface approaches the limiter.

In some implementations, the implant is configured such that, upon the wing reaching the deflection-limit, the interface contacts the limiter.

In some implementations, the limiter is shaped to define a cradle such that, upon the wing reaching the deflection-limit, the interface becomes temporarily seated within the cradle.

In some implementations, the implant includes a spring configured to strain as the interface approaches the limiter.

In some implementations, the implant includes a spring configured to bias the interface away from the limiter.

In some implementations, the limiter extends from the interface away from the wing.

In some implementations, the limiter is disposed on an opposite side of the interface from the wing.

In some implementations, the limiter is shaped such that anchoring of the interface to the site presses the limiter against tissue of the chamber.

In accordance with some implementations, a system and/or an apparatus (which can be used with a valve of a heart, e.g., of a living subject or of a simulation, the valve having an annulus, a first leaflet and an opposing leaflet opposing the first leaflet) includes an implant. In some implementations, the implant can include, among other components, a wing, and/or an interface. The wing can have a root portion and/or a tip portion.

In some implementations, the interface is coupled to the root portion of the wing. In some implementations, the interface can be configured to receive the anchor.

In some implementations, the interface is configured to be anchored to a site in the chamber, such that the implant is in a position in which the wing extends over the first leaflet toward the opposing leaflet.

In some implementations, the wing can be configured, responsively to a cardiac cycle of the heart, to deflect, in a reciprocating manner, in an upstream direction and in a downstream direction, the wing being configured to define a deflection-limit, and to become resistant to deflection in the upstream direction upon reaching the deflection-limit.

In some implementations, the implant is sterile.

In some implementations, the wing can be configured to become resistant to deflection in the upstream direction by the tip portion of the wing contacting the root portion of the wing upon the wing reaching the deflection-limit.

In some implementations, the wing includes a hinge that articulatably couples the root portion of the wing to the tip portion of the wing.

In some implementations, the hinge can be configured with a range of motion that defines the deflection-limit of the wing.

In some implementations, the tip portion includes at least a first part and a second part, the second part is deflectable with respect to the first part, and/or upon the wing reaching the deflection-limit, the second part contacts the first part.

In some implementations, the first part is closer than the second part to the root portion.

In some implementations, the first part is closer than the second part to the interface.

In some implementations, the tip portion further includes a third part, the third part is deflectable with respect to the second part, and/or upon the wing reaching the deflection-limit, the third part contacts the second part.

In some implementations, the second part is closer than the third part to the root portion.

In some implementations, the second part is closer than the third part to the interface.

In some implementations, at least one of the first part, the second part, and the third part has a different flexibility from at least another of the first part, the second part, and the third part.

In some implementations, the wing includes a flexible frame, the frame being more flexible to deflection in the downstream direction than to deflection in the upstream direction.

In some implementations, the frame has a plurality of notches cut therein.

In some implementations, the implant is configured such that deflection of the wing in the downstream direction causes the notches to widen, and deflection of the wing in the upstream direction causes the notches to narrow.

In some implementations, the notches are notches of a first set of notches, the frame has a second set of notches cut therein. In some implementations, the implant is configured such that: (i) deflection of the frame in the downstream direction causes the first set of notches to widen and the second set of notches to narrow, and/or (ii) deflection of the frame in the upstream direction causes the first set of notches to narrow and the second set of notches to widen.

In some implementations, the notches are on an upstream side of the frame.

In some implementations, the notches are notches of a first set of notches, and the frame has a second set of notches cut therein, the second set of notches being on a downstream side of the frame.

In some implementations, the frame is configured such that flexing of the frame in a first direction widens the notches of the first set and narrows the notches of the second set, and flexing of the frame in a second direction narrows the notches of the first set and widens the notches of the second set.

In accordance with some implementations, a system and/or an apparatus (which can be used with a valve of a heart, e.g., of a living subject or of a simulation, the valve having an annulus, a first leaflet and an opposing leaflet opposing the first leaflet) includes an implant, a delivery tool, a first anchor, and a second anchor. In some implementations, the implant can include a wing, a first interface and a second interface.

In some implementations, the wing can have a root portion and/or a tip portion, and can define a first face (e.g., a contact face), and a second face (e.g., an opposing face, a face opposite to the first face, etc.).

In some implementations, the first interface can define a first longitudinal axis and the second interface can define a second longitudinal axis, each of the first and second interfaces: disposed at the root portion, and coupled to the wing such that the first longitudinal axis is nonparallel to the second longitudinal axis.

In some implementations, the delivery tool includes, among other components, a catheter, a first shaft, a second shaft, a first driver and a second driver. The catheter is transluminally advanceable to the chamber.

In some implementations, the first and second shafts can be engaged a corresponding one of the first and second interfaces, and/or configured, via the engagement with the corresponding interfaces, to (i) deploy the implant out of the catheter, and/or (ii) position the implant in a position.

In some implementations, in the position, the first interface is at a first site upstream of the valve, the second interface is at a second site upstream of the valve, and/or the wing can extend over the first leaflet toward the opposing leaflet, with the contact face facing the first leaflet.

In some implementations, each driver can be engaged with a corresponding one of the first and second anchors, and configured to secure the implant in the position by screwing: the first anchor along the first longitudinal axis to anchor the first interface to tissue at the first site, and the second anchor along the second longitudinal axis to anchor the second interface to tissue at the second site.

In some implementations, the implant is sterile. In some implementations, the first anchor and the second anchor are sterile. In some implementations, the delivery tool is sterile.

In some implementations, for each of the first and second interfaces, the interface has a proximal end that is orthogonal to the longitudinal axis of the interface.

In some implementations, each of the first and second interfaces has a circular proximal end.

In some implementations, the root portion of the wing defines a plane that is oblique to both the first longitudinal axis and the second longitudinal axis.

In some implementations, while the first and second shafts each engage the corresponding interfaces, each of the respective shafts is oblique to the plane defined by the root portion of the wing.

In some implementations, an angle between the first longitudinal axis and the plane defined by the root portion of the wing is equal to an angle between the second longitudinal axis and the plane defined by the root portion of the wing.

In some implementations, an angle between the first longitudinal axis and the plane defined by the root portion of the wing is unequal to an angle between the second longitudinal axis and the plane defined by the root portion of the wing.

In some implementations, the implant defines a first angle between the first longitudinal axis and a region of the plane that is disposed between the first and second interfaces, and a second angle between the second longitudinal axis and the region of the plane. In some implementations, the first angle is greater than the second angle.

In some implementations, the first angle and the second angle are both acute.

In some implementations, the first angle is obtuse.

In some implementations, the second angle is acute.

In some implementations, the first interface includes a first cylindrical tube extending along the first longitudinal axis, and/or the second interface includes a second cylindrical tube extending along the second longitudinal axis.

In some implementations, each of the first and second cylindrical tubes has a circular cross-section that is transverse to the respective longitudinal axis, and/or a non-circular, elliptical distal end.

In some implementations, each of the first and second interfaces has a distal end that is oblique to the longitudinal axis of the respective interface.

In some implementations, the distal end of each of the first and second interfaces is parallel with a plane defined by the root portion of the wing.

In some implementations, each of the first and second interfaces has a proximal end that is oblique with respect to the plane defined by the root portion.

In some implementations, the anchor has an anchor head, from which a tissue-engaging element extends, and/or for each of the first and second anchors, the respective driver can be configured to screw the anchor along the respective longitudinal axis until the anchor head abuts the proximal end of the respective interface.

In accordance with some implementations, a system and/or an apparatus (which can be used with tissue, e.g., of a living subject or of a simulation) includes an anchor, an implant and a delivery tool. The implant can include a ratcheting interface that can be configured to be anchored to a site of the tissue.

In some implementations, the delivery tool includes, among other components, a catheter, a shaft, and a driver. The catheter is transluminally advanceable to the chamber.

In some implementations, the shaft can be engaged with the interface, and/or configured, via the engagement with the interface, to (i) deploy the implant out of the catheter, and/or (ii) position the implant in a position.

In some implementations, in the position, the interface can be at a site, and the driver can be engaged with the anchor, and/or can be configured to anchor the interface to the site by helically advancing the anchor distally through the interface and into tissue at the site.

In some implementations, the interface can be configured to inhibit non-helical advancement of the anchor distally through the interface and facilitate non-helical withdrawal of the anchor proximally through the interface.

In some implementations, the implant is sterile. In some implementations, the anchor is sterile. In some implementations, the delivery tool is sterile.

In some implementations, the interface includes: a tubular anchor receiver defining a lumen, and/or a tab that protrudes into the lumen such that application of a non-helical distalward force to the anchor causes the anchor to abut the tab in a manner that inhibits the non-helical distal advancement.

In some implementations, the tab can be configured to deflect outwardly in response to application of a non-helical proximal force to the anchor, facilitating the non-helical proximal withdrawal.

In some implementations, the anchor includes a helical tissue-engaging element, and/or the tab can be configured such that application of the non-helical distal force to the anchor causes the helical tissue-engaging element to abut the tab in a manner that inhibits the non-helical distal advancement.

In some implementations, the helical tissue-engaging element is configured to helically slide over the tab during helical distal advancement of the anchor through the interface.

In some implementations, the tab is configured such that application of a non-helical proximal force to the anchor causes the helical tissue-engaging element to deflect the tab outwardly, facilitating the non-helical proximal withdrawal.

In some implementations, the tab is configured such that application of a non-helical proximal force to the anchor causes the helical tissue-engaging element to ratchet proximally past the tab and through the lumen, facilitating the non-helical proximal withdrawal.

In accordance with some implementations, a system and/or an apparatus (which can be used with a valve of a heart, e.g., of a living subject or of a simulation) includes an implant and a delivery tool. In some implementations, the implant can include a wing that extends from a root portion of the wing to a tip portion of the wing, and/or that defines a first face (e.g., a contact face), and a second face (e.g., an opposing face, a face opposite to the first face, etc.).

In some implementations, the wing has a compressed state and an expanded state.

In some implementations, the wing includes a flexible frame that includes a shape-memory material and biases the wing toward assuming the expanded state. In some such implementations, the wing and/or flexible frame is self-expandable and can expand into an expanded state when the wing is advanced out of a catheter.

In some such implementations, wing and/or frame is mechanically expandable and can be actuated to expand into an expanded state when or after the wing is advanced out of the catheter.

In some implementations, the wing can include an expansion element, coupled to the wing, and having: a compact state, and an extended state in which the expansion element resists compression of the wing toward the compressed state.

The valve can have a first leaflet and an opposing leaflet, and/or the heart can have a chamber upstream of the valve.

In some implementations, the delivery tool includes, among other components, a catheter, a shaft, and a driver. In some implementations, the catheter is transluminally advanceable to the chamber while the catheter houses the implant while the wing is in the compressed state and the expansion element is in the compact state.

In some implementations, the catheter is transluminally advanceable to the chamber while the shaft is disposed within the catheter, and the shaft is engaged to the implant.

In some implementations, the delivery tool is configured to deploy the implant out of the catheter such that, within the chamber, the wing assumes the expanded state and the expansion element assumes the extended state.

In some implementations, the delivery tool is configured to position the implant in a position in which the wing extends over the first leaflet toward the opposing leaflet, and the first face or contact face faces the first leaflet.

In some implementations, the implant is sterile. In some implementations, the delivery tool is sterile.

In some implementations, the expansion element is configured to resist transition from the extended state toward the compact state.

In some implementations, the expansion element includes a spring.

In some implementations, the expansion element includes a plurality of subunits, and the expansion element is configured such that extending the expansion element into the extended state causes the subunits to fit together.

In some implementations, the expansion element includes a plurality of subunits, configured to lock together upon the expansion element assuming the extended state.

In some implementations, the expansion element is straighter in the extended state than in the compact state.

In some implementations, the expansion element includes a hinge, and the expansion element can be configured such that straightening the hinge straightens the expansion element.

In some implementations, the delivery tool further includes an extension actuator, the extension actuator configured to transluminally extend the expansion element from the compact state to the extended state.

In some implementations, the implant is configured such that extension of the expansion element from the compact state to the extended state applies an expansion force to the wing, the expansion force facilitating expansion of the wing from the compressed state to the expanded state.

In some implementations, the implant includes a pair of interfaces at the root portion of the wing. In some implementations, the shaft bifurcates at a distal portion of the shaft into two branches, each of the branches being engaged with a corresponding one of the interfaces.

In some implementations, the expansion element is configured to push the interfaces away from each other as the expansion element extends toward its extended state.

In some implementations, the expansion element is coupled to the wing via the pair of interfaces.

In some implementations, the extension actuator is disposed between the branches.

In accordance with some implementations, a system and/or an apparatus (which can be used with a valve of a heart, e.g., of a living subject or of a simulation, the valve having an annulus, a first leaflet and an opposing leaflet opposing the first leaflet) includes an anchor, an implant, and/or a delivery tool. In some implementations, the implant can include, among other components, a wing, a frame that provides mechanical support to the wing and/or an interface at the root portion.

In some implementations, the wing can have a root portion and/or a tip portion, and a flexible sheet covering the frame, and extending beyond the frame to define lateral flaps.

In some implementations, the delivery tool includes, among other components, a catheter, a shaft, and a driver. The catheter can be configured to be transluminally advanceable to the chamber.

In some implementations, the shaft can be engaged with the interface, and/or configured, via the engagement with the interface, to (i) deploy the implant out of the catheter, and/or (ii) position the implant in a position.

In some implementations, in the position, the interface can be at a site upstream of the valve, and/or the wing can extend over the first leaflet toward the opposing leaflet, and the lateral flaps extend over the first leaflet toward respective commissures of the valve.

In some implementations, the driver can be engaged with the anchor, and/or can be configured to secure the implant in the position by using the anchor to anchor the interface to tissue of the heart at the site.

In some implementations, the implant is sterile. In some implementations, the anchor is sterile. In some implementations, the delivery tool is sterile.

In some implementations, the wing is more flexible at the lateral flaps than at a medial region in which the frame is disposed.

In some implementations, the flexible sheet has a shape that resembles that of a manta ray.

In some implementations, each of the lateral flaps defines a lateral extremity between a root portion of the lateral flap and a tip portion of the lateral flap.

In some implementations, the lateral extremity is angular.

In accordance with some implementations, a system and/or an apparatus (which can be used with a valve of a heart, e.g., of a living subject or of a simulation) includes an implant, the implant including a wing and an interface at the root portion. In some implementations, the wing extends from a root portion of the wing to a tip portion of the wing, the root portion being stiffer than the tip portion, and the wing defines a first face (e.g., a contact face), and a second face (e.g., an opposing face, a face opposite to the first face, etc.).

The valve can have a first leaflet and an opposing leaflet, and/or the heart can have a chamber upstream of the valve.

In some implementations, the implant is configured to be implanted in a position in which the interface is at a site upstream of the valve, the wing extends over the first leaflet toward the opposing leaflet, and the first face/contact face faces the first leaflet.

In some implementations, the implant is sterile.

In some implementations, the wing includes a flexible frame that provides mechanical support to the root portion of the wing.

In some implementations, the tip portion of the wing includes a flexible sheet.

In some implementations, the flexible sheet includes a polymer.

In some implementations, the wing includes a flexible frame that provides mechanical support to the wing.

In some implementations, the flexible frame defines less open space at the root portion of the wing than at the tip portion of the wing.

In some implementations, members of the frame are thicker at the root portion of the wing than at the tip portion of the wing.

In some implementations, members of the frame are spaced more closely to each other at the root portion of the wing than at the tip portion of the wing.

In some implementations, the flexible frame includes a wire frame, and the wire frame includes thicker wires at the root portion of the wing than at the tip portion of the wing.

In some implementations, the frame at the root portion of the wing includes a first material, and the frame at the tip portion of the wing includes a second material. In some implementations, the first material is stiffer than the second material.

In some implementations, the flexible frame includes a wire frame, and the wire frame is more densely populated with wires at the root portion of the wing than at the tip portion of the wing.

In some implementations, the wire frame includes thicker wires at the root portion of the wing than at the tip portion of the wing.

In some implementations, the wing includes a mesh (e.g., formed from wire).

In some implementations, the wing further includes a flexible frame over which the mesh is disposed.

In some implementations, the mesh has a weave that is more densely woven at the root portion than at the tip portion.

In some implementations, the mesh includes thicker wire at the root portion than at the tip portion.

In some implementations, the wing defines a flex element, the flex element coupling the tip portion of the wing to the root portion of the wing.

In some implementations, the implant is configured such that, while the implant is secured in the position, flexing of the flex element facilitates deflection of the tip portion with respect to the root portion in response to a cardiac cycle of the heart.

In accordance with some implementations, a method (which can be used to treat a valve of a heart, e.g., of a living subject or of a simulation, the valve can have an annulus, a first leaflet, and an opposing leaflet) includes advancing to the chamber a catheter, a shaft, and an implant. In some implementations, the implant includes an interface, engaged with a distal end of the shaft, as well as a flexible wing coupled to the interface.

In some implementations, the method includes using the shaft to deploy the implant out of the catheter and into the chamber and positioning the implant in a position in which the interface is at a site on the annulus and the wing extends over the first leaflet toward the opposing leaflet.

In some implementations, the method includes anchoring the interface at the site.

In some implementations, the method includes, subsequently, releasing the distal end of the shaft from the interface by pulling on a ripcord, and/or subsequently, withdrawing the catheter and the shaft from the subject.

In some implementations, the method further includes sterilizing the implant.

In some implementations, the method further includes sterilizing the catheter and the shaft.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations, a method (which can be used to treat a valve of a heart, e.g., of a living subject or of a simulation, the valve can have an annulus, a first leaflet, and an opposing leaflet) includes advancing to the chamber, within a catheter: a shaft, and an implant. In some implementations, the implant includes an interface, engaged with a distal end of the shaft, and a flexible wing coupled to the interface.

In some implementations, the method includes using the shaft, deploying the implant out of the catheter and into the chamber.

In some implementations, the method includes positioning the implant in a position in which the interface is at a site on the annulus and the wing extends over the first leaflet toward the opposing leaflet.

In some implementations, the method includes anchoring the interface at the site.

In some implementations, the method includes subsequently, releasing the distal end of the shaft from the interface by pulling on a ripcord, and/or subsequently, withdrawing the catheter and the shaft from the subject.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations, a system and/or an apparatus (which can be used with tissue, e.g., of a living subject or of a simulation) includes a first anchor and a second anchor, an implant including a first interface and a second interface, and a delivery tool.

In some implementations, the delivery tool includes, among other components, a catheter, a first driver, a second driver and a shaft, extending distally through the catheter.

In some implementations, the catheter is transluminally advanceable to the tissue, and a distal part of the shaft bifurcates into a first branch and a second branch, each branch disposed alongside each other within the catheter.

In some implementations, the first and second branches can be engaged to a corresponding one of the first and second interfaces.

In some implementations, the first driver can extend distally through the shaft, and into the first branch where a drive head of the first driver is engaged with the first anchor and can be configured to anchor the first interface to the tissue by driving the anchor distally through the first interface and into the tissue.

In some implementations, the second driver can extend extending distally through the shaft alongside the first driver, and into the second branch where a drive head of the second driver is engaged with the second anchor and can be configured to anchor the second interface to the tissue by driving the anchor distally through the second interface and into the tissue.

In some implementations, the implant is sterile. In some implementations, the first anchor and the second anchor are sterile. In some implementations, the delivery tool is sterile.

In some implementations: the first branch has a first width, the second branch has a second width, and/or a portion of the shaft, proximal from the first and second branches, is narrower than the sum of the first and second widths.

In some implementations: the catheter defines a first lumen, and a second lumen alongside the first lumen, and/or each of the first and second drivers extend, from the proximal portion to the distal portion, within a respective one of the lumens.

In some implementations, the delivery tool further comprises a controller configured to operate the first driver and the second driver.

In some implementations, the controller is transitionable between: a first setting in which the controller operates the first and second driver simultaneously, and a second setting in which the controller operates only one of the first and second drivers at a given time.

In some implementations, any of the above implants can include a leg or extension that extends from the tip of the wing to an end portion of the leg. In some implementations, when the implant is implanted, the leg or extension extends from the wing of the implant such that, upon implantation, the leg or extension protrudes into the chamber downstream of the valve being treated.

In some implementations, the leg or extension is configured to bias the wing of the implant toward a particular position and/or orientation, and/or is configured to inhibit the wing from prolapsing into the atrium upstream of the valve being treated.

In some implementations, the leg is configured to maintain contact between the wing and leaflet as the leaflet oscillates throughout multiple cardiac cycles.

In accordance with some implementations, a method useable with a valve of a real or simulated heart (e.g., the valve can have a first leaflet and an opposing leaflet, and/or the heart can have a first chamber upstream of the valve and a second chamber downstream of the valve) includes, within a catheter, advancing to the first chamber: a shaft, and/or an implant that includes: an interface, engaged with a distal end of the shaft, and/or a flexible wing coupled to the interface.

In some implementations, the method can include using the shaft: deploying the implant out of the catheter and into the first chamber, and/or anchoring the implant.

In some implementations, the implant can be implanted in a position in which: the interface is at a site in the first chamber, the wing extends over the first leaflet toward the opposing leaflet, and responsively to a cardiac cycle of the heart, the wing deflects, in a reciprocating manner, in an upstream direction and in a downstream direction.

In some implementations, the method includes intracardially adjusting (e.g., subsequently to implantation) a deflection-range of the wing.

In some implementations, the method further includes sterilizing the implant, the shaft and the catheter.

In some implementations, the site is at an annulus of the valve, and/or anchoring the implant in the position includes anchoring the interface to the annulus of the valve.

In some implementations, the interface is coupled to a root portion of the wing, and/or anchoring the interface to the annulus includes anchoring the interface to the annulus such that the root portion is disposed at the annulus and the wing extends, from the root portion, over the first leaflet toward the opposing leaflet.

In some implementations, the implant further includes a limiter that defines a deflection-limit of the wing during the cardiac cycle of the heart by inhibiting deflection of the wing in the upstream direction beyond the deflection-limit, and/or adjusting the deflection-range of the wing includes intracardially adjusting the deflection-limit of the wing by adjusting the limiter.

In some implementations, anchoring the implant in the position includes driving an anchor into tissue at the site, and/or adjusting the limiter includes adjusting the limiter by applying torque to the anchor.

In some implementations, the limiter includes a tether, coupled to the wing, and/or adjusting the deflection-range of the wing includes adjusting the deflection-limit of the wing by intracardially adjusting tension on the tether.

In some implementations, the method further includes anchoring the tether to tissue of the second chamber prior to adjusting the tension.

In some implementations, a portion of the tether is wound around a rotatable spool, and/or adjusting tension on the tether includes, using an extracorporeal controller, adjusting tension on the tether via the catheter by rotating the spool.

In some implementations, intracardially adjusting tension on the tether includes intracardially sliding the tether with respect to the wing.

In some implementations, a first portion of the tether is coupled to the wing, and/or adjusting tension on the tether includes passing a second portion of the tether, in an upstream direction, through a root portion of the wing.

In some implementations, adjusting tension on the tether includes passing the second portion of the tether, in the upstream direction, through the interface.

In some implementations, anchoring the implant in the position includes anchoring the interface to tissue at the site by driving an anchor into the tissue, the anchor having an anchor head, and a tissue-engaging element that extends from the anchor head to define an anchor axis of the anchor, and/or adjusting the limiter includes deflecting the limiter with respect to the anchor axis.

In some implementations, deflecting the limiter includes changing a curvature of the limiter.

In some implementations, deflecting the limiter includes bringing the limiter into greater contact with the wing.

In some implementations, deflecting the limiter includes deflecting the limiter such that a portion of the limiter contacts the wing upon the wing reaching the deflection-limit.

In some implementations, deflecting the limiter includes deflecting the limiter such that the portion of the limiter does not contact the wing during ventricular diastole of the cardiac cycle.

In some implementations, anchoring the implant in the position includes driving an anchor into tissue at the site, and/or adjusting the limiter includes adjusting the limiter by driving the anchor deeper into the tissue at the site.

In some implementations, the anchor has an anchor head and a tissue-engaging element, the tissue-engaging element extending from the anchor head to define an anchor axis of the anchor, and/or adjusting the limiter includes deflecting the limiter with respect to the anchor axis.

In some implementations, the limiter defines a backstop portion, and/or adjusting the limiter includes pressing the backstop portion against tissue of the first chamber.

In some implementations, the backstop portion defines a spring, and/or pressing the backstop portion against the tissue of the first chamber includes tensioning the spring.

In some implementations, the backstop portion is an inflatable backstop portion, and/or pressing the backstop portion against the tissue of the first chamber includes pressing the backstop portion against the tissue by inflating the backstop portion.

In some implementations, the implant includes a tether, coupled to the wing, and/or intracardially adjusting the deflection-range of the wing includes, intracardially adjusting the deflection-range of the wing by adjusting tension on the tether.

In some implementations, the tether is coupled to a tip portion of the wing, and/or adjusting tension on the tether includes adjusting deflectability of the tip portion of the wing.

In some implementations, the tether is coupled to the wing, and/or the method further includes anchoring the tether to tissue of the second chamber.

In some implementations, the tether defines a rail portion that is slidably coupled to a proximal portion of the tether.

In some implementations, the step of anchoring includes anchoring a first part of the rail portion to trabeculae at a first site of the second chamber, and/or anchoring a second part of the rail portion to trabeculae at a second site of the second chamber.

In some implementations, a first portion of the tether is coupled to the wing, and/or adjusting tension on the tether includes passing a second portion of the tether, in an upstream direction, through a root portion of the wing.

In some implementations, adjusting tension on the tether includes passing the second portion of the tether, in the upstream direction, through the interface.

In some implementations, adjusting the deflection-range of the wing by adjusting tension on the tether includes pivoting the wing with respect to the interface by adjusting tension on the tether.

In some implementations, anchoring the implant in the position includes anchoring the interface to the site by driving, into tissue at the site, an anchor that defines: (i) an anchor head, and/or (ii) a tissue-engaging element extending from the anchor head along an anchor axis.

In some implementations, pivoting the wing with respect to the interface by adjusting tension on the tether includes pivoting the wing with respect to the anchor axis by adjusting tension on the tether.

In some implementations, the interface is an adjustable interface, and/or adjusting the deflection-range of the wing includes intracardially adjusting the deflection-range by adjusting the interface.

In some implementations, the adjustable interface defines a seat, anchoring the implant includes seating the seat against tissue at the site in the first chamber, and/or adjusting the interface includes adjusting an angle between a root portion of the wing and the seat of the interface.

In some implementations, the step of anchoring includes, using an anchor, anchoring the implant in the position.

In some implementations, the anchor defines an anchor head and a tissue-engaging element, the tissue-engaging element extending from the anchor head along an anchor axis.

In some implementations, adjusting the interface includes adjusting an angle between the root portion of the wing and the anchor axis.

In some implementations, the adjustable interface includes an adjustment mechanism.

In some implementations, adjusting the angle between the root portion of the wing and the seat of the interface includes adjusting the angle between the root portion of the wing and the seat of the interface by actuating the adjustment mechanism.

In some implementations, the adjustable interface includes a base to which the root portion of the wing is fixedly coupled.

In some implementations, adjusting the angle between the root portion of the wing and the seat of the interface includes adjusting the angle between the base and the seat of the interface, by actuating the adjustment mechanism.

In some implementations, the adjustment mechanism includes a lead screw, and/or actuating the adjustment mechanism includes rotating the lead screw.

In some implementations, the step of anchoring includes, using an anchor, anchoring the implant in the position.

In some implementations, the anchor defines an anchor head and a tissue-engaging element, the tissue-engaging element extending from the anchor head along an anchor axis.

In some implementations, screwing the lead screw includes screwing the lead screw along a lead screw axis that is offset with respect to the anchor axis.

In some implementations, the step of anchoring includes, using an anchor, anchoring the implant in the position.

In some implementations, the anchor defines an anchor head and a tissue-engaging element, the tissue-engaging element extending from the anchor head along an anchor axis.

In some implementations, screwing the lead screw includes screwing the lead screw along a lead screw axis that is colinear with the anchor axis.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations, a method useable with a valve of a real or simulated heart (e.g., the valve can have an annulus, a first leaflet, and an opposing leaflet, and the heart can have a chamber upstream of the valve) includes advancing an implant to the heart (e.g., to a chamber of the heart). In some implementations, the implant includes: a wing, extending from a root portion of the wing to a tip portion of the wing, an interface at the root portion of the wing, and/or a shape-memory member, coupled to the wing.

In some implementations, the method includes positioning the implant in a position in which: the interface is at a site in the chamber, and/or the wing extends, from the site, over the first leaflet toward the opposing leaflet.

In some implementations, the method includes anchoring the interface to the site.

In some implementations, the method includes, while the implant remains in the position, inducing the shape-memory member to chronically change a size of the wing by temporarily heating the shape-memory member.

In some implementations, the method further includes sterilizing the implant.

In some implementations, the step of inducing includes, while the implant remains in the position, inducing the shape-memory member to chronically change a width of the wing by temporarily heating the shape-memory member.

In some implementations, the step of inducing includes, while the implant remains in the position, inducing the shape-memory member to chronically change a length of the wing by temporarily heating the shape-memory member.

In some implementations, a first end of the shape-memory member is coupled to the root portion of the wing, and/or a second end of the shape-memory member is coupled to the tip portion of the wing.

In some implementations, the step of inducing includes, while the implant remains in the position, inducing the shape-memory member to chronically change a distance between the first end of the shape-memory member and the second end of the shape-memory member by temporarily heating the shape-memory member.

In some implementations, the step of heating includes temporarily heating the shape-memory member by applying electrical power to the shape-memory member.

In some implementations, applying the electrical power to the shape-memory member includes wirelessly applying the electrical power to the shape-memory member.

In some implementations, applying the electrical power to the shape-memory member includes applying the electrical power to the shape-memory member via a catheter.

In some implementations, the wing includes a locking mechanism that configures the wing to chronically remain at the changed size, and/or the method further includes temporarily unlocking the locking mechanism.

In some implementations, the shape-memory member is a first shape-memory member, the locking mechanism includes a second shape-memory member, and/or temporarily unlocking the locking mechanism includes temporarily heating the second shape-memory member.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations, a method usable or for use with a valve of a real or simulated heart (e.g., the valve can have an annulus, a first leaflet, and/or an opposing leaflet, and the heart can have a chamber upstream of the valve) includes advancing an implant to the chamber. In some implementations, the implant includes one or more of a wing, extending from a root portion of the wing to a tip portion of the wing, an interface at the root portion of the wing, and/or a shape-memory member, coupled to the wing.

In some implementations, the method includes positioning the implant in a position in which: the interface is at a site in the chamber, and/or the wing extends, from the site, over the first leaflet toward the opposing leaflet.

In some implementations, the method includes anchoring the interface to the site.

In some implementations, the method includes, while the implant remains in the position, inducing the shape-memory member to chronically change a size of the wing by temporarily heating the shape-memory member.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations, a system and/or an apparatus useable with a valve of a real or simulated heart (e.g., the valve can have an annulus, a first leaflet, and/or an opposing leaflet, and the heart can have a first chamber upstream of the valve and a second chamber downstream of the valve). In some implementations, the system/apparatus can include an implant, the implant including one or more of: a flexible wing, the wing: extending from a root portion of the wing to a tip portion of the wing; and/or a limb/extension coupled to the wing.

In some implementations, the root portion of the wing is configured to be placed against a site on the annulus, adjacent a root of the first leaflet, in a manner that supports the wing extending, from the root portion of the wing, over the first leaflet toward the opposing leaflet.

In some implementations, the limb or extension is shaped such that, when the root portion of the wing is placed against the site, the limb/extension extends away from the wing to contact tissue of the heart adjacent a root of the opposing leaflet, in a manner that moderates deflection of the wing with respect to the site in an upstream direction.

In some implementations, the implant is sterile.

In some implementations, the implant is configured such that when the root portion of the wing is placed against the site, and the limb/extension extends away from the wing to contact tissue of the heart, the tip portion of the wing deflects with respect to the root portion of the wing, reciprocatingly in the upstream direction and in a downstream direction, responsively to a cardiac cycle of the heart.

In some implementations, the limb/extension is shaped such that, when the root portion of the wing is placed against the site, the limb/extension extends away from the root portion of the wing to contact tissue of the heart in a manner that moderates deflection of the wing with respect to the site in the upstream direction.

In some implementations, the limb/extension is shaped such that, when the root portion of the wing is placed against the site, the limb/extension extends away from the tip portion of the wing to contact tissue of the heart in a manner that moderates deflection of the wing with respect to the site in the upstream direction.

In some implementations, the limb/extension is shaped such that, when the root portion of the wing is placed against the site, the limb/extension extends away from the wing to contact tissue of the annulus in a manner that moderates deflection of the wing with respect to the site in the upstream direction.

In some implementations, the limb/extension defines an arm that is shaped such that, when the root portion of the wing is placed against the site, the arm is disposed against an atrial surface of the annulus in a manner that moderates deflection of the wing with respect to the site in the upstream direction.

In some implementations, the implant further includes an interface at the root portion of the wing, the interface configured to be secured to the site on the annulus by driving an anchor into tissue at the site.

In some implementations, the arm includes an anchor receiver, the anchor receiver configured to be secured to the atrial surface of the annulus, when the root portion of the wing is placed against the site, by driving an anchor through the anchor receiver and into tissue at the atrial surface of the annulus.

In some implementations, the arm is shaped such that, when the root portion of the wing is placed against the site, the anchor receiver is disposed adjacent a commissure of the valve.

In some implementations, the arm is shaped such that, when the root portion of the wing is placed against the site, the anchor receiver is disposed adjacent to a root portion of the first leaflet.

In some implementations, the arm is shaped such that, when the root portion of the wing is placed against the site, the anchor receiver is disposed adjacent a root portion of the opposing leaflet.

In some implementations, the limb/extension defines a leg that is shaped such that, when the root portion of the wing is placed against the site, the leg contacts tissue of the ventricle in a manner that moderates deflection of the wing with respect to the site in the upstream direction.

In some implementations, the implant further includes an interface at the root portion of the wing, the interface configured to be secured to the site on the annulus by driving an anchor into tissue at the site.

In some implementations, the leg that is shaped such that, when the root portion of the wing is placed against the site, the leg contacts tissue of an underside of the valve in a manner that moderates deflection of the wing with respect to the site in the upstream direction.

In some implementations, the leg is shaped such that, when the root portion of the wing is placed against the site, the leg is disposed adjacent a commissure of the valve.

In some implementations, the leg is shaped such that, when the root portion of the wing is placed against the site, the leg is disposed in a subannular groove of the valve.

In some implementations, the implant further includes an atrial support, the atrial support coupled to the wing and configured such that, when the root portion of the wing is placed against the site, the atrial support presses against an atrial surface of the annulus in a manner that presses the leg against the tissue of the ventricle.

In some implementations, the atrial support is shaped to circumscribe the atrial surface of the annulus.

In some implementations, the atrial support is defined by a pair of arms that extend, from the root portion, in opposite directions around the atrial surface of the annulus.

In some implementations, the ventricle is a left ventricle, the valve is a mitral valve, the first leaflet is a posterior leaflet of the mitral valve, and the opposing leaflet is an anterior leaflet of the mitral valve.

In some implementations, the leg is shaped such that, when the root portion of the wing is placed against the site, the leg contacts tissue of the left ventricle behind the anterior leaflet.

In some implementations, the leg is shaped such that, when the root portion of the wing is placed against the site, the leg contacts a fibrous trigone of the left ventricle.

In some implementations, the wing has a compressed state. In some implementations, the wing is biased to expand into an expanded state. In some implementations, the wing can be actuated to expand to an expanded state.

In some implementations, the limb/extension includes an annular support. In some implementations, the annular support is coupled to the root portion of the wing. In some implementations, the annular support is configured such that: in the compressed state of the wing, the wing has a hinged coupling to the annular support that facilitates articulation, at the hinged coupling, of the wing with respect to the annular support. In some implementations, expansion of the wing toward the expanded state inhibits the articulation by restraining the hinged coupling.

In some implementations: the implant further includes an interface at the root portion of the wing.

In some implementations, the wing defines a contact face, and an opposing face opposite to the contact face.

In some implementations, the system further includes: an anchor, and/or a delivery tool. In some implementations, the delivery tool includes a catheter, transluminally advanceable to the atrium with the implant housed in the catheter while the wing is in the compressed state.

In some implementations, the delivery tool includes a driver, configured to: deploy the implant out of the catheter such that, within the first chamber, the wing assumes the expanded state. In some implementations, the driver is configured to position the implant in a position in which: the wing extends over the first leaflet toward the opposing leaflet, and/or the contact face faces the first leaflet.

In some implementations, while the implant is positioned in the position and the wing is in the expanded state, secure the interface to the annulus by driving the anchor through the interface and into tissue of the annulus.

In some implementations, the annular support is shaped such that, while the implant is secured to the annulus and the wing is in the expanded state, the annular support is disposed against an atrial surface of the annulus such that, the restrained hinged coupling inhibits deflection of the root portion of the wing with respect to the annulus.

In some implementations: the interface is a first interface; the annular support includes a first annular arm that extends away from the hinged coupling to the first interface; and/or the annular support further includes a second annular arm that: is coupled to the hinged coupling, and/or extends away from the hinged coupling to a second interface.

In some implementations, the first annular arm is joined to the second annular arm, at the hinged coupling.

In some implementations, the implant is configured such that the hinged coupling includes a sleeve defining an aperture.

In some implementations, the implant is configured such that while the wing is in the compressed state, a thin portion of the annular support is disposed within the aperture.

In some implementations, the implant is configured such that expansion of the wing toward the expanded state slides the sleeve from the thin portion to a thick portion of the annular support. In some implementations, the thick portion has a cross-section that is dimensioned to restrain the hinged coupling by fitting the aperture.

In some implementations, the thick portion of the annular support has an oblong cross-section that is dimensioned to restrain the hinged coupling by fitting the aperture.

In some implementations, the sleeve is a first sleeve defining a first aperture. In some implementations, the hinged coupling further includes a second sleeve defining a second aperture.

In some implementations, the annular support includes a pair of annular arms. In some implementations, each annular arm has: a thin portion at which the annular arms are joined, and/or a thick portion that extends away from the thin portion.

In some implementations, expansion of the wing toward the expanded state slides each sleeve from the thin portion to the thick portion of a respective annular arm, thereby restraining the hinged coupling.

In accordance with some implementations, a system and/or an apparatus useable with a valve of a real or simulated heart (e.g., the valve can have a first leaflet and an opposing leaflet, and the heart can have a chamber upstream of the valve) includes an implant, wherein the implant including a wing, extending from a root portion of the wing to a tip portion of the wing. In some implementations, the implant can include an interface at the root portion of the wing.

In some implementations, the interface is configured to be anchored to a site in the first chamber such that the implant is secured in a position in which: the wing extends over the first leaflet toward the opposing leaflet, and/or responsively to a cardiac cycle of the heart, the wing deflects, in a reciprocating manner, in an upstream direction and in a downstream direction.

In some implementations, the system/apparatus includes an adjustment member that is adjustable in a manner that adjusts a deflection-range of the wing.

In some implementations, the implant is sterile.

In some implementations, the adjustment member is an adjustment mechanism that is actuatable by application of torque.

In some implementation, the adjustment member includes an adjustable limiter that defines a deflection-limit of the wing during the cardiac cycle of the heart by inhibiting deflection of the wing in the upstream direction beyond the deflection-limit, and/or the limiter is adjustable in a manner that intracardially adjusts the deflection-limit of the wing.

In some implementations, the system/apparatus further includes a catheter, and/or an extracorporeal controller, the controller configured to adjust the limiter via the catheter.

In some implementations, the limiter defines a backstop portion, and/or is adjustable by pressing the backstop portion against tissue of the first chamber.

In some implementations, the backstop portion defines a spring, and/or the limiter can be configured such that tensioning the spring presses the backstop portion against the tissue of the first chamber.

In some implementations, the backstop portion is an inflatable backstop portion, and/or the limiter can be configured such that inflating the backstop portion presses the backstop portion against the tissue of the first chamber.

In some implementations, the limiter is configured such that such that a portion of the limiter contacts the wing upon the wing reaching the deflection-limit.

In some implementations, the limiter is configured such that the portion of the limiter does not contact the wing during ventricular diastole of the cardiac cycle.

In some implementations, the system/apparatus further includes: an anchor and/or a driver, engaged with the anchor.

In some implementations, the system/apparatus is configured to secure the implant in the position by using the anchor to anchor the interface to the site by driving the anchor into tissue at the site, and/or adjust the limiter by driving the anchor deeper into the tissue at the site.

In some implementations, the driver is configured to secure the implant in the position, and to anchor the interface to the site, by driving the anchor into tissue at the site.

In some implementations, the anchor includes an anchor head, and a tissue-engaging element that extends from the anchor head to define an anchor axis of the anchor, and/or the limiter can be configured such that driving the anchor deeper into the tissue at the site adjusts the limiter by deflecting the limiter with respect to the anchor axis.

In some implementations, the driver is configured to drive the anchor deeper into the tissue at the site by applying torque to the anchor.

In some implementations, the limiter is configured such that driving the anchor deeper into the tissue at the site adjusts the limiter by bringing the limiter into greater contact with the wing.

In some implementations, the limiter is configured such that driving the anchor deeper into the tissue at the site adjusts the limiter by changing a curvature of the limiter.

In some implementations, the limiter includes a tether, the tether coupled to the wing, and/or the limiter is configured such that intracardially adjusting tension on the tether adjusts the deflection-limit of the wing.

In some implementations, the limiter is configured such that intracardially adjusting tension on the tether, by intracardially sliding the tether with respect to the wing, adjusts the deflection-limit of the wing.

In some implementations, a portion of the tether is wound around a rotatable spool, and/or the limiter is configured such that intracardially adjusting tension on the tether, by rotating the spool, adjusts the deflection-limit of the wing.

In some implementations, a first portion of the tether is coupled to the wing, and/or the tether is configured such that passing a second portion of the tether, through the root portion of the wing in the upstream direction, adjusts tension on the tether.

In some implementations, the tether is configured such that passing the second portion of the tether, through the interface in the upstream direction, adjusts tension on the tether.

In some implementations, the adjustment member includes a tether, and/or the implant is configured such that adjusting tension on the tether adjusts the deflection-range of the wing.

In some implementations, the system/apparatus further includes: a catheter, and/or an extracorporeal controller, the controller configured to adjust the tension on the tether via the catheter.

In some implementations, the tether is coupled to the tip portion of the wing, and/or the implant is configured such that adjusting tension on the tether adjusts deflectability of the tip portion of the wing.

In some implementations, a first portion of the tether is coupled to the wing, and/or the implant is configured such that passing a second portion of the tether through the root portion of the wing adjusts the deflection-range of the wing.

In some implementations, the implant is configured such that passing a second portion of the tether through the interface adjusts the deflection-range of the wing.

In some implementations, the implant is configured such that adjusting tension on the tether adjusts the deflection-range of the wing by pivoting the wing with respect to the interface.

In some implementations, the system/apparatus further includes an anchor, the anchor having an anchor head and a tissue-engaging element that extends from the anchor head to define an anchor axis.

In some implementations, the implant is configured such that adjusting tension on the tether adjusts the deflection-range of the wing by pivoting the wing with respect to the anchor axis.

In some implementations, the adjustment member is defined by the interface, which is an adjustable interface including an adjustment mechanism that is adjustable in a manner that adjusts the deflection-range of the wing.

In some implementations, the system/apparatus further includes: a catheter, and/or an extracorporeal controller, the controller configured to adjust the adjustment mechanism via the catheter.

In some implementations, the adjustment mechanism: (i) defines a seat, configured to be seated against tissue at the site, and/or (ii) can be configured to adjust the deflection-range of the wing by adjusting an angle between the root portion of the wing and the seat.

In some implementations, the system/apparatus further includes an anchor having an anchor head and a tissue-engaging element that extends from the anchor head to define an anchor axis.

In some implementations, the adjustment mechanism is configured to adjust the deflection-range of the wing by adjusting an angle between the root portion of the wing and the anchor axis.

In some implementations, the system/apparatus further includes: a catheter, and/or an extracorporeal controller, the controller configured to adjust the adjustment mechanism via the catheter.

In some implementations, the adjustment mechanism: (i) includes a base to which the root portion of the wing is fixedly coupled, and/or (ii) can be configured to adjust the deflection-range of wing by adjusting the angle between the base and the seat.

In some implementations, the adjustment mechanism: (i) includes a lead screw, and/or (ii) can be configured such that rotation of the lead screw adjusts the angle between the base and the seat.

In some implementations, the system/apparatus further includes an anchor, having an anchor head and a tissue-engaging element that extends from the anchor head to define an anchor axis of the anchor, wherein the lead screw defines a lead screw axis that is offset with respect to the anchor axis.

In some implementations, the system/apparatus further includes an anchor, having an anchor head and a tissue-engaging element that extends from the anchor head to define an anchor axis of the anchor, wherein the lead screw defines a lead screw axis that is colinear with the anchor axis.

In accordance with some implementations, a system and/or an apparatus usable with a valve of a real or simulated heart (e.g., the valve can have an annulus, a first leaflet, and/or an opposing leaflet, and the heart can have a chamber upstream of the valve) includes an implant. In some implementations, the implant can include a flexible wing extending from a root portion of the wing to a tip portion of the wing, and a pair of arms that are coupled to the wing and that are divergently away from the wing.

In some implementations, each of the arms has an anchor point configured to be anchored to the annulus such that the arm arcs from the anchor point along the annulus to the wing.

In some implementations, while the wing is secured in a position in which the root portion is disposed against the annulus, the wing extends, from the root portion, over the first leaflet toward the opposing leaflet.

In some implementations, responsively to a cardiac cycle of the heart, the wing deflects, in a reciprocating manner, in an upstream direction and in a downstream direction.

In some implementations, the implant is sterile.

In some implementations, each arm of the pair of arms arcs away from each other, along a face of the wing.

In some implementations, each arm of the pair of arms arcs away from each other, and away from a face of the wing.

In some implementations, each arm is: coupled to the root portion of the wing, and/or arcs divergently away from the root portion of the wing.

In some implementations, the system/apparatus further includes a pair of anchors, each of the anchors having an anchor head and a tissue-engaging element that extends from the anchor head to define an anchor axis of the anchor that is generally perpendicular to a portion of the arm.

In some implementations, each arm is articulatably coupled by a hinge to the wing such that, while the wing is secured in the position by the anchor points being anchored to the annulus, the arms articulate with respect to the wing in response to reciprocating deflection of the wing.

In some implementations, each arm is articulatably coupled to the wing such that, while the wing is secured in the position by the anchor points being anchored to the annulus, an angle defined by the pair of arms becomes more acute as the wing deflects in the upstream direction.

In some implementations, the implant further includes an interface at the root portion of the wing, the interface configured to secure the root portion to the annulus by the interface being anchored to the annulus.

In some implementations, the system/apparatus further includes a plurality of anchors, each anchor defining an anchor head and a tissue-engaging portion extending from the anchor head along an anchor axis.

In some implementations, the plurality of anchors includes: (i) a root anchor configured to anchor the interface to the annulus by being driven into tissue of the annulus along a root anchor axis, and/or (ii) a pair of arm anchors, each arm anchor configured to anchor, to the annulus, the anchor point of a respective one of the arms by being driven into tissue of the annulus along an arm anchor axis.

In some implementations, the wing is secured in the position by the plurality of anchors, upstream deflection of the wing is closer to being parallel to the root anchor axis than to either of the arm anchor axes.

In some implementations, while the wing is secured in the position by the plurality of anchors, upstream deflection of the wing is in direction that is generally parallel to the root anchor axis.

In some implementations, the implant further includes a limiter.

In some implementations, the limiter: coupled to the wing, and/or configured to inhibit deflection of the wing.

In some implementations, the limiter is configured to define a deflection-limit of the wing during the cardiac cycle of the heart by inhibiting deflection of the wing in the upstream direction beyond the deflection-limit.

In some implementations, the limiter defines a backstop portion that is shaped to press against tissue of the chamber upon anchoring of the anchor receivers to the annulus.

In accordance with some implementations, a system and/or an apparatus useable with a valve of a heart of a living subject or simulation subject (e.g., the valve can have an annulus, a first leaflet, and/or an opposing leaflet opposing the first leaflet, and the heart can have a first chamber upstream of the valve and a second chamber downstream of the valve) includes an implant. In some implementations, the implant can include a flexible wing, extending from a root portion of the wing to a tip portion of the wing; and/or a leg, extending from the wing to an end portion of the leg.

In some implementations, implant is configured to be secured in a position in which the root portion is disposed against a site at an atrial surface of the annulus, the wing extends, from the root portion, over the first leaflet toward the opposing leaflet, and/or the leg extends away from the wing to press against an underside of the valve.

In some implementations, the implant is sterile.

In some implementations, the implant further includes an interface at the root portion of the wing.

In some implementations, the system/apparatus further includes an anchor, and/or a delivery tool.

In some implementations, the delivery tool includes a catheter, transluminally advanceable to the first chamber, and configured to house the implant.

In some implementations, the delivery tool includes a shaft, housing the anchor, engaged with the interface.

In some implementations, the shaft is configured, via the engagement with the interface, to, while the anchor remains within the shaft: (i) deploy the implant out of the catheter such that, within the first chamber, the wing extends away from the interface, and/or (ii) position the implant in a position in which: (a) the interface is at a site of the annulus, (b) the wing extends over the first leaflet toward the opposing leaflet, and/or (c) the leg extends, from the tip portion, away from the wing and toward a tissue of the second chamber.

In some implementations, the delivery tool includes a driver, engaged with the anchor, and configured to secure the implant in the position by using the anchor to anchor the interface to the annulus.

In some implementations, the implant is configured such that when the root portion of the wing is disposed against the site, and the leg extends away from the wing to press against the underside of the valve, the tip portion of the wing deflects with respect to the root portion of the wing, reciprocatingly in the upstream direction and in the downstream direction, responsively to a cardiac cycle of the heart.

In some implementations, the leg is shaped such that, when the root portion of the wing is disposed against the site, the leg extends away from the root portion of the wing to press against the underside of the valve in a manner that moderates deflection of the wing with respect to the site in the upstream direction.

In some implementations, the leg is shaped such that, when the root portion of the wing is disposed against the site, the leg extends away from the tip portion of the wing to contact tissue of the heart in a manner that moderates deflection of the wing with respect to the site in the upstream direction.

In some implementations, the leg is shaped such that, when the root portion of the wing is disposed against the site, the leg presses against the underside of the valve in a manner that moderates deflection of the wing with respect to the site in the upstream direction.

In some implementations, the implant further includes an interface at the root portion of the wing, the interface configured to be secured to the site by driving an anchor into tissue at the site.

In some implementations, the leg is shaped such that, when the root portion of the wing is placed against the site, the leg presses against tissue adjacent a commissure of the valve.

In some implementations, the leg is shaped such that, when the root portion of the wing is placed against the site, the leg presses against tissue at a subannular groove of the valve.

In some implementations, the implant further includes an atrial support, the atrial support coupled to the wing and configured such that, when the root portion of the wing is placed against the site, the atrial support presses against the atrial surface of the annulus in a manner that presses the leg against tissue of the second chamber.

In some implementations, the atrial support is shaped to circumscribe the atrial surface of the annulus.

In some implementations, the atrial support is defined by a pair of arms that extend, from the root portion, in opposite directions around the atrial surface of the annulus.

In some implementations, the second chamber is a left ventricle, the valve is a mitral valve, the first leaflet is a posterior leaflet of the mitral valve, and/or the opposing leaflet is an anterior leaflet of the mitral valve.

In some implementations, the leg is shaped such that, when the root portion of the wing is placed against the site, the leg presses against tissue of the left ventricle behind the anterior leaflet.

In some implementations, the leg is shaped such that, when the root portion of the wing is placed against the site, the leg presses against a fibrous trigone of the left ventricle.

In accordance with some implementations, a system and/or an apparatus (e.g., usable or for use with a valve of a real or simulated heart, the valve having a first leaflet and an opposing leaflet, the heart having a chamber upstream of the valve) can include an implant that can include a flexible wing extending from a root portion of the wing to a tip portion of the wing; and/or a limiter, coupled to the wing.

In some implementations, the implant is configured to be anchored to a site in the chamber. In some implementations, the implant is secured in a position in which the wing extends over the first leaflet toward the opposing leaflet and/or responsively to a cardiac cycle of the heart, the wing deflects, in a reciprocating manner, in an upstream direction and in a downstream direction.

In some implementations, the limiter inhibits deflection of the wing in the upstream direction beyond the deflection-limit by providing an opposing force upon the wing reaching the deflection-limit.

In some implementations, the implant is sterile.

In some implementations, a frame of the wing is laser-cut from a piece of sheet-metal.

In some implementations, the frame is shaped to define a buttress at the root portion of the wing, such that the root portion of the wing is stiffer than the tip portion of the wing.

In some implementations, the implant further includes a pair of arced arms, each arm coupled to the wing at a first portion of the arm, and shaped such that, when the implant is secured in the position, a second portion of the arm contacts tissue of the chamber.

In some implementations, the pair of arms are symmetrically away from the wing.

In some implementations, the pair of arms are asymmetrically away from the wing.

In some implementations, each arm is coupled to the wing in a manner that allows the arm to pivot with respect to the wing.

In some implementations, the pair of arms are asymmetrically away from the wing such that, when the arms pivot toward each other, the arms become nested with respect to each other.

In some implementations, each arm defines an anchor receiver configured to be anchored to the site by advancing an anchor through the anchor receiver and into tissue of the chamber.

In some implementations, each arm is shaped such that, when the implant is secured in the position, each anchor receiver is disposed adjacent a respective commissure of the valve.

In some implementations, when the implant is configured such that, when the implant is secured in the position by advancing an anchor through the anchor receiver and into tissue of the chamber, the root portion of the wing maintains contact with the site as the wing deflects in response to the cardiac cycle.

In some implementations, the implant is configured such that, when the implant is secured in the position by advancing an anchor through the anchor receiver and into tissue of the chamber, an angle defined by the arms becomes more acute as the wing deflects in the upstream direction.

In some implementations, the limiter defines a backstop portion that is shaped to press against tissue of the chamber upon the wing reaching the deflection-limit.

In some implementations, the backstop is shaped to define an anchor receiver, and the implant is configured to be secured to the site by advancing an anchor through the anchor receiver and into tissue at the site.

In some implementations, the limiter further defines a plurality of ribs that extend from the backstop portion and along the wing, from the root portion of the wing toward the tip portion of the wing.

In some implementations, while the implant is secured in the position, the limiter inhibits deflection of the wing in the upstream direction beyond the deflection-limit by the ribs providing the opposing force upon the wing reaching the deflection-limit.

In some implementations, the limiter defines a pair of arms, each arm arcing away from the backstop portion.

In some implementations, each arm defines an anchor receiver configured to be anchored to the site by advancing an anchor through the anchor receiver and into tissue of the chamber.

In some implementations, each arm is shaped such that, when the implant is secured in the position, each anchor receiver is disposed adjacent a respective commissure of the valve.

In accordance with some implementations, a system useable with a valve of a real or simulated heart (e.g., the valve can have a first leaflet and an opposing leaflet, and the heart can have a chamber upstream of the valve) includes an anchor, and an implant including a wing extending from a root portion of the wing to a tip portion of the wing, and an anchor receiver at the root portion of the wing.

In some implementations, the implant is configured to be anchored to a site in the chamber by the anchor extending through the anchor receiver and into tissue at the site.

In some implementations, the system includes a delivery tool including a catheter, transluminally advanceable to the chamber, and a shaft disposed within the catheter. The shaft can be engaged with the implant, and configured, via the engagement with the implant, to deploy the implant out of the catheter.

In some implementations, the shaft is configured, via the engagement with the implant, to position the implant in a position in which the anchor receiver is at the site, and the wing extends over the first leaflet toward the opposing leaflet.

In some implementations, an adjustment rod is reversibly coupled to the wing such that, while the anchor extends through the anchor receiver and into the tissue at the site, axial movement of the adjustment rod adjusts a position of the wing by sliding the anchor receiver with respect to the tissue and the anchor.

In some implementations, the anchor, the implant and the delivery tool are sterile.

In accordance with some implementations, a system useable with a valve of a real or simulated heart (e.g., the valve can have a first leaflet and an opposing leaflet, and the heart can have a chamber upstream of the valve) includes an anchor having an anchor head having a diameter, and a tissue-engaging element that extends from the anchor head.

In some implementations, the system includes an implant including an anchor receiver defining an oblong opening delimited by a rim. In some implementations, the implant can be configured to be anchored to a site in the heart by the anchor head being seated against the rim of the opening while the tissue-engaging element extends through the opening and into tissue at the site.

In some implementations, the opening has a first dimension that is smaller than the diameter, and a second dimension, transverse to the first dimension, that is greater than the diameter.

In some implementations, the anchor and the implant are sterile.

In some implementations, the system includes a delivery tool including a catheter, transluminally advanceable to the chamber.

In some implementations, the system includes a delivery tool including a shaft. In some implementations, the shaft can be engaged with the implant. In some implementations, the shaft can be disposed within a separate catheter.

In some implementations, the shaft is configured, via the engagement with the implant, to: (i) deploy the implant out of the catheter, and/or (ii) position the implant in a position in which the implant is at the site, and the implant extends over the first leaflet toward the opposing leaflet.

In some implementations, an adjustment rod is reversibly coupled to the implant such that, while the tissue-engaging element extends through the opening and into tissue at the site, axial movement of the adjustment rod adjusts a position of the implant by sliding the implant with respect to the tissue and the anchor.

In some implementations, the second dimension is oriented along a length of the implant.

In some implementations, the implant is configured such that when the tissue-engaging element extends through the opening, to a first depth of tissue at the site, the implant is slidable along the second dimension, relative to the tissue and the anchor.

In some implementations, when the tissue-engaging element extends through the opening, to a second, greater depth of tissue at the site: the anchor head is seated against the rim, and/or the implant ceases to be slidable with respect to the anchor.

In accordance with some implementations, a system (e.g., usable or for use with a valve of a real or simulated heart) can include an anchor, having an anchor head, and a tissue-engaging element that extends from the anchor head.

In some implementations, the system includes an implant including an implant body, an interface having a diameter, and an anchor receiver defining an oblong opening delimited by a rim.

In some implementations, the opening has a first dimension that is smaller than the diameter, and a second dimension, transverse to the first dimension, that is greater than the diameter. In some implementations, the anchor receiver is configured to be anchored to a site in the heart by the tissue-engaging element of the anchor extending through the interface and the anchor receiver, into tissue at the site.

In some implementations, the anchor and the implant are sterile.

In some implementations, the implant is configured to be anchored to the site by the anchor head seating the interface against the rim of the opening while the tissue-engaging element extends through the interface and the opening, and into tissue at the site.

In some implementations, the system includes a delivery tool including a catheter, transluminally advanceable to the heart.

In some implementations, the system includes a delivery tool that includes a shaft, the shaft engaged with the interface. In some implementations, the shaft is configured, via the engagement with the interface, to: (i) deploy the implant out of the catheter, and/or (ii) position the implant in a position in which the anchor receiver is at the site. In some implementations, the shaft can be disposed within a catheter.

In some implementations, an adjustment rod is reversibly coupled to the implant such that, while the tissue-engaging element extends through the interface and into the tissue at the site, axial movement of the adjustment rod adjusts the position of the implant by sliding the implant body and the anchor receiver with respect to the tissue and the anchor.

In some implementations, the adjustment rod is reversibly coupled to the implant such that, while the tissue-engaging element extends through the interface and into the tissue at the site, axial movement of the adjustment rod adjusts the position of the implant by sliding the implant body and the anchor receiver with respect to the interface, the tissue and the anchor.

In some implementations, the interface includes: (i) a collar having the diameter, and/or (ii) a neck that is narrower than the collar.

In some implementations, the implant is configured to be anchored to the site by the anchor head seating the collar against the rim of the opening while the anchor receiver circumscribes the neck of the interface.

In some implementations, the collar is a first collar, the interface further includes a second collar, and/or the implant is configured to be anchored to the site by sandwiching the anchor receiver between the first collar and the second collar.

In accordance with some implementations, a method (e.g., usable or for use with a valve of a real or simulated heart) can include advancing to the heart: an anchor, the anchor including an anchor head and a tissue-engaging element that extends from the anchor head; and an implant, the implant including an anchor receiver.

In some implementations, the anchor receiver defines an oblong opening delimited by a rim, the oblong opening having a major axis.

In some implementations, the method includes advancing the tissue-engaging element of the anchor through the anchor receiver and into tissue at a site of the heart to a first tissue-depth, and while the tissue-engaging element remains within the tissue, sliding the implant along the major axis with respect to the anchor. The method can include subsequently, locking the implant to the anchor such that the implant ceases to be slidable with respect to the anchor.

In some implementations, the method further includes sterilizing the anchor and the implant.

In some implementations, the step of locking includes: (i) advancing the tissue-engaging element of the anchor further through the anchor receiver and into tissue at the site, to a second tissue-depth, and/or (ii) seating the anchor head against the rim.

In accordance with some implementations, a method usable with a valve of a real or simulated heart can include advancing to the heart an anchor, the anchor including an anchor head and a tissue-engaging element that extends from the anchor head. In some implementations, the method can include advancing to the heart an implant which can include an implant body, an interface having a diameter, and an anchor receiver defining an oblong opening delimited by a rim. In some implementations, the oblong opening has a major axis.

In some implementations, the method includes anchoring the implant to a site in the heart by advancing the tissue-engaging element of the anchor, through the interface and the anchor receiver, and into tissue at the site to a first tissue-depth.

In some implementations, the method can include subsequently sliding the implant body, with respect to the interface, along the major axis, and/or locking implant body to the interface by advancing the tissue-engaging element of the anchor, further through the interface and the anchor receiver, and into tissue at the site, to a second tissue-depth, and/or using the anchor head, seating the interface against the rim.

In some implementations, the method further includes sterilizing the anchor and the implant.

In some implementations, the interface includes a first collar and a second collar, and/or the step of seating the interface includes, using the anchor head, sandwiching the anchor receiver between the first collar and the second collar.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations, a method useable with a valve of a real or simulated heart can include advancing to the heart an anchor, the anchor including an anchor head and a tissue-engaging element that extends from the anchor head.

In some implementations, the method can include advancing to the heart an implant, the implant including an implant body, an interface having a diameter, and/or an anchor receiver defining an oblong opening delimited by a rim, the oblong opening having a major axis.

In some implementations, the method includes anchoring the implant to a site in the heart by advancing the tissue-engaging element of the anchor, through the interface and the anchor receiver, and into tissue at the site to a first tissue-depth.

In some implementations, the method can include subsequently sliding the implant body, with respect to the interface, along the major axis, and/or locking implant body to the interface by advancing the tissue-engaging element of the anchor, further through the interface and the anchor receiver, and into tissue at the site, to a second tissue-depth, and/or using the anchor head, seating the interface against the rim.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations, a system usable with a valve of a real or simulated heart, e.g., the valve can have a first leaflet and an opposing leaflet, and the heart can have a chamber upstream of the valve. In some implementations, the system can include an implant, the implant including a wing extending from a root portion of the wing to a tip portion of the wing, and/or an interface at the root portion of the wing. In some implementations, the interface can be configured to be anchored to a site in the chamber.

In some implementations, a bulking element can be coupled (e.g., fixedly coupled, connected, etc.) to a portion of the wing (e.g., the tip portion of the wing, a mid-portion of the wing, a proximal portion of the wing, a distal portion of the wing, etc.).

In some implementations, the system includes a delivery tool that can include a catheter, transluminally advanceable to the chamber, and a shaft disposed within the catheter.

In some implementations, the shaft is engaged with the interface and configured, via the engagement with the interface, to deploy the implant out of the catheter, and/or position the implant in a position in which the interface is at the site, the wing extends over the first leaflet, and the tip portion is disposed between the first leaflet and the opposing leaflet.

In some implementations, the system includes an actuator, operatively coupled to the bulking element such that actuation of the actuator can change a bulkiness of the tip portion.

In some implementations, the implant and the delivery tool are sterile.

In some implementations, the actuator is extracorporeally controllable to transition the bulking element from a delivery state to an actuated state.

In some implementations, the bulking element includes a braided structure, the braided structure having a delivery state and an actuated state, and/or by transitioning from the delivery state to the actuated state, the braided structure becomes shorter and wider.

In accordance with some implementations, a method (e.g., usable or for use with a valve of a real or simulated heart, the valve having a first leaflet and an opposing leaflet, the heart having a first chamber upstream of the valve and a second chamber downstream of the valve) can include within a catheter, advancing to the first chamber: a shaft, and/or an implant that includes an interface, engaged with a distal end of the shaft, and/or a flexible wing coupled to the interface.

In some implementations, the wing extends from a root portion of the wing to a tip portion of the wing, and a bulking element is coupled (e.g., fixedly coupled, connected, etc.) to the wing (e.g., to a tip portion of the wing, to a mid-region of the wing, to an end of the wing, to a distal portion of the wing, to a proximal portion of the wing, etc.).

In some implementations, the shaft can be used to deploy the implant out of the catheter and into the first chamber, and/or to anchor the implant in a position in which the interface is at a site in the first chamber, and the wing extends over the first leaflet toward the opposing leaflet. Responsively to a cardiac cycle of the heart, the wing deflects, in a reciprocating manner, in an upstream direction and in a downstream direction.

In some implementations, an actuator can be used to actuate the bulking element in a manner that changes a bulkiness of the implant (e.g., a tip portion, a mid-portion, an end portion, a distal portion, a proximal portion, etc.).

In some implementations, the method further includes sterilizing the catheter, the shaft and the implant.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations, a system (e.g., useable with or for use with a valve of a real or simulated heart) can include an implant that can be configured to be transluminally implanted in the heart. In some implementations, the implant can include a wing extending from a root portion of the wing to a tip portion of the wing, and/or a shape-memory member, coupled to the wing.

In some implementations, the shape-memory member is configured to be intracardially heated to a temperature greater than 40 degrees C.

In some implementations, temporary heating of the shape-memory member to the temperature resizes the wing to a size, and the wing is configured to retain the size after cessation of the temporary heating.

In some implementations, the implant is sterile.

In some implementations, the wing is configured such that temporary heating of the shape-memory member to the temperature resizes the wing by changing a shape of the shape-memory member.

In some implementations, the implant includes a power source that is configured to heat the shape-memory member.

In some implementations, the implant further includes an antenna that is configured to wirelessly receive power that heats the shape-memory member.

In accordance with some implementations, a system useable with a valve of a real or simulated heart (e.g., the valve can have a first leaflet and an opposing leaflet, and the heart can have a chamber upstream of the valve) can include an implant, the implant including a wing extending from a root portion of the wing to a tip portion of the wing, an interface at the root portion of the wing, and/or a shape-memory member.

In some implementations, the shape-memory member is coupled to the wing, and/or configured such that temporarily heating the shape-memory member chronically changes a size of the wing.

In some implementations, the system includes an anchor and a delivery tool that can include a catheter, transluminally advanceable to the chamber and a shaft disposed within the catheter.

In some implementations, the shaft can be engaged with the interface and configured, via the engagement, to deploy the implant out of the catheter, and/or position the implant in a position in which the interface is at a site upstream of the valve, and the wing extends over the first leaflet toward the opposing leaflet.

In some implementations, the delivery tool includes a driver, engaged with the anchor and configured to secure the implant in the position by using the anchor to anchor the interface to tissue of the heart at the site. In some implementations, the delivery tool can be electrically connected to the shape-memory member and/or can be configured, via the electrical connection, to electrically heat the shape-memory member.

In some implementations, the implant, the anchor and the delivery tool are sterile.

In some implementations, the implant is configured such that temporarily heating the shape-memory member causes a change in shape of the shape-memory member that chronically changes the size of the wing.

In some implementations, the implant is configured such that heating the shape-memory member causes a change in shape of the shape-memory member.

In some implementations, the implant includes a lock, the lock configured to transition between: (i) a locked state in which the change in shape of the shape-memory member does not change a size of the wing, and/or (ii) an unlocked state in which the change in shape of the shape-memory member changes the size of the wing.

In accordance with some implementations, a system (e.g., usable or for use with a valve of a real or simulated heart) can include an implant, an anchor, and/or a delivery tool. In some implementations, the implant can include an interface. In some implementations, the anchor can include an anchor head, and/or a tissue-engaging element. In some implementations, the tissue-engaging element can extend from the anchor head.

In some implementations, the delivery tool can include a catheter, a shaft, and/or a driver. The catheter can be transluminally advanceable to the heart.

In some implementations, the shaft can be disposed within the catheter, with a distal end portion of the shaft engaged with the interface and configured, via the engagement, to (i) deploy the implant out of the catheter, and/or (ii) position the implant such that the interface is disposed at a site in the heart.

In some implementations, the driver can be engageable or engaged with the anchor and configured to secure the implant at the site by using the anchor to anchor the interface to tissue of the heart at the site.

In some implementations, a distal segment of the shaft can have (i) a rigid state, and (ii) a flexible state in which the distal segment is more flexible than when the distal segment assumes the rigid state, and/or can be transitionable between the rigid state and the flexible state while the distal end portion remains connected to the interface.

In some implementations, at least one of the implant, the anchor, and the delivery tool is sterile.

In some implementations, the shaft defines a shaft-lumen. In some implementations, the driver can be slidably advanceable through the shaft-lumen such that a drive head of the driver is engaged with the anchor head.

In some implementations, the driver can include a driveshaft that is more flexible than the distal segment of the shaft while the distal segment assumes the rigid state.

In some implementations, the distal segment has an outer diameter that is no more than 10 percent greater than an outer diameter of a proximal portion of the shaft.

In some implementations, the distal segment of the shaft can include a spring. The distal segment can be transitionable between the rigid state and the flexible state by altering tension on the spring.

In some implementations, the distal segment of the shaft includes a tether. The distal segment can be transitionable between the rigid state and the flexible state by altering tension on the tether.

In some implementations, the distal segment of the shaft is transitionable between the rigid state and the flexible state while the driver remains engaged with the anchor.

In some implementations, the distal segment of the shaft is transitionable between the rigid state and the flexible state while the interface is anchored to tissue of the heart at the site.

In some implementations, the distal segment of the shaft includes a hinge. The distal segment can be transitionable between the rigid state and the flexible state by regulating articulation of the hinge.

In some implementations, the system is configured such that transitioning the distal segment from the rigid state to the flexible state increases an articulation-range of the hinge along an articulation axis.

In some implementations, the hinge is a first hinge, and the articulation axis is a first articulation axis. In some implementations, the distal segment can further include a second hinge configured to articulate along a second articulation axis that is nonparallel to the first articulation axis.

In some implementations, the distal segment is configured such that transitioning the distal segment from the rigid state to the flexible state increases a second articulation-range of the hinge along the second articulation axis.

In some implementations, the second articulation axis is orthogonal to the first articulation axis.

In some implementations, the distal segment is transitionable between the rigid state and the flexible state by moving the anchor longitudinally through the distal segment.

In some implementations, the distal segment is transitionable from the rigid state to the flexible state by anchoring the interface to tissue of the heart at the site.

In accordance with some implementations, a method useable with a valve of a real or simulated heart can include, using a catheter, advancing an implant that includes an interface to the heart. In some implementations, the implant can be deployed out of a distal opening of the catheter using a shaft, while a distal end portion of the shaft is coupled to the interface.

In some implementations, a driver that is engaged to an anchor can be used to anchor the interface to tissue at a site of the heart by driving an anchor into the tissue.

In some implementations, subsequently to the step of deploying, and while the distal end portion of the shaft remains coupled to the interface, a distal segment of the shaft can be transitioned from a rigid state to a flexible state in which the distal segment is more flexible than when the distal segment assumes the rigid state.

In some implementations, the method can further include subsequently disengaging the driver from the anchor, and/or decoupling the distal end portion of the shaft from the interface.

In some implementations, the step of transitioning is subsequent to the step of anchoring.

In some implementations: the distal segment of the shaft includes a docking station at which the distal end portion of the shaft is reversibly coupled to a proximal portion of the shaft, and/or the step of transitioning includes transitioning the distal segment of the shaft from the rigid state to the flexible state by decoupling the distal end portion of the shaft from the proximal portion of the shaft.

In some implementations: the shaft includes a tether, and/or the step of transitioning includes transitioning the distal segment of the shaft from the rigid state to the flexible state by decoupling the distal end portion of the shaft from the proximal portion of the shaft by reducing tension on the tether.

In some implementations, the method further includes, prior to the step of disengaging, recoupling the distal end portion of the shaft to the proximal portion of the shaft by increasing tension on the tether.

In some implementations, the method further includes sterilizing the implant, the shaft and the catheter.

In some implementations, the shaft includes a tether. In some implementations, the step of transitioning can include transitioning the distal segment from the rigid state to the flexible state by adjusting tension on the tether.

In some implementations, the distal segment of the shaft includes a spring. In some implementations, the step of transitioning can include transitioning the distal segment from the rigid state to the flexible state by adjusting tension on the spring.

In some implementations, the method further includes, using the shaft, prior to the step of anchoring, positioning the interface at the site prior to anchoring the interface to the tissue.

In some implementations, the step of positioning includes positioning the interface at the site while the distal segment of the shaft assumes the rigid state.

In some implementations, the method further includes, subsequently to the step of anchoring, re-transitioning the distal segment from the flexible state to the rigid state, and/or withdrawing the shaft and the driver from the subject.

In some implementations, the method further includes, prior to the step of re-transitioning, assessing function of the valve.

In some implementations, the step of assessing is prior to the step of disengaging.

In some implementations, the step of assessing is prior to the step of decoupling.

In some implementations, the site is a first site, and/or the method further includes, responsively to the step of assessing, (i) using the driver, removing the anchor from the tissue at the first site, (ii) using the shaft, redeploying the implant to a second site of the heart, and/or (iii) using the driver, anchoring the interface to tissue at the second site of the heart by driving the anchor into the tissue at the second site.

In some implementations, the method further includes, wherein the step of re-transitioning is prior to the step of re-anchoring.

In some implementations, the step of transitioning includes transitioning the distal segment from the rigid state to the flexible state by driving the anchor through the interface and into the tissue.

In some implementations, the distal segment includes a hinge. In some implementations, the step of transitioning can include increasing a range of articulation of the hinge along an articulation-axis.

In some implementations, the hinge is a first hinge having a first range of articulation along a first articulation-axis, and the distal segment further includes a second hinge having a second range of articulation along a second articulation-axis. In some implementations, the step of transitioning can further include increasing the second range of articulation of the second hinge.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations, a method (e.g., useable or for use with a valve of a real or simulated heart of a subject (e.g., living subject or simulation)) can include, using a catheter, advancing an implant including an interface to the heart. In some implementations, the method can include, using a shaft, a distal end portion of the shaft coupled to the interface, deploying the implant out of a distal opening of the catheter.

In some implementations, a driver that is engaged to an anchor, can be used to anchor the interface to tissue at a site of the heart by driving an anchor into the tissue.

In some implementations, the method can further include, subsequently to the step of deploying, (i) while the distal end portion of the shaft remains coupled to the interface, transitioning a distal segment of the shaft from a rigid state to a flexible state in which the distal segment is more flexible than when the distal segment assumes the rigid state, and/or, (ii) subsequently (a) disengaging the driver from the anchor; and/or (b) decoupling the distal end portion of the shaft from the interface.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations, a system useable with a valve of a real or simulated heart (e.g., a valve having a first leaflet and an opposing leaflet, the heart having a chamber upstream of the valve) can include an implant, an anchor, and/or a delivery tool.

In some implementations, the implant can include a wing, and/or an interface. In some implementations, the wing can extend from a root portion of the wing to a tip portion of the wing.

In some implementations, the interface can be at the root portion of the wing. In some implementations, interface can be at an edge (e.g., upper edge, proximal edge, etc.) of the implant.

In some implementations, the anchor can be rotatably coupled and axially fixed to the interface. In some implementations, the anchor can include an anchor head, and/or a tissue-engaging element extending from the anchor head to define an anchor axis of the anchor.

In some implementations, the delivery tool can include a catheter, a shaft, and/or a driver. The catheter can be transluminally advanceable to the chamber.

In some implementations, the shaft can be disposed within the catheter, engaged with the interface and configured, via the engagement, to: (i) deploy the implant out of the catheter, and/or (ii) position the implant in a position in which the interface is at a site upstream of the valve, and the wing extends over the first leaflet toward the opposing leaflet.

In some implementations, the driver can be engaged with the anchor and configured to secure the implant in the position using the anchor to anchor the interface to tissue of the heart at the site.

In some implementations, at least one of the implant, the anchor, and the delivery tool is sterile.

In accordance with some implementations, a system (e.g., usable or for use with a real or simulated heart of a real or simulated subject) can include an implant, an anchor, and/or a delivery tool.

In some implementations, the implant can include an interface, an anchor receiver, and/or a wing, coupled to the interface and to the anchor receiver.

In some implementations, the delivery tool can include a catheter, a shaft, and/or a driver. The catheter can be transluminally advanceable to the heart and define a distal opening and a lateral opening.

In some implementations, the shaft can be disposed within the catheter, engaged with the interface and configured, via the engagement, to: (i) deploy the implant out of the distal opening of the catheter, and/or (ii) position the implant such that the anchor receiver is disposed at a site of the heart.

In some implementations, the driver can be engaged with the anchor and configured to secure the implant to the heart by: (i) advancing the anchor out of the lateral opening of the catheter and toward the anchor receiver, and/or (ii) driving the anchor through the anchor receiver and into tissue of the heart at the site.

In some implementations, at least one of the implant, the anchor, and the delivery tool is sterile.

In some implementations, the implant has a compressed state and an expanded state. In some implementations, the implant includes a flexible frame that includes a shape-memory material and biases the implant toward assuming the expanded state. In some implementations, the implant and/or frame can be actuated (e.g., mechanically actuated, etc.) to expand the implant and/or frame to the expanded state.

In some implementations, the implant can include an expansion element, having: (i) a compact state, and/or (ii) an extended state in which the expansion element resists compression of the implant toward the compressed state.

In some implementations, the catheter is configured to house the implant while the implant is in the compressed state and the expansion element is in the compact state.

In some implementations, the shaft is configured, via the engagement to deploy the implant out of the distal opening of the catheter such that, within the heart, the implant assumes the expanded state and the expansion element assumes the extended state.

In some implementations, the implant is configured such that extension of the expansion element from the compact state to the extended state applies an expansion force to the implant, the expansion force facilitating expansion of the implant from the compressed state to the expanded state.

In some implementations, the expansion element is configured to resist transition from the extended state toward the compact state.

In some implementations, the expansion element includes a spring.

In some implementations, the expansion element includes a plurality of subunits, and the expansion element is configured such that extending the expansion element into the extended state causes the subunits to fit together.

In some implementations, the expansion element includes a plurality of subunits, configured to lock together upon the expansion element assuming the extended state.

In some implementations, the expansion element is straighter in the extended state than in the compact state.

In some implementations, the expansion element includes a hinge, and the expansion element can be configured such that straightening the hinge straightens the expansion element.

In some implementations, the delivery tool further includes an extension actuator, the extension actuator configured to transluminally extend the expansion element from the compact state to the extended state.

In some implementations, the anchor is a first anchor, and the system further includes a second anchor. The anchor receiver is a first anchor receiver, and the implant can further include a second anchor receiver.

In some implementations, the driver is a first driver, and the delivery tool can further include a second driver. In some implementations, the lateral opening is a first lateral opening, and the catheter can further define a second lateral opening opposite the first lateral opening.

In some implementations, the first and second drivers can each be engaged with a respective anchor and configured to secure the implant to the heart by: (i) advancing out of one of the lateral openings and toward one of the anchor receivers, and/or (ii) driving one of the first and second anchors through one of the anchor receivers and into tissue of the heart at the site.

In some implementations, the first and second drivers are configured to diverge away from each other as the first and second drivers advance out of one of the lateral openings and toward the anchor receivers.

In some implementations, the catheter further includes a gate at the lateral opening, the gate including a shape-memory material. In some implementations, the catheter can be configured to transition between: (i) a delivery state in which the gate is closed, and/or (ii) a deployment state in which the gate is open.

In some implementations, the system is configured such that deploying the implant out of the distal opening facilitates transitioning the catheter from the delivery state to the deployment state.

In some implementations, the catheter is configured such that while the catheter is in the deployment state, the open gate guides the driver and the anchor out of the lateral opening of the catheter and toward the anchor receiver.

In accordance with some implementations, a method (e.g., usable or for use with a real or simulated heart of a living subject or simulation) can include advancing to the heart an implant compressed within a catheter. In some implementations, the implant can include an interface, an anchor receiver, and/or a wing, coupled to the interface and to the anchor receiver.

In some implementations, a shaft that is coupled to the interface can be used to deploy the implant out of a distal opening of the catheter such that the wing expands within the heart.

In some implementations, a driver can be used (i) to advance an anchor out of a lateral opening of the catheter and to the anchor receiver, and/or (ii) to anchor the implant to tissue of the heart by driving a tissue-engaging element of the anchor through the anchor receiver and into the tissue.

In some implementations, the method further includes sterilizing the implant and the catheter.

In some implementations, the step of deploying includes deploying the implant out of a distal opening of the catheter in a direction that is generally parallel to a longitudinal axis of a distal portion of the shaft. In some implementations, the step of advancing includes advancing the anchor out of the lateral opening of the catheter in a direction that is oblique with respect to the longitudinal axis of the distal portion of the shaft.

In some implementations, the driver is a first driver, the lateral opening is a first lateral opening of the catheter, the anchor receiver is a first anchor receiver, and the implant further includes a second anchor receiver.

In some implementations, the step of advancing can include, using the first driver and a second driver: (i) advancing a first anchor out of the first lateral opening of the catheter and to the first anchor receiver, and/or (ii) advancing a second anchor out of a second lateral opening of the catheter and to the second anchor receiver.

In some implementations, the step of anchoring can include anchoring the implant to tissue of the heart by driving each anchor through a respective anchor receiver and into tissue of the heart.

In some implementations, the step of advancing includes advancing the first driver and the second driver divergently away from each other.

In some implementations, the catheter further includes a shape-memory gate at the lateral opening. In some implementations, the catheter can be transitioned from a delivery state in which the gate is closed, to a deployment state in which the gate is open.

In some implementations, the step of transitioning includes transitioning the catheter from the delivery state to the deployment state by deploying the implant out of the distal opening of the catheter.

In some implementations, the step of transitioning includes transitioning the catheter from the delivery state to the deployment state by proximally retracting the driver within the catheter.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations, a method (e.g., usable or for use with a real or simulated heart of a living subject or simulation) can include advancing to the heart an implant compressed within a catheter, the implant including: (i) an interface, (ii) an anchor receiver, and/or (iii) a wing, coupled to the interface and to the anchor receiver.

In some implementations, the method can include, using a shaft coupled to the interface, deploying the implant out of a distal opening of the catheter such that the wing expands within the heart.

In some implementations, the method can include, using a driver: (i) advancing an anchor out of a lateral opening of the catheter and to the anchor receiver, and/or (ii) anchoring the implant to tissue of the heart by driving a tissue-engaging element of the anchor through the anchor receiver and into the tissue.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations, a system (e.g., usable or for use with a real or simulated heart of a living subject or a simulation) can include an implant, an anchor, a catheter, a latch, a shaft, a driver, and/or an insert.

In some implementations, the implant can include an interface. The catheter can be transluminally advanceable to the heart.

In some implementations, the shaft can be: (i) disposed within the catheter, (ii) reversibly engaged to the interface via the latch, and/or (iii) configured, via the engagement, to: (a) deploy the implant out of the catheter, and/or (b) position the implant in a position in which the interface is disposed at a site of the heart.

In some implementations, the driver can be disposed within the catheter, and configured to anchor the implant in the position by driving the anchor into tissue at the site.

In some implementations, the insert can be disposed between the shaft and the interface, and slidable in a manner that disengages the shaft from the interface by displacing the latch.

In some implementations, at least one of the implant, the anchor, the catheter, and the shaft is sterile.

In some implementations, the shaft is shaped to define the latch.

In some implementations, the driver is shaped to define the insert.

In some implementations, the driver is configured to anchor the implant in the position by driving the anchor through the interface and into tissue at the site.

In some implementations, the system is configured such that the driver extends distally, from outside the subject, within the catheter and through the shaft.

In some implementations, the interface is shaped to define a window. In some implementations, the system can be configured such that while the shaft is engaged to the interface, the latch is disposed within the window. In some implementations, the insert can be slidable in a manner that disengages the shaft from the interface by displacing the latch from within the window.

In some implementations, the anchor defines an anchor head and a helical tissue-engaging element that extends away from the anchor head along an anchor axis.

In some implementations, the system is configured such that the insert is rotatable with respect to the latch about the anchor axis in a manner that disengages the shaft from the interface by displacing the latch.

In some implementations, the system is configured such that the insert is slidable with respect to the latch along the anchor axis in a manner that disengages the shaft from the interface by displacing the latch.

In some implementations, the latch includes a shape-memory material having a compressed shape and a relaxed shape. In some implementations, the insert can be slidable in a manner that disengages the shaft from the interface by causing the latch to transition between the compressed shape and the relaxed shape. In some implementations, the insert can be slidable in a manner that disengages the shaft from the interface by causing the latch to disengage from the interface.

In some implementations, the insert includes an intervening tube disposed between the shaft and the interface.

In some implementations, the intervening tube is slidable with respect to the shaft and the interface.

In some implementations, the intervening tube circumscribes a portion of the driver.

In some implementations, the intervening tube circumscribes a portion of the anchor.

In some implementations, the intervening tube circumscribes a portion of the interface.

In accordance with some implementations, a method (e.g., usable or for use at a real or simulated heart of a living subject or a simulation) can include advancing to the heart: (i) an anchor, (ii) an implant defining an interface, (iii) a catheter housing the implant, (iv) a latch, (v) a shaft reversibly engaged to the interface via the latch, and/or (vi) an insert disposed between the shaft and the interface. In some implementations, the implant can be deployed out of the catheter.

In some implementations, the shaft can be used to position the implant such that the interface is disposed at a site of the heart.

In some implementations, a driver can be used to secure the implant in the position by driving a portion of the anchor through the interface and into tissue at the site. In some implementations, the shaft can subsequently be disengaged from the implant by sliding the insert between the shaft and the interface.

In some implementations, the method further includes sterilizing the implant, the anchor, the shaft and the catheter.

In some implementations, the step of disengaging includes displacing the latch from the interface.

In some implementations, the interface is shaped to define a window. The step of disengaging can include displacing the latch from within the window.

In some implementations, the latch can include a shape-memory material having a compressed shape and a relaxed shape. In some implementations, the step of disengaging can include displacing the latch from the interface by sliding the insert between the shaft and the interface in a manner that allows the latch to transition between the compressed shape and the relaxed shape.

In some implementations, the step of disengaging includes displacing the latch from the interface by sliding the insert between the shaft and the interface in a manner that expands the latch laterally with respect to the interface.

In some implementations, the anchor defines an anchor head, and/or an anchor axis along which a helical tissue-engaging element extends from the anchor head. In some implementations, the step of disengaging can include displacing the latch from the interface by sliding the insert with respect to the latch along the anchor axis.

In some implementations, the anchor defines an anchor head, and/or an anchor axis along which a helical tissue-engaging element extends from the anchor head.

In some implementations, the step of disengaging can include displacing the latch from the interface by rotating the insert with respect to the latch about the anchor axis.

In some implementations, the anchor is a first anchor, the interface is a first interface, and the implant further includes a second interface that defines a longitudinal axis along which the second interface can be configured to receive a second anchor.

In some implementations, the latch can be a first latch, the shaft can be a first branch of the shaft, and/or the insert can be a first insert.

In some implementations, the method can include advancing to the heart: (i) the second anchor, (ii) a second latch, (iii) a second branch of the shaft reversibly engaged to the second interface via the second latch, and/or (iv) a second insert disposed between the second branch of the shaft and the second interface.

In some implementations, the step of disengaging can include disengaging the shaft from the implant by: (a) rotating the first insert with respect to the first latch in a first direction about the longitudinal axis of the first interface, and/or (b) rotating the second insert with respect to the second latch in a second, opposite direction about the longitudinal axis of the second interface.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations, a method (e.g., usable or for use at a real or simulated heart of a living subject or a simulation) can include advancing to the heart: (i) an anchor, (ii) an implant defining an interface, (iii) a catheter housing the implant, (iv) a latch, (v) a shaft reversibly engaged to the interface via the latch, and/or (vi) an insert disposed between the shaft and the interface. The method can include deploying the implant out of the catheter.

In some implementations, the method can include, using the shaft, positioning the implant such that the interface is disposed at a site of the heart.

In some implementations, the method can include, using a driver, securing the implant in the position by driving a portion of the anchor through the interface and into tissue at the site.

In some implementations, the method can include subsequently, disengaging the shaft from the implant by sliding the insert between the shaft and the interface.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations, a system (e.g., usable or for use with a valve of a real or simulated heart of a subject (e.g., living subject or simulation), such as a valve having a first leaflet and an opposing leaflet, the heart having a chamber upstream of the valve) can include an anchor and/or an implant.

In some implementations, the implant can include a wing, and/or an anchor receiver. In some implementations, the wing can extend from a root portion of the wing to a tip portion of the wing.

In some implementations, the anchor receiver can be configured to promote tissue ingrowth thereon.

In some implementations, the anchor receiver can be coupled to the root portion (e.g., at or near an edge of the implant, or another location) of the wing such that anchoring of the anchor receiver to an annulus of the valve positions the wing such that: (i) the wing extends over the first leaflet toward the opposing leaflet, and/or (ii) responsively to a cardiac cycle of the heart, the wing deflects, in a reciprocating manner, in an upstream direction and in a downstream direction.

In some implementations, the implant can define an obstacle that can be configured to inhibit the tissue ingrowth from progressing from the anchor receiver toward the tip portion of the wing.

In some implementations, at least one of the implant and the anchor is sterile.

In some implementations, the wing defines a contact face, and an opposing face opposite to the contact face. In some implementations, the obstacle can include a cage.

In some implementations, the cage is disposed on the opposing face, at the root portion of the wing. In some implementations, the cage can include a barrier configured to mechanically inhibit the tissue ingrowth from progressing from the anchor receiver toward the tip portion of the wing.

In some implementations, the barrier is a bilayer barrier including an interface-facing layer of the barrier including a material that promotes tissue growth thereupon. In some implementations, the bilayer barrier can be an opposing layer including material that inhibits tissue growth thereupon.

In some implementations, the cage defines a backstop portion that is shaped to press against tissue of the chamber upon anchoring of the anchor receiver to the annulus.

In some implementations, the obstacle is configured to inhibit the tissue ingrowth from progressing from the anchor receiver toward the tip portion of the wing by reducing contact between the root portion of the wing and the first leaflet.

In some implementations, the wing is shaped to define the obstacle such that, while the anchor receiver is anchored to the annulus the root portion of the wing curves in the upstream direction, away from the first leaflet. In some implementations, the tip portion of the wing can curve in the downstream direction, toward the first leaflet.

In some implementations, the wing defines a contact face, and an opposing face opposite to the contact face. In some implementations, the obstacle can include a stilt attached to the contact face at the root portion of the wing, the stilt configured such that while the wing extends over the first leaflet toward the opposing leaflet, the stilt inhibits contact between the root portion of the wing and the first leaflet.

In accordance with some implementations, a system (e.g., usable or for use with a real or simulated heart of a subject (e.g., living subject or simulation)) can include an implant, and/or an anchor. In some implementations, the implant can include an anchor receiver.

In some implementations, the anchor can include an anchor head, and/or a helical tissue-engaging element that extends distally from the anchor head along an anchor axis, and configured to be screwed through the anchor receiver and into tissue by rotation of the anchor head in a rotational direction at least until the anchor head reaches the anchor receiver.

In some implementations, the anchor head and the anchor receiver can be shaped such that, upon the anchor head reaching the anchor receiver, further rotation of the anchor head in the rotational direction pushes the anchor receiver distally with respect to the anchor.

In some implementations, at least one of the implant and the anchor is sterile.

In some implementations, the system can include a delivery tool including a catheter, transluminally advanceable to the heart, a shaft disposed within the catheter, a distal end portion of the shaft engaged with the interface and configured, via the engagement, to: (i) deploy the implant out of the catheter, and/or (ii) position the implant such that the interface is disposed at a site in the heart.

In some implementations, the delivery tool can include a driver configured to secure the implant at the site by using the anchor to anchor the anchor receiver to tissue of the heart at the site.

In some implementations, the anchor head and the anchor receiver are shaped such that, upon the anchor head reaching the anchor receiver, further rotation of the anchor head in the rotational direction pushes the anchor head proximally away from the anchor receiver.

In some implementations, the anchor head and the anchor receiver are each shaped to define complementarily undulating surfaces along which the anchor head interfaces with the anchor receiver.

In accordance with some implementations, a method (e.g., useable or for use at a real or simulated heart of a subject (e.g., living subject or simulation)) can include advancing to the heart: (i) an anchor including an anchor head and a tissue-engaging portion extending from the anchor head, and/or (ii) an implant compressed within a catheter, the implant including an anchor receiver.

In some implementations, a shaft, coupled to the anchor receiver, can be used to deploy the implant out of a distal opening of the catheter such that the anchor receiver is disposed at a site of the heart.

In some implementations, a driver can be used to screw the anchor through the anchor receiver and into tissue at the site by rotating the anchor head at least until the anchor head reaches the anchor receiver.

In some implementations, after the anchor head reaches the anchor receiver, the anchor receiver can be pushed into tissue at the site by further rotating the anchor head.

In some implementations, the method further includes sterilizing the shaft, the anchor and the implant.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations, a method (e.g., usable or for use at a real or simulated heart of a subject (e.g., living subject or simulation)) can include advancing to the heart: (i) an anchor including an anchor head and a tissue-engaging portion extending from the anchor head, and/or (ii) an implant compressed within a catheter, the implant including an anchor receiver.

In some implementations, the method can comprise, using a shaft coupled to the anchor receiver, deploying the implant out of a distal opening of the catheter such that the anchor receiver is disposed at a site of the heart.

In some implementations, the method can comprise, using a driver, screwing the anchor through the anchor receiver and into tissue at the site by rotating the anchor head at least until the anchor head reaches the anchor receiver.

In some implementations, the method can comprise, after the anchor head reaches the anchor receiver, pushing the anchor receiver into tissue at the site by further rotating the anchor head.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations, a system (e.g., usable or for use at a real or simulated tissue of a subject (e.g., living subject or simulation)) can include an implant. In some implementations, the implant can include a frame and an interface, the interface including: (i) a first collar, configured to interface with the anchor head, (ii) a second collar, and/or (iii) a neck portion, connecting the first collar to the second collar, and extending through a loop portion of the frame.

In some implementations, the interface can have a loose state in which the loop portion is loosely coupled to the interface.

In some implementations, the interface can have a tight state in which the loop portion is sandwiched between the first collar and the second collar.

In some implementations, the implant is sterile.

In some implementations, the system further includes an anchor, wherein the interface can be configured to transition from the loose state to the tight state by advancing the anchor distally through the interface.

In some implementations, the anchor includes an anchor head and a tissue-engaging portion extending away from the anchor head along an anchor axis.

In some implementations, the system is configured such that while the interface is in the loose state, at least a portion of the tissue-engaging portion protrudes distally through the interface.

In some implementations, the anchor includes an anchor head and a tissue-engaging portion extending away from the anchor head along an anchor axis.

In some implementations, the interface is configured to transition from the loose state to the tight state by advancing the tissue-engaging portion distally through the interface such that: (i) the anchor head presses distally against the interface, and/or (ii) the tissue-engaging portion passes distally through the interface.

In some implementations, the system is configured such that a deflectability of the implant along the anchor axis is reduced as the interface transitions from the loose state to the tight state.

In some implementations, the system can include a delivery tool including (i) a catheter, transluminally advanceable to the site, (ii) a shaft disposed within the catheter, a distal end portion of the shaft engaged with the interface and configured, via the engagement, to: (a) deploy the implant out of the catheter, and/or (b) position the implant such that the interface is disposed at the site.

In some implementations, the delivery tool can also include a driver configured to: (i) secure the implant at the site, and/or (ii) transition the interface from the loose state to the tight state by advancing the anchor distally through the interface.

In some implementations, the system is configured such that while the interface is in the loose state the drive head is engaged with anchor head. In some implementations, the system is configured such that while the interface is in the loose state, the shaft is engaged with the interface.

In some implementations, the interface can be configured to transition from the loose state to the tight state while the drive head remains engaged with anchor head. In some implementations, the interface can be configured to transition from the loose state to the tight state while the shaft remains engaged with the interface.

In some implementations, the system is configured such that while the interface is in the loose state, the implant is deflectable with respect to the interface along an assessment deflection-range that is generally equal to a deployment deflection-range along which the implant is deflectable while the system assumes a deployment state in which: (i) the interface is in the tight state, and/or (ii) the shaft is disengaged from the interface.

In accordance with some implementations, a method (e.g., usable or for use at a real or simulated tissue of a subject (e.g., living subject or simulation)) can include using a catheter, advancing to a site of the tissue an anchor, and/or an implant including a frame and an interface.

In some implementations, the interface can include (i) a first collar, configured to interface with the anchor head, (ii) a second collar, and/or (iii) a neck portion, connecting the first collar to the second collar, and extending through a loop portion of the frame.

In some implementations, the driver can be used to transition the interface from a loose state in which the loop portion is loosely coupled to the interface, to a tight state in which the loop portion is sandwiched between the first collar and the second collar.

In some implementations, the method further includes sterilizing the anchor and the implant.

In some implementations, the anchor includes an anchor head and a tissue-engaging portion extending away from the anchor head along an anchor axis.

In some implementations, the step of transitioning can include transitioning the interface from the loose state to the tight state by advancing the anchor through the interface and into tissue at the site.

In some implementations, the step of transitioning includes transitioning the interface from the loose state to the tight state such that the anchor head presses distally against the interface.

In some implementations, the step of transitioning includes reducing a deflectability of the implant along the anchor axis.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations, a method (e.g., usable or for use at a real or simulated tissue of a subject (e.g., living subject or simulation)) can include using a catheter, advancing an anchor, and/or an implant to a site of the tissue. In some implementations, the implant can include a frame and an interface.

In some implementations, the interface can include: (i) a first collar, configured to interface with the anchor head, (ii) a second collar, and/or (iii) a neck portion, connecting the first collar to the second collar, and extending through a loop portion of the frame.

In some implementations, a driver can be used to transition the interface from a loose state in which the loop portion is loosely coupled to the interface, to a tight state in which the loop portion is sandwiched between the first collar and the second collar.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations, a system (e.g., usable or for use with a valve of a real or simulated heart of a subject (e.g., living subject or simulation), such as a valve having a first leaflet and an opposing leaflet, the heart having a chamber upstream of the valve) can include an anchor, an implant, and/or a delivery tool.

In some implementations, the anchor defines an anchor axis.

In some implementations, the implant can include a wing, and/or an interface at a root portion of the wing.

In some implementations, the delivery tool can include a catheter, transluminally advanceable to the heart, a shaft disposed within the catheter, a coupling, and/or a driver.

In some implementations, the coupling can be attached to a distal end of the shaft, the shaft being configured, via engagement of the coupling with the interface, to deploy the implant out of the catheter.

In some implementations, the shaft is configured, via engagement of the coupling with the interface, to position the implant in a position in which: (i) the wing extends over the first leaflet toward the opposing leaflet, and/or (ii) responsively to a cardiac cycle of the heart, the wing deflects, in a reciprocating manner, in an upstream direction and in a downstream direction.

In some implementations, the driver is configured to secure the implant in the position by driving the anchor into tissue of the heart.

In some implementations, the delivery tool is configured to transition the system between (i) a first state, in which the coupling is engaged with the interface, (ii) a second state, in which the coupling is engaged with the interface, and the wing has greater deflectability with respect to the anchor axis than in the first state, and/or (iii) a deployed state, in which the coupling is disengaged from the interface.

In some implementations, at least one of the implant, the anchor and the delivery tool is sterile.

In some implementations, the shaft has a proximal portion, proximal from the coupling and the distal end of the shaft. In some implementations, while the system is in the first state, the distal end of the shaft is secured to the proximal portion of the shaft.

In some implementations, the delivery tool is configured to transition the system into the second state by releasing the distal end of the shaft from the proximal portion of the shaft.

In some implementations, the delivery tool is configured such that while the delivery tool is in the deployed state, the distal end of the shaft is secured to the proximal portion of the shaft.

In some implementations: the shaft further includes a tether, and/or the delivery tool is configured to transition between the first state and the second state by adjusting tension on the tether.

In some implementations, the distal end of the shaft is reversibly coupled to the proximal portion of the shaft, such that: increasing tension on the tether secures the distal end of the shaft to the proximal portion of the shaft, and/or reducing tension on the tether decouples the distal end of the shaft from the proximal portion of the shaft.

In some implementations, the deflectability of the wing along the anchor axis while the system is in the second state is generally equal to the deflectability of the wing along the anchor axis while the system is in the deployed state.

In some implementations, the coupling has an outer diameter that is no more than 10 percent greater than an outer diameter of a proximal portion of the shaft.

In some implementations, the coupling includes a spring. In some implementations, the system can be transitionable between the first state and the second state by altering tension on the spring.

In some implementations, the coupling includes a tether. In some implementations, the system can be transitionable between the first state and the second state by altering tension on the tether.

In some implementations, the system is transitionable between the first state and the second state while the driver is engaged with the anchor.

In some implementations, the system is transitionable between the first state and the second state while the interface is anchored to tissue of the heart.

In some implementations, the coupling includes a hinge. In some implementations, the system can be transitionable between the first state and the second state by regulating articulation of the hinge.

In some implementations, the system is configured such that transitioning the system from the second state to the first state increases an articulation-range of the hinge along an articulation axis.

In some implementations, the hinge is a first hinge, and the articulation axis is a first articulation axis.

In some implementations, the coupling can further include a second hinge configured to articulate along a second articulation axis that is nonparallel to the first articulation axis.

In some implementations, the coupling is configured such that transitioning the system from the second state to the first state increases a second articulation-range of the hinge along the second articulation axis.

In some implementations, the second articulation axis is orthogonal to the first articulation axis.

In some implementations, the system is transitionable between the first state and the second state by moving the anchor longitudinally through the coupling.

In some implementations, the system is transitionable from the second state to the first state by anchoring the interface to tissue of the heart.

In some implementations, the interface includes a first collar, a second collar, and/or a neck portion. In some implementations, the neck portion can connect the first collar to the second collar, to which a loop portion of the wing is coupled.

In some implementations, the system is configured such that, while the system is in the first state, the loop portion is sandwiched between the first collar and the second collar. In some implementations, while the system is in the second state, the loop portion can be loosely coupled to the interface.

In some implementations, the system is configured to be transitioned from the second state to the first state by advancing the anchor distally through the interface.

In some implementations, the anchor includes an anchor head and a tissue-engaging portion extending away from the anchor head.

In some implementations, the system is configured to transition from the second state to the first state by advancing the tissue-engaging portion distally through the interface such that the anchor head presses distally against the interface.

In some implementations, the system is configured such that while the interface is in the second state, at least a portion of the anchor protrudes distally through the interface.

In accordance with some implementations, a system (e.g., useable or for use with a real or simulated tissue of a subject (e.g., living subject or simulation)) can include an anchor, and/or an implant.

In some implementations, the anchor can define an anchor head and a helical tissue-engaging element extending distally from the anchor head along an anchor axis.

In some implementations, the implant, the implant including an interface configured to be anchored to a site of the tissue by advancing the tissue-engaging element helically through the interface and into the tissue.

In some implementations, the interface can include a tubular anchor receiver defining a lumen, and/or a stopper disposed within the lumen. In some implementations, the stopper can define a window dimensioned to facilitate helical advancement of the tissue-engaging element therethrough, until the anchor head meets the stopper.

In some implementations, the stopper can define a wall configured to inhibit non-helical advancement of the anchor distally through the interface.

In some implementations, at least one of the anchor and the implant is sterile.

In accordance with some implementations, a system and/or an apparatus (e.g., usable or for use with a real or simulated cardiovascular system of a subject (e.g., living subject or simulation)) can include an anchor and/or a driver.

In some implementations, the anchor can include a helical tissue-engaging element defining an anchor axis of the anchor, having a distal point, and configured to be: (i) screwed into tissue of the cardiovascular system by rotation of the anchor in a first rotational direction about the anchor axis, and/or (ii) unscrewed from the tissue by rotation of the anchor in a second rotational direction about the anchor axis, the second rotational direction being opposite to the first rotational direction.

In some implementations, the anchor can include an anchor head, attached to a proximal end of the tissue-engaging element, and shaped to define: (i) a smooth forward-torque face, facing in the second rotational direction, and/or (ii) an anchor hook facing in the first rotational direction around the anchor axis.

In some implementations, the driver can include a driveshaft, and/or a drive head that defines: (i) a smooth driver screw-in surface, facing in the first rotational direction, and/or (ii) a driver hook, facing in the second rotational direction, and shaped complementarily to the anchor hook.

In some implementations, the driver is configured to screw the helical tissue-engaging element into the tissue by applying torque, in the first rotational direction, to the anchor head by pressing the driver screw-in surface against the forward-torque face while pressing the anchor head distally.

In some implementations, the driver is configured to unscrew the helical tissue-engaging element from the tissue by applying torque, in the second rotational direction, to the anchor head by hooking the driver hook into the anchor hook and pressing the driver hook against the anchor hook while pulling the anchor hook proximally.

In some implementations, the anchor is sterile.

In some implementations, the anchor head and the tissue-engaging element are configured to be cut from a unitary piece of stock tubing.

In some implementations, the anchor head and the drive head are configured to be cut from a unitary piece of stock tubing.

In accordance with some implementations, a system (e.g., usable or for use with a tissue of a subject) can include an anchor and/or a delivery tool.

In some implementations, the anchor can have a helical tissue-engaging element that has a distal tip.

In some implementations, the anchor can have an anchor head, at a proximal end of the tissue-engaging element, the anchor head being shaped to define: (i) a forward-torque face, and/or (ii) a reverse-torque face.

In some implementations, the delivery tool can include a catheter, transluminally advanceable to the tissue, and/or a driver, including a driveshaft, and a drive head at a distal end of the driveshaft, the drive head being shaped such that while the driveshaft is under compression, rotation of the drive head in a forward rotational direction screws the tissue-engaging element into the tissue by applying forward torque to the forward-torque face.

In some implementations, the drive head can be shaped such that rotation of the drive head in a reverse rotational direction: (i) hooks the drive head onto the anchor head in a manner that facilitates tensioning of the driveshaft while the drive head remains in contact with the anchor head, and/or (ii) unscrews the tissue-engaging element from the tissue by applying reverse torque to the reverse-torque face while the drive head remains hooked onto the anchor head and the driveshaft is under tension.

In some implementations, the drive head can be shaped such that tensioning the driveshaft while the drive head is not hooked onto the anchor head pulls the drive head away from the anchor head.

In some implementations, at least one of the anchor and the catheter is sterile.

In some implementations, the reverse-torque face of the anchor head can be configured to hook the drive head in a manner that maintains contact between the anchor head and the drive head while the driveshaft is under tension.

In some implementations, the forward-torque face of the anchor head defines a smooth face that is closer to being perpendicular to the forward torque than to being parallel to the forward torque.

In some implementations, the driver is configured to (i) screw the tissue-engaging element into the tissue, (ii) hook the drive head onto the anchor head, and (iii) unscrew the tissue-engaging element from the tissue, and (iv) disengage from the anchor head, without any change of conformation of the anchor head.

In some implementations, the driver is configured to (i) screw the tissue-engaging element into the tissue, (ii) hook the drive head onto the anchor head, and (iii) unscrew the tissue-engaging element from the tissue, and (iv) disengage from the anchor head, without any change of conformation of the drive head.

In accordance with some implementations, a method (e.g., usable or for use at a real or simulated tissue of a subject (e.g., living subject or simulation)) can include using a driver that includes a driveshaft and a drive head at a distal end of the driveshaft, advancing to the tissue an anchor that has: (i) an anchor head defining: (a) a forward-torque face, and/or (b) a reverse-torque face, and/or (ii) a helical tissue-engaging element extending from the anchor head.

In some implementations, the anchor can be anchored into the tissue of the heart by, while the drive head is engaged with the anchor head, driving the helical tissue-engaging element into the tissue by using the driver to apply forward torque to the forward-torque face.

In some implementations, the drive head can be disengaged from the anchor head. The step of disengaging may not include changing a shape or a conformation of the drive head or the anchor head.

In some implementations, the method further includes sterilizing the anchor.

In some implementations, the method further includes subsequently unscrewing the tissue-engaging element from the tissue by hooking the drive head onto the anchor head, and/or by applying reverse torque to the reverse-torque face while pulling the anchor proximally using the drive head hooked onto the anchor head.

In some implementations, the step of hooking includes hooking the drive head onto the anchor head by rotating the driver in a reverse rotational direction.

In some implementations, the step of hooking includes hooking the drive head onto the anchor head by sliding the drive head proximally with respect to the anchor head.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations, a method (e.g., usable or for use at a real or simulated tissue of a subject (e.g., living subject or simulation)) can include using a driver that includes a driveshaft and a drive head at a distal end of the driveshaft, advancing to the tissue an anchor that has: (i) an anchor head defining: (a) a forward-torque face, and/or (b) a reverse-torque face, and/or (ii) a helical tissue-engaging element extending from the anchor head.

In some implementations, the anchor can be anchored into the tissue of the heart by, while the drive head is engaged with the anchor head, driving the helical tissue-engaging element into the tissue by using the driver to apply forward torque to the forward-torque face.

In some implementations, the drive head can be disengaged from the anchor head. In some implementations, the step of disengaging may not include changing a shape or a conformation of the drive head or the anchor head.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations, a system (e.g., usable or for use at a real or a simulated tissue of a subject (e.g., living subject or simulation)) can include an implant, an elongate anchor, and/or a delivery tool. In some implementations, the implant can include an interface and an anchor receiver. In some implementations, the delivery tool can extend from a proximal portion to a distal portion.

In some implementations, the delivery tool can include a catheter housing the implant, the catheter transluminally advanceable to the tissue.

In some implementations, the delivery tool can include a shaft, extending distally through the catheter, the shaft configured to: (i) deploy the implant out of the catheter, and/or (ii) position the implant such that the interface and the anchor receiver are disposed against a surface of the tissue.

In some implementations, the delivery tool is configured to anchor the implant to the tissue by driving the anchor through the interface and a surface of the tissue, along a curved path within the tissue, such that a distal part of the anchor exits the tissue and is received by the anchor receiver.

In some implementations, at least one of the implant, the anchor, and the delivery tool is sterile.

In some implementations, a distal part of the shaft extends distally through the catheter, the distal part of the shaft bifurcating into a first branch and a second branch, each branch disposed alongside each other within the catheter. In some implementations, the first branch can be engaged with the interface. In some implementations, the second branch can be engaged with the anchor receiver.

In some implementations, the delivery tool further includes a flexible needle housing the anchor, the needle being deliverable, via the shaft, through the interface and the surface of the tissue, and along the curved path within the tissue to the anchor receiver.

In some implementations, the anchor includes a shape-memory material. In some implementations, the needle is configured to restrain the anchor in a compressed state. In some implementations, the needle can be retractable with respect to the anchor, such that retracting the needle releases the anchor from the compressed state to an expanded state.

In accordance with some implementations, a method (e.g., usable or for use with tissue of a real or simulated heart of a subject (e.g., living subject or simulation)) can include transluminally advancing to the heart, within a catheter: (i) an implant including an interface and an anchor receiver, and/or (ii) a shaft coupled to the interface.

In some implementations, the shaft can be used to deploy the implant out of a distal opening of the catheter such that the interface and the anchor receiver are disposed against a surface of the tissue.

In some implementations, the implant can be anchored to the tissue by driving an anchor through the interface and a surface of the tissue, along a curved path within the tissue, such that a distal part of the anchor exits the tissue and is received by the anchor receiver.

In some implementations, the system further includes sterilizing the implant, the shaft and the catheter.

In some implementations, the anchor includes a shape-memory material. In some implementations, the step of driving the anchor can include driving the anchor within a flexible needle, through the interface and a surface of the tissue, along a curved path within the tissue, such that a distal part of the anchor exits the tissue and is received by the anchor receiver.

In some implementations, the needle can subsequently be retracted proximally from the anchor such that the anchor transitions from a compressed state to an expanded state.

In some implementations, a distal part of the shaft bifurcates into a first branch coupled to the interface, and a second branch coupled to the anchor receiver.

In some implementations, the step of advancing can include transluminally advancing the first branch and the second branch of the distal part of the shaft alongside each other within the catheter.

In some implementations, the method further includes sterilizing the implant, the shaft and the catheter.

In accordance with some implementations, a system and/or an apparatus includes an implant for use with a valve of a heart of a subject, the valve defining an annulus and having a first leaflet and an opposing leaflet, the heart having a chamber upstream of the valve.

In some implementations, the implant includes a wing extending from a root portion of the wing to a tip portion of the wing.

In some implementations, the wing has a compressed state and/or an expanded state. In some implementations, the wing is biased to expand into an expanded state. In some implementations, the wing is mechanically expandable to transition into an expanded state.

In some implementations, an interface, connected to the root portion of the wing, is configured to be anchored to tissue of the annulus such that the wing extends over the first leaflet toward the opposing leaflet.

In some implementations, the implant is configured such that: while the wing is in the compressed state, the implant has a hinged coupling between the root portion and the interface that facilitates articulation, at the hinged coupling, of the root portion with respect to the interface. In some such implementations, expansion of the wing into the expanded state inhibits the articulation by restraining the hinged coupling.

In some implementations, the implant is sterile.

In accordance with some implementations, a system includes an implant for use with a valve of a heart of a subject, the valve defining an annulus and having a first leaflet and an opposing leaflet, the heart having a chamber upstream of the valve.

In some implementations, the implant includes a wing extending from a root portion of the wing to a tip portion of the wing, and/or an interface. In some such implementations, the system includes an anchor and/or a delivery tool.

In some implementations, the delivery tool includes a catheter, transluminally advanceable to the chamber, and configured to house the implant while the wing is in a compressed state. In some implementations, the delivery tool includes a shaft, engaged with the interface, and configured, via the engagement with the interface, to deploy the implant out of the catheter and into the chamber.

In some implementations, the delivery tool includes a driver configured to secure the implant in the chamber, in the expanded state, by using the anchor to anchor the interface to tissue of the heart.

In some implementations, the implant is configured such that while the wing is in the compressed state, the root portion has a hinged coupling to the interface that facilitates articulation, at the hinged coupling, of the root portion with respect to the interface.

In some implementations, the wing is biased to expand into an expanded state upon being deployed, and/or, and/or expansion of the wing into the expanded state inhibits the articulation by restraining the hinged coupling.

In some implementations, at least one of the implant, the anchor, and the delivery tool is sterile.

In accordance with some implementations, a system includes an implant or device for use with a valve of a heart of a subject, the valve defining an annulus and having a first leaflet and an opposing leaflet, the heart having a chamber upstream of the valve.

In some implementations, the implant/device includes a wing extending from a root portion of the wing to a tip portion of the wing. In some implementations, the wing has a compressed state and/or an expanded state. In some implementations, the wing is biased to expand into an expanded state. In some implementations, the wing can be actuated to expand into an expanded state.

In some implementations, the implant/device includes an annular support, connected to the root portion of the wing.

In some implementations, the implant/device is configured such that: in the compressed state of the wing, the wing has a hinged coupling to the annular support that facilitates articulation, at the hinged coupling, of the wing with respect to the annular support. In some such implementations, expansion of the wing toward the expanded state inhibits the articulation by restraining the hinged coupling.

In some implementations, the implant is sterile.

In some implementations, the wing defines a contact face, and an opposing face opposite to the contact face.

In some implementations, the system further includes: an anchor, and/or a delivery tool. In some implementations, the delivery tool includes a catheter, transluminally advanceable to the chamber with the implant housed in the catheter while the wing is in the compressed state.

In some implementations, the delivery tool comprises a driver, configured to deploy the implant out of the catheter such that, within the chamber, the wing assumes the expanded state. In some implementations, the driver is configured to position the implant in a position in which: the wing extends over the first leaflet toward the opposing leaflet, and/or the contact face faces the first leaflet.

In some implementations, the implant/device defines an interface. In some implementations, the driver is configured to, while the implant is positioned in the position and the wing is in the expanded state, secure the interface to the annulus by driving the anchor through the interface and into tissue of the annulus.

In some implementations, the annular support is shaped such that, while the implant is secured to the annulus and the wing is in the expanded state, the annular support is disposed against an atrial surface of the annulus such that, the restrained hinged coupling inhibits deflection of the root portion of the wing with respect to the annulus.

In some implementations: the interface is a first interface; the annular support includes a first annular arm that extends away from the hinged coupling to the first interface; and/or the annular support further includes a second annular arm that: is coupled to the hinged coupling, and/or extends away from the hinged coupling to a second interface.

In some implementations, the first annular arm is joined to the second annular arm, at the hinged coupling.

In some implementations, the implant/device is configured such that: the hinged coupling includes a sleeve defining an aperture, and while the wing is in the compressed state, a thin portion of the annular support is disposed within the aperture.

In some implementations, the implant/device is configured such that expansion of the wing toward the expanded state slides the sleeve from the thin portion to a thick portion of the annular support, the thick portion having a cross-section that is dimensioned to restrain the hinged coupling by fitting the aperture.

In some implementations, the thick portion of the annular support has an oblong cross-section that is dimensioned to restrain the hinged coupling by fitting the aperture.

In some implementations, the sleeve is a first sleeve defining a first aperture, and the hinged coupling further includes a second sleeve defining a second aperture.

In some implementations, the annular support includes a pair of annular arms, each annular arm having: a thin portion at which the annular arms are joined, and/or a thick portion that extends away from the thin portion.

In some implementations, expansion of the wing toward the expanded state slides each sleeve from the thin portion to the thick portion of a respective annular arm, thereby restraining the hinged coupling.

In some implementations, the annular support includes an expansion element having: a compact state, and/or an extended state in which the expansion element resists compression of the wing toward the compressed state.

In some implementations, the annular support is configured such that extension of the expansion element from the compact state to the extended state applies an expansion force to the wing, the expansion force facilitating expansion of the wing from the compressed state to the expanded state.

In some implementations, the expansion element is configured to resist transition from the extended state toward the compact state.

In some implementations, the expansion element includes a spring.

In some implementations, the expansion element includes a plurality of subunits, and the expansion element is configured such that extending the expansion element into the extended state causes the subunits to fit together.

In some implementations, the expansion element includes a plurality of subunits, configured to lock together upon the expansion element assuming the extended state.

In some implementations, the expansion element is straighter in the extended state than in the compact state.

In some implementations, the expansion element includes a hinge, and the expansion element is configured such that straightening the hinge straightens the expansion element.

In some implementations, the delivery tool further includes an extension actuator, the extension actuator configured to transluminally extend the expansion element from the compact state to the extended state.

In accordance with some implementations, a system includes an implant/device for use with a valve of a heart of a subject, the valve having a first leaflet and an opposing leaflet, the heart having an upstream chamber upstream of the valve and a downstream chamber downstream of the valve. In some implementations, the implant/device is transitionable between a compressed state and an expanded state.

In some implementations, the implant/device includes a wing extending from a root portion of the wing to a tip portion of the wing. In some implementations, the wing defines a contact face, and an opposing face opposite to the contact face.

In some implementations, the implant/device and/or wing includes one or more arms (e.g., one arm, a pair of arms, three arms, etc.). In some implementations, each arm of the one or more arms is: fastened to the tip portion of the wing, and/or extending away from the wing and the other arm of the pair to define a lateral portion of the arm that is disposed laterally from the wing.

In some implementations, the system includes a delivery tool including a catheter, transluminally advanceable to the chamber, the catheter housing the implant while the implant is in the compressed state.

In some implementations, the system (e.g., the delivery tool, etc.) is configured to deploy the implant out of the catheter and position the implant in a position in which: the implant is in its expanded state, the wing extends, from the root portion, over the first leaflet toward the opposing leaflet.

In some implementations, the system (e.g., the delivery tool, etc.) is configured to deploy the implant out of the catheter and position the implant in a position in which: the implant is in its expanded state, the wing extends, from the root portion, over the first leaflet toward the opposing leaflet, the lateral portion of each arm presses, at a respective lateral site lateral from the wing, in an upstream direction against a downstream side of the first leaflet in a manner that presses the contact face against an upstream side of the first leaflet between the lateral sites.

In some implementations, at least one of the implant and the delivery tool is sterile.

In some implementations: the catheter is configured to house the implant while the implant is in the compressed state, and/or the implant includes a flexible frame biased to expand the implant into the expanded state upon deployment from the catheter.

In some implementations, the delivery tool further includes a shaft, reversibly engageable to the implant and configured to: deploy the implant from the catheter, position the implant in the position, and/or while the implant is in the position, release the implant.

In some implementations, the implant is configured to maintain the position upon the shaft releasing the implant, while the implant is in the position, by pinching the first leaflet between the wing and the lateral portion of each arm.

In some implementations, while the implant is positioned in the position: the lateral portion of each arm presses, at a respective lateral site lateral from the wing, in an upstream direction against a downstream side of the first leaflet in a manner that presses the contact face of the root portion of the wing against an upstream side of the first leaflet between the lateral sites, and/or the tip portion of the wing deflects in concert with the lateral portion of each arm and with tissue of the lateral sites, responsively to a cardiac cycle of the heart, in a reciprocating manner, in the upstream direction and in the downstream direction.

In some implementations, while the implant is positioned in the position, the pressing of the contact face of the root portion of the wing, against the upstream side of the first leaflet between the lateral sites, inhibits deflection of the root portion in the upstream direction.

In some implementations, while the implant is positioned in the position, the pressing of the contact face of the root portion of the wing, against the upstream side of the first leaflet between the lateral sites defines a deflection-limit of the wing during the cardiac cycle by inhibiting deflection of the wing in the upstream direction beyond the deflection-limit.

In accordance with some implementations, a system for use with a heart of a subject includes: an anchor; an implant/device, including an interface; and/or a delivery tool. In some implementations, the delivery tool includes a driver, engaged with the anchor.

In some implementations, the delivery tool includes a shaft including: a coupling engageable to the interface, and/or a lock at a distal portion of the shaft.

In some implementations, the lock includes a first unit and a second unit, the lock having a locked state in which the first unit is mated with the second unit, and/or having an unlocked state in which the first unit is separated and translatable away from the second unit.

In some implementations, the delivery tool is configured to, while the lock is locked, via engagement of the coupling with the interface, position the implant within the heart, and/or use the driver to secure the implant to tissue of the heart by driving the anchor into the tissue.

In some implementations, the delivery tool is configured to reversibly and repeatedly transition the lock between the locked state and the unlocked state; and/or while the implant remains secured to the tissue, disengage the coupling from the interface.

In some implementations, at least one of the implant, the anchor, and the delivery tool is sterile.

In some implementations, the coupling is configured to remain engaged to the interface while the lock transitions between the locked state and the unlocked state.

In some implementations, the delivery tool is configured to disengage the coupling from the interface while the lock is locked.

In some implementations, the delivery tool is configured to disengage the coupling from the interface while the lock is unlocked.

In some implementations, the distal portion of the shaft, at which the lock is disposed, is disposed proximally of the coupling.

In some implementations, a distal end portion of the shaft defines the coupling.

In some implementations, transitioning the lock from the locked state to the unlocked state, while the coupling remains engaged to the interface and the implant remains secured to the tissue, facilitates deflection of the implant responsively to a cardiac cycle of the heart.

In some implementations: the heart has a valve having a first leaflet and an opposing leaflet, and/or a chamber upstream of the valve, and the implant includes a wing defining a contact face and an opposing face opposite to the contact face. In some implementations, the shaft can be configured, via engagement of the coupling to the interface, to position the implant in a position in which the wing extends over the first leaflet toward the opposing leaflet.

In some implementations, the system can be configured such that transitioning the lock from the locked state to the unlocked state, while the implant remains in the position, the coupling remains engaged to the interface and the implant remains secured to the tissue, facilitates deflection of the wing, in an upstream direction and in a downstream direction, responsively to the cardiac cycle.

In some implementations: the interface of the implant is disposed at a root portion of the wing, and the wing extends, away from the interface to a tip portion of the wing, over the first leaflet toward the opposing leaflet.

In some implementations, the delivery tool is configured to use the driver to secure the root portion of the wing to tissue of the chamber by driving the anchor through the interface and into the tissue of the chamber.

In some implementations, the delivery tool is configured to transition the lock from the locked state to the unlocked state while the root portion of the wing remains secured to the tissue of the chamber, in a manner that facilitates deflection of the tip portion of the wing, in the upstream direction and in the downstream direction, responsively to the cardiac cycle.

In some implementations, the system includes a pair of anchors. In some implementations, the interface is a first interface, and the implant further includes a second interface. In some implementations, the coupling includes: a first branch engageable to the first interface along a first-branch axis, and/or a second branch engageable to the second interface, along a second-branch axis that is nonparallel to the first axis.

In some implementations, the delivery tool includes a pair of drivers, each driver configured to drive one of the anchors along a respective axis, through a respective interface and into the tissue.

In some implementations, a distal end portion of the shaft defines the coupling, and/or bifurcates into the first branch and the second branch, distally of the lock.

In some implementations, while the lock is in the locked state, the coupling is secured to a proximal portion of the shaft. In some implementations, while the lock is in the unlocked state, the coupling is released from the proximal portion of the shaft.

In some implementations, the delivery tool includes a catheter, configured to house the implant and the shaft such that the first branch of the coupling and the second branch of the coupling are oriented along a proximal shaft axis of the proximal portion of the shaft.

In some implementations, the coupling includes a shape-memory material that biases the first branch and the second branch to flex away from each other upon release from the catheter.

In some implementations, the first branch and the second branch are configured to flex away from each other upon release from the catheter such that the first-branch axis and the second-branch axis are each oblique to the proximal shaft axis.

In some implementations, the delivery tool includes a tether, and/or can be configured such that intracardially adjusting tension on the tether transitions the lock between the locked state and the unlocked state.

In some implementations, the delivery tool is configured such that while the lock is in the unlocked state: the first unit is separated from the second unit, and/or the tether connects the first unit to the second unit.

In accordance with some implementations, a method (e.g., usable or for use with tissue of a real or simulated heart of a subject (e.g., of a living subject or of a simulation)) can include transluminally advancing to the heart, within a catheter: (i) an implant including an interface and/or (ii) a shaft coupled to the interface. In some implementations, the shaft includes a lock at a distal portion of the shaft, the lock having a first unit and a second unit.

In some implementations, the method includes, while the lock is locked such that the first unit is mated with the second unit: deploying the implant out of the catheter, and/or using a driver engaged with an anchor, securing the interface to the tissue by driving the anchor through the interface and into the tissue.

In some such implementations, the method includes unlocking the lock such that the first unit is separated from the second unit, disengaging the coupling from the interface, and/or withdrawing the shaft from the subject.

In some implementations, the method further includes sterilizing the implant, the anchor, the shaft and the catheter.

In some implementations, the step of disengaging includes disengaging the coupling from the interface while the lock remains unlocked.

In some implementations, the method further includes, prior to the step of disengaging, relocking the lock such that the first unit is mated with the second unit.

In some implementations, the step of disengaging includes disengaging the coupling from the interface while the lock remains locked.

In some implementations: the shaft includes a tether, adjustably coupled to the lock, and/or the step of unlocking includes unlocking the lock by intracardially reducing tension upon the tether.

In some implementations, the method further includes, prior to the step of disengaging, relocking the lock, such that the first unit is mated with the second unit, by increasing tension on the tether.

In some implementations, the method further includes, prior to the step of disengaging, and while the lock remains unlocked, assessing function of the implant.

In some implementations, the step of assessing includes assessing function of the implant while the driver remains engaged with the anchor. In some implementations, the method further includes, prior to the step of assessing, disengaging the driver from the anchor.

In some implementations, the step of assessing includes assessing deflection of the implant responsively to a cardiac cycle of the heart.

In some implementations: the heart has: a valve having a first leaflet and an opposing leaflet, and/or a chamber upstream of the valve, and/or the implant includes a wing defining a contact face and an opposing face opposite to the contact face.

In some implementations, the step of securing includes securing the interface to tissue of the chamber by driving the anchor through the interface and into the tissue of the chamber.

In some implementations, the step of unlocking includes facilitating deflection of the wing, in an upstream direction and in a downstream direction, responsively to the cardiac cycle. In some implementations, the step of assessing includes assessing deflection of the wing in the upstream direction and in the downstream direction, responsively to the cardiac cycle.

In some implementations: the interface is disposed at a root portion of the wing, and the step of securing includes securing the root portion of the wing to the tissue of the chamber by driving the anchor through the interface and into the tissue of the chamber. In some implementations, this is done such that the wing extends, away from the interface to a tip portion of the wing, over the first leaflet toward the opposing leaflet.

In some implementations, the step of unlocking includes unlocking the lock while the root portion of the wing remains secured to the tissue of the chamber, thereby facilitating deflection of the tip portion of the wing, in the upstream direction and in the downstream direction, responsively to the cardiac cycle.

In some implementations, the step of assessing includes assessing deflection of the tip portion of the wing in the upstream direction and in the downstream direction, responsively to the cardiac cycle.

In some implementations, the step of disengaging and the step of withdrawing include, responsively to the step of assessing: disengaging the coupling from the interface, and/or withdrawing the shaft from the subject.

In some implementations, the method further includes, prior to the step of disengaging, relocking the lock such that the first unit is mated with the second unit.

In some implementations, the method further includes, prior to the step of disengaging and responsively to the step of assessing: using the driver, removing the anchor from the tissue; using the shaft, repositioning the implant; and/or repeating the step of securing and the step of assessing.

In accordance with some implementations, a system includes an implant or device for use with a valve of a heart of a subject, the valve having a first leaflet and an opposing leaflet, the heart having a chamber upstream of the valve. In some implementations, the implant/device includes: an interface, a flexible wing, coupled to the interface. In some implementations, the flexible wing has a contact face and an opposing face opposite the contact face.

In some implementations, a beam is connected to (and/or integral with) the wing along a part of the wing. In some implementations, a line is coupled to the beam such that tensioning the line strains the beam.

In some implementations, the system includes an anchor and/or a delivery tool. In some implementations, the delivery tool includes a catheter, transluminally advanceable to the chamber, and configured to house the implant.

In some implementations, the delivery tool includes a shaft, engaged with the interface, and configured, via the engagement with the interface, to: deploy the implant out of the catheter such that, within the chamber, the wing extends away from the interface, and/or position the implant in a position in which the interface is at a site in the heart, the wing extends over the first leaflet toward the opposing leaflet, and the contact face faces the first leaflet.

In some implementations, a driver is engaged with the anchor and configured to secure the implant in the position by using the anchor to anchor the interface to tissue of the heart.

In some implementations, the delivery tool can be configured to intracardially reshape the wing by straining the beam by tensioning the line.

In some implementations, at least one of the implant, the anchor, and the delivery tool is sterile.

In some implementations, the line extends from a proximal portion of the line at a proximal portion of the delivery tool to a distal portion of the line coupled to the beam.

In some implementations, the proximal portion of the delivery tool can be configured to intracardially reshape the wing by straining the beam by tensioning the proximal portion of the line.

In some implementations, the line extends from the proximal portion of the line, through the interface and to the distal portion of the line.

In some implementations, the wing defines a root portion at which the interface is disposed, and a tip portion along which the beam is connected to the wing.

In some implementations, the beam is connected to the wing along a portion of a perimeter of the tip portion of the wing.

In some implementations, the line extends from a proximal portion of the line at a proximal portion of the delivery tool to the beam at the tip portion of the wing.

In some implementations, the proximal portion of the delivery tool is configured to intracardially reshape the tip portion of the wing by straining the beam by tensioning the proximal portion of the line.

In some implementations, the delivery tool is configured to intracardially change a radius of curvature of the wing along a normal plane of the wing extending from the root portion to the tip portion by tensioning the line.

In some implementations, the delivery tool is configured to intracardially increase the radius of curvature of the wing along the normal plane of the wing extending from the root portion to the tip portion by tensioning the line.

In some implementations, the delivery tool is configured to intracardially decrease the radius of curvature of the wing along the normal plane of the wing extending from the root portion to the tip portion by tensioning the line.

In some implementations: the beam is a rigid beam, and/or the beam is connected to the wing along the portion of the perimeter of the tip portion of the wing such that the tip portion has a greater stiffness, along the normal plane of the wing extending from the root portion to the tip portion, than the root portion.

In some implementations, a distal portion of the line is generally parallel to the normal plane of the wing extending from the root portion to the tip portion.

In some implementations, the delivery tool is configured to intracardially reshape the wing by reshaping the beam by tensioning the line.

In some implementations: the wing includes a braided mesh, and/or the delivery tool is configured to intracardially reshape the wing by reorienting a weave of the braided mesh by reshaping the beam by tensioning the line.

In some implementations, the line is a first line connected to a first portion of the beam, the implant further includes a second line, connected to a second portion of the beam, and/or tensioning the first line and the second line reshapes the wing by changing a distance between the first portion of the beam and the second portion of the beam.

In some implementations, the delivery tool is configured to intracardially change a width of the wing by changing a radius of curvature of the beam by tensioning the line.

In some implementations, the delivery tool is configured to intracardially reduce the width of the wing by decreasing the radius of curvature of the beam by tensioning the line.

In accordance with some implementations, a method (e.g., usable or for use with simulated tissue of a real or simulated heart of a subject (e.g., living subject or simulation)) can include transluminally advancing to the heart, within a catheter: (i) an implant including an interface and an anchor receiver, and/or (ii) a shaft coupled to the interface.

In some implementations, the method can include, using the shaft, deploying the implant out of a distal opening of the catheter such that the interface and the anchor receiver are disposed against a surface of the tissue.

In some implementations, the method can include anchoring the implant to the tissue by driving an anchor through the interface and a surface of the tissue, along a curved path within the tissue, such that a distal part of the anchor exits the tissue and is received by the anchor receiver.

Any of the above method(s) and any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. in this summary can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

The concepts herein will be more fully understood from the following detailed description of implementations thereof, taken together with the drawings, in which:

### BRIEF DESCRIPTION OF THE DRAWINGS

FIGS. 1, 2A-B and 3A-E are schematic illustrations showing a system comprising a delivery tool and an implant, including aesthetic features thereof, in accordance with some implementations;

FIGS. 4-5 are schematic illustrations showing implants that define lateral flaps, including aesthetic features thereof, in accordance with some implementations;

FIGS. 6-7 are schematic illustrations showing implants that comprise a first wing and a second wing, including aesthetic features thereof, in accordance with some implementations;

FIGS. 8A-B, 9A-B and 10A-B are schematic illustrations showing implants that comprise extension elements, including aesthetic features thereof, in accordance with some implementations;

FIGS. 11-12 are schematic illustrations showing distal portions of delivery tools, including aesthetic features thereof, in accordance with some implementations;

FIGS. 13A-G are schematic illustrations showing use of a distal portion a delivery tool to implant a ratcheting interface of an implant to tissue, including aesthetic features thereof, in accordance with some implementations;

FIGS. 14-18 are schematic illustrations showing frames of implants, including aesthetic features thereof, in accordance with some implementations;

FIG. 19 shows schematic illustrations of an implant comprising a wing comprising a mesh, including aesthetic features thereof, in accordance with some implementations;

FIGS. 20-26, are schematic illustrations showing implants that comprise flex elements, including aesthetic features thereof, in accordance with some implementations;

FIGS. 27-30 are schematic illustrations showing implants comprising tip portions and root portions that can articulate in relation to each other, including aesthetic features thereof, in accordance with some implementations;

FIGS. 31-44 are schematic illustrations showing implants that comprise a limiter, including aesthetic features thereof, in accordance with some implementations;

FIG. 45 shows schematic illustrations of an implant comprising a wing that defines a deflection-limit of the wing, including aesthetic features thereof, in accordance with some implementations;

FIGS. 46A-B, 47-48 and 49A-B, 50-52, 53, 54A-B, 55A-B and 56-58 are schematic illustrations showing implants, including aesthetic features thereof, that comprise annular arms, in accordance with some implementations;

FIGS. 59A-C, 60A-B, 61A-B and 62A-B are schematic illustrations showing implants, including aesthetic features thereof, that comprise adjustable limiters, in accordance with some implementations;

FIGS. 63A-63B, 64A-64B, 65 and 83A-B are schematic illustrations showing implants, including aesthetic features thereof, that comprise tethers, in accordance with some implementations;

FIGS. 66A-B, 67A-B, 68-69, 70A-B and 71 are schematic illustrations showing implants, including aesthetic features thereof, that each comprise a leg, in accordance with some implementations;

FIGS. 72, 73A-C, 74A-D and 75A-D are schematic illustrations showing implants, including aesthetic features thereof, comprising limiters that are adjustable by changing tension on a tether, in accordance with some implementations;

FIGS. 76A-B, 77A-B, 78A-B, 79A-C, 80A-C, 81A-D, 82A-B and 84A-B are schematic illustrations showing adjustable implants, including aesthetic features thereof, in accordance with some implementations;

FIGS. 85A-E, 86A-C, 87A-D and 88A-C are schematic illustrations showing adjustable shafts, including aesthetic features thereof, in accordance with some implementations;

FIGS. 89A-C are schematic illustrations showing an implant and an anchor, including aesthetic features thereof, in accordance with some implementations;

FIGS. 90A-C, 91A-D, 92A-B and 93A-E are schematic illustrations showing use of delivery tools to deploy implants, including aesthetic features thereof, in accordance with some implementations;

FIGS. 94A-E, 95, 96A-E and 97A-C are schematic illustrations showing use of anchors for use with drivers, including aesthetic features thereof, in accordance with some implementations;

FIG. 98 shows schematic illustrations of a system for driving anchors, including aesthetic features thereof, in accordance with some implementations;

FIGS. 99-102 are schematic illustrations showing implants, including aesthetic features thereof, in accordance with some implementations;

FIGS. 103A-C are schematic illustrations showing implantation of an implant, including aesthetic features thereof, in accordance with some implementations;

FIGS. 104A-B are schematic illustrations showing an implant, including aesthetic features thereof, in accordance with some implementations;

FIGS. 105A-E and 106A-E, which are schematic illustrations showing a distal portion of a system for delivering an implant to the heart, including aesthetic features thereof, in accordance with some implementations;

FIGS. 107, 108A-B and 109A-B, are schematic illustrations showing use of an adjustable implant, including aesthetic features thereof, in accordance with some implementations; and

FIGS. 110A-C and 111A-B are schematic illustrations showing implants, including aesthetic features thereof, in accordance with some implementations.

### DETAILED DESCRIPTION

The described systems, apparatuses, devices, methods, etc. should not be construed as limiting in anyway. Instead, the present disclosure is directed toward all novel and nonobvious features and aspects of the various disclosed implementations and applications, alone and in various combinations and sub-combinations with one another. The disclosed systems, apparatuses, devices, methods, etc. are not limited to any specific aspect, feature, or combination thereof, nor do the disclosed systems, apparatuses, devices, methods, etc. require that any one or more specific advantages be present or problems be solved.

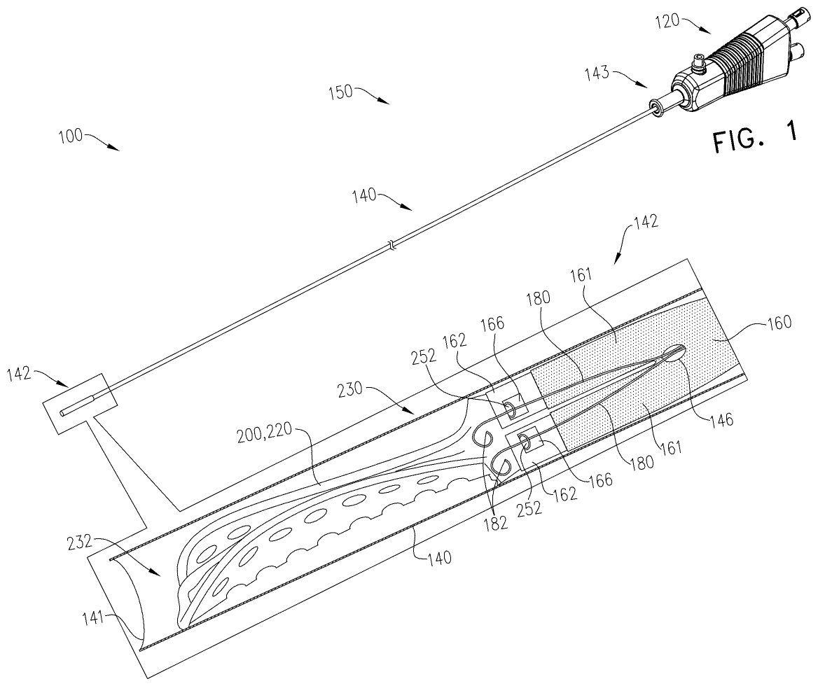

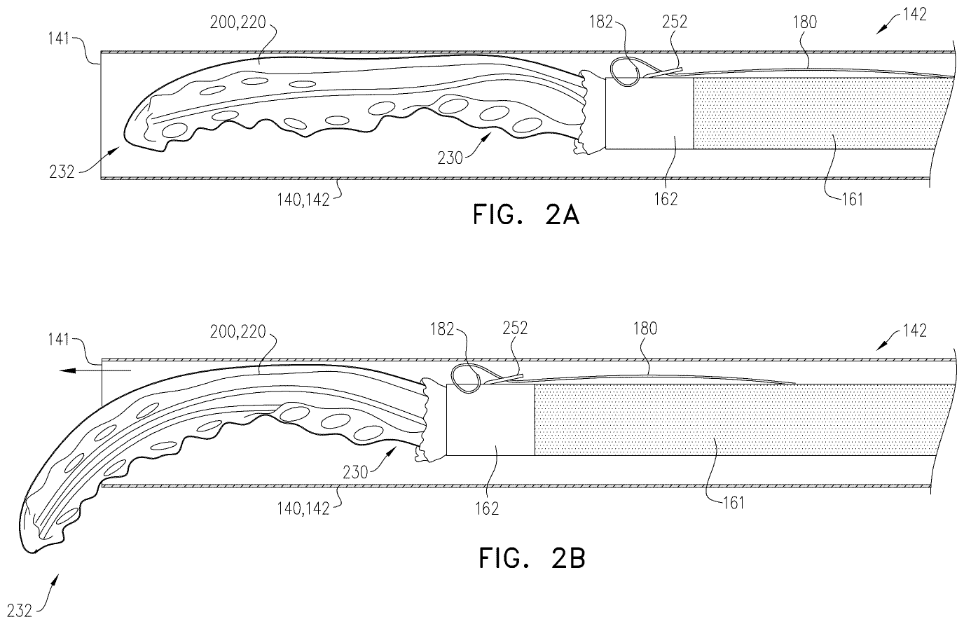

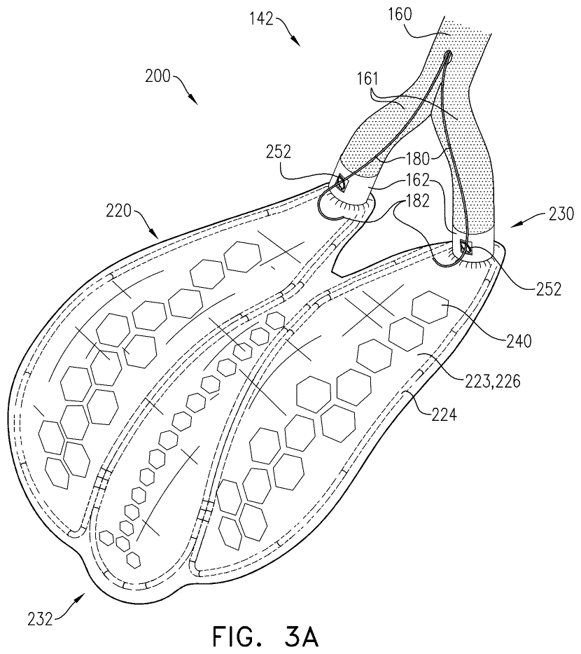

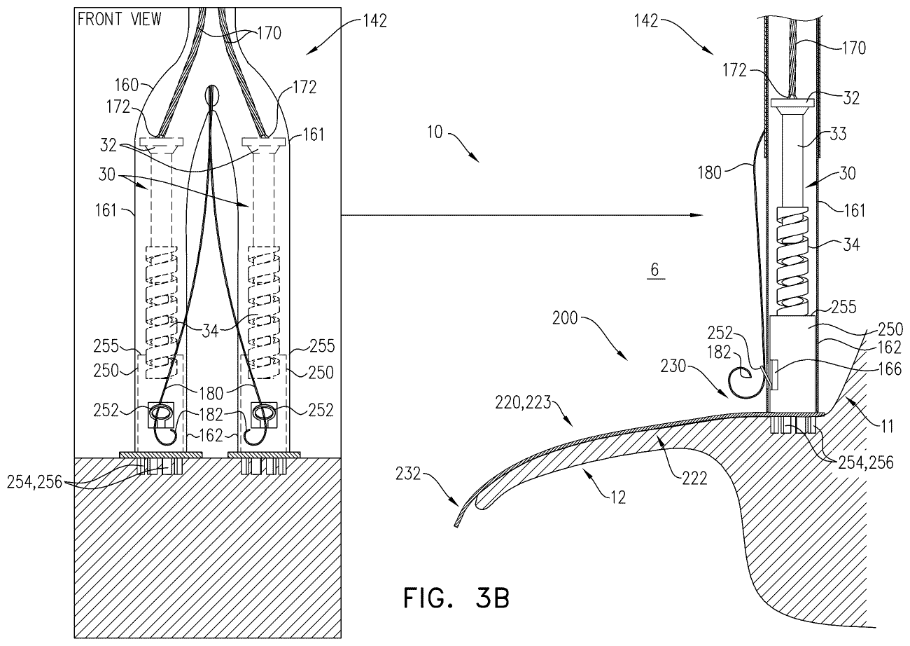

Reference is made to FIGS. 1, 2A-B and 3A-E, which are schematic illustrations showing a system 100 comprising a delivery tool 150 and an implant 200, in accordance with some implementations. As described hereinbelow, delivery tool 150 can be used to deliver implant 200 to a heart (e.g., to a native valve 10 thereof) of a subject (e.g., a living subject, a simulation, etc.).

As shown, delivery tool 150 can comprise a controller 120 for transluminally operating and/or steering the tool, e.g., from outside of the subject. Controller 120 can therefore be disposed at a proximal (e.g., extracorporeal) portion 143 of the tool, and a delivery catheter 140 extends from the proximal portion to a distal portion 142 of the tool. In some implementations, catheter 140 houses a shaft 160 that extends, within the catheter, from proximal portion 143 to distal portion 142.

In some implementations, catheter 140 is itself steerable (e.g., can include one or more pullwires that can be tensioned by controller 120 to bend a distal portion of the catheter). In some implementations, catheter 140 is configured to be passively steered, e.g., by being advanced within and/or through an outer catheter (not shown).

In some implementations, and as shown in the inset of FIG. 1, shaft 160 bifurcates at a distal portion of the shaft, into two branches 161 that are disposed, alongside each other, within catheter 140 at distal portion 142 of tool 150. In some implementations, each branch 161 is narrower than portions of shaft 160 that are proximal from the branches. In some implementations, and as shown in greater detail in the inset of FIG. 3B, shaft 160 (e.g., proximal from branches 161) is narrower than the combined widths of branches 161. This arrangement can advantageously allow shaft 160 to be advanced to the heart within a catheter 140 that, along most of the catheter's length (e.g., from controller 120 to distal portion 142 of delivery tool 150), is narrower than the distal portion of the tool.

As shown, each branch 161 of shaft 160 can be engaged with implant 200. In some implementations, and as shown in greater detail in FIG. 3B, each branch 161 is engaged, at a distal end portion 162 thereof, to a corresponding interface or anchor receiver 250 of implant 200. In some implementations, and as shown, each distal end portion 162 defines a window 166 through which a ring 252, defined by respective interfaces 250, protrudes.

In some implementations, one or more ripcords 180 extend from proximal portion 143 (e.g., from controller 120) to distal portion 142, where they maintain engagement between shaft 160 (e.g., branches 161 thereof) and interfaces 250. In some implementations, and as shown, ripcords 180 can extend within shaft 160, and out of an opening 146 defined by the shaft. For example, and as shown, ripcords 180 can extend distally from opening 146 and through rings 252, such that a distal portion 182 of each ripcord is disposed distally of respective rings, reversibly securing implant 200 to shaft 160. Ripcords 180 can be retracted transluminally (e.g., extracorporeally, using controller 120) to release implant 200 from shaft 160, as described hereinbelow with reference to FIG. 3D. In some implementations, a single common ripcord is provided, which branches at its distal end so as to serve as two ripcords.

Implant 200 comprises a flexible wing 220 that is shown in FIGS. 1 and 2A compressed (e.g., assuming a compressed state) within catheter 140, for delivery to the heart. As shaft 160 is slid distally within catheter 140 (FIG. 2B), wing 220 exits a distal end 141 of the catheter, which allows the wing to progressively expand to an expanded state shown in FIG. 3A. As shown, wing 220 extends from a root portion 230 of the wing, at which interfaces 250 are located, to a tip portion 232 of the wing.

In some implementations, and as shown, wing 220 comprises a flexible frame 224 (e.g., a wire frame, such as a hollow, tubular wire frame) that provides mechanical support to the wing.

In some implementations, frame 224 comprises a shape-memory material (e.g., a shape-memory wire) that biases the wing toward assuming the expanded state, e.g., upon being exposed from catheter 140.

In some implementations, frame 224 can be configured to be mechanically expandable such that it can be actuated to transition to the expanded state.

In some implementations, and as shown, wing 220 comprises a flexible sheet 226. In some implementations, and as shown, wing 220 can comprise sheet 226 and frame 224, with the sheet covering the frame. In some implementations, sheet 226 defines holes 240 therethrough that facilitate flow of blood through the sheet, and therefore through the wing.

FIG. 3B shows wing 220 in the expanded state and having been positioned using shaft 160 within an atrium 6 of the heart. As shown, interfaces 250 are positioned at an annulus 11 of a mitral valve 10 of the heart, such that wing 220 extends over a native leaflet (e.g., a posterior leaflet 12) toward an opposing leaflet (e.g., an anterior leaflet 14). With wing 220 so positioned, a first face or contact face 222 of the wing faces (e.g., contacts) posterior leaflet 12, and a second face or opposing face 223 faces atrium 6.

In some implementations, and as shown in FIG. 3B, tool 150 comprises a pair of drivers 170 that extend from proximal portion 143, through shaft 160 and to distal portion 142. Each driver, at a distal end thereof, has a drive head 172 that engages a corresponding anchor 30 (e.g., with an anchor head 32 thereof). As shown, drive heads 172 and/or anchors 30 are disposed at (e.g., within) corresponding branches 161. System 100 can be provided in this configuration, e.g., implant 200 is advanced by tool 150 with drivers 170 and/or anchors 30 in this position.

As shown in FIG. 3C, while each interface 250 is disposed at annulus 11, drivers 170 are used to secure implant 200 to annular tissue by advancing (e.g., screwing) each anchor 30 through an interface 250, such that a portion (e.g., a helical tissue-engaging element 34) of each anchor exits a distal end portion 162 of one of the shaft's branches 161 and enters the annular tissue. In some implementations, and as shown, helical tissue-engaging element 34 advances through annular tissue until an anchor head 32 abuts interface 250, securing the interface—and thereby implant 200—to the tissue. Thus, each interface 250 can be considered to serve as an anchor receiver. In some implementations, and as shown, interface 250 is tubular, having a proximal end and a distal end. In some implementations, anchor head 32 can press against the proximal end of the tubular interface. In some implementations, and as shown, interface 250 defines teeth 254, and the pressing that anchor head 32 applies to the interface can advantageously cause the teeth to protrude into the tissue.

As shown in FIG. 3D, after interface 250 is anchored to the annular tissue, ripcords 180 can then be retracted (e.g., pulled using controller 120), thereby releasing distal end portions 162 of the shaft's branches 161 from respective interfaces 250. Distal portion 142 of delivery tool 150 can then be retracted, thereby withdrawing catheter 140 and shaft 160 from the subject while implant 200 remains secured to the annular tissue (FIG. 3E).

In some implementations, drive heads 172 remain within branches 161 during withdrawal of tool 150.

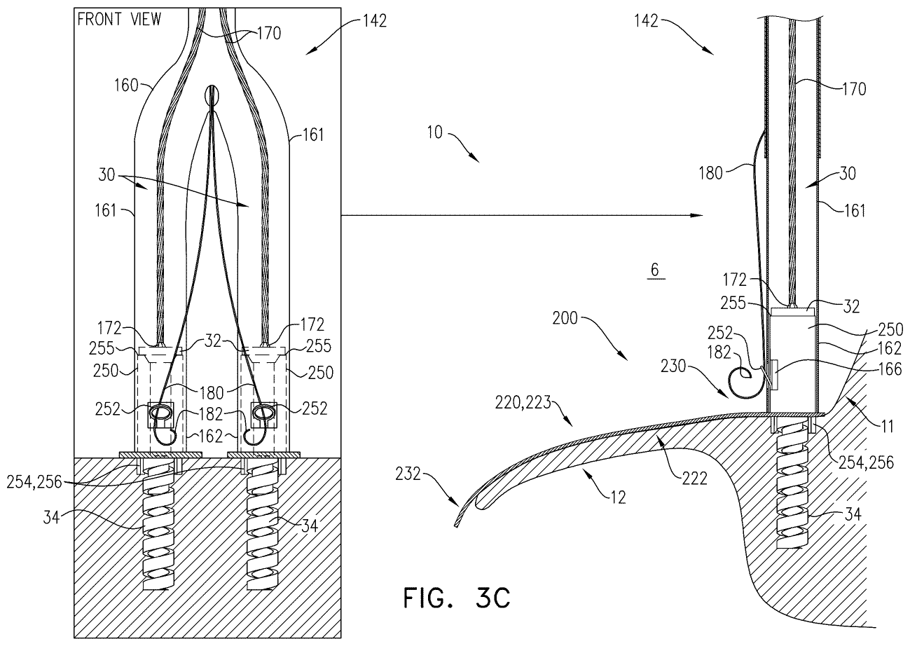

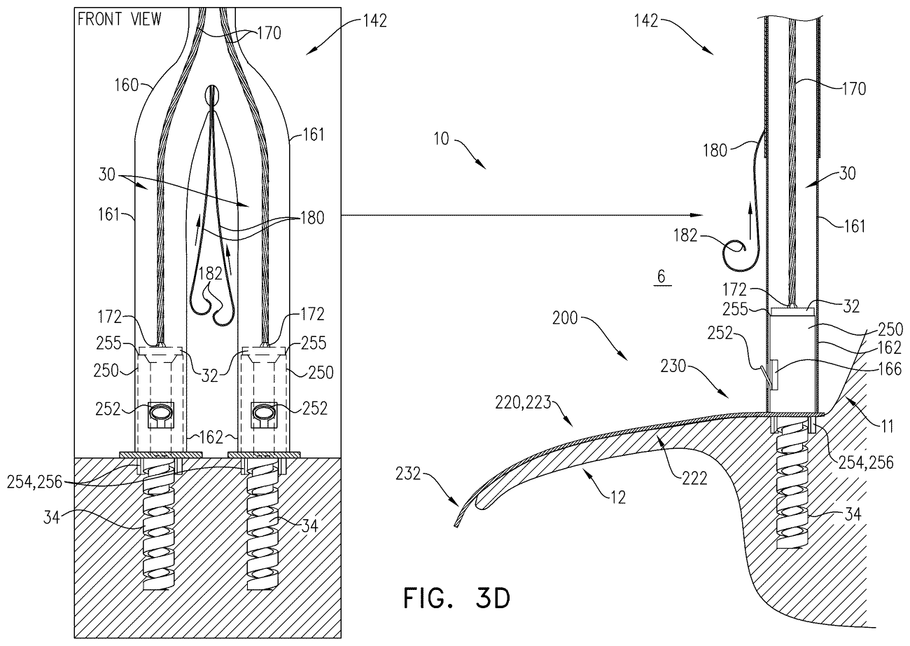

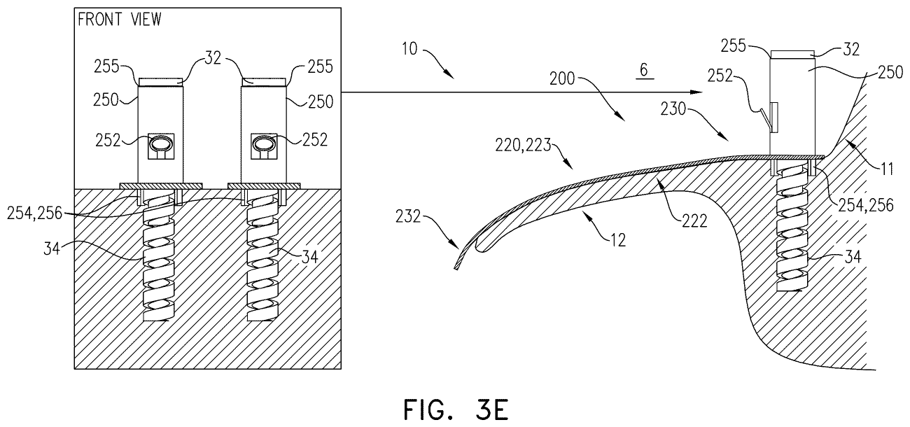

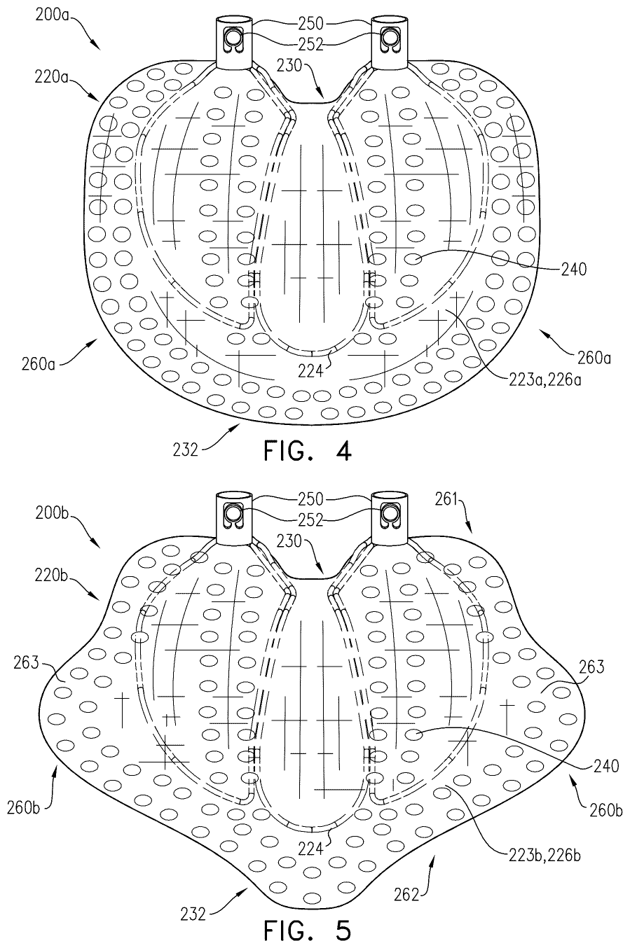

Reference is made to FIGS. 4-5, which are schematic illustrations showing implants 200a, 200b in accordance with some implementations. Implants 200a, 200b can be considered to be variants of implant 200, and can be similar, at least in their general purpose, e.g., being anchored to a tissue of the heart so as to repair the function of the native leaflet, to implant 200 disclosed hereinabove, mutatis mutandis, except that flexible sheets 226a, 226b of implants 200a, 200b each define lateral flaps 261a, 261b.

In some implementations, flexible sheets 226a, 226b of implants 200a, 200b are shaped such that, when the implants are positioned at mitral valve 10 as shown in FIG. 3B, lateral flaps 260a, 260b extend laterally over posterior leaflet 12, e.g., bilaterally, toward respective commissures of the valve. In some implementations, wings 220a, 220b are more flexible at lateral flaps 260a, 260b than at a medial region 264a, 264b in which the frame is disposed. The greater flexibility of lateral flaps 260a, 260b facilitates fitting of the lateral flaps into the commissures, and in some implementations, the lateral flaps can be curved so as to further facilitate fitting into the commissures.

In some implementations, and as shown in FIG. 5, lateral flaps 260b define a lateral extremity (e.g., an angular extremity) 263 between root portion 261 and tip portion 262 of each lateral flap. For example, and as shown, lateral extremities 263 can be shaped so as to give sheet 226b a shape that resembles that of a manta ray.

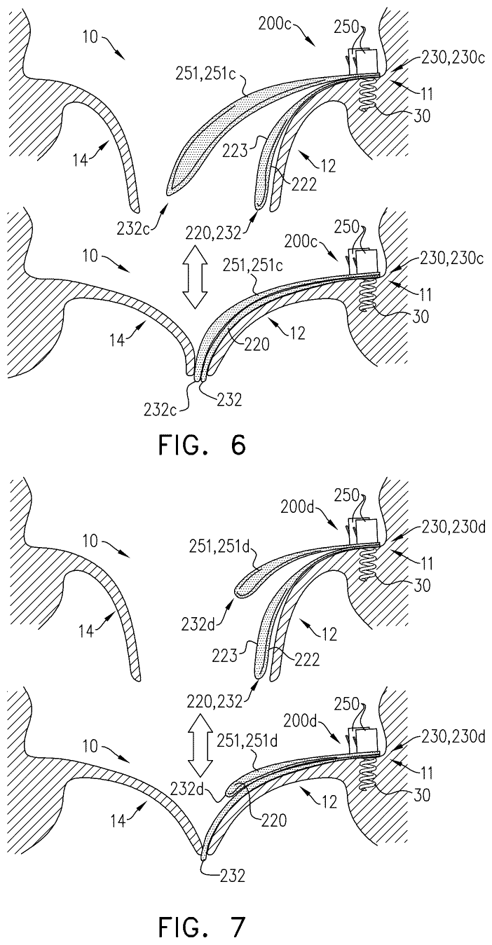

Reference is made to FIGS. 6-7, which are schematic illustrations showing implants 200c and 200d, in accordance with some implementations. Implants 200c, 200d can be considered to be variants of implant 200, and can be similar, at least in their general purpose, e.g., being anchored to a tissue of the heart so as to repair the function of the native leaflet, to implant 200 disclosed hereinabove, mutatis mutandis, except that implants 200c, 200d each comprise a second wing (e.g., a secondary wing) 251, in addition to a first wing, such as wing 220. Thus, the second wing of implant 200c is designated 251c, and the second wing of implant 200d is designated 251d.

FIGS. 6 and 7 show root portions 230, 230c, 230d of respective wings 220, 251c, and 251d secured to annulus 11 via interface 250. In this way, for each implant, second wing 251 extends from the root portion over second face/opposing face 223 of wing 220 to a tip portion 232c, 232d of the second wing.

In some implementations, and as shown in FIGS. 6-7, wing 220 is more flexible than second wing 251 e.g., such that wing 220 deflects (e.g., pivots relative to interface 250) farther into the ventricle during diastole than does second wing 251 (upper frames of FIGS. 6 and 7). In some implementations, wing 251 has holes therethrough, to facilitate antegrade blood flow despite the wing deflecting little or not at all. In some implementations, wing 251 comprises merely a frame with no covering. It is to be noted that such an arrangement can facilitate antegrade blood flow between wings 251 and 220 during diastole.

During ventricular systole (lower frames of FIGS. 6-7), wing 220 deflects toward opposing leaflet 14 (e.g., as described hereinabove for other implants), and also toward second wing 251, which facilitates (e.g., controls or regulates) this movement of wing 220. For example, second wing 251 can be sufficiently stiff (e.g., stiffer than wing 220) to inhibit wing 220 deflecting (e.g., prolapsing) into the atrium, and/or to support a prolapsing portion of native leaflet 12 during ventricular systole.

In some implementations in which wing 220 defines holes therethrough, wing 220 deflecting into contact with second wing 251 during systole obstructs blood flow through the holes. That is, during systole, wing 220 coapts and/or seals against leaflet 14 and second wing 251. In some implementations, wing 220 also defines holes therethrough in order to facilitate antegrade blood flow during diastole. However, such holes through wing 220 can be offset with respect to (e.g., not overlapping with) the holes of second wing 251 such that, upon coaptation between the two wings, the two wings collectively obstruct blood flow through the implant.

In some implementations, wings 220 and 251 can have substantially the same length, width, and/or shape as each other. This is shown for wing 251c of implant 200c. In some implementations, during systole, second wing 251 becomes sandwiched between wing 220 and opposing leaflet 14. In some implementations, second wing 251 can have a different shape and/or a different length and/or width from wing 220.

In the example shown by implant 200d, second wing 251d is shorter than wing 220 (e.g., tip portion 232d of the second wing is closer to interface 250 than is tip portion 232 of wing 220).

In some implementations, wing 251d can inhibit deflection of wing 220 while allowing tip portion 232 of wing 220 to behave in its flexible manner, and to coapt optimally with leaflet 14. For example, and as shown for implant 200d, during systole wing 251 may not become sandwiched between wing 220 and opposing leaflet 14, but rather becomes sandwiched directly between leaflets 12 and 14.

Due to the above-described effect of second wing 251, in some implementations it can be considered to be a limiter. Moreover, wing 251 (and/or features thereof) and other limiters described elsewhere herein (and/or features thereof) can be interchangeable.

In some implementations, wing 220 and second wing 251c, 251d are disposed within a flexible pouch (not shown) that allows wing 220 to deflect toward and away from the second wing. In some implementations, the pouch is more flexible than the respective wings, e.g., such that the pouch expands during diastole, and contracts during systole.

In some implementations, the pouch defines holes (e.g., on opposing sides of the pouch, one of which covers first face/contact face 222 of wing 220, the other of which covers second wing 251c, 251d). In some implementations, the holes facilitate antegrade blood flow during diastole. In some implementations, deflection of wing 220 away from second wing 251 expands the pouch, drawing in blood from outside of the pouch (e.g., inflating the pouch) during diastole, while deflection of wing 220 toward from second wing 251 can compress the pouch, ejecting blood out of the pouch (e.g., deflating the pouch) during systole.

In some implementations in which the pouch defines holes, the pouch aids in inhibiting retrograde blood flow during systole. In some implementations, the opposing sides of the pouch move toward each other during ventricular systole in a manner that inhibits blood flow through the holes. For example, the holes on one side of the pouch can be offset with respect to (e.g., not overlapping with) the holes on the other side of the pouch.

In some implementations, implants 200c, 200d comprise a third wing (not shown) that is also coupled to interface 250 at a root portion of the third wing, and extending over second wing 251c, 251d to a tip portion of the third wing. In some implementations, the third wing is shorter and/or less flexible than second wing 251c, 251d. For example, the third wing can be the stiffest of the three wings, and wing 220 can be the most flexible of the three wings.

In some implementations, second wing 251c, 251d is deflectable toward and away from the third wing, similarly to how wing 220 is deflectable toward and away from the second wing 251c, 251d. In some implementations, second wing 251c, 251d deflects away from the third wing during ventricular diastole, e.g., while wing 220 deflects away from second wing 251c, 251d. In some implementations, wing 220 deflects into contact with second wing 251c, 251d during ventricular systole, e.g., while second wing 251c, 251d deflects into contact with the third wing.

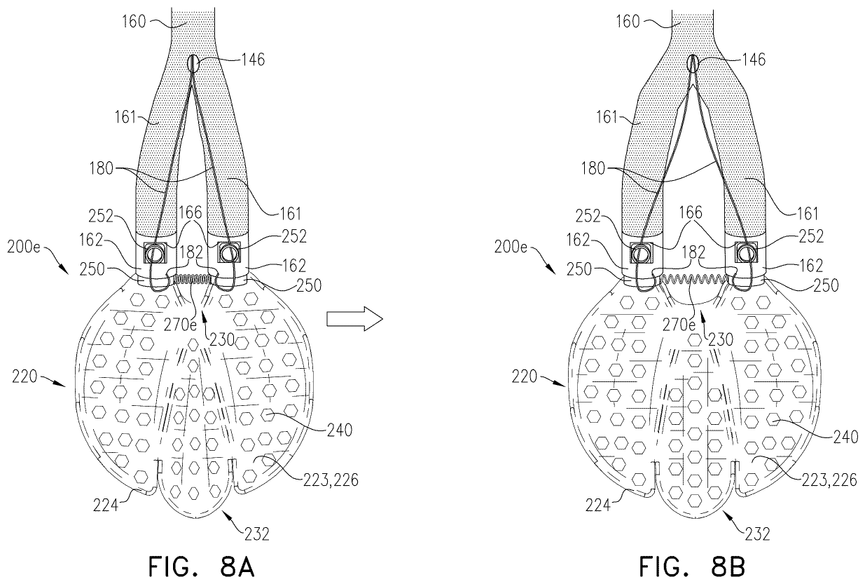

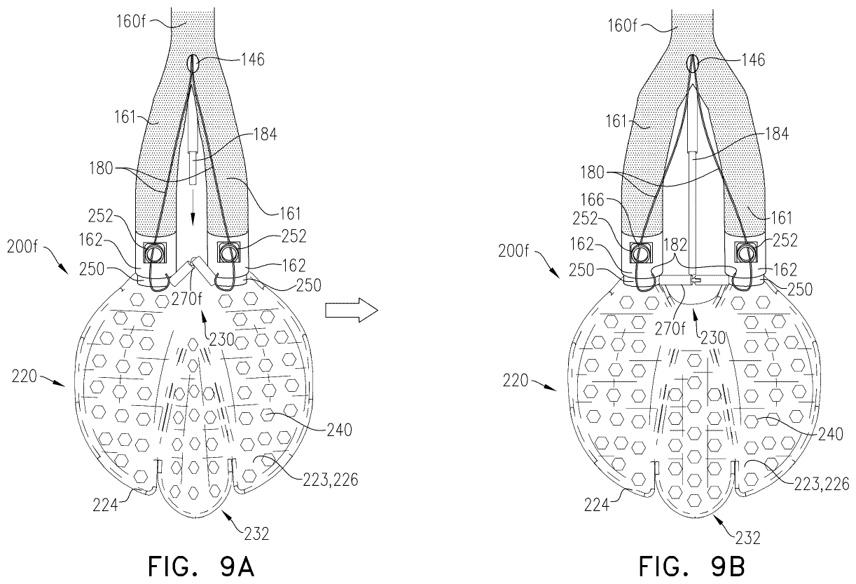

Reference is made to FIGS. 8A-B, 9A-B and 10A-B, which are schematic illustrations showing implants 200e, 200f, 200g, in accordance with some implementations. Implants 200e, 200f, 200g can be considered to be variants of implant 200, and can be similar, at least in their general purpose, e.g., being anchored to a tissue of the heart so as to repair the function of the native leaflet, to implant 200 disclosed hereinabove, mutatis mutandis, except that implants 200e, 200f, 200g each comprise an expansion element 270e, 270f, 270g that is coupled to wing 220.

In some implementations, implants 200e, 200f, 200g are delivered within catheter 140 while wing 220 assumes its compressed state (FIGS. 1, 2A) and the corresponding expansion element (e.g., expansion element 270e, 270f, or 270g) assumes a compact state. Wing 220 can comprise a shape-memory frame 224 that biases the wing toward assuming the expanded state, and expansion elements 270e, 270f, 270g are coupled to the wing such that as wing 220 transitions from the compressed state to the expanded state, each expansion element 270e, 270f, 270g transitions from the compact state to an extended state.

In some implementations, despite shape-memory frame 224 biasing the wing toward assuming the expanded state, in some cases implant 200 can encounter impeding forces (e.g., from the surrounding anatomy) that can resist the shape-memory expansion of frame 224 toward its expanded state, and/or can distort the shape of the wing even after the wing has reached its expanded state. For example, such forces may oppose movement of interfaces 250 away from each other. In some implementations, expansion elements 270e, 270f, and 270g are configured to counteract such impeding forces, thereby facilitating expansion of wings 220e, 220f, and 220g to their respective expanded states, and/or subsequently maintaining the wing in its expanded state.

FIGS. 8A, 9A and 10A each show wing 220 having assumed a state that is between the compressed and expanded states. That is, although implants 200e, 200f, 200g are shown having been deployed from within catheter 140, wing 220 is nonetheless not fully expanded. FIGS. 8B, 9B and 10B each show wing 220 having fully expanded, with a respective expansion element 270e, 270f, 270g having extended from a compact state to an extended state. Note that interfaces 250 are further from each other in FIGS. 8B, 9B, and 10B than in FIGS. 8A, 9A, and 10A.

In some implementations, expansion elements 270e, 270f, 270g apply an expansion force to wing 220 as the expansion element extends towards its extended state. In some implementations, the expansion force pushes interfaces 250 away from each other, e.g., the expansion element can be coupled to interfaces 250, as shown. Alternatively or in addition, expansion elements 270e, 270f, 270g can push or pull on portions of frame 224 and/or on branches 161 of shaft 160.

In some implementations, the expansion element is straighter in its extended state than in its compact state, and is straightened from the compact state to the extended state. For example, the expansion element can comprise a hinge that is articulated (expansion element 270f, FIGS. 9A-B) and/or subunits that become aligned collinearly (expansion element 270g, FIGS. 10A-B) to straighten the expansion element from the compact state to the extended state.

In some implementations, the expansion element can be a locking expansion element, e.g., comprising subunits that lock together upon expansion element 270f, 270g assuming the extended state, as shown in FIGS. 9B and 10B. In some implementations, the expansion element can provide only such locking, and not an expansion force.

In some implementations, the expansion element is a “passive” expansion element that applies the expansion force to wing 220 without needing to be actuated. Expansion element 270e is an example of such a passive expansion element. In some implementations, and as shown, expansion element 270e can comprise a spring (FIGS. 8A-B) that relaxes (e.g., expands) toward its extended state, e.g., is strained (e.g., compressed) while in its compact state.

In some implementations, the expansion element is an “active” expansion element that applies the expansion force to wing 220 upon actuation of the expansion element. Expansion element 270f is an example of such an active expansion element. In some implementations, delivery tool 150 comprises an extension actuator 184 (FIGS. 9A-B) that is used to transluminally actuate the expansion element from the compact state to the extended state, facilitating expansion of wing 220 from the compressed state to the expanded state. In some implementations, extension actuator 184 is used by extending it to push against the expansion element. In the particular configuration shown, extension actuator 184 extends from shaft 160 between branches 161 of the shaft.

FIGS. 10A-B show an extension actuator 186 that comprises a line that is pulled (e.g., from outside of the subject, such as via controller 120) in a manner that causes the subunits of expansion element 270g to fit together.

In some implementations, and as shown, expansion element 270e, 270f, 270g is longer in the extended state than in the compact state.

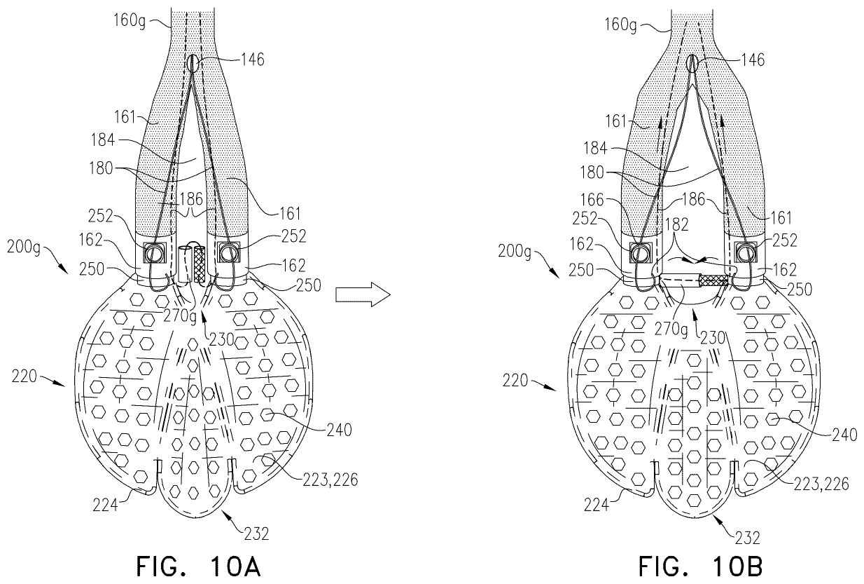

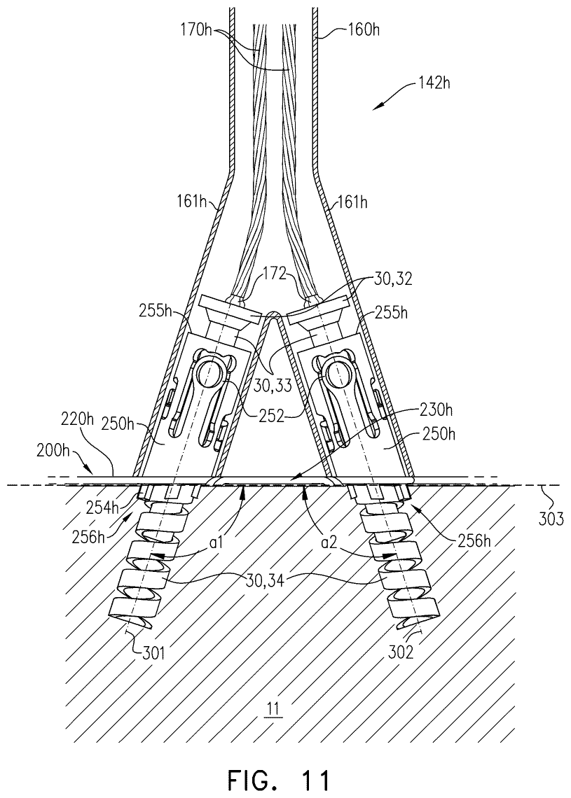

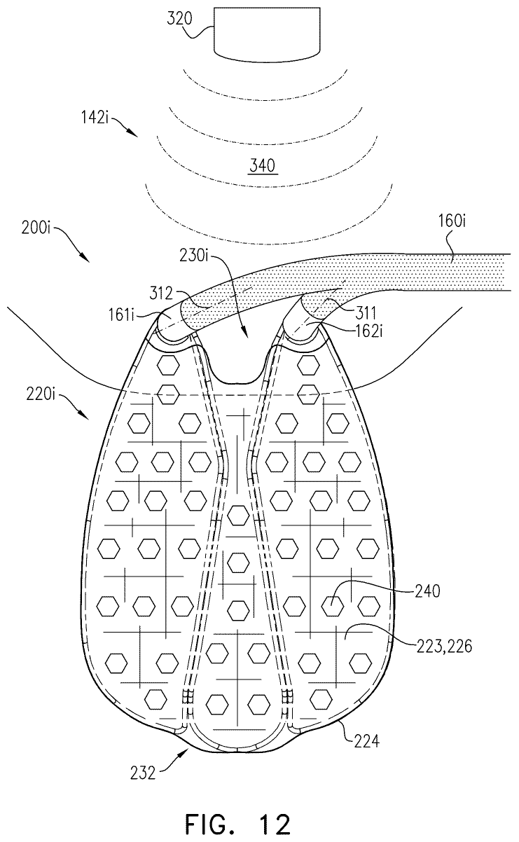

Reference is made to FIGS. 11-12, which are schematic illustrations showing distal portions 142h, 142i of delivery tools 150h, 150l, in accordance with some implementations. Delivery tools 150h, 150i can be considered to be variants of delivery tool 150, and can be similar, at least in their general purpose, e.g., to deliver implant 200 or any of the variants thereof described herein, to the heart of a subject (e.g., a living subject, a simulation, etc.), mutatis mutandis, except that distal portions 142h, 142i of delivery tools 150h, 150i (e.g., drivers 170h, 170i thereof) are used to anchor respective implants 200h, 200i to tissue (e.g., to tissue of annulus 11) by screwing a pair of anchors 30 along nonparallel axes. Accordingly, implants 200h, 200i each have a pair of interfaces 250h, 250i that are coupled to respective wings 220h, 220i such that each pair of interfaces define nonparallel longitudinal axes 301 & 302, or 311 & 312.

In some implementations, interfaces 250h, 250i are configured to facilitate screwing a pair of anchors 30, along the nonparallel axes, into respective sites of the tissue. In some implementations, and as shown, delivery tool 150h, 150i is configured for drive heads 172 to fit into anchors 30 (e.g., into respective anchor heads 32 of the anchors) that are positioned along respective axes 301, 302. In some implementations, shaft branches 161h each engage a corresponding interface 250h at an angle that is oblique to plane 303, e.g., since drivers are sufficiently flexible to fit into anchor heads 32 that are parallel to proximal ends 255 of interfaces 250h.

As shown in FIG. 11, at least one of the anchors will typically be screwed through interface 250h, 250i and into the tissue along an axis 301, 302 that is oblique to a plane 303 defined by root portion of wing 220h, 220l. In some implementations, and as shown in FIG. 11, each interface 250h has a proximal end 255 (e.g., a circular proximal end) that is orthogonal to respective axes 301, 302 and/or oblique with respect to plane 303. In some implementations, and as shown, a helical tissue engaging element 34 extends (e.g., via a driver-shaft 33) from anchor head 32, and driver 170 is used to screw anchor 30 at least until the anchor head abuts a proximal end 255h of respective interface 250h. In some implementations, anchor 30 can continue to be screwed, even after anchor head 32 abuts proximal end 255h of interface 250h. That is, continued rotation of anchor head 32 with respect to interface 250 can continue to advance tissue-engaging element 34 further into tissue of annulus 11, thereby closing a gap that can be present between implant 200h and the tissue.

In some implementations, and as shown in FIG. 11, each interface 250h comprises a cylindrical tube, extending along a respective axis 301, 302, that has a circular cross-section is transverse to the respective axis. In some implementations, each interface 250h has a non-circular, elliptical distal end 256 (defined by teeth 254). In some implementations, and as shown in FIG. 11, a distal end 256h of interface 250h is oblique to axis 301, 302 and/or parallel with plane 303.

In some implementations, and as shown in FIG. 11, both axes 301, 302 are oblique to plane 303. In some implementations, and as shown, an angle a1 between axis 301 and a region of plane 303 disposed between (e.g., delimited by) interfaces 250h is equal to an angle a2 between axis 302 and the region of the plane. For example, angle a1 and angle a2 can both be obtuse.

The configuration described with reference to FIG. 11 can advantageously facilitate anchoring using two anchors through diverging branches of a bifurcated tube, e.g., by allowing the anchors to be driven into tissue obliquely, despite the head of each anchor pressing flat against a respective surface of the implant (proximal end 255h of interface 250h). That is, the shape of interface 250h allows it to serve as a geometric “adapter”.

In some implementations, and as shown in FIG. 12, system 100 further comprises at least one imaging device (e.g., an ultrasound transceiver and/or a fluoroscope) that is used to visualize implant 200i and/or surrounding tissue (e.g., tissue of annulus 11). In some implementations, imaging device 320 transmits and/or receives imaging energy 340, which is used to facilitate determining that implant 200i is in a desired position (e.g., prior to screwing anchors 30 into the tissue). As shown, shaft 160i (e.g., branches 161i thereof) are skewed aside, in order to reduce disruption of the travel of imaging energy 340 to and from imaging device 320, e.g., to reduce “shadowing” caused by the presence of the shaft of the delivery tool. In some implementations, skewed branches 161i of shaft 160i facilitate visualizing the implant while imaging device 320 faces (e.g., orthogonally faces) a plane defined by root portion 230i of wing 220l. It is to be noted that such an advantage can be provided even when system 100 does not include an imaging device, e.g., when a separate imaging device is used.

As shown, axes 311 & 312, along which distal end portions 162 of shaft branches 161i respectively extend, can be oblique to the plane defined by root portion 230i of wing 220i (e.g., analogous to plane 303, shown in FIG. 11). In some implementations, a first angle, between axis 311 and a region of the plane disposed between distal end portions 162l, is unequal to a second angle, between axis 312 and the region of the plane. In some implementations, the first angle is greater than the second angle. For example, the first angle can be obtuse, and/or the second angle can be acute.

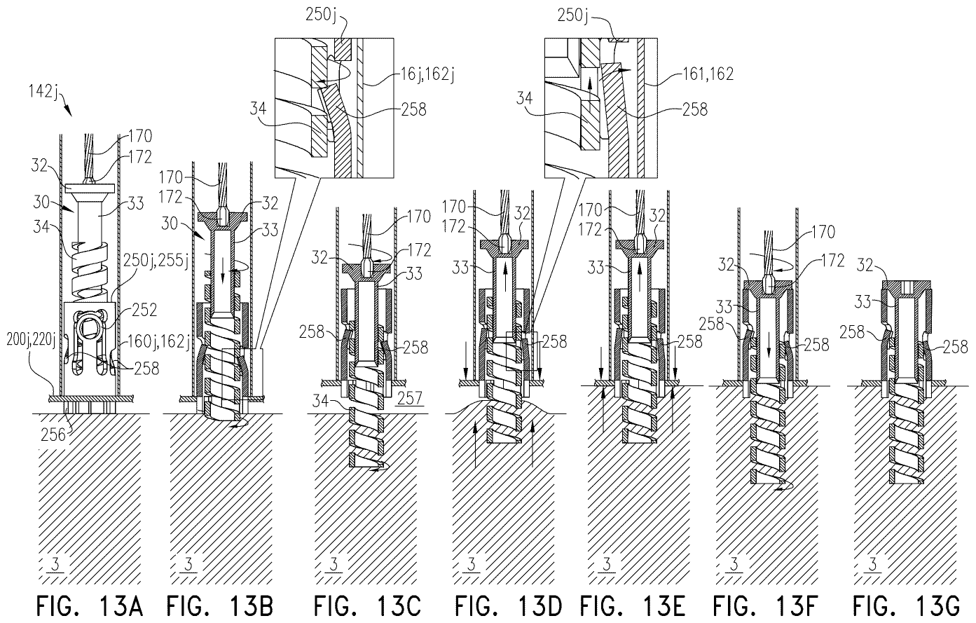

Reference is made to FIGS. 13A-G, which are schematic illustrations showing an interface 250j that has a ratcheting feature, and the use of a delivery tool 150j to anchor interface 250j of an implant 200j to tissue 3 of a subject (e.g., a living subject, a simulation, etc.), in accordance with some implementations. Implant 200j has a wing 220j. Delivery tool 150j can be considered to be a variant of delivery tool 150, and can be similar, at least in its general purpose, e.g., to deliver an implant to tissue of a subject, mutatis mutandis. Implant 200j can be implant 200 or any of the variants thereof described herein, and can be similar, at least in its general purpose, e.g., being anchored to a tissue of the heart so as to repair the function of the native leaflet, to implant 200 disclosed hereinabove, mutatis mutandis, except that interface 250j is a ratcheting interface. Furthermore, anchor 30 can be any screw-in (e.g., helical) anchor, e.g., the anchor may not require any additional feature in order to cooperate with ratcheting interface 250j.

Interface 250j is a tubular anchor receiver defining a lumen. As described hereinbelow, interface 250j interacts with anchor 30 so as to facilitate anchoring of implant 200j to tissue of the subject by (i) inhibiting non-helical advancement of the anchor distally through the lumen, but (ii) facilitating non-helical withdrawal of the anchor proximally through the interface. In some implementations, driver 170 (e.g., drive head 172 thereof) is coupled to anchor head 32 in a manner that facilitates non-helical withdrawal of anchor 30, e.g., by pulling the driver proximally.

FIGS. 13A-G show use of driver 170 and a shaft 160j to screw anchor 30, via interface 250j, into tissue 3. FIG. 13A is a perspective view of distal portion 142j disposed at tissue 3, such that implant 200j (e.g., a distal end 256 of interface 250j) is adjacent to (e.g., abutting) the tissue. FIGS. 13B-G are cross-sectional views of distal portion 142j that show use of driver 170 to helically advance anchor 30, such that part of helical tissue-engaging element 34 of the anchor penetrates tissue 3.

In some implementations, and as shown in the inset of FIG. 13B, tabs 258 protrude (e.g., are biased to protrude) into the lumen of interface 250j. In this way, were a non-helical distal force to be applied to anchor 30, the non-helical distal force would cause helical tissue-engaging element 34 to abut tab 258, inhibiting non-helical distal advancement of anchor 30 through interface 250j. However, tab 258 is dimensioned and positioned to allow helical tissue-engaging element 34 to slide helically over and/or past the tab during helical distal advancement of anchor 30 through interface 250j, as shown in the inset of FIG. 13B. Thus, tissue-engaging element acts as an external screw thread while tabs 258 act as an internal screw thread.

In some implementations, during the screwing-in of anchor 30 into tissue 3, a gap 257 may open up between the tissue and the implant (e.g., the interface thereof). For example, during screwing-in, tissue-engaging element 34 can screw through the interface faster than into the tissue. Interface 250j is configured to address this. In order to close gap 257, driver 170 can be pulled proximally and/or shaft 160 can be pushed distally against interface 250j and/or another part of implant 200j. FIGS. 13D-E show simultaneous pulling and pushing.

In some implementations, and as shown in the inset of FIG. 13D, application of a sufficient non-helical proximal force to the anchor, via driver 170, causes tabs 258 to transiently deflect outwardly as helical tissue-engaging element 34 withdraws through interface 250j. Such a non-helical proximal force can be provided by the above-described pulling on driver 170 and/or pushing on interface 250j. In some implementations, and as shown, each turn of helical tissue-engaging element 34 transiently deflects tabs 258 outwardly as the turn passes the tab, allowing anchor 30 to be withdrawn through interface 250j. This can be described as helical tissue-engaging element 34 ratcheting proximally past tab 258 (e.g., similar to the tightening of a cable tie) and through the lumen of interface 250j.

As shown, pushing shaft 160 distally while pulling proximally on driver 170 causes interface 250j (and therefore implant 200j) to advance distally and/or tissue 3 to be drawn proximally, thereby closing gap 257, such that distal end 256 of interface 250j is again adjacent (e.g., abutting) the tissue. In some implementations, teeth 254 penetrate a portion of tissue 3, as shown in FIG. 13E.

Screwing-in can be optionally paused during the ratcheting steps shown in FIGS. 13D-E.

FIG. 13F shows helical advancement of anchor 30 having been resumed, until anchor head 32 abuts interface 250j (e.g., proximal end 255 thereof). At this stage, delivery tool 150j can be removed (FIG. 13G), leaving interface 250j of implant 200j secured to tissue 3 by anchor 30.

It is to be understood that interface 250j, its ratcheting features, and/or the techniques described hereinabove for use therewith, can be used, mutatis mutandis with implants other than implant 200 and variants thereof.

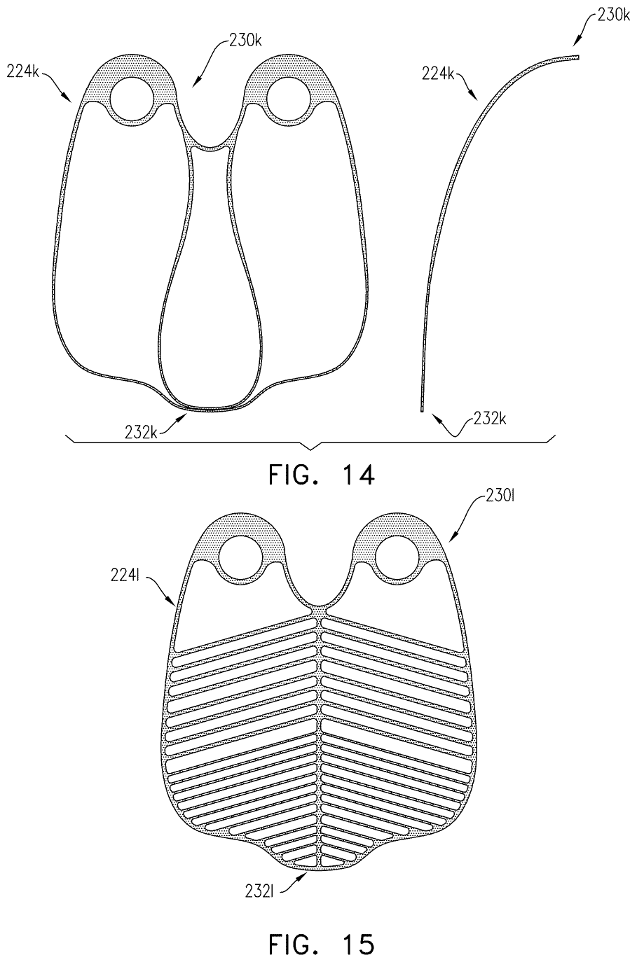

Reference is made to FIGS. 14-18, which are schematic illustrations showing frames 224k, 224l, 224m, 224n for use with implants, in accordance with some implementations. Frames 224k, 224l, 224m, 224n can be considered to be variants of frame 224, and can be similar, at least in their general purpose, e.g., to provide mechanical support to implant 200 disclosed hereinabove or to any variant thereof, mutatis mutandis, except that each frame 224k, 224l, 224m, 224n has a root portion 230k, 230l, 230m, 230n that is stiffer than a respective tip portion 232k, 232l, 232m, 232n.

In some implementations, frame 224k, 224l, 224m, 224n comprises a wire frame (e.g., a flexible wire frame), and in the example shown in FIGS. 14, wire frame 224k comprises thicker wires at a root portion 230k than at a tip portion 232k. In some implementations, the thicker wires of the frame at root portion 230k are stiffer than the wires of frame at the tip portion.

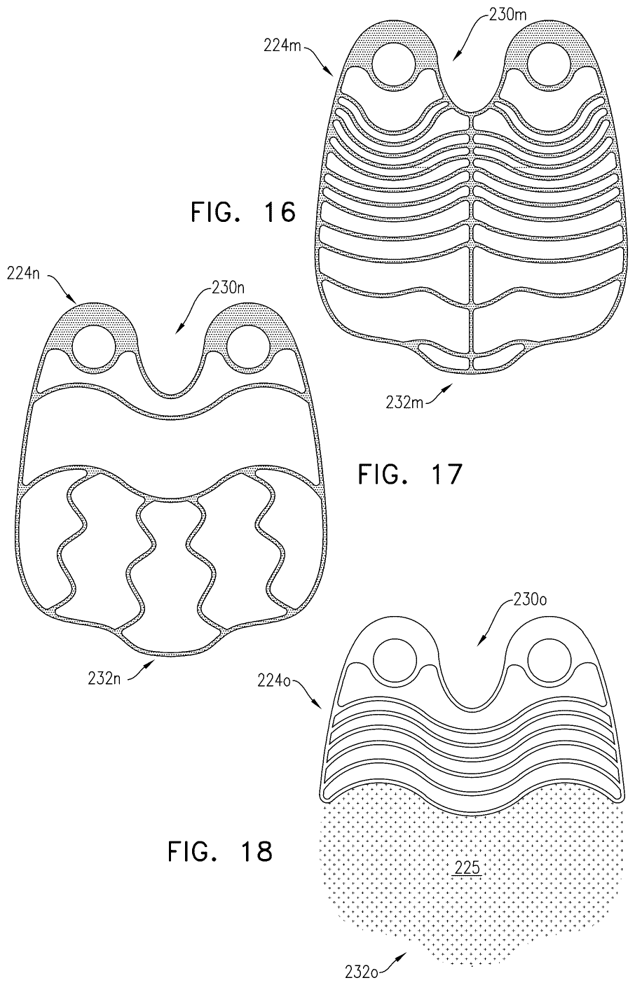

In some implementations, the root portion of the frame is stiffer than the tip portion of the frame because a greater amount of material comprises the structural members of the frame at the root portion (e.g., after cutting the frame out of a metal sheet) than at the tip portion. In some implementations, and as shown in FIGS. 15-16, members of frame 224l, 224m can be: (i) thicker at root portion 230l of frame 224l than at tip portion 232l of the frame (FIG. 15) and/or (ii) spaced more closely to each other at root portion 230m of frame 224m than at tip portion 232lm of the frame (FIG. 16).

In some implementations, and as shown in FIG. 17, members of frame 224n at tip portion 232n of wing 220n are shaped to facilitate longitudinal flexing of the members of the frame.

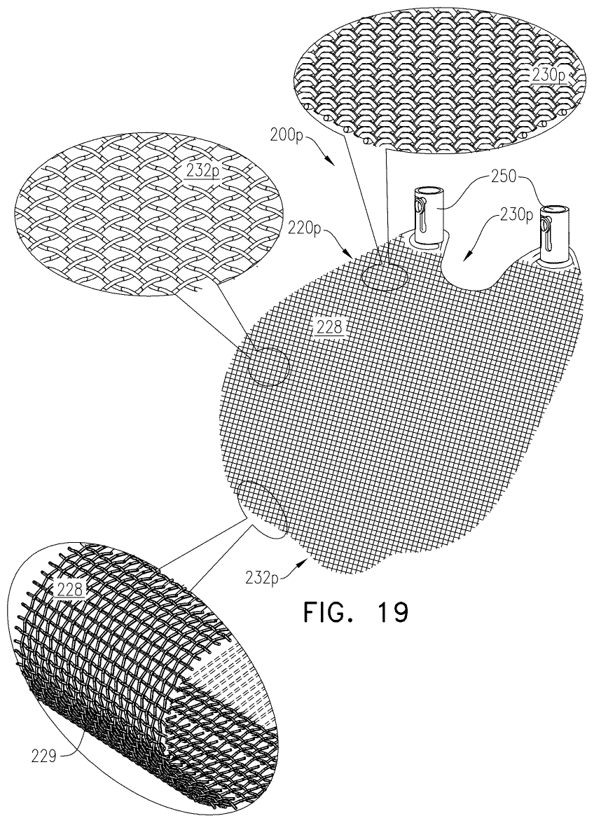

In some implementations, frame 224 comprises a stiffer material at the root portion than at the tip portion. In some implementations, and as shown in FIG. 18, frame 2240 has a root portion 2300, whereas tip portion 2320 of the wing comprises a flexible sheet 225 (e.g., a polymer sheet)—but no frame.

FIGS. 14-17 show the frames of respective wings, omitting flexible sheet 226, which can be disposed over the frame. Similarly, sheet 226 can be disposed over frame 2240 of wing 2200, e.g., in addition to sheet 225. In some implementations, for wing 2200, sheet 226 extends over sheet 225. In some implementations, sheet 226 can extend beyond frame 2240 to define sheet 225, e.g., the region labeled 225 can merely represent a portion of sheet 226 that extends beyond (e.g., is not supported by) frame 2240. In this manner, region 225 can be analogous to lateral extremity 263, but at tip portion 2320 of the implant, rather than at a lateral region. In some implementations, the wings to which the frames of FIGS. 14-17 belong do not include a sheet, e.g., the wing consists substantially of the frame alone. Similarly, frame 2240 may not be covered by a sheet, e.g., region 225 can comprise a sheet that is attached to the edge of the frame.

Reference is made to FIG. 19, which shows schematic illustrations of an implant 200p comprising a wing 220p, in accordance with some implementations. Implant 200p can be considered to be a variant of implant 200, and can be similar, at least in its general purpose, e.g., being anchored to a tissue of the heart so as to repair the function of the native leaflet, to implant 200 disclosed hereinabove, mutatis mutandis, except that implant 200p comprises a mesh 228. Mesh 228 can be formed from a wire, such as a metallic wire (e.g., comprising Nitinol or steel). Mesh 228 can be woven. Mesh 228 can be braided. Mesh 228 can be included in addition to or instead of a frame, and/or a flexible sheet. For example, wing 220p can comprise mesh 228 supported by a frame (not shown), mesh 228 covered by a flexible sheet (not shown), or mesh 228 substantially alone, e.g., as shown.

In some implementations, mesh 228 is stiffer at root portion 230p of wing 220p than at tip portion 232p of the wing. In some implementations, and as shown, this is achieved by mesh 228 being woven more densely at a root portion 230p of the wing than at a tip portion 232p of the wing. Alternatively or additionally, mesh can comprise thicker wires at root portion 230p than at tip portion 232p.

In some implementations, and as shown, wing 220p has two layers of mesh 228. For example, wing 220p can be formed by folding the mesh. In the example shown, wing 220p is formed by folding mesh 228 at an edge 229, e.g., at a tip of the wing. Folding mesh 228 at the tip of the wing can advantageously confer atraumatic properties on the tip, e.g., such that wing 220p presents a rounded edge to the opposing leaflet, e.g., rather than a sharp edge. This can be particularly advantageous due to the wing being made from wire.

In some implementations, interfaces 250 are attached to both layers of the mesh. In some implementations, this attachment secures the mesh in its double-layer configuration.

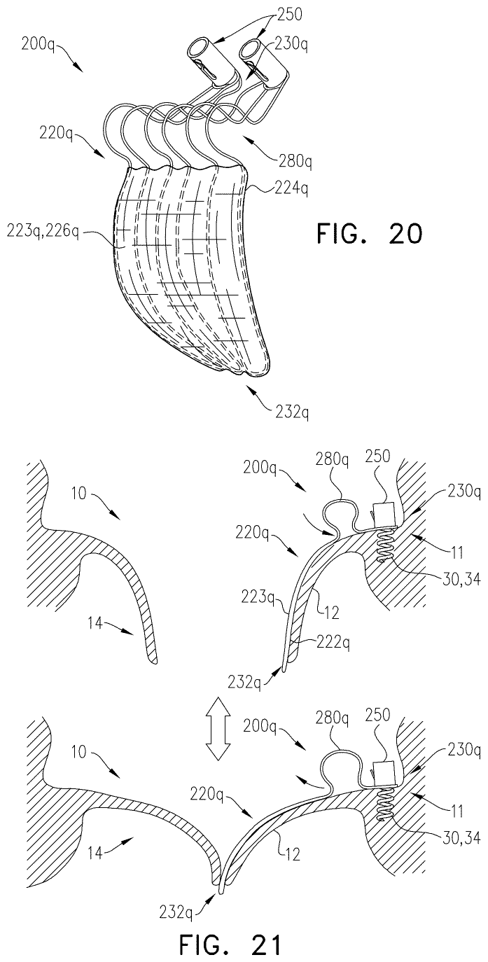

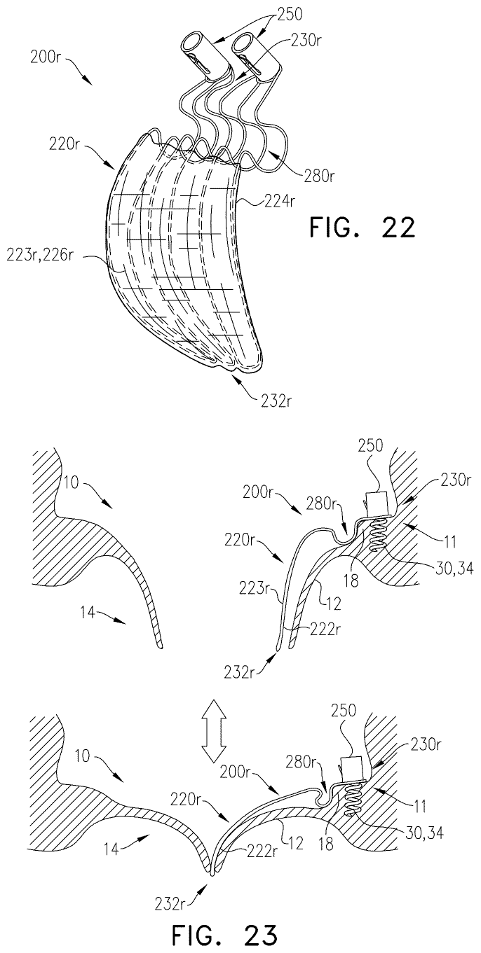

Reference is made to FIGS. 20-26, which are schematic illustrations showing implants 200q, 200r, 200s, 200t, 200u, in accordance with some implementations. Implants 200q, 200r, 200s, 200t, 200u can be considered to be variants of implant 200, and can be similar, at least in their general purpose, e.g., being anchored to a tissue of the heart so as to repair the function of the native leaflet, to implant 200 disclosed hereinabove, mutatis mutandis, except that implants 200q, 200r, 200s, 200t, 200u comprise a flex element 280q, 280r, 280s, 280t, 280u that couples root portion 230q, 230r, 230s, 230t, 230U of wing 220q, 220r, 220s, 220t, 220u to a respective tip portion 232q, 232r, 232s, 232t, 232U of the wing. In some implementations, and as shown in FIGS. 20 and 22, a flexible sheet 226q, 226r is disposed over frame 224q, 224r, e.g., over a portion of the frame not including flex elements 280q, 280r.

Movement of the native tissue (e.g., tissue of valve 10) during the cardiac cycle can include movement of leaflet 12, relative to annulus 11. Since the implant can be secured to annulus 11 at the root portion of the wing, which extends over leaflet 12 to the tip portion of the wing, implants having wings whose tip portions can move in relation to their root portions can be better able to move in concert with the native tissue. As shown in FIGS. 21 and 23, flexing of flex element 280q, 280r, 280s, 280t, 280u facilitates movement (such as articulation and/or deflection, e.g., pivoting) of the tip portion with respect to the root portion during the cardiac cycle.

In some implementations, the flex element has a relaxed state and oscillates toward and away from the relaxed state as the heart cycles between diastole and systole. For example, the flex element can transition away from its relaxed state (e.g., can become strained) as the heart cycles into systole, and toward the relaxed state as the heart cycles into diastole. That is, the flex element can bias the wing to be deflected into the ventricle and away from the opposing leaflet.

In some implementations, and as shown, frame 224q, 224r, 224s, 224t, 224u defines flex element 280q, 280r, 280s, 280t, 280u. For example, the flex element can be a flexure or a hinge, e.g., a living hinge.

In some implementations, and as shown, flex element 280q, 280r is a protrusion of frame 224q, 224r that protrudes from wing 220q, 220r, e.g., as loop and/or torsion spring.

Flex element 280q protrudes from the second face/opposing face 223q of wing 220q, opposite the wing's contact face 222q, as shown in FIG. 21. This can advantageously reduce a likelihood of the flex element protruding against leaflet 12 and/or interfering with the functionality of the leaflet.

Flex element 280r protrudes from the first face/contact face 222r of wing 220r, opposite the wing's opposing face 223r, as shown in FIG. 23. This can advantageously facilitate optimal positioning of the implant. For example, flex element 280r can be positioned within implant 200r such that abutment of the flex element against hinge-point 18 (e.g., using shaft 160 of delivery tool 150, as described hereinabove) positions interface 250 at an appropriate site of annulus 11 for anchoring thereto.

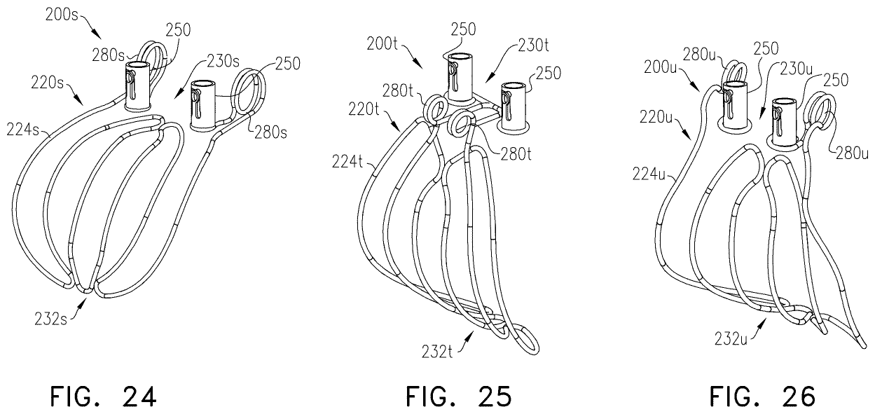

Reference is again made to FIGS. 24-26, which show implants 200s, 200t, 200u without sheets covering respective frames 224s, 224t, 224u, for clarity. In some implementations, and as shown, flex element 280s, 280t, 280u is a torsion spring defined by frame 224s, 224t, 224U that connects tip portion 232s, 232t, 232U to root portion 230s, 230t, 230u. The torsion springs provide a combination of flexibility and strength to wings 220s, 220t, 220u. That is, the torsion springs typically (i) allow tip portions 232s, 232t, 232u to deflect in relation to root portions 230s, 230t, 230u during the cardiac cycle, while (ii) applying tension, e.g., downstream tension, to tip portions 232s, 232t, 232u. This combination of flexibility and strength facilitates supporting leaflet 12 (e.g., a prolapsed or flailing leaflet) over the cardiac cycle. For example, when root portion 230s, 230t, 230u is secured to annulus 11, flex element 280s, 280t, 280u can (i) allow tip portion 232s, 232t, 232u to deflect in concert with leaflet 12, while (ii) applying tension to the tip portion that keeps the tip portion downstream of a plane of coaptation between native leaflets 12, 14.

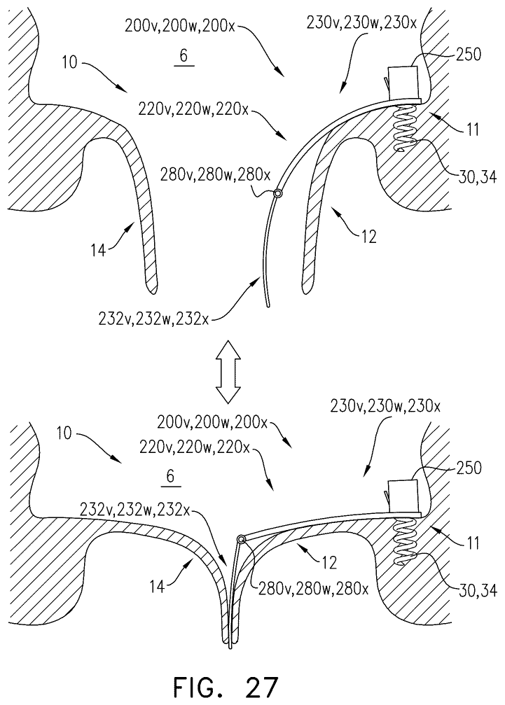

Reference is made to FIGS. 27-30, which are schematic illustrations showing implants 200v, 200w, 200x in accordance with some implementations. Implants 200v, 200w, 200x can be considered to be variants of implant 200, and can be similar, at least in their general purpose, e.g., being anchored to a tissue of the heart so as to repair the function of the native leaflet, to implant 200 disclosed hereinabove, mutatis mutandis. Wings 220v, 220w, 220x of implants 200v, 200w, 200x comprise tip portions 232v, 232w, 232x and root portions 230v, 230w, 230x that can articulate in relation to each other, e.g., in response to the forces exerted upon the implant during the cardiac cycle when anchor receiver 250 is secured to annulus 11. FIG. 27 is intended to broadly represent such articulation of all of these implants.

In some cases, injury or disease can cause different portions of native leaflet 12 to react differently to forces exerted upon the leaflet during the cardiac cycle (e.g., prolapse and/or flailing can be localized to discrete portions of the native leaflet). In some such cases, movement of tip portions 232v, 232w, 232x and root portions 230v, 230w, 230x in relation to each other can facilitate function of native valve 10. For example, root portion 230v, 230w, 230x being anchored directly to annulus 11 (and optionally, being stiffer than tip portion 232v, 232w, 232x) can provide greater support to a portion of leaflet experiencing prolapse, while articulation of the tip portion with respect to the root portion can improve coaptation of a flailing portion of the leaflet.

In some implementations, coaptation of anterior leaflet 14 with posterior leaflet 12 and/or with wing 220v, 220w, 220x can also be facilitated by articulation of tip portion 232v, 232w, 232x at flex element 280s, 280t (e.g., a hinge, such as a ball-and-socket hinge shown in FIG. 27).

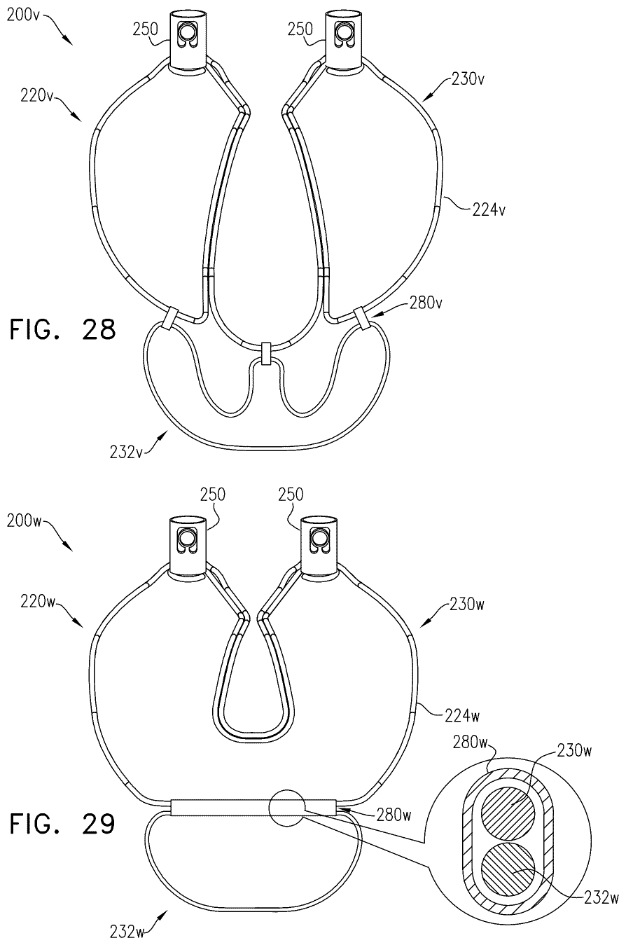

In some implementations, and as shown in FIG. 28, flex element 280v comprises one or more connectors, such as rings or sutures. Alternatively or in addition, the flex element can comprise interlocking loops, e.g., a pair of interlocking loops, including one loop that is defined by the tip portion, and another tip that is defined by the root portion, such as links in a chain.

In some implementations, and as shown in FIG. 29, flex element 280w comprises an elongate connector, such as a tube through which respective portions of tip portion 232w and root portion 230w (e.g., respective portions of frames 224w of the tip portion and of the root portion) extend alongside each other, such that the tip portion and root portion can articulate in relation to each other.

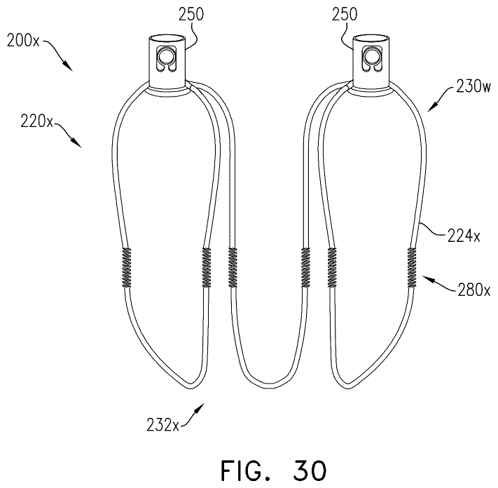

In some implementations, and as shown in FIG. 30, flex element 280x comprises a plurality of coiled wires connecting tip portion 232x to root portion 230x. In some implementations, the coiled wires of flex element 280x flex in response to the forces exerted upon the implant during the cardiac cycle, such that the tip portion and root portion articulate in relation to each other.

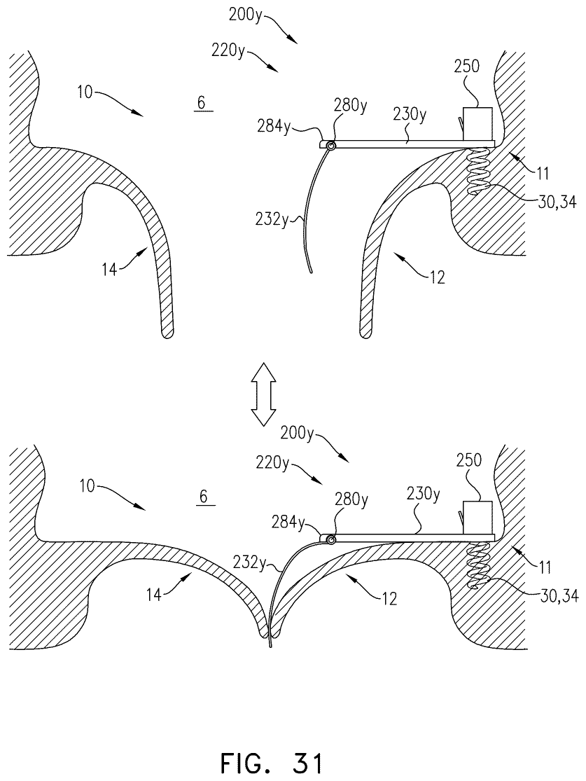

Reference is made to FIGS. 31-33, which are schematic illustrations showing implants 200y, 200z, in accordance with some implementations. Implants 200y, 200z can be considered to be variants of implant 200, and can be similar, at least in their general purpose, e.g., being anchored to a tissue of the heart so as to repair the function of the native leaflet, to implant 200 disclosed hereinabove, mutatis mutandis.

In some implementations, and similarly to implants 200v, 200w, 200x described hereinabove, implants 200y, 200z comprise tip portions 232y, 232z and root portions 230y, 230z that can articulate in relation to each other when anchor receiver 250 is secured to annulus 11, e.g., in response to the forces exerted upon the implant during the cardiac cycle. However, in contrast to implants 200v, 200w, 200x, implants 200y, 200z each comprise a limiter 284y, 284z that limits a range of motion across which respective tip portions and root portions articulate in relation to each other. In some implementations, the wing comprises flex element 280y, 280z (e.g., a hinge, as shown in FIGS. 31-33) that articulatably couples the root portion of the wing to the tip portion of the wing such that a range of motion of the hinge defines a deflection-limit (e.g., a discrete deflection-limit) of the wing—or at least of the tip portion of the wing.

In some implementations limiter 284y, 284z becomes resistant to upstream articulation of tip portion 232y, 232z when the tip-portion reaches a deflection-limit. In some implementations, limiter 284y is defined by a region of root portion 230y of wing 220y, e.g., that overhangs tip portion 232y. As shown in FIG. 31, tip portion 232y articulates via flex element (e.g., hinge) 280y with respect to root portion 230y, and limiter 284y limits articulation of tip portion 232y past the deflection-limit (bottom frame of FIG. 31) by the tip portion contacting limiter 284 upon wing 220y reaching the deflection-limit.

Although root portion 230y can be stiffer than tip portion 232y, and although, for the sake of clarity, FIG. 31 shows root portion 230y as stationary throughout the cardiac cycle, it is to be understood that, at least in some implementations, root portion 230y can also deflect and/or flex responsively to the cardiac cycle.

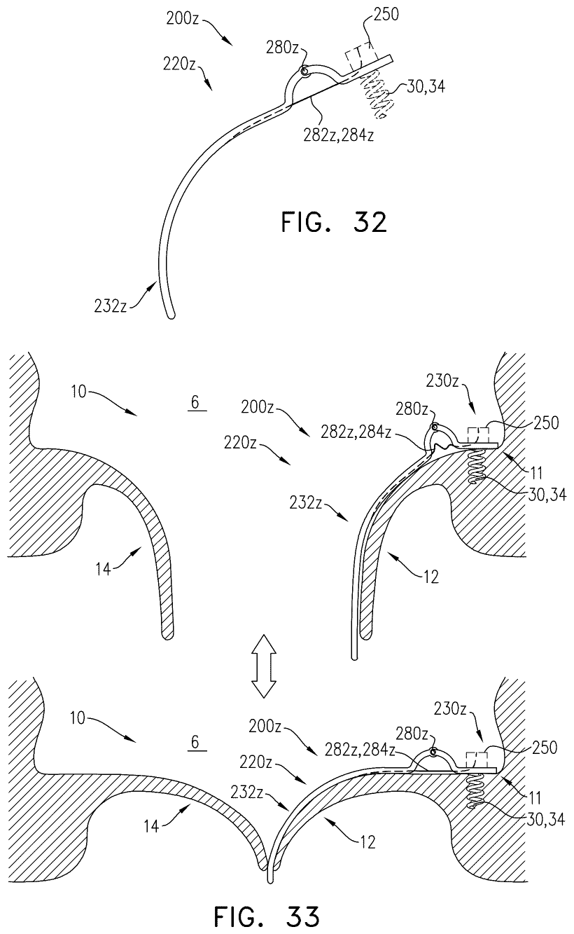

In some implementations, and as shown in FIGS. 32-33, limiter 284z of implant 200z comprises a tether 282z that limits the range of motion of flex element 280z. In some implementations, tether 282z becomes tensioned as wing 220z (e.g., tip portion 232z thereof) reaches the deflection-limit, e.g., halting articulation of tip portion 232z past the deflection-limit. In some implementations, the deflection-limit is adjustable (e.g., transluminally adjustable using the delivery tool) by adjusting an effective length of, and/or tension on, tether 282z. For example, the deflection-limit can be reduced by tensioning tether 282z, e.g., by pulling proximally on the tether, or by winding the tether onto a spool, e.g., a spool at interface 250.

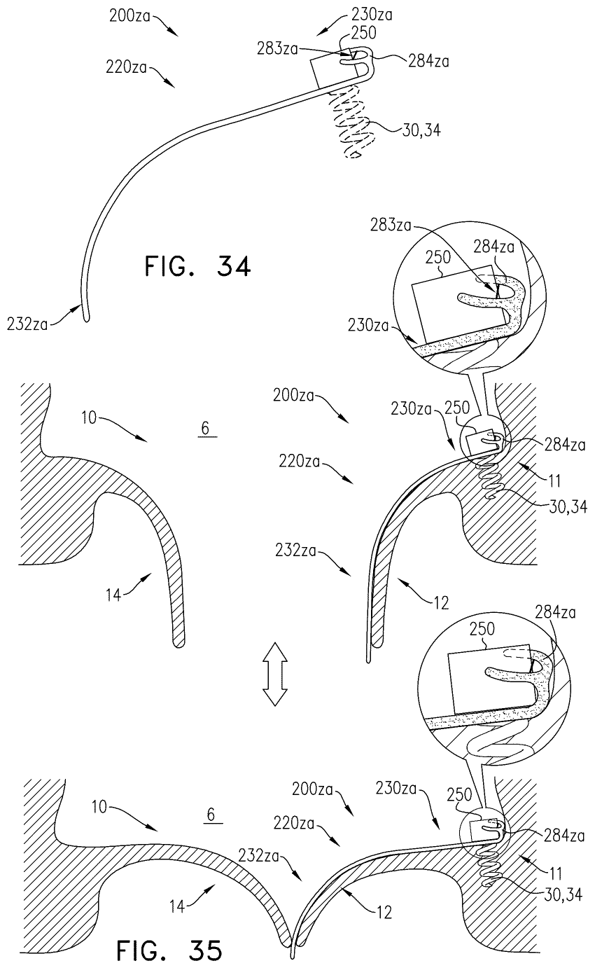

Reference is made to FIGS. 34-44, which are schematic illustrations showing implants 200za, 200zb, 200zc, 200zd, 200ze, 200ze, 200zf, 200zg, 200zh, 200zi in accordance with some implementations. Implants 200za, 200zb, 200zc, 200zd, 200ze, 200ze, 200zf, 200zg, 200zh, 200zi can be considered to be variants of implant 200, and can be similar, at least in their general purpose, e.g., being anchored to a tissue of the heart so as to repair the function of the native leaflet, to implant 200 disclosed hereinabove, mutatis mutandis.

Similarly to limiters 284y, 284z of implants 200y, 200z, that become resistant to upstream articulation of tip portion 232y, 232z when the tip-portion reaches a deflection-limit, limiters 284za, 284zb, 284zc, 284zd, 284ze, 284ze, 284zf, 284zg, 284zh, 284zi of implants 200za, 200zb, 200zc, 200zd, 200ze, 200ze, 200zf, 200zg, 200zh, 200zi inhibit upstream deflection of the wing 220za, 220zb, 220zc, 220zd, 220ze, 220ze, 220zf, 220zg, 220zh, 220zi of the implant when the wing reaches the deflection-limit. However, in contrast to limiters 284y, 284z of implants 200y, 200z, limiters 284za-zi are configured for wings that do not necessarily comprise flex elements. As described hereinbelow, limiters 284za-zi provide an opposing force upon their corresponding wing reaching a deflection-limit, thereby inhibiting upstream deflection of the wing beyond the deflection-limit (e.g., into atrium 6). In some implementations, limiters 284za-zi limit a range of motion of respective tip portions 232za, 232zb, 232zc, 232zd, 232ze, 232ze, 232zf, 232zg, 232zh, 232zi of the wing, such that the deflection-limit is downstream of a line of coaptation shared by the posterior leaflet and anterior leaflet 14.

In some implementations, limiters 284za-zi inhibit upstream deflection of their corresponding wing only when the wing reaches the deflection-limit. For example, contact can be made with the limiter upon the wing reaching the deflection-limit. Such contact can be between the wing and the limiter, or between another component of the implant (e.g., interface 250) and the limiter.

In some implementations, limiters 284za-zi also partially inhibit upstream deflection of the wing prior to the wing reaching the deflection-limit. In some implementations, the opposing force that these limiters apply to the wing is strengthened as the wing reaches and/or passes the deflection-limit.

In some implementations, the limiter is coupled to the interface of the implant. Limiters 284za, 284zb, and 284zc are examples of this. In some implementations, the limiter extends from the interface and away from the wing, e.g., on an opposite side of the interface from the wing. In this way (e.g., as shown in FIG. 35), the wing is deflectable toward and away from the limiter during the cardiac cycle.

In some implementations, interface 250 approaches the limiter as the wing approaches the deflection-limit. Limiters 284za and 284ab are examples of this—as can be limiter 284c. In some implementations, and as shown in the bottom frame of FIG. 35, interface 250 can contact limiter 284za (e.g., the interface can become temporarily seated within a cradle 283za defined by limiter 284za) upon wing 220za reaching the deflection-limit.

In some implementations, interface 250 is anchored to annulus 11 such that tissue of the annulus and/or atrium 6 provides support to the limiter. That is, some of the opposing force can be a reference force provided by tissue of the heart. For example, the limiter can be shaped such that the limiter is pressed against the tissue of atrium 6 as interface 250 is anchored to the site.

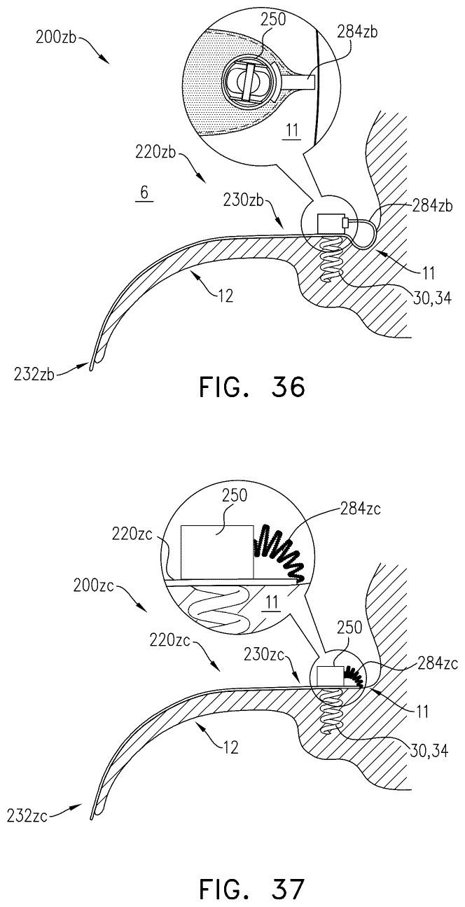

In some implementations, as shown in FIGS. 36-37, limiter 284zb, 284zc applies the opposing force to wing 220zb, 220zc via interface 250. For example, the limiter can define a tensioned loop 284zb (FIG. 36) and/or a compression spring 284zc (FIG. 37) that applies the opposing force to the wing, via interface 250. In some implementations in which the limiter comprises a spring, the spring is configured to bias interface 250 toward opposing leaflet 14, and/or to strain as the interface moves away from the opposing leaflet, thereby strengthening the opposing force as the wing nears and/or passes the deflection-limit.

In some implementations, limiter 284zb, 284zc applies the opposing force to wing 220zb, 220zc not only upon the wing reaching the deflection-limit, but also when the wing is downstream of the deflection-limit. In some implementations, the opposing force that the limiter applies upon the wing is strengthened as the wing reaches and/or passes the deflection-limit.

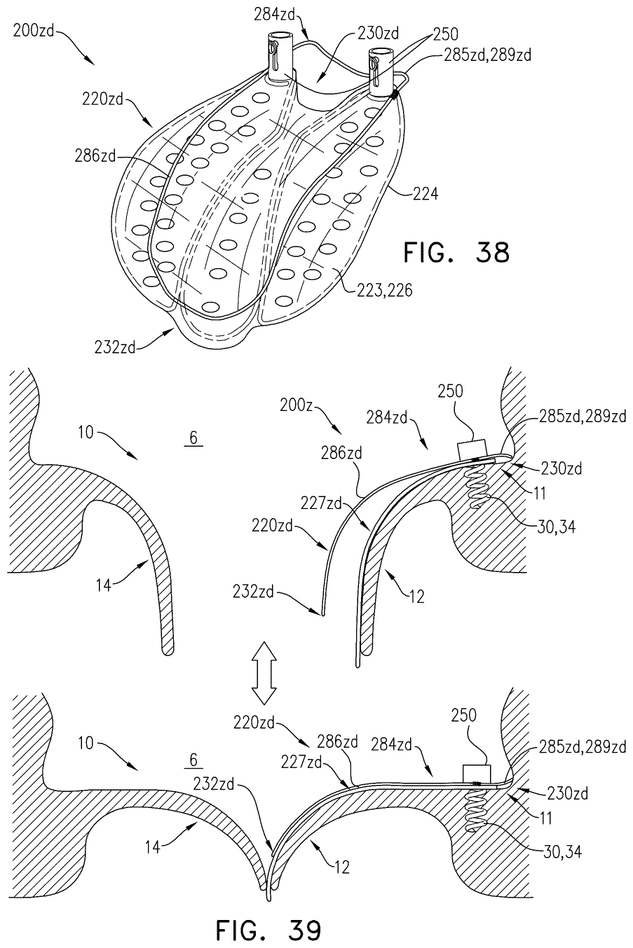

FIGS. 38-44 show limiters 284zd, 284ze, 284ze, 284zf, 284zg, 284zh, 284zi that each comprise a respective wire frame 289zd, 289ze, 289ze, 289zf, 289zg, 289zh, 289zi. Limiters 284zd, 284ze, 284ze, 284zf, 284zg, 284zh, 284zi can each define a backstop portion 285zd, 285ze, 285zf, 285zg, 285zh, 285zi that is shaped to press against tissue of atrium 6 when interface 250 is anchored to annulus 11, e.g., by extending from the interface away from the wing. This is shown in FIG. 39 for limiter 284zd.

In some implementations, and as shown in the upper frame of FIG. 39, wing 220zd contacts limiter 284zd when the wing reaches the deflection-limit. In some implementations, the deflection-limit is defined by a relative position between limiter 284zd (e.g., a limiter-tip portion 286zd) and wing 220zd. For example, and as shown, limiter 284zd contacts a contact-portion 227zd (e.g., a contact-portion between root portion 230zd and tip portion 232zd of the wing) when wing 220zd reaches the deflection-limit. For example, and as shown, limiter 284zd defines a wing-facing cross-brace that lies in contact with the wing, widthways across the wing when the wing reaches the deflection-limit.

In some implementations, the limiter can comprise a second wing, such as second wings 251c, 251d described hereinabove with reference to FIGS. 6-7. In some implementations, second wings 251c, 251d serving as limiters are stiffer than wing 220. In some implementations, second wings 251c, 251d serving as limiters shorter than (FIG. 7) and/or narrower than wing 220.

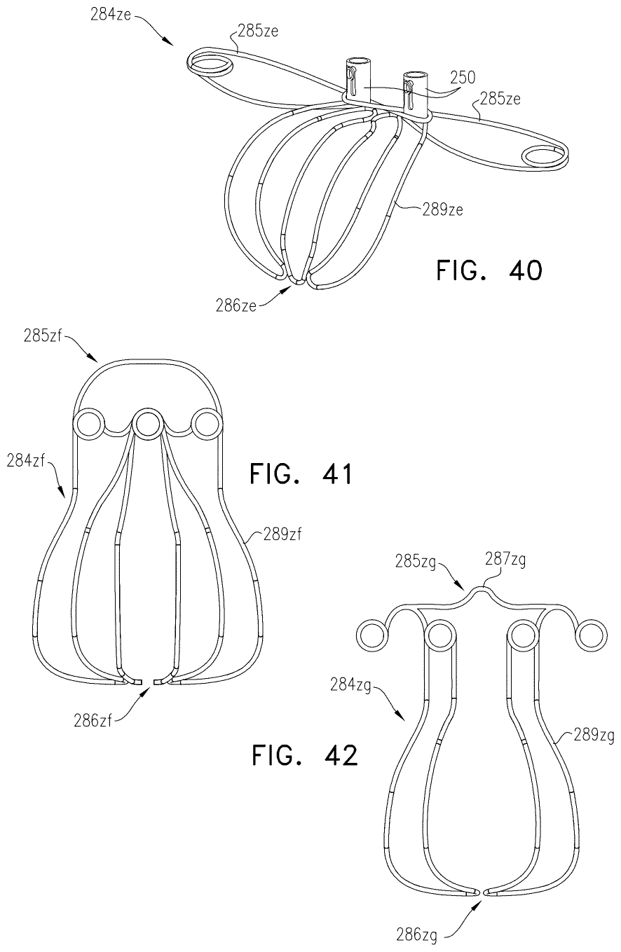

FIGS. 40-44 show limiters 284ze, 284zf, 284zg, 284zh, 284zi for use with implant wing 220 or any of the variants thereof described herein. As shown, limiters 284ze, 284zf, 284zg, 284zh, 284zi are shaped to extend from interface 250 (shown in FIG. 40) to respective backstop portions 285ze, 285zf, 285zg, 285zh, 285zi, and to extend from the interface in an opposite direction toward respective limiter-tip portions 286ze, 286zf, 286zg, 286zh, 286zi. In this way, interfaces 250 can be used to fasten limiters 284ze, 284zf, 284zg, 284zh, 284zi to the wing.

In some implementations, and as shown, backstop portions 285ze, 285zf, 285zg, 285zh, 285zi of limiters 284ze, 284zf, 284zg, 284zh, 284zi are shaped to receive additional anchors 30, such that the backstop portion can be anchored directly to annulus 11, e.g., via additional interfaces 250 (not shown). In some implementations, and as shown in FIGS. 40 and 42, backstop portion 285ze, 285zg, is wider than tip portion 286ze, 286zg, e.g., for greater stability of limiters 284ze, 284zg.

In some implementations, and as shown in FIG. 40, backstop portion 285ze of limiter 284ze is joined to tip portion 296ze of the limiter in a manner that at least partially insulates the backstop portion from forces (e.g., upstream forces, or wrenching forces oblique to a direction in which anchor 30 is anchored into the tissue) that can be applied to the tip portion of the limiter during the cardiac cycle. In some implementations, and as shown, backstop portion 285ze of limiter 284ze is joined to tip portion 296ze via interface 250, such that a wrenching force can move tip portion 286ze with respect to the interface, yet without necessarily moving backstop portion 285ze with respect to the interface.

In some implementations, the backstop portion of the limiter defines a backstop cross-brace (e.g., along a width of the limiter). In some implementations, the backstop cross-brace presses against tissue of atrium 6 upon anchoring of interface 250 to annulus 11, e.g., as shown in FIG. 39 for 284zd. Backstop portions 285zf, 285zg, and 285zh can also be considered to define a backstop cross-brace.

In some implementations, backstop portion 285zg, 285zi (e.g., the backstop cross-brace, as shown) of limiter 284zg, 284zi is shaped to define an annular-facing protrusion 287zg, 287zi. In some implementations, annular-facing protrusion 287zg, 287zi contacts tissue of annulus 11 and/or atrium 6 when interface 250 secured to the annulus, facilitating transfer of the reference force from tissue of the heart to the limiter.

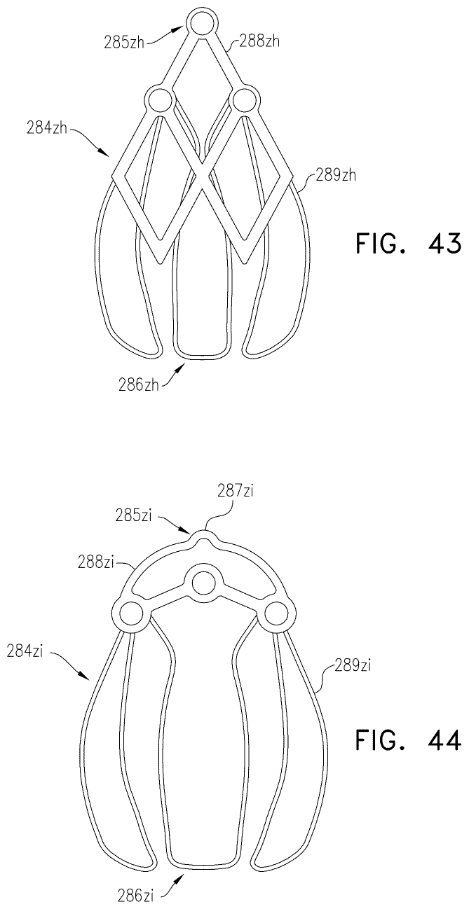

In some implementations, and as shown in FIGS. 43-44, limiters 284zh, 284zi comprise a sheet metal portion 288zh, 288zi (e.g., a sheet metal portion cut to define a plurality of adjoining cells, as shown in FIG. 43) that is coupled to a wire portion 289zh, 289zi. In some implementations, sheet metal portion 288zh, 288zi applies the opposing force to the wing (e.g., to the root portion of the wing) as the wing reaches and/or passes the deflection-limit. In some implementations, wire portion 289zh, 289zi comprises or is formed from material (e.g., a shape-memory alloy) that is more flexible than sheet metal portion 288zh, 288zi.

In some implementations, sheet metal portion 288zh, 288zi is stiffer than wire portion 289zh, 289zi, such that limiter 284zh, 284zi provides firmer support to root portion 230 of wing than to tip portion 232 of the wing. Similarly to as described hereinabove regarding implants 200c, 200d, with reference to FIGS. 6-7, sheet metal portion 288zh, 288zi can be sufficiently stiff to inhibit wing 220 deflecting (e.g., prolapsing) into the atrium, and/or to support a prolapsing portion of native leaflet 12 during ventricular systole.

In some implementations, wire portion 289zh is deflectable with respect to sheet metal portion 288zh. Therefore, since sheet metal portion 288zh is shorter than wire portion 289zh (e.g., the wire portion, but not the sheet metal portion, extends all the way to limiter-tip portion 286zh of limiter 284zh), the limiter can inhibit deflection of wing 220 while allowing tip portion 232 of the wing to behave in its flexible manner, and to coapt optimally with leaflet 14. In some implementations, as the native valve transitions from diastole to systole, wing 220 moves upstream (e.g., pushed by leaflet 12) such that the wing first contacts wire portion 289zh of limiter 284zh, pushing the wire portion toward, and eventually into contact with, sheet metal portion 288zh. Thus, the resistance to deflection of the leaflet that is provided by the implant can gradually (e.g., cumulatively) increase throughout systole, e.g., with some resistance occurring upon the wing contacting wire portion 289zh, and with some more resistance occurring upon the wire portion contacting sheet metal portion 288zh.

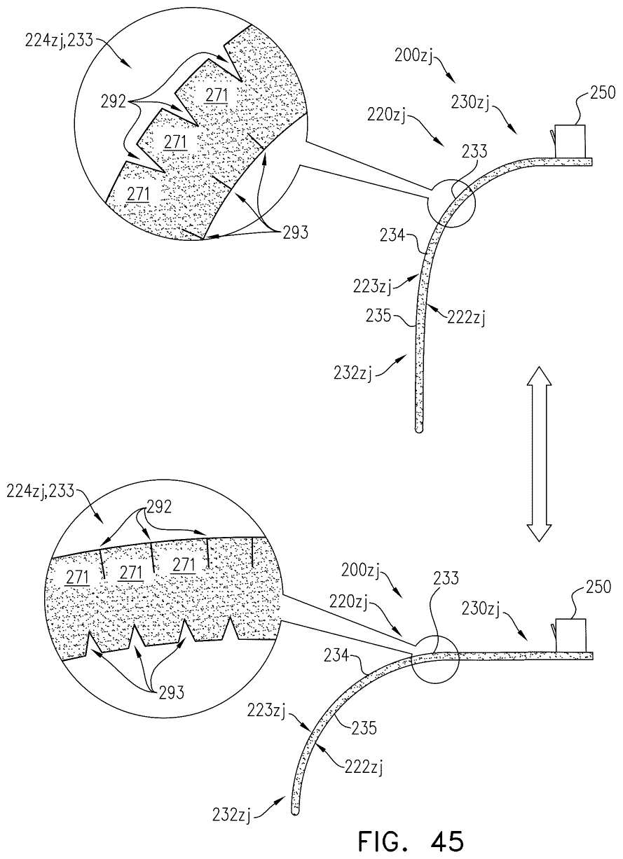

Reference is made to FIG. 45, which shows schematic illustrations of implant 200zj, in accordance with some implementations. Implant 200zj can be considered to be a variant of implant 200, and can be similar, at least in its general purpose, e.g., being anchored to a tissue of the heart so as to repair the function of the native leaflet, to implant 200 or any of the variants thereof disclosed herein, mutatis mutandis, except that wing 220zj of implant 200zj defines a deflection-limit of the wing, similarly to the limiters described hereinabove. However, in contrast to the limiters that are discrete elements of respective implants, wing 220zj limits its own deflection. That is, while interface 250 of implant 200zj is anchored to annulus 11, wing 220zj becomes resistant to deflection in the upstream direction upon reaching the deflection-limit.

Other frames 224 described hereinabove can be formed from one or more wires. Frame 224zj can be formed similarly, but its wire(s) can be hollow, e.g., can be elongate tubes.

In some implementations, and as shown, frame 224zj defines notches 292, e.g., that are cut into the upstream side of the frame, e.g., the side that serves as or faces toward second face/opposing face 223zj of wing 220zj. These notches can also be called upstream notches. As shown, downstream deflection of wing 220zj causes upstream notches 292 to widen (upper frame of FIG. 45). In some implementations, upstream notches 292 facilitate deflection of frame 224zj in the downstream direction to a greater degree than the frame would deflect in the absence of the upstream notches.

Frame 224zj can be considered to define multiple parts 271, between notches 291. Thus, during deflection in the downstream direction, parts 271 become further from each other.

Upstream deflection of wing 220zj causes upstream notches 292 to narrow. The lower frame of FIG. 45 shows upstream notches 292 having narrowed to a point at which the notches have closed and parts 271 contact each other. Once upstream notches 292 close, contact between parts 271 causes an increase in the resistance of frame 224zj, and therefore wing 220zj, to upstream deflection. In some implementations, the degree of upstream deflection at which upstream notches 292 close, such that parts 271 contact each other, is the deflection-limit (e.g., an upstream deflection-limit) of wing 220zj.

In some implementations, upstream notches 292 are different in number, proximity, size, and/or shape in different regions of wing 220zj. Examples of such regions are a first region 233, a second region 234 and a third region 235. The differences in the upstream notches can confer, on the different parts of the wing, different flexibilities, different deflection-limits, and even different shapes at the deflection-limit. For example, region 233 (e.g., closer to the root portion of the wing) can be stiffer and/or have a lower deflection-limit than region 234.

In some implementations, and as shown, frame 224zj (e.g., first region 233 of tip portion 232zj, as shown) also defines notches 293 that are cut into the downstream side of the frame, e.g., the side that serves as or faces toward first face/contact face 222zj of wing 220zj. These notches can also be called downstream notches. As shown in the lower frame of FIG. 45, upstream deflection of wing 220zj causes downstream notches 293 to widen. In some implementations, downstream notches 293 facilitate deflection frame 224 to deflect in the upstream direction to a greater degree than the frame would deflect in the absence of the downstream notches.

Downstream deflection of wing 220zj causes downstream notches 293 to narrow. The upper frame of FIG. 45 shows downstream notches 293 having narrowed to a point at which the notches have closed. Once upstream notches 292 close, frame 224zj, and therefore wing 220zj, typically resists downstream deflection to a greater degree. In some implementations, the degree of downstream deflection at which downstream notches 293 close is a second deflection-limit (e.g., a downstream deflection-limit) of wing 220zj.

In some implementations, downstream notches 293 are different in number, proximity, size, and/or shape in different regions (e.g., regions 233, 234, 235) of wing 220zj. The differences in the downstream notches can confer, on the different parts of the wing, different flexibilities, different downstream deflection-limits, and even different shapes at the downstream deflection-limit. For example, region 233 (e.g., closer to the root portion of the wing) can be more flexible and/or have a higher downstream deflection-limit than region 234.

In some implementations in which frame 224zj defines upstream notches 292 and downstream notches 293, the upstream notches and the downstream notches widen and narrow in a reciprocating manner. That is, the frame is configured (e.g., upstream and downstream notches are dimensioned) such that, as shown in FIG. 45: (i) upstream deflection of wing 220zj widens downstream notches 293 and narrows upstream notches 292, and (ii) downstream deflection of the wing widens upstream notches 292 and narrows downstream notches 293. In some implementations, frame 224zj is more flexible to downstream deflection than to upstream deflection, e.g., due to upstream notches 292 being larger or more numerous than downstream notches 293.

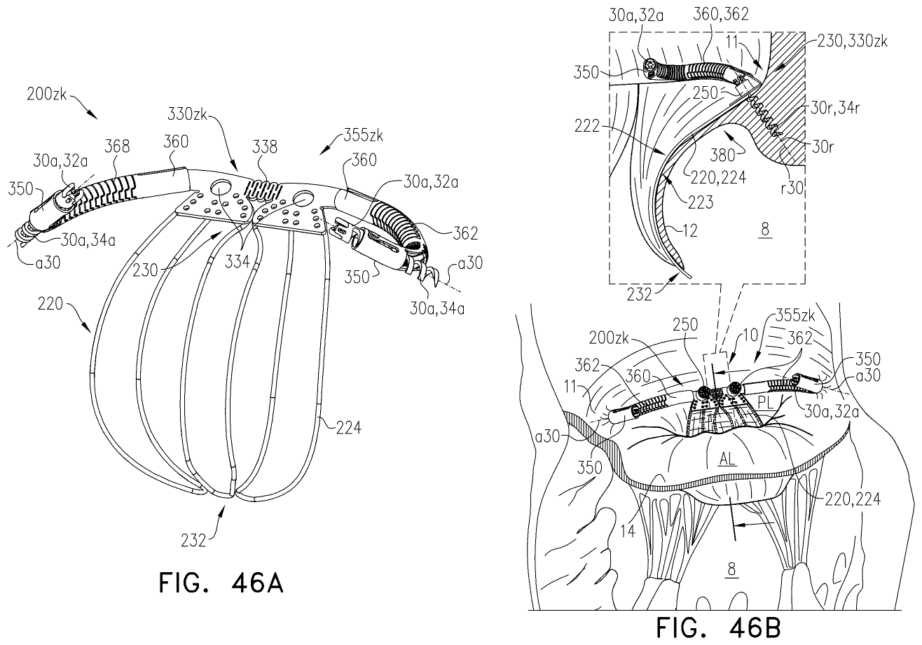

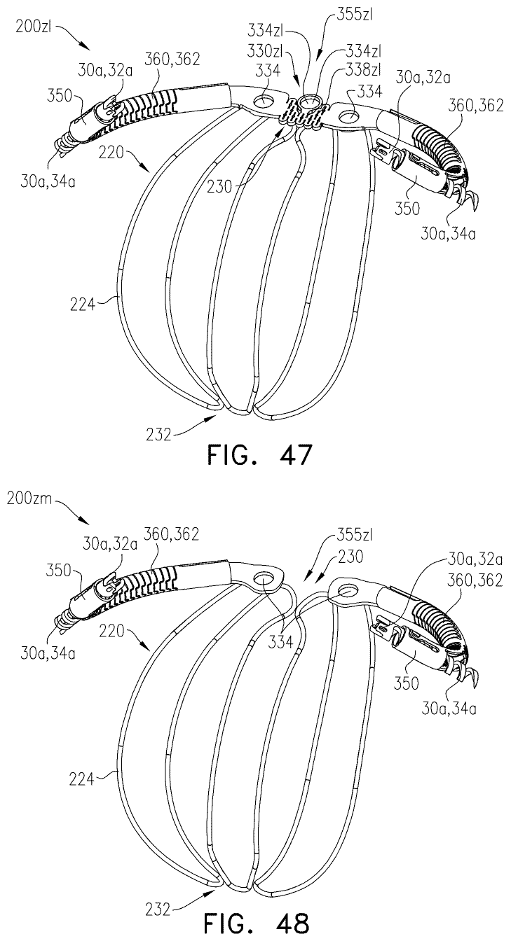

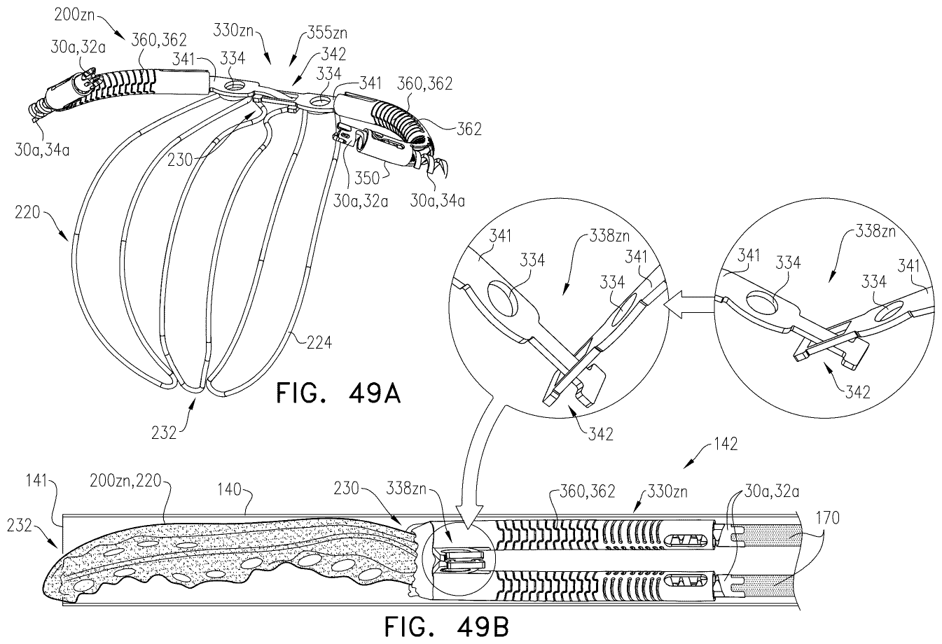

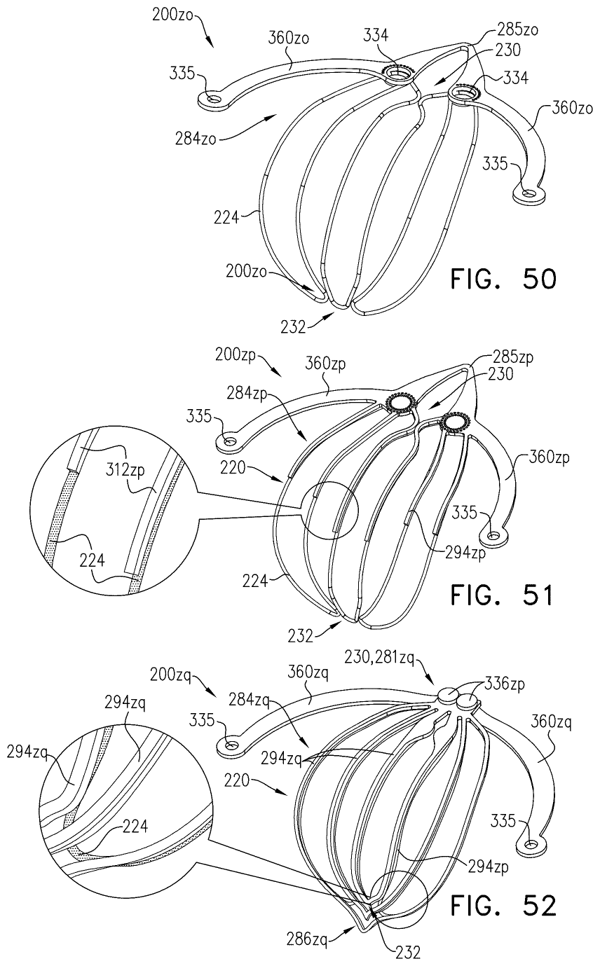

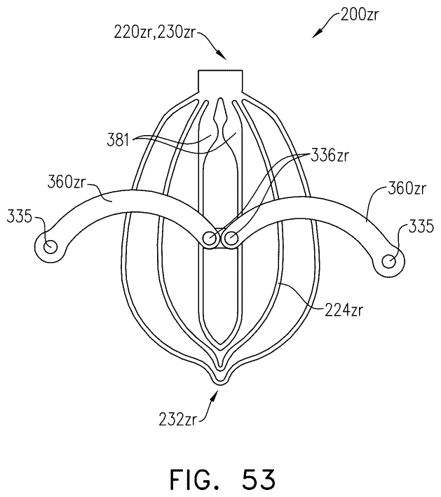

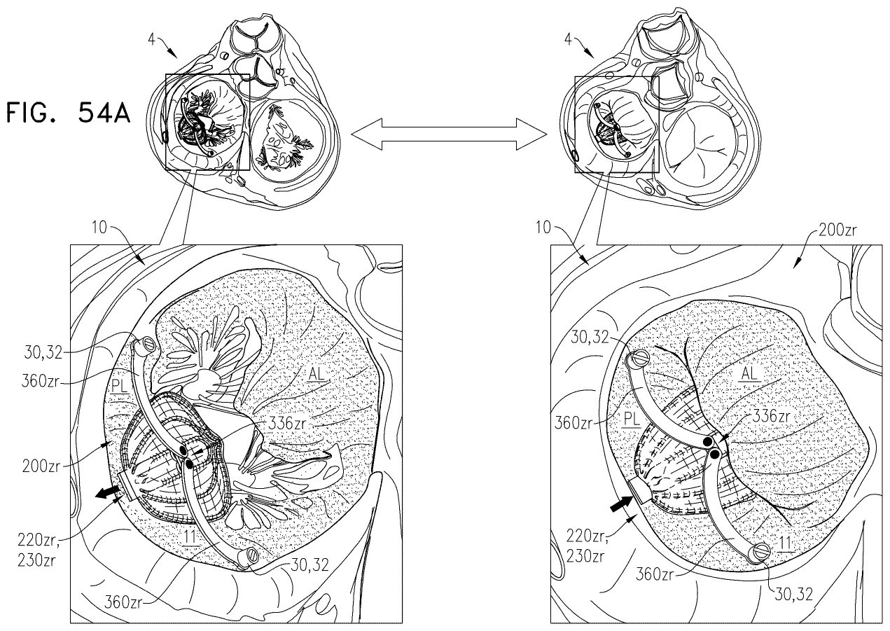

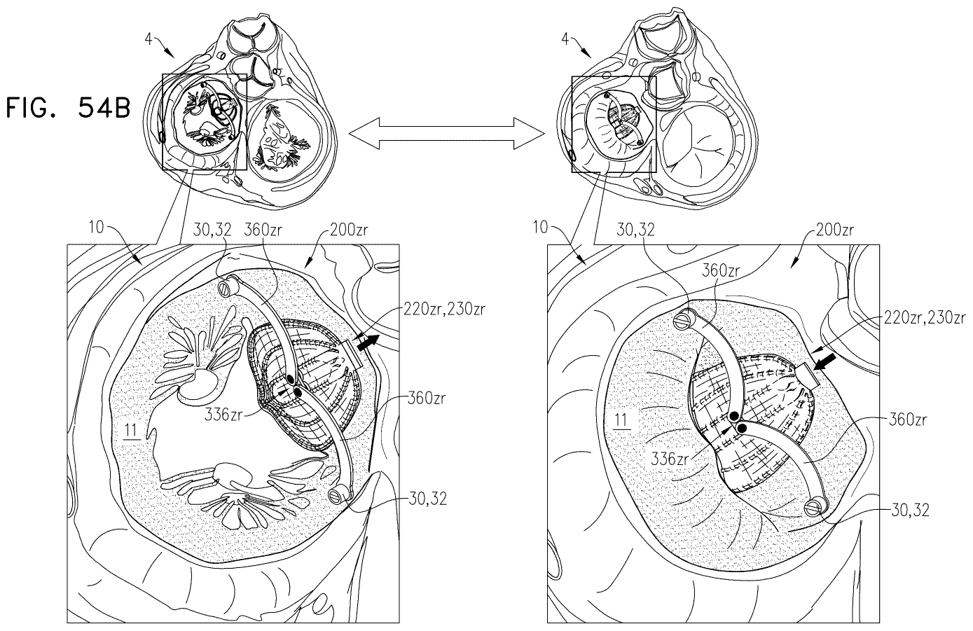

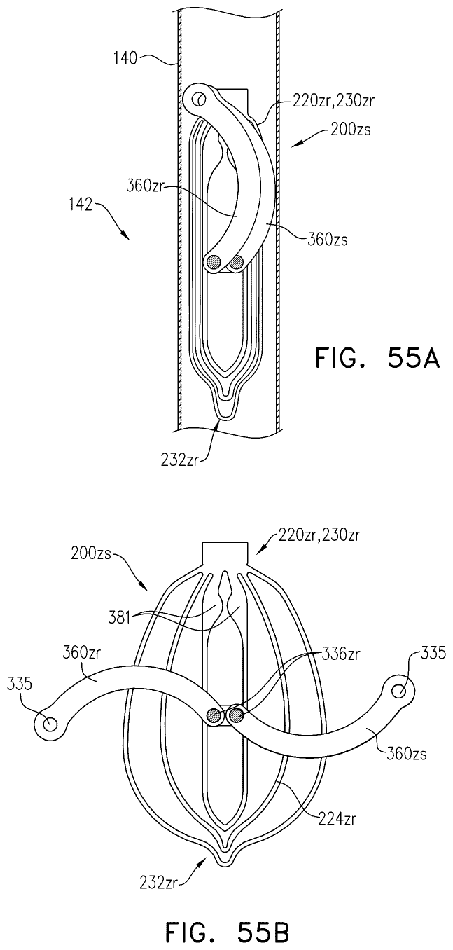

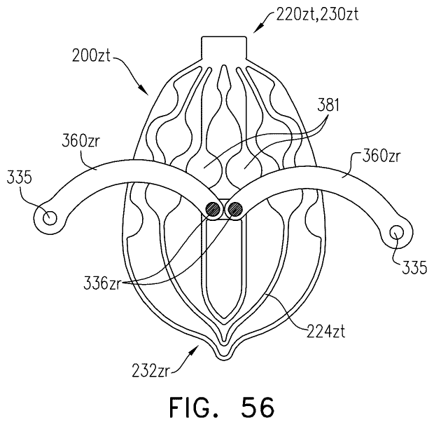

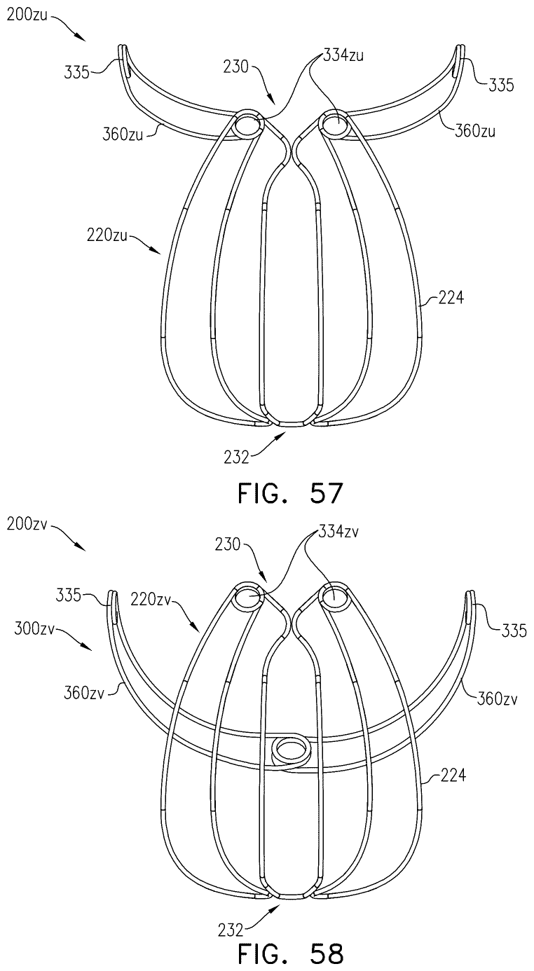

Reference is made to FIGS. 46A-B, 47-48 and 49A-B which are schematic illustrations showing implants 200zk, 200zl, 200zm, 200zn, in accordance with some implementations. Implants 200zk, 200zl, 200zm, 200zn can be considered to be variants of implant 200, and can be similar, at least in their general purpose, e.g., being anchored to a tissue of the heart so as to repair the function of the native leaflet, to implant 200 or any of the variants thereof disclosed herein, mutatis mutandis, except that implants 200zk, 200zl, 200zm, 200zn each comprise a pair of annular arms 360 that are away from wing 220.

In some implementations, and as shown in FIG. 46A, annular arms 360 (e.g., arms for placement along the annulus) are each coupled to root portion 230 of wing 220 (which is shown without sheet 226), e.g., via a root brace 330 that supports the root portion of the wing. Annular arms 360 may be considered to serve as an annular support 355zk of the implant (e.g., a support for placement at the annulus). In some implementations, root brace 330 defines a joint 338 that is shaped to facilitate folding of the root brace along the joint, and thereby to facilitate folding of the implant.

Typically for such implementations, each arm 360 (e.g., an articulated portion 362 thereof) arcs divergently away from root portion 230 to an anchor receiver, e.g., to an interface 350 that is generally similar to interfaces 250 described hereinabove, mutatis mutandis. In some implementations, and as shown, arms 360 (e.g., articulated portions 362 thereof) comprise hypodermic tubing that is selectively cut in order to allow the articulated portion to assume its arced shape.

In some implementations, and as shown in FIG. 46A, arm anchors 30a (typically similar to anchor 30 described hereinabove) are held within interface 350 at an angle that is not parallel to arms 360. In some implementations, and as shown, a tissue-engaging element 34a of each anchor 30a extends from an anchor head 32a along an anchor axis a30 that is generally perpendicular to a portion of arm 360. For example, and as shown, each interface 350 can define a longitudinal axis that is also perpendicular to the portion of arm 360.

Typically for such implementations, and as shown in FIG. 46B, implant 200zk is implanted at mitral valve 10, with interfaces 350 anchored to annulus 11 by arm anchor 30a. As shown, wing 220 is secured in position such that root portion 230 is disposed against annulus 11, and the wing extends, from the root portion, over posterior leaflet 12 of mitral valve 10, toward the opposing, anterior leaflet 14. In this way, the wing can reciprocatingly deflect upstream and downstream in response to the cardiac cycle of the heart.

In some implementations, and as shown, each arm 360 arcs from the interface, along an atrial side of annulus 11, and to root portion 230 of wing 220. In this way, each arm 360 extends along (e.g., in contact with) annulus 11, from root portion 230 toward interface 350. Contact between arm 360 and tissue of annulus 11 moderates upstream deflection of wing 220, e.g., by keeping root portion 230 of wing 220 in contact with the annulus as tip portion 232 of the wing deflects during the cardiac cycle.

In some implementations, using anchors 30a to anchor interfaces 350 to an atrial surface of annulus 11 (e.g., adjacent to commissures of valve 10) is sufficient to keep root portion 230 of wing 220 in contact with annulus 11 during the cardiac cycle.

In some implementations, in addition to arm anchors 30a, root portion 230 of wing 220 is also anchored to annulus 11. In some implementations, and as shown in FIG. 46B, root anchors 30r (e.g., anchors 30 described hereinabove) are driven through interfaces 250 at respective anchor receivers 334 (FIG. 46A) into tissue of annulus 11.

Typically for such implementations, each root anchor 30r defines a root anchor axis r30 along which the root anchor is driven into tissue of annulus 11. In some implementations, while wing 220 is secured in position by anchors 30a, 30r, upstream deflection of the wing is in a direction (arrow 380 in the inset of FIG. 46B) that is closer to being parallel to root anchor axes r30 than to arm anchor axes a30. For example, wing 220 can deflect upstream in a direction that is generally parallel to root anchor axes r30, but oblique to arm anchor axes a30.

FIG. 47 shows implant 200zl, which is generally identical to implant 200zk, except that root brace 330zl of implant 200zl is shaped to define, alongside joint 228zl, an anchor receiver 334zl. In some implementations, and as shown, anchor receiver 334zl supplements anchor receivers 334 described hereinabove. In this way, root portion 230 of wing 220 can be anchored to annulus 11 by three root anchors 30r. Alternatively, anchor receiver 334zl can be anchored to the annulus instead of anchor receivers 334. In some implementations, implant 200zl can define anchor receivers 334zl, but not anchor receivers 334.

FIG. 48 shows implant 200zm, which is generally identical to implant 200zk, except that implant 200zm does not include a root brace 330, or a discrete joint. Typically for such implementations, flexibility of implant 200zm, e.g., of wing 220 thereof, is sufficient to facilitate folding of the implant.

FIGS. 49A-B show implant 200zn, which is generally identical to implant 200zk, except that root brace 330zn of implant 200zn defines a hinge 342 that facilitates folding of the implant, e.g., in order to fit the implant into distal portion 142 of delivery tool 150, as shown in FIG. 49B. In some implementations and as shown, hinge 342 comprises two buckles 341 that articulate with respect to each other to facilitate folding of the hinge and therefore of implant 200zn.

Reference is made to FIGS. 50-52, which are schematic illustrations showing implants 200zo, 200zp, 200zq, in accordance with some implementations. Implants 200zo, 200zp, 200zq can be considered to be variants of implant 200, and can be similar, at least in their general purpose, e.g., being anchored to a tissue of the heart so as to repair the function of the native leaflet, to implant 200 or any of the variants thereof disclosed herein, mutatis mutandis.

In some implementations, and as shown, implants 200zo, 200zp, 200zq each comprise a limiter 284zo, 284zp, 284zq that is coupled to wing 220 (shown without sheet 226) and configured to inhibit deflection of the wing. As shown in FIGS. 50-51, limiters 284zo, 284zp of implants 200zo, 200zp each comprise a backstop portion 285zo, 285zp that is shaped to press against tissue of atrium 6 e.g., against an atrial surface of annulus 11, when root portion 230 of wing 220 is secured to the annulus. In some implementations, and as shown in FIG. 50, limiter 284zo defines anchor receivers 334 to which interfaces 250 can be fitted for anchoring root portion 230 to tissue of annulus 11. Thus, when root portion 230 is secured in place by anchoring the anchor receivers to tissue of annulus 11, arms 360zo, 360zp can transfer force that tissue of the chamber applies to backstop portion 285zo, 285zp to wing 220, thereby limiting deflection of the wing.

In some implementations, and as shown, arms 360zo, 360zp 360zq of implants 200zo, 200zp, 200zq extend from backstop portion 285zo, 285zp to anchor receivers 335. In some implementations, when root portion 230 is secured in place, anchor receivers 335 can be anchored to tissue adjacent to commissures of valve 10.

As shown in FIG. 51, limiter 284zp of implant 200zp defines ribs 294zp that extend from backstop portion 285zp along wing 220 (e.g., along corresponding portions of the wing's frame 224), from root portion 230 toward tip portion 232. In some implementations, and as shown, wing 220 can deflect such that limiter 284zp (e.g., ribs 294zp thereof) contacts the wing during the cardiac cycle.

In some implementations, contact between limiter 284zp and wing 220 can be maintained throughout the cardiac cycle.

Alternatively, wing 220 can deflect upstream to contact limiter 284zp only during part of the cardiac cycle, e.g., during ventricular systole. For example, contact between wing 220 and limiter 284zp can define the wing's deflection-limit, such that the wing contacts the limiter 284zp upon the wing reaching the wing's deflection-limit. As described hereinabove with reference to implant 200zd (FIGS. 38-39), limiter 284zp can inhibit deflection of wing 220 in the upstream direction beyond the deflection-limit.

As shown in FIG. 52, implant 200zq is in many ways similar to implant 200zp, except that ribs 294zq of limiter 284zp extend further away from root portion 230 of wing, e.g., to a limiter-tip portion 286zq that is disposed over the wing's tip portion 232. Additional support that ribs 294zq provide to tip portion 232 can make implant 200zq particularly suitable for implantation at a valve having a leaflet 12 experiencing flailing and/or prolapse at a tip portion of the leaflet.

In some implementations, and as shown, rather than defining a backstop portion, a limiter-root portion 281zq of limiter 284zq is coupled to wing 220 at swivel-hinges 336zq that allow arms 360zq to rotate with respect to the wing. Rotation of arms 360zq with respect to wing 220 while anchor receivers 335 are be anchored to tissue adjacent to commissures of valve 10 can facilitate movement of the wing in concert with tissue of annulus 11 as the valve cycles between systole and diastole, thereby maintaining contact between root portion 230 and the annulus during the cardiac cycle.

Reference is made to FIGS. 53, 54A-B and 55-56, which are schematic illustrations showing implants 200zr, 200zs, 200zt, in accordance with some implementations. Implants 200zr, 200zs, 200zt can be considered to be variants of implant 200, and can be similar, at least in their general purpose, e.g., being anchored to a tissue of the heart so as to repair the function of the native leaflet, to implant 200 or any of the variants thereof disclosed herein, mutatis mutandis.

As shown, implants 200zr, 200zs, 200zt comprise wing 220zr that is generally similar to wing 220, except that wing 220zr can be manufactured by alternative means, e.g., wing 220zr can be laser-cut from a piece of sheet-metal, whereas wing 220 typically comprises a wire frame 224. Notwithstanding the differences between wing 220 and wing 220zr, wing 220zr can be used in conjunction with other implementations disclosed herein, mutatis mutandis.

In some implementations, and as shown, frame 224zr define buttresses 381 at root portion 230zr of wing 220zr. As shown, buttresses 381 are wider than frame 224zr at tip portion 232zt of wing 220zt. Buttresses 381 provide stiffness to root portion 230zt of wing 220zt, such that the root portion is less likely to deflect in response to the forces applied to the wing during the cardiac cycle. In some implementations, buttresses 381 serve as limiters, by providing the opposing force upon wing 220zr reaching the deflection-limit.

As shown, arms 360zr, 360zs are coupled to wing 220zr via swivel-hinges 336zr, at a portion of the wing that is between the wing's root portion 230zt and the wing's tip portion 232zr. As described hereinabove with reference to implant 200zq, swivel-hinges 336zr allow arms 360zr to pivot with respect to the wing. In some implementations, and as shown, arms 360zr are symmetrically away from wing 220zr.

FIGS. 54A-B show implant 200zr implanted at the posterior leaflet (FIG. 54A) or anterior leaflet (FIG. 54B) of mitral valve 10, with anchor receivers 335 of arms 360zr anchored to annular tissue adjacent the valve's commissures. In some implementations, and as shown, as wing 220zr deflects: (i) downstream during ventricular diastole (left frames of FIGS. 54A-B) and (ii) upstream during ventricular systole (right frames of FIGS. 54A-B), arms 360zr responsively articulate with respect to wing 220zr, maintaining contact between the wing's root portion and annulus 11. In some implementations, and as shown in right frames of FIGS. 54A-B, an angle defined by arms 360zr becomes more acute as the wing deflects in the upstream direction.

FIGS. 55A-B shows implant 200zs, which is generally identical to implant 200zr, except that one of arms 360zr is replaced in implant 200zs with arm 360zs. As shown in FIG. 55B, respective arms 360zr, 360zs are asymmetrically away from swivel-hinges 336zr, such that when the arms pivot toward each other, the arms become nested with respect to each other. As shown in FIG. 55A, nested arms 360zr, 360zs conserve space when implant 200zs is compressed within distal portion 142 of delivery catheter 140.

FIG. 56 shows implant 200zt, which is generally identical to implant 200zr, except that frame 224zt defines additional buttresses 381 at root portion 230zt of wing 220zt, which provide additional stiffness to root portion 230zt of wing 220zt, such that the root portion is less likely to deflect in response to the forces applied to the wing during the cardiac cycle.

Reference is made to FIGS. 57-58, which are schematic illustrations showing implants 200zu, 200zv, in accordance with some implementations. Implants 200zu, 200zv can be considered to be variants of implant 200, and can be similar, at least in their general purpose, e.g., being anchored to a tissue of the heart so as to repair the function of the native leaflet, to implant 200 or any of the variants thereof disclosed herein, mutatis mutandis.

As shown, implant 200zu is in certain ways similar to implant 200zk. That is, implant 200zu comprises a pair of arms 360zu that each arc divergently away from root portion 230 (e.g., from anchor receivers 334zu thereof) of wing 220 (shown without sheet 226), to a respective anchor receiver 335. However, in contrast to arms 360 of implant 200zk that are shown comprising cut hypodermic tubing, arms 360zu comprise wire, e.g., wire that is malleable into the arced shape of the arms shown in FIG. 57.

In some implementations, the arms 360zu are such that when anchor receivers 335 are anchored to tissue of the mitral valve's annulus 11 (e.g., via interfaces, not shown), root portion 230 of wing 220 is disposed against the annulus and the wing extends over posterior leaflet 12 and toward anterior leaflet 14. In this way, wing 220 can reciprocatingly deflect upstream and downstream in response to the cardiac cycle. In some implementations, anchor receivers 334zu of wing 220 are also anchored to annulus 11 (e.g., via interfaces, not shown).

As shown in FIG. 58, implant 200zv is in many ways similar to implant 200zu, except that arms 360zv are coupled to wing 220 between root portion 230 and tip portion 232. In this respect, implant 200zv is similar to implant 200zr. However, in contrast to implant 200zr that is cut from a piece of sheet-metal, implant 200zu comprises wing 220, whose frame 224 is manufactured from flexible wire. Arms 360zu also comprise flexible wire, and are fixedly coupled to wing 220, as opposed to arms 360zr, which are connected to wing 220zr by swivel-hinges 336zr. Furthermore, the curve along which arms 360zv arc differs from the curve along which arms 360zr arc. That is, whereas arms 360zr curve along a face of wing 220zr, arms 360zv curve away from the face of wing 220.

In some implementations, the arced shape of arms 360zv is such that when anchor receivers 335 are anchored to annulus 11 (e.g., adjacent to the valve's commissures), root portion 230 of wing 220 is disposed against the annulus and the wing extends over posterior leaflet 12 and toward anterior leaflet 14. In this way, wing 220 can reciprocatingly deflect upstream and downstream in response to the cardiac cycle. In some implementations, anchor receivers 334zv of wing 220zv are also anchored to annulus 11 (e.g., via interfaces 250, not shown).

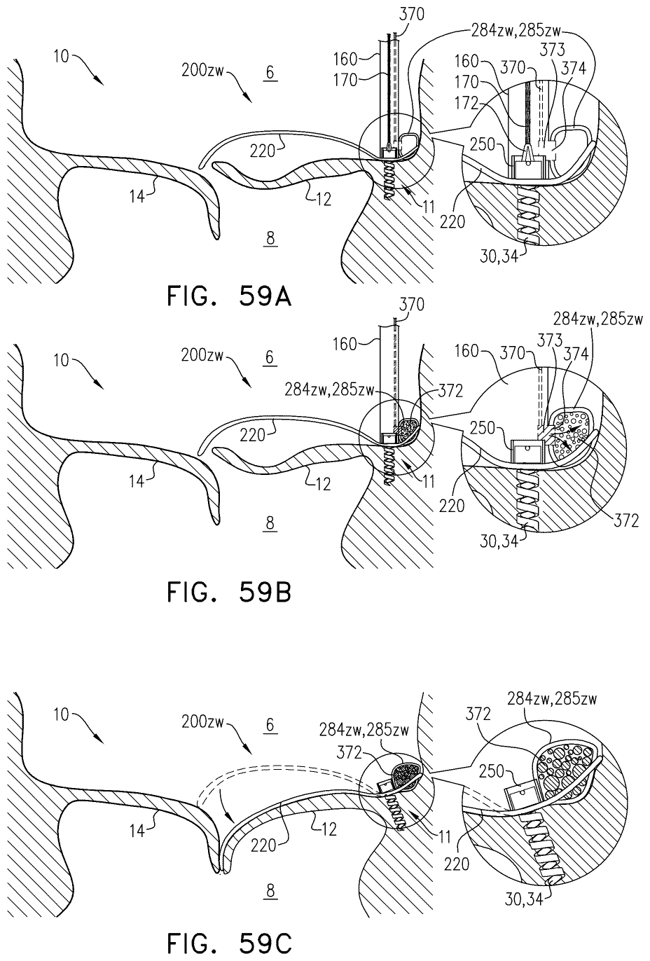

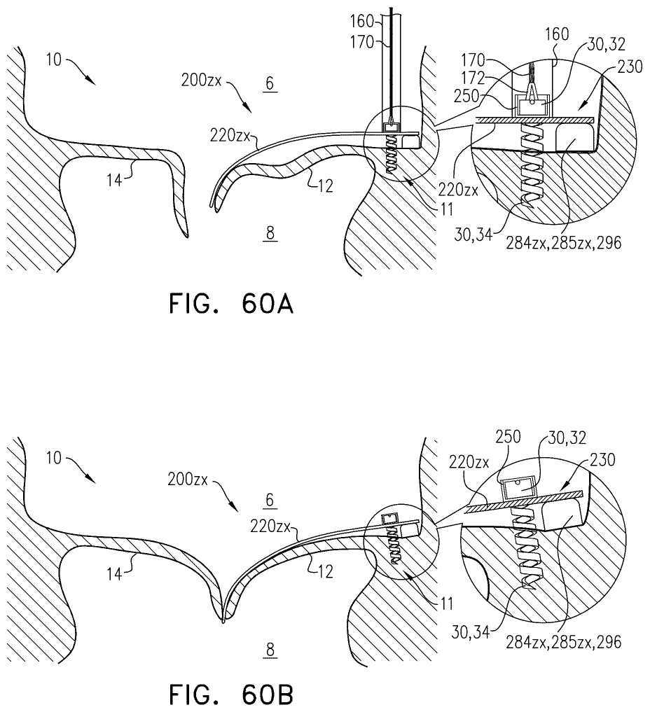

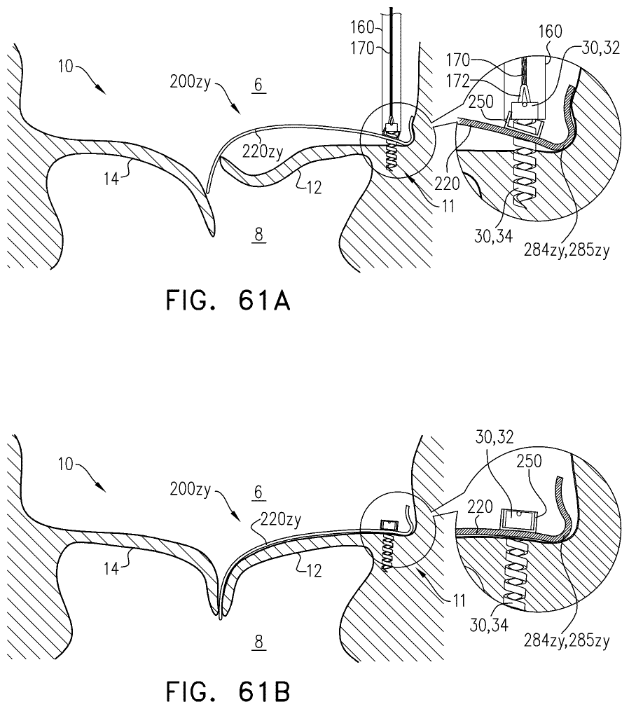

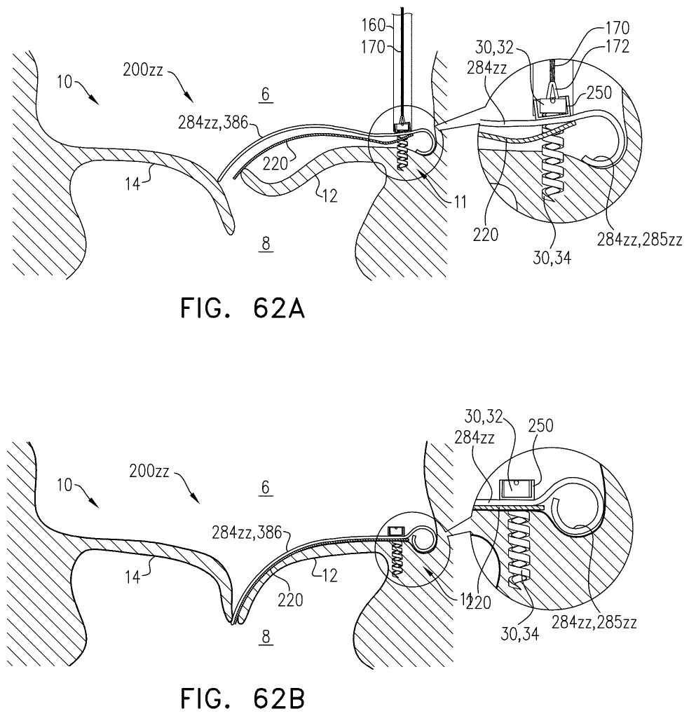

Reference is made to FIGS. 59A-C, 60A-B, 61A-B and 62A-B, which are schematic illustrations showing implants 200zw, 200zx, 200zy, 200zz that comprise respective limiters 284zw, 284zx, 284zy, 284zz, in accordance with some implementations.

Implants 200zw, 200zx, 200zy, 200zz can be considered to be variants of implant 200, and can be similar, at least in their general purpose, e.g., being anchored to a tissue of the heart so as to repair the function of the native leaflet, to implant 200 or any of the variants thereof disclosed herein, particularly implants 200zb and 200zc described hereinabove with reference to FIGS. 36-37. Similarly to as described hereinabove regarding limiters 284ab, 284zc of implants 200zb and 200zc, when interface 250 of implant 200zw, 200zx, 200zy, 200zz is anchored to annulus 11, limiters 284zw, 284zx, 284zy, 284zz apply an opposing force that opposes upstream deflection of wing 220 by pressing backstop portions 285zw, 285zx, 285zy, 285zz against tissue of atrium 6 (e.g., tissue of annulus 11).

In some implementations, limiters 284zw, 284zx, 284zy, 284zz are adjustable (e.g., intracardially adjustable via catheter 140 using controller 120), in order to change a magnitude of the opposing force. In some implementations, backstop portion 285zw, 285zx, 285zy, 285zz is adjustably pressed against tissue of annulus 11, thereby adjusting the magnitude of the opposing force that limiter 284zw, 284zx, 284zy, 284zz applies to wing 220.

In some implementations, adjusting limiters 284zw, 284zx, 284zy, 284zz adjusts the wing's deflection-limit. In some implementations, adjusting limiters 284zw, 284zx, 284zy, 284zz also adjusts magnitude of the opposing force when the wing is downstream of the deflection-limit. Typically for such implementations, the opposing force that limiters 284zw, 284zx, 284zy, 284zz apply upon wing 220 is strengthened as the wing reaches and/or passes the deflection-limit.

As shown in FIGS. 59A-C, limiter 284zw has an inflatable backstop portion 285zw. FIG. 59A shows implant 200zw implanted at mitral valve 10, with wing 220 disposed over a flailing posterior leaflet 12 and backstop 285zw not yet actively inflated. Although implant 200zw is shown in FIG. 59A having been implanted in a generally desirable position, wing 220 does not apply sufficient opposing force to leaflet 12, in order to bring the leaflet into coaptation with the opposing, anterior leaflet 14.

FIGS. 59B-C show adjustment of limiter 284 by inflating backstop portion 285zw. As shown, shaft 160 of delivery tool 150 houses an inflation tube 370 defining a nozzle 373 that is positioned at an opening 374 of backstop portion 285zw. FIG. 59B shows delivery of an expanding material (e.g., a water-absorbing hydrogel 372 that expands upon absorption of liquid) from nozzle 373 of inflation tube 370, through opening 374 and into backstop portion 285zw.

FIG. 59C shows inflation tube 370 having been withdrawn, together with shaft 160. In some implementations, detaching nozzle 373 of inflation tube 370 from opening 374 of backstop portion 285zw causes the backstop portion's opening to close, e.g., to automatically self-seal.

FIG. 59C also shows hydrogel 372 having expanded, e.g., by absorbing liquid from blood that flows through atrium 6. As shown, expansion of backstop portion 285zw presses one side of the backstop portion against an atrial surface of annulus 11, and another side of the backstop portion against implant 200zw (e.g., against interface 250 and/or wing 220 thereof). Inflating backstop portion 285zw thereby pushes against implant 200zw, changing an angle at which wing 220 extends over posterior leaflet 12 and increasing the opposing force that wing 220 applies to the posterior leaflet, which brings the posterior leaflet and the wing into coaptation with anterior leaflet 14.

FIGS. 60A-B, 61A-B and 62A-B show limiters 284zx, 284zy, 284zz that are adjustable by adjusting a depth to which anchor 30 is driven into the tissue. FIGS. 60A, 61A and 62A each show a respective implant 200zx, 200zy, 200zz secured to annulus 11 by anchor 30, such that the anchor's tissue-engaging element 34 is partially anchored within the tissue.

In some implementations, and as shown, driving anchor 30 deeper into tissue of annulus 11 adjusts limiters 284zx, 284zy, 284zz, which increases the opposing force that wing 220, 220zx, 220zy applies to leaflet 12 (e.g., adjusting the wing's deflection-limit), thereby bringing the leaflets into coaptation with each other and/or with the wing during ventricular systole (FIGS. 60B, 61B, 62B).

In some implementations, while tissue-engaging element 34 is partially anchored into tissue of annulus 11, an imaging device is used to image leaflet coaptation and/or retrograde blood flow through valve 10. If the imaging demonstrates that so implanting implant 200zx, 200zy, 200zz resulted in a desirable change in leaflet coaptation or retrograde blood flow, the user can decide to withdraw delivery tool 150, leaving the implant secured to annulus 11 by tissue-engaging element 34 being partially anchored within the tissue. However, if the imaging indicates that increasing the opposing force that wing 220, 220zx, 220zy applies to leaflet 12 can improve leaflet coaptation and/or retrograde blood flow through valve 10, the user can adjust the implant by anchoring (e.g., by applying torque to anchor 30) tissue-engaging element 34 deeper into tissue of annulus 11.

FIGS. 60B, 61B and 62B respectively show implant 200zx, 200zy, 200zz secured to annulus 11 such that the tissue-engaging element 34 is more deeply anchored (e.g., fully anchored) within tissue of the annulus. Anchoring the implant more deeply increases the opposing force that wing 220, 220zx, 220zy applies to posterior leaflet 12 by changing an orientation of the wing with respect to the leaflet.

In some implementations, and as shown, root portion 230 of wings 220zx, 220zy comprise limiters 284zx, 284zy that define backstop portion 285zx, 285zy that are pressed against tissue of annulus 11 as anchor 30 is anchored deeper into the tissue.

In some implementations, interface 250 acts as a fulcrum of limiter 284zx, 284zy that transforms resistance that backstop portion 285zx, 285zy encounters from tissue of annulus 11 on one side of the fulcrum, into the opposing force that wing 220zx, 220zy applies to leaflet 12 on the other side of the fulcrum, thereby bringing posterior leaflet 12 into coaptation with the wing and anterior leaflet 14 (FIGS. 60B, 61B).

In some implementations, and as shown in FIGS. 60A-B, backstop portion 285zx comprises a stopper that keeps root portion 230 of wing 220 from sitting flush on tissue of annulus 11 when the implant is secured to annulus. In some implementations, stopper 296 supplements resistance that backstop portion 285zx, 285zy encounters from tissue of annulus 11, thereby increasing the opposing force that wing 220zx, 220zy applies to leaflet 12 on the other side of the fulcrum.

In some implementations, and as shown in FIGS. 61A-B, backstop portion 285zy defines a spring that is deformed (e.g., changes its curvature) as anchor 30 is anchored further into tissue of annulus 11. Deforming the spring on one side of the fulcrum increases tension on the other side of the fulcrum, causing tip portion 232 of wing 220 to deflect further downstream, as shown in FIG. 61B.

As shown in FIGS. 62A-B, implant 200zz is in many ways similar to implants 200zx, 200zy, with the exception that limiter 284zz is distinct from wing 220 and defines a wing portion 386 that extends from interface 250, over wing 220 and toward opposing leaflet 14, as well as a backstop portion 285zz on the opposite side of the interface. Limiter 284zz functions similarly to limiters 284zx, 284zy, such that that further anchoring anchor 30 presses backstop portion 285zz against tissue of annulus, which in turn increases the opposing force that wing portion 386 applies to wing 220. In some implementations, and as shown in FIG. 62B, further anchoring the anchor sandwiches wing portion 386 between interface 250 and wing 220.

In some implementations, and similarly to the spring of backstop portion 285zy, the spring of backstop portion 285zz is tensioned as anchor 30 is further anchored into the tissue. As shown, further anchoring anchor 30 into the tissue deforms backstop portion 285zy, thereby increasing the opposing force that wing portion 286 applies to wing 220, e.g., bringing wing 220 into greater contact with proximal leaflet 12.

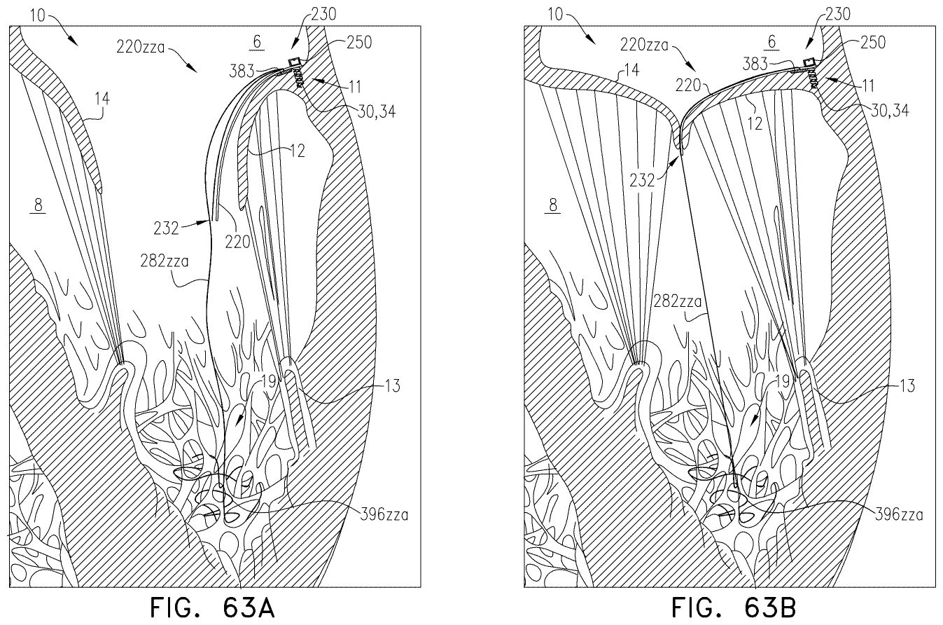

Reference is made to FIGS. 63A-63B, 64A-64B, 65 and 83A-B, which are schematic illustrations showing implants 200zza, 200zzb, 200zzc, 200zzt, in accordance with some implementations.

Implants 200zza, 200zzb, 200zzc, 200zzt can be considered to be variants of implant 200, and can be similar, at least in their general purpose, e.g., being anchored to a tissue of the heart so as to repair the function of the native leaflet, to implant 200 or any of the variants thereof disclosed herein, mutatis mutandis, except that implants 200zza, 200zzb, 200zzc, 200zzt each comprise a tether 282zza, 282zzb, 282zzc, 282zzt that is coupled to wing 220 and anchored to tissue of ventricle 8.

In some implementations, and similarly to tether 282z of implant 200z described hereinabove with reference to FIGS. 32-33, tether 282zza, 282zzb, 282zzc, 282zzt becomes tensioned as wing 220 deflects in the upstream direction, inhibiting the wing's upstream deflection. In some implementations, tether 282zza, 282zzb, 282zzc, 282zzt becomes tensioned as wing 220 reaches the deflection-limit, such that the tether halts upstream deflection of the wing past the deflection-limit. Typically for such implementations, and in contrast to tether 282z, tethers 282zza, 282zzb, 282zzc, 282zzt halt upstream deflection of wing 220 past the deflection-limit independently of separate limiters described hereinabove. Tethers 282zza, 282zzb, 282zzc, 282zzt can therefore serve, in and of themselves, as limiters upon being anchored to tissue of ventricle 8.

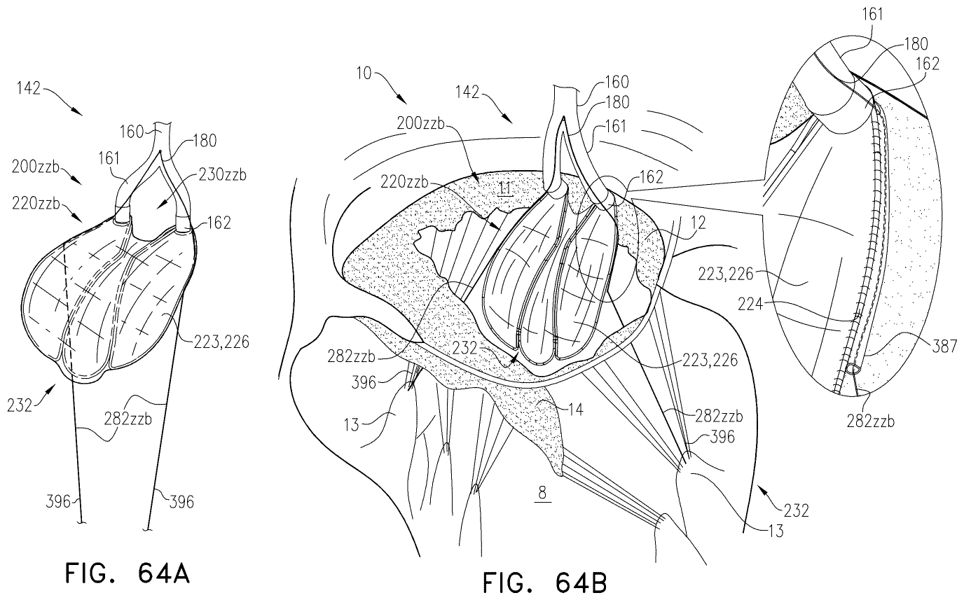

As shown, each tether 282zza, 282zzb, 282zzc, 282zzt extends along wing 220 (e.g., tethers 282zza, 282zzt extend from root portion 230 to tip portion, as shown in FIGS. 63A-B and 83A-B, and tethers 282zzb, 282zzc extend along root portion, as shown in FIGS. 64A-B and 65), to a respective distal portion of the tether that is typically anchored to tissue of ventricle 8.

In some implementations, and as shown in FIGS. 63A-B and 83A-B, tether 282zza, 282zzt is fastened to root portion 230 of wing 220 (e.g., by a pledget 383), and extends from the wing to a distal portion 396zza, 396zzt of the tether. In some implementations, implant 200zza, 200zzt is implanted with tether 282zza, 282zzt already fastened to the wing.

Alternatively, tether 282zza, 282zzt can be fastened to root portion 230 of wing 220 after deploying implant 200zza, 200zzt, e.g., after using anchor 30 to secure the wing's root portion to annular tissue. In some implementations, tether 282zza, 282zzt can be used to adjust the wing's deflection-limit after securing root portion 230 to annulus 11. In some implementations, tether 282zza, 282zzt is fastened to both wing 220 and to leaflet 12 (not shown), e.g., by pledget 383 at least partially penetrating the leaflet.

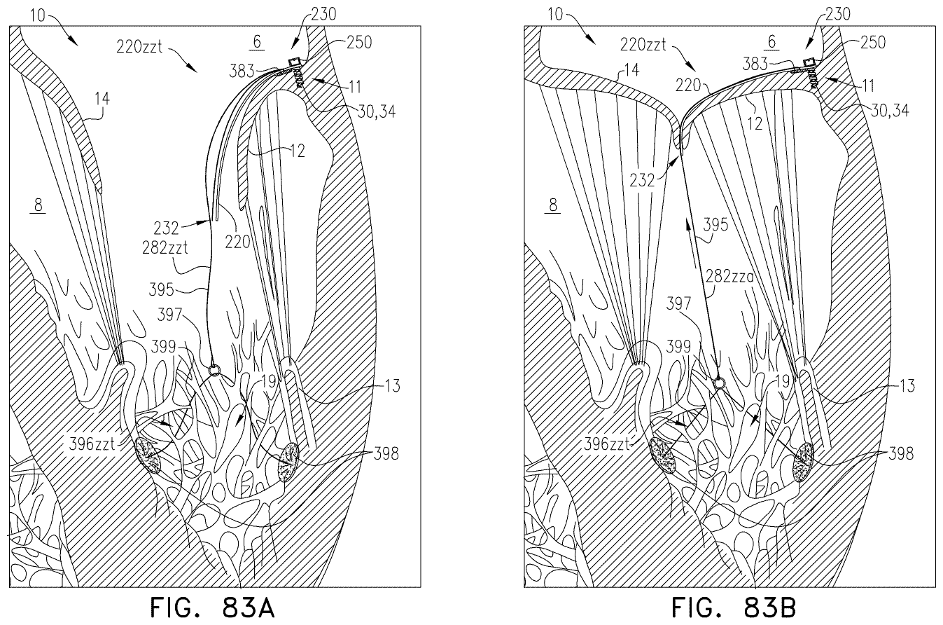

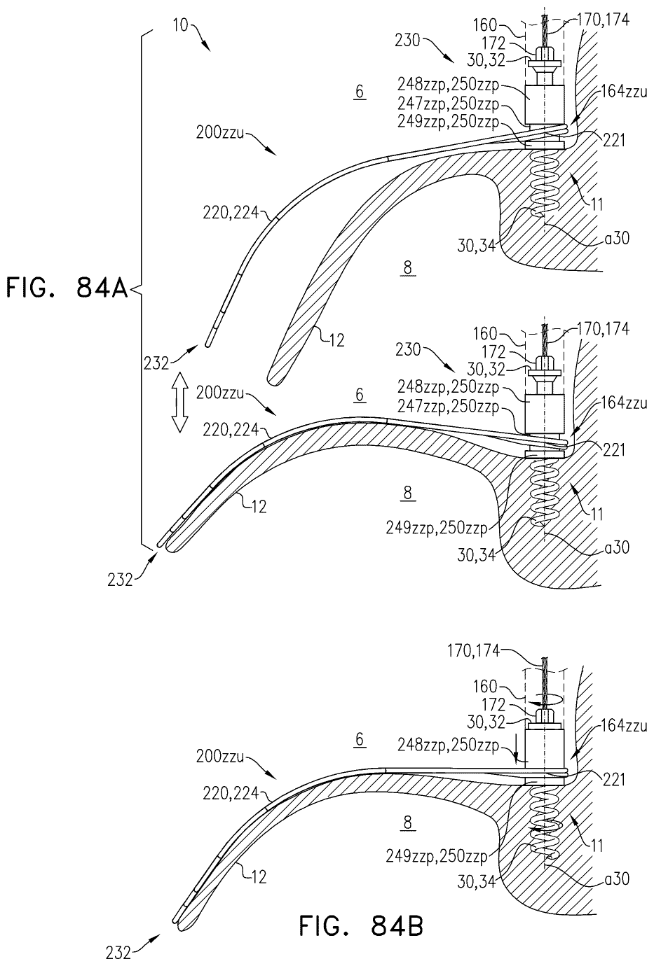

In some implementations, and as shown, tether 282zza, 282zzt extends from root portion 230 to tip portion 232, and from the tip portion to tissue of ventricle 8 to which a distal portion 396zza, 396zzt of the tether is anchored. Typically for such implementations, tether 282zza, 282zzt is tensioned prior to anchoring distal portion 396zza, 396zzt to tissue of ventricle 8, such that the tether inhibits upstream deflection of the wing. In some implementations, tether 282zza, 282zzt is tensioned to set the wing's deflection-limit, such that tip portion 232 of wing 220 contacts the tether upon the wing reaching the deflection-limit, e.g., during ventricular systole (FIGS. 63B, 83B).

In some implementations, and as shown in FIGS. 63A-B, distal portion 396zza comprises a shape-memory material that, upon release (e.g., from a restriction sleeve, not shown), assumes a twisted shape that becomes entangled within ventricular trabeculae 19. Alternatively or in addition, distal portion 396zza can comprise an expandable disc or bulb, which can be a braided structure (e.g., similar to bulking element 420 described hereinbelow with reference to FIGS. 82A-B) that is intracardially expanded while the distal portion is disposed between ventricular trabeculae 19. In this way, distal portion 396zza is anchored to ventricle 8 without puncturing tissue of the ventricle. Alternatively or in addition, distal portion 396zza can be anchored to ventricular tissue, e.g., to a papillary muscle 13 thereof.

In some implementations, and as shown in FIGS. 83A-B, distal portion 396zzt defines a rail portion 399 that extends between two ventricular sites. For example, each end of the rail portion can have an atraumatic anchor 398 (e.g., an expandable disc or bulb) that anchors the end to trabeculae 19 at a respective ventricular site. A proximal portion 395 of tether 282zzt is coupled to rail portion 399. This coupling can be a slidable coupling 397, e.g., with proximal portion 395 looping around the rail portion, or defining an eyelet that is threaded onto the rail portion, e.g., as shown. This slidable coupling can allow proximal portion 395 and distal portion 396zzt to dynamically arrange themselves according to the geometry and forces within the ventricle.

As shown in FIG. 83B, when the heart cycles into ventricular systole, tension on proximal portion 395 is transferred through coupling 397 and to anchors 398, e.g., at an angle that is oblique with respect to a proximal portion 395 of tether 282zzt. This can draw anchors 398 (and thereby the ventricular walls) toward each other, which can advantageously provide additional mechanical support to the ventricle during ventricular systole.

In some implementations, and as shown in FIGS. 64A-B and 65, tether 282zzb, 282zzc (e.g., a pair of tethers 282zzb, 282zzc) is disposed along root portion 230zzb, 230zzc of wing 220zzb, 220zzc. In some implementations, and as shown, tether 282zzb, 282zzc extends to a distal portion 396 of the tether that is anchored to tissue of ventricle 8, e.g., to papillary muscle 13, as shown in FIGS. 64A-B. Alternatively or in addition, distal portion 396 of tether 282zzb, 282zzc can be anchored to tissue of atrium 6, e.g., to a fibrous trigone adjacent the heart's aortic valve (not shown).

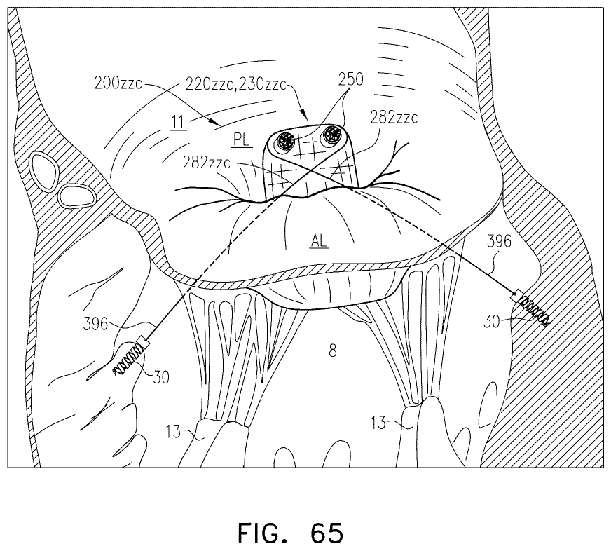

In some implementations, and as shown in FIGS. 64A-B, a portion of each tether 282zzb is disposed within a sleeve 387 at the wing's root portion 230zzb, e.g., along lateral edges of the root portion. Alternatively, and as shown in FIG. 65, tethers 282zzc laterally cross over root portion 230zzc of the wing. Laterally crossing over root portion 230zzc can facilitate alignment of tether 282zzc over wing 220zzc.

In some implementations, a force that tether 282zzb, 282zzc applies to root portion 230zzb, 230zzc of wing 220zzb, 220zzc keeps the root portion in contact with annulus 11 as the wing (e.g., tip portion 232 thereof) deflects in response to the cardiac cycle. In some implementations, tether 282zzb, 282zzc is not disposed along tip portion 232 of wing 220zzb, 220zzc, such that the tether will not impede deflection of the wing's tip portion.

In some implementations, the force that tether 282zzb, 282zzc applies to root portion 230zzb 230zzc of the wing makes the root portion effectively stiffer than tip portion 232, which can facilitate function of native valve 10. For example, support that tether 282zzb, 282zzc provides to root portion 230, 230zzb can provide greater support to a portion of leaflet experiencing prolapse, while less impeded deflection of tip portion 232 with respect to the root portion can improve coaptation of a flailing portion of the leaflet.

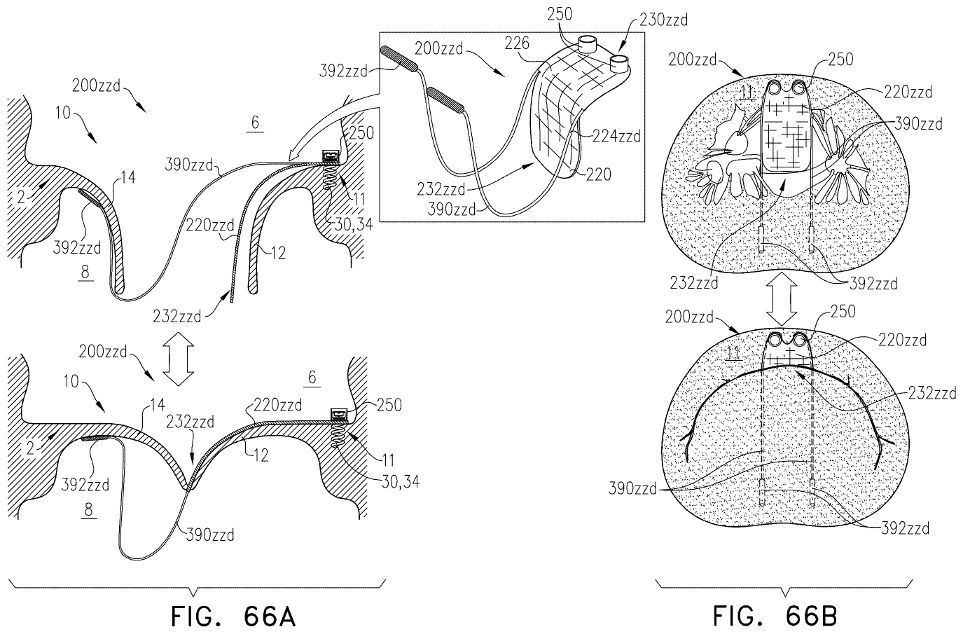

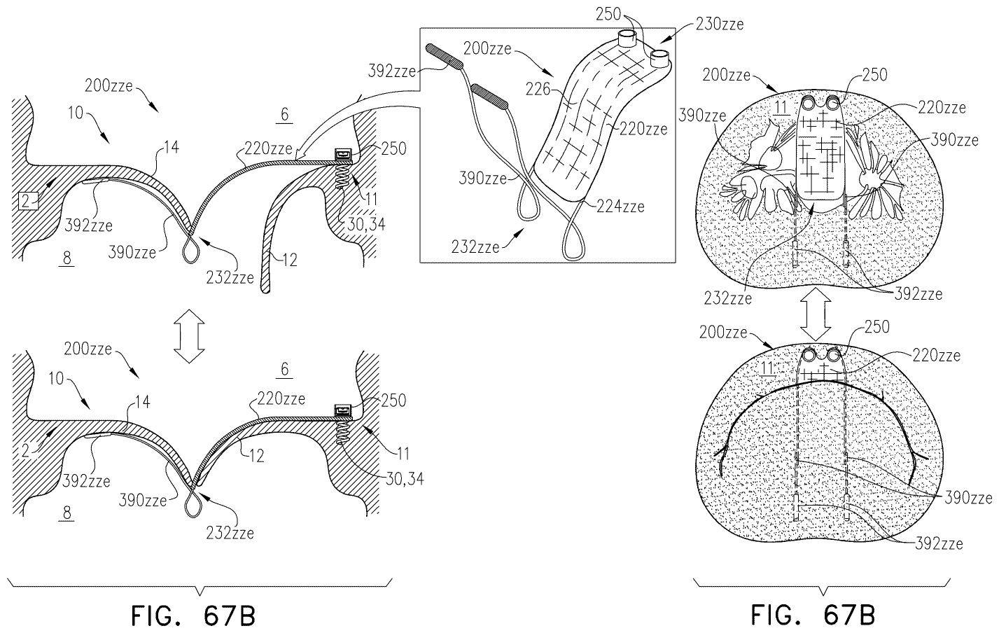

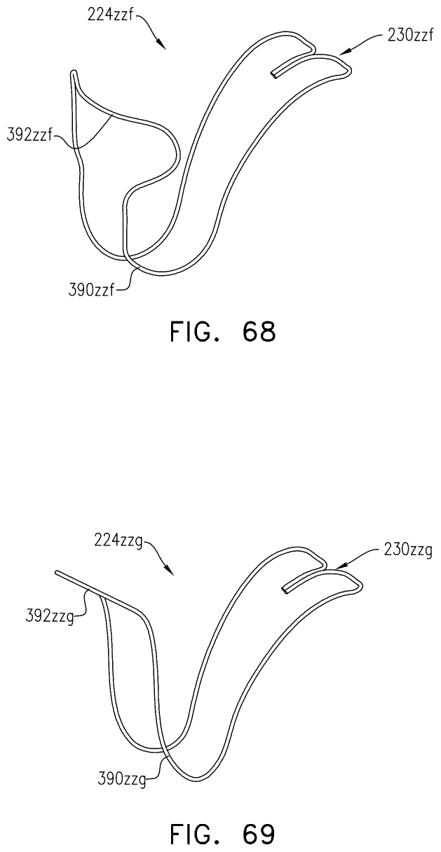

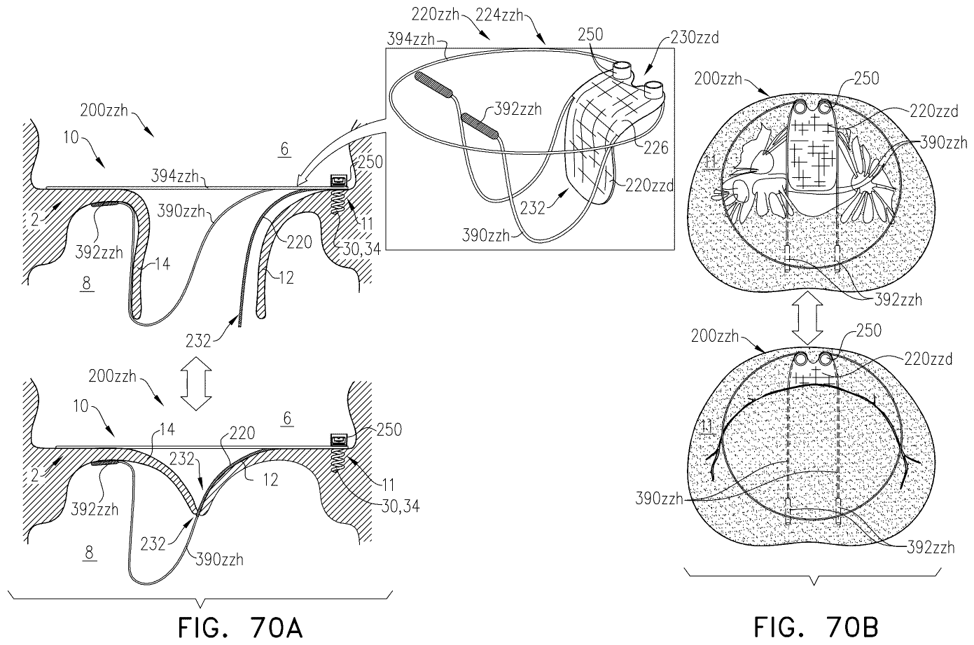

Reference is made to FIGS. 66A-B, 67A-B, 68-69, 70A-B and 71 which are schematic illustrations showing implants 200zzd, 200zze, 200zzf, 200zzg, 200zzh, 200zzi, in accordance with some implementations. Implants 200zzd, 200zze, 200zzf, 200zzg, 200zzh, 200zzi can be considered to be variants of implant 200, and can be similar, at least in their general purpose, e.g., being anchored to a tissue of the heart so as to repair the function of the native leaflet, to implant 200 or any of the variants thereof disclosed herein, mutatis mutandis, except that implants 200zzd, 200zze, 200zzf, 200zzg, 200zzh, 200zzi each comprise a leg 390zzd, 390zze, 390zzf, 390zzg, 390zzh, 390zzi that extends away from wing 220zzd, 220zze.

FIGS. 66A-B, FIGS. 67A-B and 70A-B each show a perspective view of implants 200zzd, 200zze, 200zzh, as well as each implant implanted at mitral valve 10 as the mitral valve cycles between ventricular diastole (upper frames) and systole diastole (lower frames). As shown, implant 200zzd, 200zze, 200zzh is implanted such that root portion 230zzd, 230zze of wing 220zzd, 220zze is secured to annulus 11 (e.g., by interface 250 being anchored to the annulus). As shown, wing 220zzd, 220zze extends from annulus 11, over posterior leaflet 12 and towards anterior leaflet 14, while leg 390zzd, 390zze contacts an underside of valve 10. In some implementations, and as shown, leg 390zzd, 390zze contacts ventricular tissue adjacent a root 2 of the anterior leaflet, e.g., tissue located behind the anterior leaflet, such as a fibrous trigone of ventricle 8 and/or a subannular groove of mitral valve 10.

In some implementations, legs 390zzd, 390zze are stiff, e.g., stiffer than frame 224 of wing 220zzd, 220zze. Stiffness of the legs can contribute to functionality of the native valve, by limiting upstream deflection of the wing 220zzd, 220zze, thereby facilitating coaptation of leaflets 12, 14. In some implementations, and as shown, legs 390zzd, 390zze are sufficiently stiff that distal portions 392zzd, 392zze of the legs retain a generally constant shape as the wing deflects in response to the cardiac cycle. Typically for such implementations, distal portions 392zzd, 392zze remain in contact with a generally constant tissue location as the wing deflects. Legs 390zzd, 390zze remaining at a constant tissue location, can make implants 200zzd, 200zze, 200zzh less traumatic to the tissue than if the implants were to repeatedly renew contact with the tissue during multiple cardiac cycles.

In some implementations, and as shown, legs 390zzd, 390zze are extensions of frame 224zzd, 224zze, e.g., such that the legs provide additional mechanical support to lateral edges of wing 220zzd, 220zze. Since legs 390zzd are typically stiffer than frame 224, coupling the wing to the legs can make the wing stiffer. In some implementations, flexible sheet 226 couples the legs to the wing, such that the extent to which the legs are covered by the sheet determines which portion of the wing is made stiffer by the legs.

One difference between implants 200zzd and 200zze lies in the part of wings 220zzd, 220zze that are coupled to legs 390zzd, 390zze, and therefore the degree to which the legs limit upstream deflection of the wing. In some implementations, and as shown in FIGS. 66A-B, sheet 226 couples legs 390zzd to the wing's root portion 230zzd but not to the wing's tip portion 232zzd, making the root portion stiffer than tip portion 232. Tip portion 232zzd is therefore freer to deflect in response to the cardiac cycle than is root portion 230, as shown in FIGS. 66A-B. Root portion 230zzd of being stiffer can facilitate function of native valve 10, e.g., by providing greater support to a portion of leaflet experiencing prolapse, while flexibility of tip portion 232zzd can improve coaptation of leaflets 12, 14.

In some implementations, and in contrast to wing 220zzd of implant 200zzd, wing 220zze of implant 200zze is coupled to legs 390zze at both root portion 230zze and at tip portion 232zze of the wing. As shown in FIGS. 67A-B, legs 390zze therefore keep both root portion 230zze and tip portion 232zze (e.g., wing 220zze in its entirety) in a generally constant position during the cardiac cycle. The additional support that legs 390zze provide to tip portion 232zze can make implant 200zze particularly suitable for implantation at a valve having a leaflet 12 experiencing flailing and/or prolapse at a tip portion of the leaflet.

FIGS. 68-69 show frames 224zzf, 224zzg of implants that are generally similar to implants 200zzd, 200zze, e.g., in that legs 390zzf, 390zzg are extensions of 224zzf, 224zzg. In contrast to legs 390zzd, 390zze that each define a pair of distal portions 392zzd, 392zze, FIGS. 68-69 show that legs 390zzf, 390zzg each define an elongate distal portion 392zzf, 392zzg that is shaped as a crossbar, e.g., to distribute a force that the distal portion can apply along the length of the crossbar. In some implementations, and as shown in FIG. 68, distal portion 392zzf of leg 390zzf can be wider than root portion 230zzf. In some implementations, leg 390zzf is shaped such that placing root portion 230zzf against annulus 11 places distal portion 392zzf into contact with tissue adjacent the commissures of mitral valve 10.

FIGS. 70A-B show implant 200zzh, which is generally identical to implant 200zzd, except that frame 224zzh of implant 200zzh comprises an atrial support 394zzh. As shown, atrial support 394zzh is coupled to wing 220zzd, e.g., to root portion 230zzd thereof, and is shaped such that when wing 220zzd is placed against the site, atrial support 394zzh presses against an atrial surface of annulus 11. In some implementations, and as shown in FIG. 70B, atrial support 394zzh is shaped to circumscribe the atrial surface of mitral valve 10.

As shown in FIG. 70A's perspective view of implant 200zzh in a resting state (e.g., without tissue of valve 10 that would apply force to the implant), distal portions 392zzh of legs 390zzh pass through atrial support 394zzh. Thus, implanting implant 200zzh by fitting anterior leaflet 14 between the distal portions 392zzh and atrial support 394zzh involves pushing the distal portions below the atrial support, such that legs 390zzh press (e.g., in the upstream direction) against tissue of ventricle 8.

FIG. 71 shows a frame 224zzi of an implant that is generally similar to implant 200zzh, in that frame 224zzi comprises legs 390zzi, as well as an annular support 394zzi that is coupled to root portion 230zzi. As shown, annular support 394zzi comprises two arms that extend in opposite directions from root portion 230zzi. In this way, when the implant's wing is placed against annulus 11, the arms will circumscribe a portion of the atrial surface of valve 10. Similarly to frames 224zzf and 224zzg shown in FIGS. 68-69, distal portion 392zzi defines a crossbar.

It is to be noted that atrial supports 394zzh, 394zzi are compatible with frames of other implants described hereinabove, including 200zze, 200zzf and 200zzg.

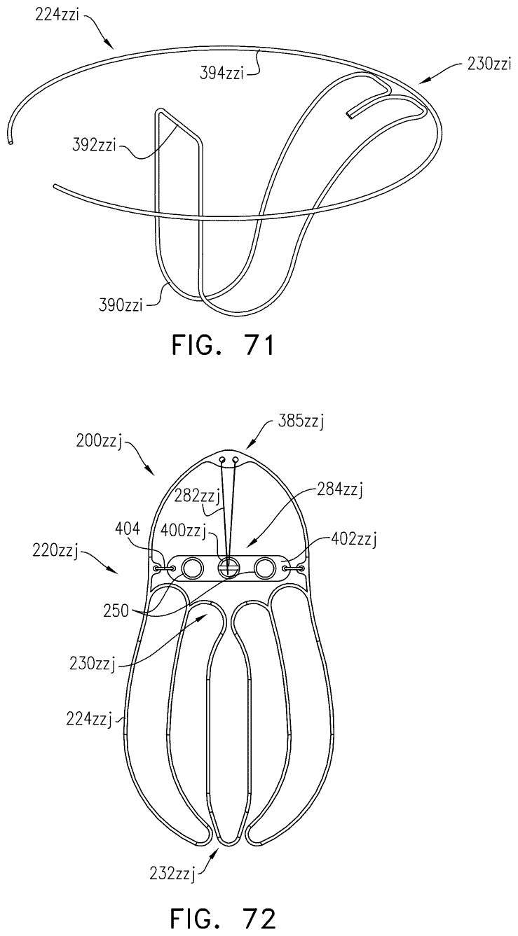

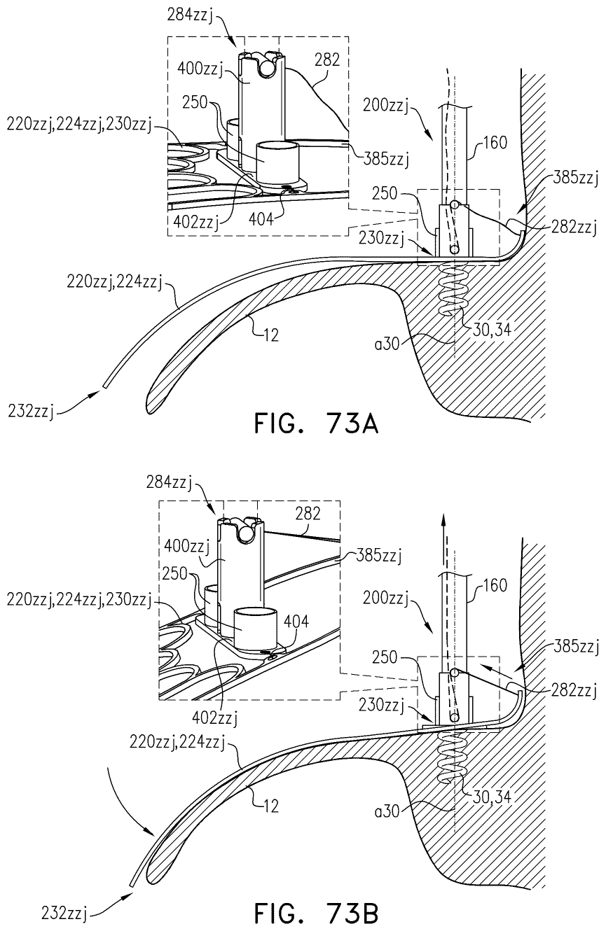

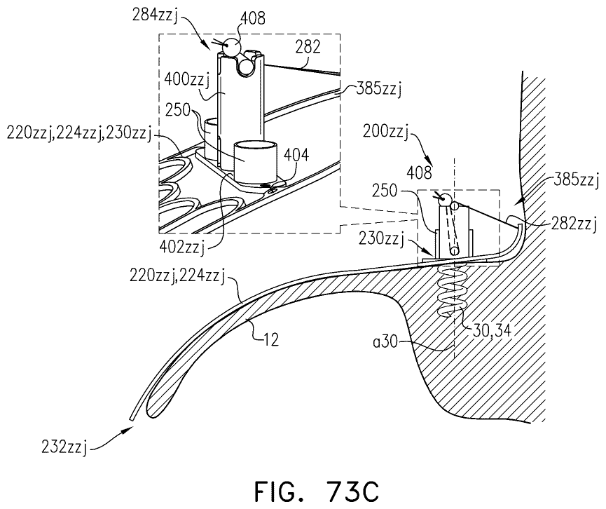

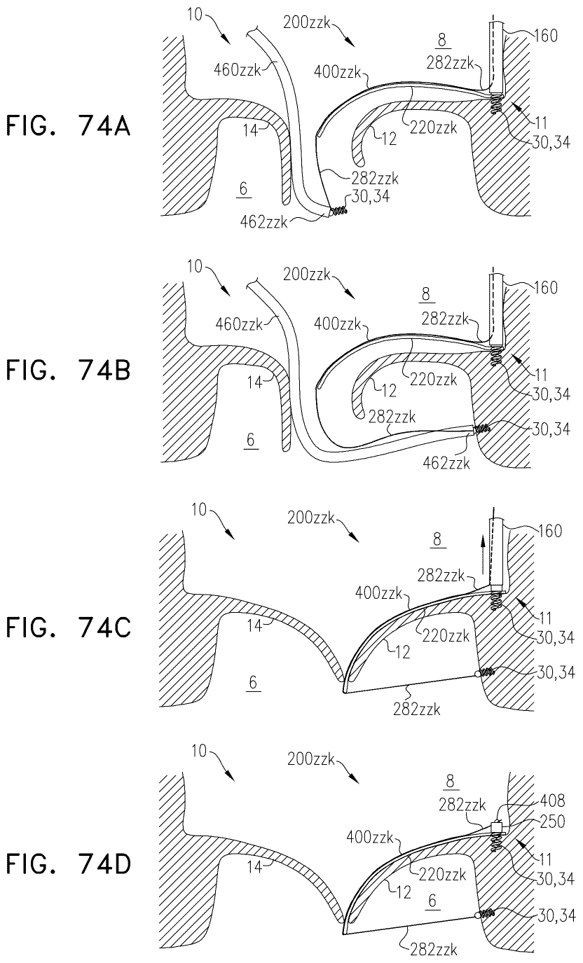

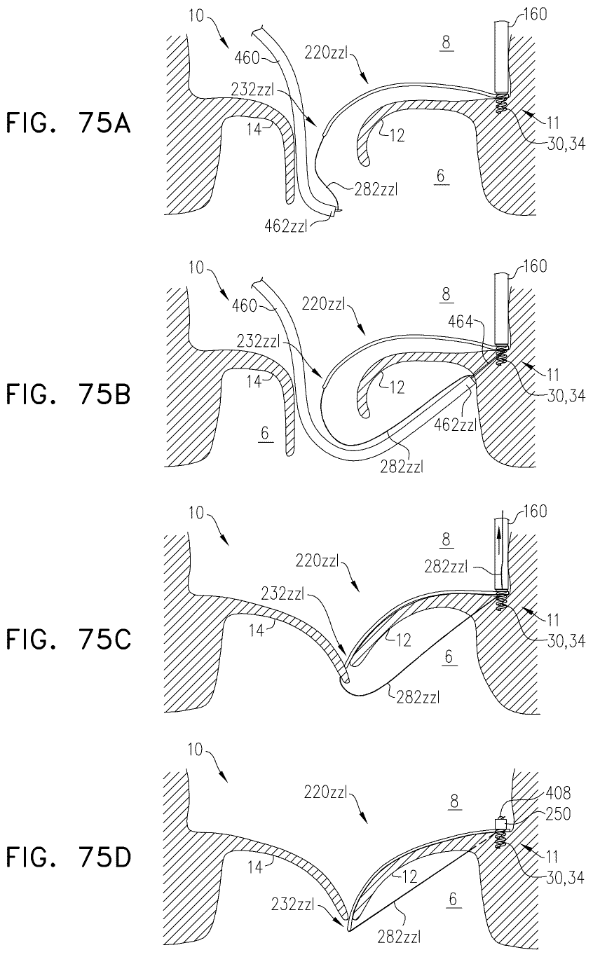

Reference is made to FIGS. 72, 73A-C, 74A-D and 75A-D, which are schematic illustrations showing implants 200zzj, 200zzk, 200zzl, in accordance with some implementations. Implants 200zzj, 200zzk, 200zzl can be considered to be variants of implant 200, and can be similar, at least in their general purpose, e.g., being anchored to a tissue of the heart so as to repair the function of the native leaflet, to implant 200 or any of the variants thereof disclosed herein, mutatis mutandis, except that implants 200zzj, 200zzk, 200zzl are each adjustable by changing tension on a tether 282zzj, 282zzk, 282zzl.

For clarity, FIGS. 72 and 73A-C show implant 200zzj without sheet 226 that covers wing 220zzj. As shown, wing 220zzj is generally similar to wing 220, except that wing 220zzj defines a backstop portion 385zzj that extends from root portion 230zzj of the wing, opposite from tip portion 232zzj. As shown in FIGS. 73A-B, tether 282zzj couples backstop portion 385zzj to sleeve 400zzj, through which the tether passes into shaft 160, e.g., such that controller 120 can be used to adjust tension on the tether.

In some implementations, and as shown, implant 200zzj comprises a seat 402 to which interface 250 (e.g., a pair of interfaces) is coupled. In this way, securing interface 250 to tissue using anchor 30 seats seat 402 against tissue of annulus 11 (FIG. 73A). Typically for such implementations, seat 402 is articulatably coupled to wing 220zzj, e.g., by sutures 404, such that the wing pivots along sutures 404 as the wing deflects during the cardiac cycle, and a range along which wing 220zzj pivots with respect to seat 402 defines the wing's range of deflection. In this way, tensioning tether 282zzj via sleeve 400 adjusts the wing's deflection-limit, such that seat 402, the sleeve and the tether can be considered components of a limiter 284zzj.

In some implementations, tension on tether 282zzj is adjusted by changing a length of the tether connecting backstop portion 385zzj to limiter 284zzj. In some implementations, the length of the tether connecting limiter 284zzj to backstop portion 385zzj is changed by intracardially rotating a spool (not shown) around which a portion of the tether is wound.

FIG. 73B shows tether 282zzj having been tensioned, such that tip portion 232zzj of wing 220zzj pivots further downstream, with respect to interface 250 and anchor axis a30, than in FIG. 73A. FIG. 73C shows shaft 160 having been withdrawn, after applying a bead 408 to tether 282zzj, which locks (e.g., reversibly locks) wing 220zzj at a desired orientation with respect to interface 250.

FIGS. 74A-D and 75A-D show implants 200zzk, 200zzl, which comprise tethers 282zzk, 282zzl that are coupled to wings 220zzk, 220zzl such that adjusting tension on the tether adjusts a deflection-range, e.g., a deflection-limit, of the wing.

FIG. 74A shows implant 200zzk anchored by anchor 30 to annulus 11 of the mitral valve, with shaft 160 coupled to the implant's interface 250. As shown, tether 282zzk extends through shaft 160 and is coupled to wing, e.g., slidably coupled to the wing through a sleeve 400zzk. As shown, prior to tensioning tether 282zzk, leaflets 12, 14 do not yet substantially coapt during ventricular systole.

In some implementations, and as shown, a second anchor 30 is (i) coupled to the distal end of tether 282zzk, and (ii) reversibly coupled to a steerable tether catheter 460zzk, e.g., to a distal end 462zzk of the tether catheter. FIG. 74B shows tether 282zzk having been anchored to tissue of ventricle 8, e.g., by advancing driver 170 through tether catheter 460zzk to drive the second anchor into the tissue. FIG. 74C shows tether 282zzk being transluminally tensioned by pulling the tether proximally such that the tether slides through sleeve 400zzk and shaft 160 and a shorter length of the tether 282zzk connects tether anchor 30 to wing 220zzk. The increased tension on tether 282zzk increases a downstream force that wing 220zzk applies to leaflet 12, bringing the leaflet and wing 220zzk into coaptation with opposing leaflet 14. As shown in FIG. 74D, shaft 160 is then withdrawn from the subject, leaving behind a bead 408 that maintains tension on tether 282zzk.

FIGS. 75A-D show implant 200zzl, which is similar to implant 200zzk, e.g., in that implant 200zzl is implanted at annulus 11 and tether 282zzk is tensioned (e.g., using a tether catheter 460zzl) to adjust the deflection-limit of wing 220zzl. In some implementations, and in contrast to implant 200zzk, one end of tether 282zzk is fixedly coupled to tip portion 232zzl of wing 220zzl. Alternatively or in addition, tether 282zzk can be coupled to other portions of wing 220zzl, mutatis mutandis.

In some implementations, and as shown in FIGS. 75A-B, tether 282zzl is coupled to distal end 462zzl of tether catheter 460zzl such that advancing the catheter's distal end 462zzl away from the wing's tip portion 232zzl (e.g., toward a root of posterior leaflet 12, as shown in FIG. 75B) causes additional length of the tether to be released from the catheter.

In some implementations, and as shown in FIG. 75B, while distal end 462zzl of tether catheter 460zzl is disposed at the root of leaflet 12, a needle 464 is advanced from tether catheter 460zzl, through tissue of the leaflet's root portion and toward interface 250. In some implementations, and as shown, tether 282zzl is passed from needle 464 and up through root portion 230zzl, e.g., through interface 250, such that the tether enters shaft 160. In this way, a snare (not shown) can be advanced via shaft 160 to pull on tether 282zzl, thereby tensioning the tether by shorting the length of tether between tip portion 232zzl and interface 250 (FIGS. 75C-D).

In some implementations, and as shown in FIG. 75D, tensioned tether 282zzl adjusts the wing's deflection-range, e.g., by causing the wing to pivot further downstream with respect to interface 250. In some implementations, tether 282zzl is tensioned such that the tether restrains tip portion 232zzl from deflecting upstream past the deflection-limit. As shown in FIG. 75D, the tensioned tether 282zzl brings leaflet 12 and wing 220zzl into coaptation with opposing leaflet 14. Shaft 160 is then withdrawn from the subject, leaving behind a bead 408 that maintains tension on tether 282zzl.

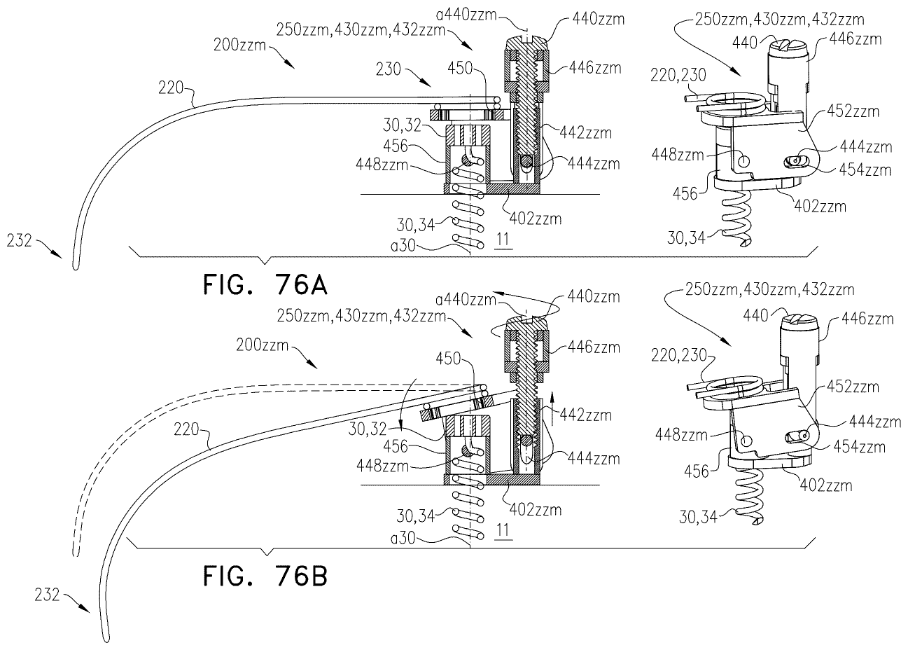

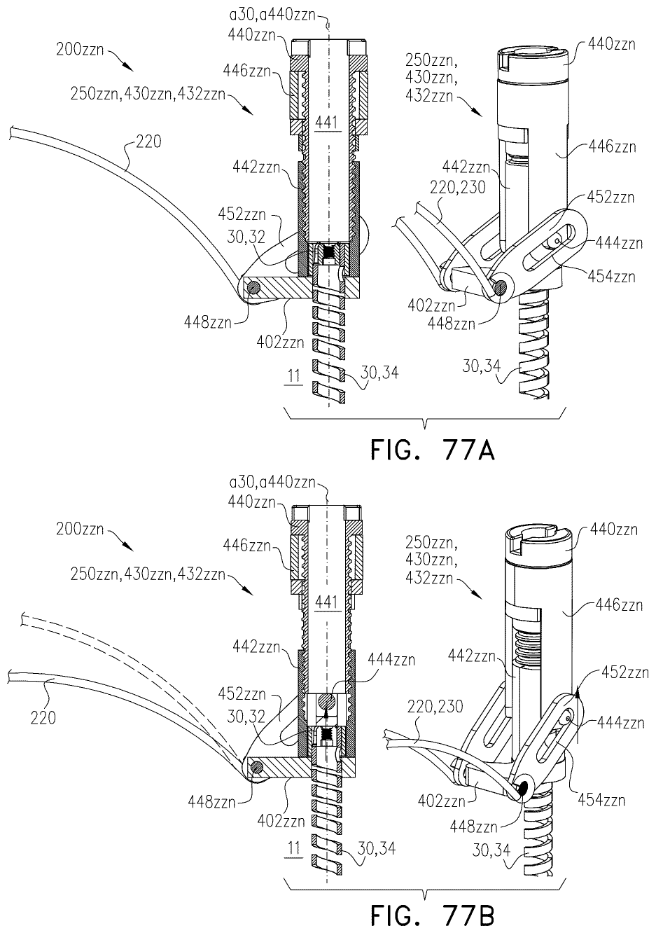

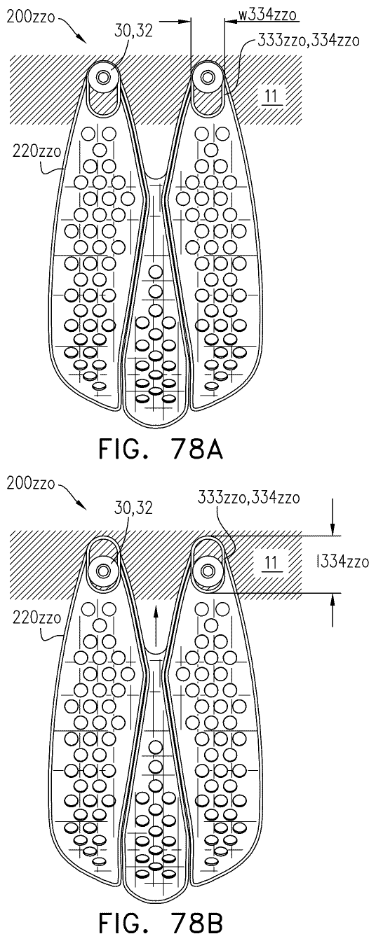

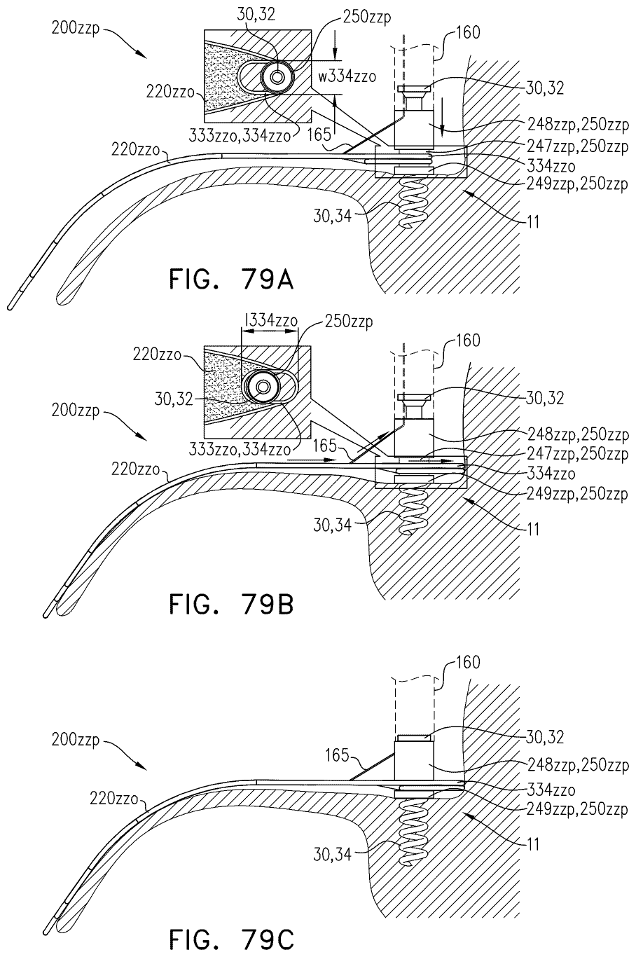

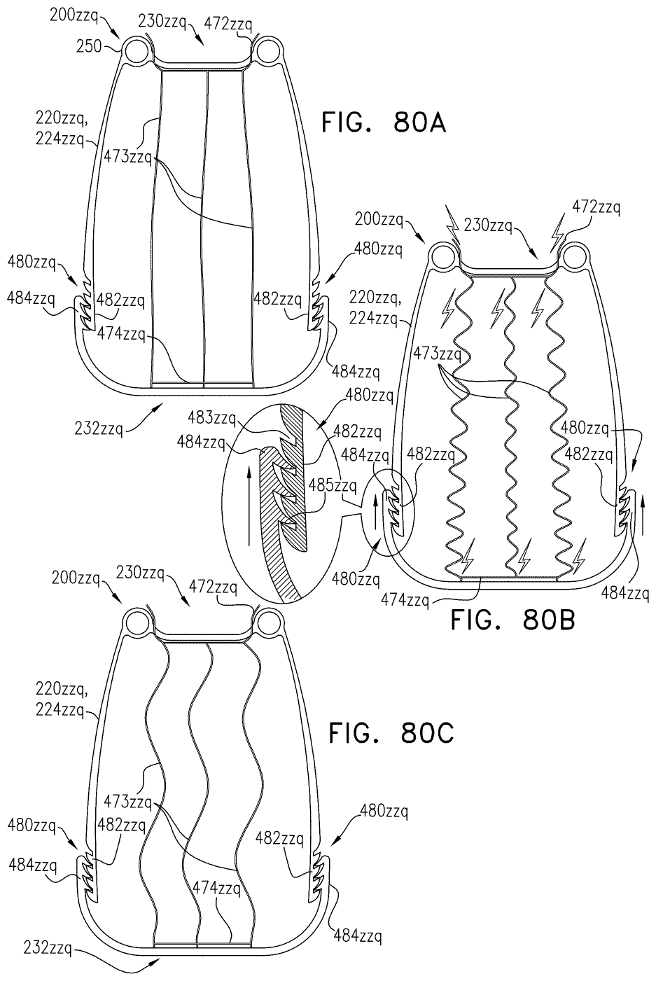

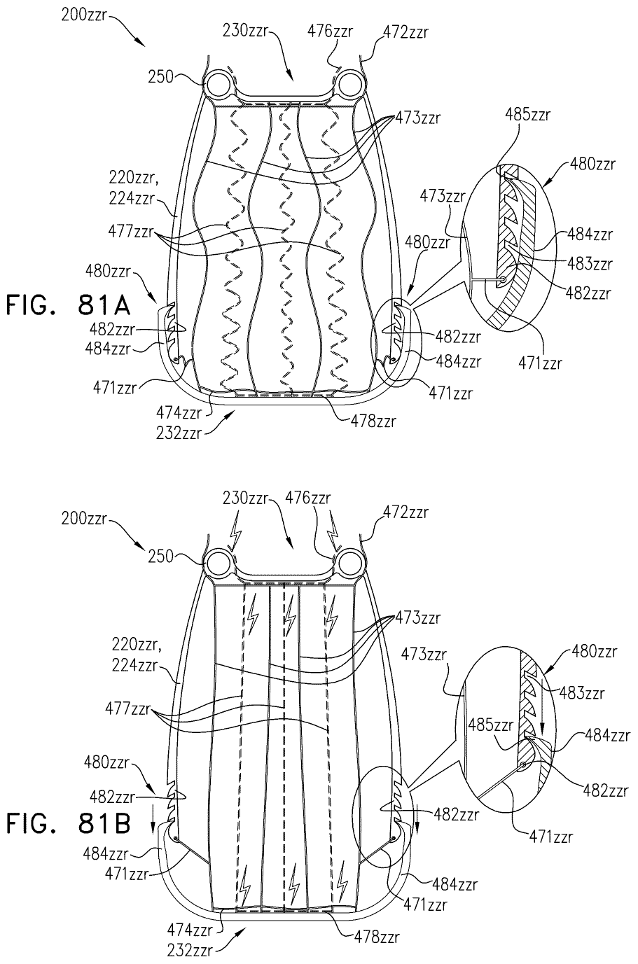

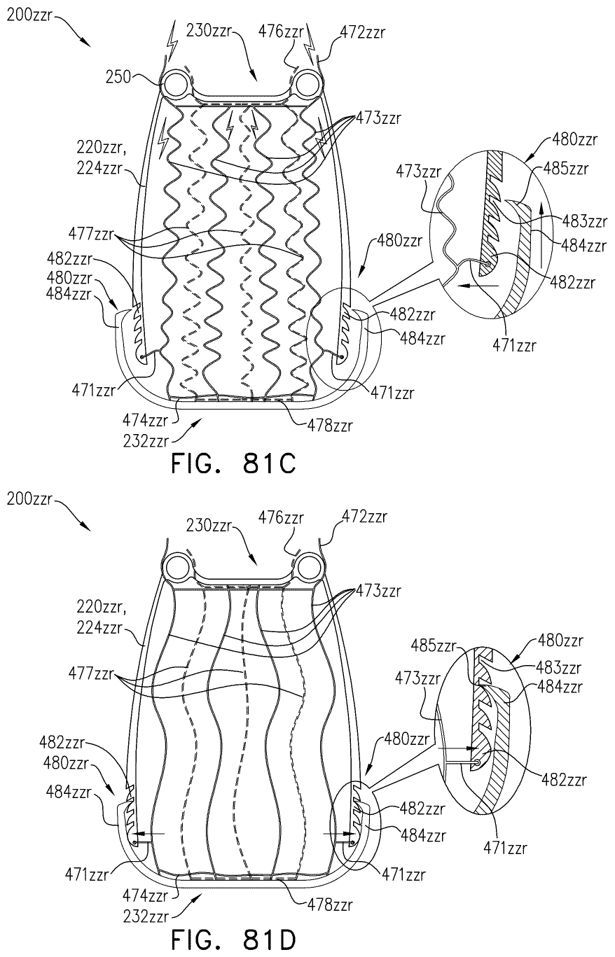

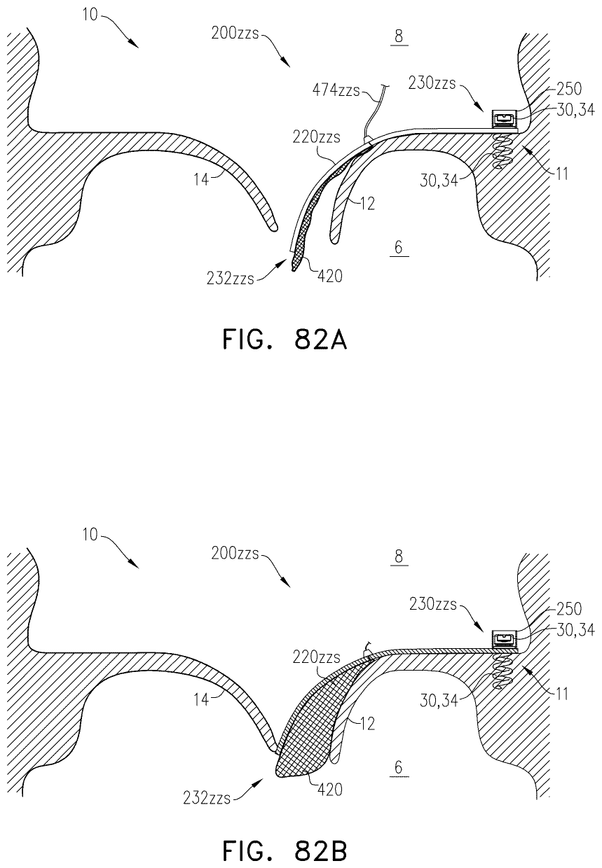

Reference is made to FIGS. 76A-B and 77A-B, which are schematic illustrations showing implants 200zzm, 200zzn comprising adjustment members 430zzm, 430zzn, in accordance with some implementations. In some implementations, and as shown, adjustment member 430zzm, 430zzn is defined by an adjustable interface 250zzm, 250zzn, comprising an adjustment mechanism 432zzm, 432zzn that is adjustable (e.g., using controller 120, via catheter 140) in a manner that adjusts the deflection-range of wing 220.

The right panes of FIGS. 76A-B and 77A-B show perspective views of implant 200zzm, 200zzn, and left panes show corresponding cross-sectional views of each implant secured by anchor 30 to tissue of annulus 11. Implants 200zzm, 200zzn are shown with wing 220 in a state of maximum upstream deflection, e.g., wherein wing 220 has reached the wing's deflection-limit, both before (FIGS. 76A, 77A) and after (FIGS. 76B, 77B) adjusting mechanism 432zzm, 432zzn.

In some implementations, and as described hereinbelow, adjusting mechanism 432zzm, 432zzn alters a deflection-angle of wing 220, e.g., an angle between root portion 230 of the wing and anchor axis a30. In some implementations, and as shown, interface 250zzm, 250zzn defines a seat 402zzm, 402zzn that remains seated (e.g., seated flush) against tissue of annulus 11 both before (FIGS. 76A, 77A) and after (FIGS. 76B, 77B) changing the wing's deflection-angle. Since seat 402zzm, 402zzn remains seated against the tissue, adjusting adjustment mechanism 432zzm, 432zzn typically changes an angle between root portion 230 of wing 220 and the seat.

As described hereinabove, adjustment mechanisms 432zzm and 432zzn are in many ways similar, and each implant can also be adjusted similarly. In some implementations, and as shown, each mechanism 432zzm, 432zzn is adjusted by screwing (e.g., using a linear actuator that is advanced via catheter 140) a lead screw 440zzm, 440zzn that is coupled to a pin 444zzm, 444zzn through a threaded piston 442zzm, 442zzn that is housed within a collar 446zzm, 446zzn. Since each screw is rotatably coupled to the pin, the pin transfers helical retraction of lead screw 440zzm, 440zzn, into upward movement of the pin. As shown in FIGS. 76B and 77B, the pin's upward movement causes the pin to slide within a groove 454zzm, 454zzn that is defined by a sideplate 452zzm, 452zzn. Sideplate 452zzm, 452zzn in turn pivots about a hinge 448zzm, 448zzn, thereby changing an angle at which a base 450, to which root portion 230 is coupled, is oriented with respect to seat 402zzm, 402zzn and anchor axis a30.

In some implementations, and as shown in FIGS. 77A-B, rather than being coupled to a base portion of the adjustment mechanism, wing 220 of implant 200zzn is coupled to sideplate 452zzn via hinge 448zzn. In this way, helically retracting lead screw 440zzn slides pin 444zzn upwards, thereby changing an angle at which wing 220 deflects from hinge 448zzn.

Another difference between adjustment mechanisms 432zzm and 432zzn lies in the orientation of lead screw 440zzm, 440zzn with respect to anchor 30. In some implementations, and as shown in FIGS. 76A-B, lead screw 440zzm is offset from anchor 30, e.g., such that a lead screw axis a440zzm is parallel to anchor axis a30. In some implementations, and as shown in FIGS. 77A-B, lead screw axis a440zzn is colinear with anchor axis a30. In some implementations, and as shown, lead screw 440zzm is accessible (e.g., accessible to unshown driver 170) through a screw lumen 441 that is defined by lead screw 440zzn. Colinear orientation of lead screw 440zzn with respect to anchor 30 can simplify adjustment of mechanism 432zzn, since the same angle of approach (e.g., using catheter 140) can be utilized both for driving anchor 30, and for adjusting lead screw 440zzn.

Reference is made to FIGS. 78A-B and 79A-C, which are schematic illustrations showing implants 200zzo, 200zzp, in accordance with some implementations.

Implants 200zzo, 200zzp can be considered to be variants of implant 200, and can be similar, at least in their general purpose, e.g., being anchored to a tissue of the heart so as to repair the function of the native leaflet, to implant 200 or any of the variants thereof disclosed herein, mutatis mutandis, except that implants 200zzo, 200zzp are adjustable by sliding the implant with respect to anchor 30. Although implants 200zzo, 200zzp each comprise a wing 220zzo, this is for illustrative purposes only, and the principle described hereinbelow according to which these implants are slidable can be applied to other implants, e.g., cardiac implants, mutatis mutandis.

In some implementations, catheter 140 is used to advance shaft 160 to the heart. As described hereinabove, shaft 160 is used to deploy implant 200zzo, 200zzp out of catheter 140, and to position the implant such that an anchor receiver 334zzo is at annulus 11 and wing 220zzo extends over a leaflet of the valve. While implant 200zzo, 200zzp is so positioned, anchor 30 can be driven into tissue of annulus 11.

In some cases, it may be desirable to adjust the position of implant 200zzo, 200zzp with respect to annulus 11, yet there may be disadvantages to removing anchor 30 from the tissue. Implants 200zzo, 200zzp are therefore adjustable with respect to annulus 11 by sliding wing 220zzo with respect to anchor head 32, without removing anchor 30 from the tissue. FIGS. 78A-B and 79A-B show respective implants 200zzo, 200zzp at an initial stage of being anchored to annulus 11. At this stage, tissue-engaging element 34 of anchor 30 extends through the anchor receiver's opening and into tissue of the annulus 11, but the anchor is not yet fully driven into tissue of the annulus 11, such that anchor receiver 334zzo is not firmly sandwiched between anchor head 32 and the tissue. At this stage, implant 200zzo is slidable with respect to anchor 30.

As shown, anchor receiver 334zzo defines an oblong opening delimited by a rim 333zzo. The oblong's opening typically defines: (i) a width w334zzo that is smaller than a diameter of anchor head 32, and (ii) a length 1334zzo (e.g., a major axis of the anchor receiver's oblong opening) that is greater than the anchor head's diameter. In this way, anchor receiver 334zzo serves as a track along which wing 220zzo can be slid along the opening's major axis, with respect to anchor head 32 (FIGS. 78A-B, 79A-B).

In some implementations, and as shown, the opening's major axis is generally parallel with a length of wing 220zzo, such that wing 220zzo is slidable along the wing's length. Alternatively, the opening's major axis can be perpendicular, or skewed with respect to the wing's length.

FIGS. 78B and 79B each show wing 220zzo having been slid along anchor receiver 334zzo with respect to anchor head 32, toward annulus 11. After so adjusting the position of wing 220zzo with respect to annulus 11, anchor 30 is typically driven further into the tissue (FIG. 79C), such that anchor head 32 is seated against rim 333zzo, firmly sandwiching anchor receiver 334zzo between anchor head 32 and the tissue. In this way, implant 200zzo is locked to anchor 30, and wing 220zzo is no longer slidable with respect to the anchor.

FIGS. 79A-C show implant 200zzp, which, like implant 200zzo, comprises wing 220zzo. In addition to the slidability of implants 200zzo, 200zzp described hereinabove, implant 200zzp further comprises a two-part interface 250zzp that facilitates sliding wing 220zzo with respect to anchor 30, and then locking the wing with respect to the anchor as the anchor is driven further into tissue of annulus 11.

In some implementations, and as shown, interface 250zzp comprises an upper collar 248zzp and a lower collar 249zzp, each of which fit around (e.g., circumscribe) the interface's neck 247zzp. FIG. 79A shows anchor receiver 334zzo fitted about the interface's neck 247zzp, and collars 248zzp, 249zzp spaced apart from each other, such that interface 250zzp is unlocked, e.g., anchor receiver 334zzo is slidable with respect to the interface's neck 247zzp.

As shown in the insets of FIGS. 79A-B, the diameter of the upper and lower collars is greater than the anchor receiver's width w334zzo, and less than the anchor receiver's length 1334zzo, such that anchor receiver 334zzo can serve as a track along which interface 250zzp slides with respect to anchors 30 during the anchoring process.

FIGS. 79A-B also show an adjustment rod 165 that extends through shaft 160 (e.g., from controller 120 and through the shaft), and that is reversibly coupled to implant 200zzp. In some implementations, controller 120 provides for extracorporeal operation of adjustment rod 165, in order to intracardially slide implant 200zzp with respect to anchor 30. Although adjustment rod 165 is not shown in FIGS. 78A-B, the rod is generally compatible with implant 200zzo or other implants, mutatis mutandis.

In some implementations, and as shown in FIG. 79B, adjustment rod 165 is coupled to wing 220zzo such that pulling the rod axially (e.g., proximally) slides the wing away from the opposing leaflet and toward annulus 11. As shown, anchor receiver 334zzo slides along the opening's major axis, and about neck 247zzp of interface 250zzp.

Typically, if the longitudinal position of wing 220zzo with respect to tissue of annulus 11 is found to be desirable, anchor 30 is driven further into the tissue, e.g., using driver 170 (not shown). As shown in FIG. 79C, tissue-engaging element 34 is driven further (e.g., fully) into tissue of annulus 11, locking interface 250zzp such that implant 200zzp is no longer slidable with respect to anchor 30.

In this way, anchor head 32 is seated against the interface, e.g., against an upstream face of upper collar 248zzp, and an opposite and a downstream face of the upper collar is seated against anchor receiver 334zzo. In some implementations, and as shown, seating upper collar 248zzp against anchor receiver 334zzo also sandwiches lower collar 249zzp between the anchor receiver's rim 333zzo and the tissue.

Reference is made to FIGS. 80A-C and 81A-D, which are schematic illustrations showing implants 200zzq, 200zzr, in accordance with some implementations.

Implants 200zzq, 200zzr can be considered to be variants of implant 200, and can be similar, at least in their general purpose, e.g., being anchored to a tissue of the heart so as to repair the function of the native leaflet, to implant 200 or any of the variants thereof disclosed herein, mutatis mutandis, except that the implants comprise wings 220zzq, 220zzr that are adjustable in size, e.g., in length.

In some implementations, and as shown, implant 200zzq, 200zzr comprises a shape-memory member 471zzr, 473zzq, 473zzr, 477zzr, that is coupled to a wing 220zzq, 220zzr. Intracardially heating shape-memory member 471zzr, 473zzq, 473zzr, 477zzr (e.g., to a temperature greater than 40 degrees C.) causes the shape-memory member to change its shape, and the reshaped shape-memory member applies a force to wing 220zzq, 220zzr that resizes the wing. In some implementations, implant 200zzq, 200zzr comprises a locking mechanism 480zzq, 480zzr that locks wing 220zzq, 220zzr such that the wing retains the new size, even after shape-memory member 471zzr, 473zzq, 473zzr, 477zzr is no longer heated. Although FIGS. 80A-C and 81A-D show adjusting the length of wings 220zzq, 220zzr, the shape-memory members can be configured to change another dimension of the wing, e.g., a width, or to change dimensions of other implants, mutatis mutandis.

In some implementations, shape-memory member 471zzr, 473zzq, 473zzr, 477zzr is electrically heated by transmitting electrical power, e.g., via delivery tool 150 such as by catheter 140, to a coupling 472zzq, 472zzr of the shape-memory member. Alternatively or in addition, electrical power can be transmitted wirelessly to an antenna (not shown) that is electrically connected to coupling 472zzq, 472zzr.

In some implementations, implant 200zzq, 200zzr includes a power source (not shown) used to electrically heat the shape-memory members. Alternatively or in addition, the power source can be external to the implant. In some implementations, and as shown, a connector 474zzq, 474zzr conducts electricity between some or all of the shape-memory members in a given implant, e.g., in order to complete an electrical circuit between the shape-memory members and the power source.

As shown in FIG. 80B, power is applied to shape-memory members 473zzq, which heats the shape-memory members, causing them to change their shape, e.g., from a generally straight shape (FIG. 80A) to an undulated shape (FIG. 80B). Since the change in shape shortens a distance spanned by each shape-memory member, shape-memory members 473zzq can be considered shortening shape memory members.

In some implementations, wing 220zzq (e.g., frame 224zzq thereof) is configured to at least partially yield to the pulling force, such that the pulling force shortens the wing. In some implementations, wing 220zzq comprises a two-part frame 224zzq having two parts that are moveable (e.g., slidable) with respect to each other. In some implementations, and as shown in FIG. 80B, shape-memory members 473zzq are coupled to wing 220zzq at root portion 230zzq and at tip portion 232zzq. Since shape-memory members 473zzq shorten as they assume the undulated shape, the shape-memory members apply a pulling force to root portion 230zzq and tip portion 232zzq that pulls the root portion and the tip portion toward each other. For example, tip portion 232zzq can slide, e.g., along a rail (not shown) that slidably connects the tip portion to the root portion 230zzq, in response to the pulling force.

In some implementations, locking mechanism 480zzq, 480zzr (i) facilitates resizing wing 220zzq, and (ii) maintains the wing in the new size. In some implementations, and as shown, locking mechanisms 480zzq, 480zzr define a lock that comprises an inner jaw 482zzq, 482zzr and an outer jaw 484zzq, 484zzr. As shown in the insets of FIGS. 80B and 81A, the inner and outer jaws are shaped to define teeth 485zzq, 485zzr and notches 483zzq, 483zzr that fit together. For example, and as shown in FIGS. 80A-C, the jaws of locking mechanism 480zzq allow tip portion 232zzq to slide toward root portion 230zzq, yet inhibit movement of the tip portion away from the root portion.

In some implementations, and as shown in FIG. 80B, sliding tip portion 232zzq toward root portion 230zzq changes an orientation of locking mechanism 480zzq. As shown in FIG. 80C, when energy is no longer applied to shape-memory members 473zzq, locking mechanism 480zzq remains in the new orientation, thereby maintaining wing 220zzq in its new size.

FIGS. 81A-D show implant 200zzr, which is in many ways similar to implant 200zzq, except that implant 200zzr is configured to facilitate bi-directional resizing (e.g., lengthening and shortening) of wing 220zzr. The following description of implant 200zzr will therefore focus on features of implant 200zzr that facilitate the bi-directional resizing. For example, inner and outer jaws 482zzr, 484zzr of implant 200zzr are shaped to define teeth and notches that fit together in an orientation opposite that of jaws 482zzq, 484zzq of locking mechanism 480zzq. Jaws 482zzr, 484zzr of implant 200zzr of locking mechanism 480zzr therefore function similarly, yet allow tip portion 232zzr to slide away from root portion 230zzr, while inhibiting movement of the tip portion toward from the root portion.

In addition to shortening shape-memory members 473zzr, implant 200zzr also comprises lengthening shape-memory members 477zzr. In some implementations, and as shown in FIG. 81A, lengthening shape-memory members 477zzr assume an undulated shape before they are heated. Typically for such implementations, and as shown in FIG. 81B, heating lengthening shape-memory members 477zzr causes the lengthening shape-memory members to straighten and therefore to lengthen. In this way, lengthening shape-memory members 477zzr pushes tip portion 232zzr away from root portion 230zzr, thereby increasing the length of wing 220zzr.

In some implementations, and as shown, implant 200zzr comprises locking mechanism 480zzr, which is shown in FIG. 81A-B in its locked state, in which jaws 482zzr, 484zzr fit each other in a manner that facilitates movement of tip portion 232zzq away from root portion 230zzq. Thus, when lengthening shape-memory members 477zzr are heated (FIG. 81B), locking mechanism 480zzr allows jaws 482zzr and 484zzr to slide away from each other, distancing tip portion 232zzr from root portion 230zzr.

In some instances, after lengthening wing 220zzr, it may be desirable to again resize the wing, e.g., such that the wing assumes a length in between the wing's original length (FIG. 81A) and the increased length (FIG. 81B). As shown in FIG. 81C, shortening shape-memory members 473zzr are therefore heated such that they assume an undulated shape which reduces their length, pulling tip portion 232zzr toward root portion 230zzr, and therefore reducing the wing's length.

As described hereinabove with reference to FIGS. 81A-B, locking mechanism 480zzr allows jaws 482zzr, 484zzr to slide away from each other when the locking mechanism is locked, yet inhibit root portion 230zzr and tip portion 232zzr from sliding toward each other. Therefore, heating shape-memory members 473zzr may not be sufficient to reduce the wing's length.

In some implementations, and as shown in FIG. 81C, unlocking locking mechanism 480zzr (e.g., distancing jaws 482zzr, 484zzr from each other) facilitates shortening the wing. In some implementations, and as shown in FIG. 81C, locking mechanism 480zzr is unlocked by heating lateral shape-memory members 471zzr, such that they assume an undulated shape that reduces their length, thereby pulling inner jaws 482zzr away (e.g., medially) from outer jaws 484zzr. For example, and as shown, lateral shape-memory members 471zzr can be heated at the same time that shortening shape-memory members 473zzr are heated, e.g., by conducting electricity to a circuit that includes both the lateral shape-memory members and the shortening shape-memory members.

As shown in FIG. 81D, power is no longer applied to lateral shape-memory members 471zzr or shortening shape-memory members 473zzr, such that locking mechanism 480zzr returns to the locked state, thereby maintaining wing 220zzq in its new size.

It is to be understood that the direction in which locking mechanisms 480zzq, 480zzr facilitate or inhibit sliding of root portion 230zzr and tip portion 232zzr, and the manner in which the jaws are moved in order to lock and unlock the locking mechanisms are merely illustrative, and that other directions or methods are contemplated, mutatis mutandis.

Reference is made to FIGS. 82A-B, which are schematic illustrations showing implant 200zzs, in accordance with some implementations of the invention.

Implant 200zzs can be considered to be a variant of implant 200, and can be similar, at least in its general purpose, e.g., being anchored to a tissue of the heart so as to repair the function of the native leaflet, to implant 200 or any of the variants thereof disclosed herein, mutatis mutandis, except that implant 200zzs comprises a bulking element 420 that is coupled (in the example shown, is fixedly coupled) to the wing (in the example shown to the tip portion 232zzs of wing 220zzs). The bulking element 420 can be coupled to the wing in a variety of ways and at a variety of different locations on the wing.

FIG. 82A shows implant 200zzs implanted at mitral valve 10, such that root portion 230zzr is secured to annulus 11 by anchor, and the wing extends over posterior leaflet 12 toward anterior leaflet 14. As shown, posterior leaflet 12 fails to coapt with anterior leaflet 14 during ventricular systole. As shown, wing 220zzs only partly compensates for the lack of coaptation, and a gap between the wing and anterior leaflet 14 enables retrograde blood flow. In some such cases, it may be determined (e.g., by imaging valvular function during implantation) that closing the gap between wing 220zzr and anterior leaflet 14 could reduce retrograde blood flow during systole.

As shown, implant 200zzs comprises an actuator 474zzs, e.g., extending within catheter 140 (not shown) to controller 120, that is coupled to bulking element 420. Operating actuator 474zzs transitions bulking element 420 from a delivery state (FIG. 82A) to an expanded, actuated state (FIG. 82B) in which the bulking element has a greater volume. In some implementations, bulking element 420 comprises a braided structure that is foreshortened by actuator 474zzs, such that the bulking element becomes both shorter and wider as it transitions to the delivery state.

In some implementations, at least a portion of bulking element 420 is disposed at tip portion 232zzs (e.g., on a downstream-facing side of the tip portion, on a distal portion, etc.), such that expanding the bulking element makes the tip portion or distal portion bulkier, such that implant or wing 220zzs coapts with anterior leaflet 14. In some implementations, actuation of bulking element 420 is reversible, e.g., if it is determined that the volume of the actuated bulking element is undesirably great, the bulking element can be at transitioned at least partially toward the delivery state, reducing the bulking element's volume.

Reference is made to FIGS. 84A-B, which are schematic illustrations showing an implant 200zzu implanted at annulus 11 of native valve 10, in accordance with some implementations of the invention. Implant 200zzu can be considered to be a variant of implant 200, and can be similar, at least in its general purpose, e.g., being anchored to a tissue of the heart so as to repair the function of the native leaflet, to implant 200 or any of the variants thereof disclosed herein.

Implant 200zzu shares certain features with implant 200zzp described hereinabove, particularly locking interface 250zzp. However, in contrast to implant 200zzo's wing 220zzo that defines elongate anchor interface 334zzo, implant 200zzu comprises wing 220 whose frame 224 defines a loop 221 that fits neck portion 247zzp of interface 250zzp more closely than does anchor interface 334zzo. In some implementations, wing 220 is therefore less slidable along its length than wing 220zzo.

In some implementations, and as described hereinbelow, the wing's deflectability can be adjusted by transitioning interface 250zzp between a loose state (FIG. 84A) and a tight state (FIG. 84B) in which the loop portion is sandwiched between collars 248zzp, 249zzp. For example, interface 250 can be transitioned between the loose state and the tight state by changing a depth to which anchor 30 is anchored through the interface and into tissue of annulus 11.

FIG. 84A shows interface 250zzp in the loose state e.g., unlocked, such that the space between collars 248zzp, 249zzp allows loop 221 to pivot with respect to neck 247zzp of interface 250zzp. Pivoting of loop 221 facilitates deflection of wing 220 downstream (upper frame of FIG. 84A) and upstream (lower frame of FIG. 84A), in response to the cardiac cycle.

In some implementations, the effect of implant 200zzu upon functioning of valve 10 is assessed (e.g., by imaging flow of blood through the valve) after anchoring tissue-engaging element 34 of anchor 30 into tissue of the annulus, yet prior to withdrawing shaft 160 and driver 170 from the heart. In some implementations, shaft 160 is kept coupled to interface 250zzp and/or driver 170 is kept coupled to anchor head 32, while the effect of implant 200zzu is determined. In this way, if implant 200zzu is determined to suboptimally impact the valve's function, the implant can be readily removed by unscrewing anchor 30 from annulus 11 and retracting shaft 160. In some such cases, implant 200zzu can then be reimplanted at an alternate site in the heart.

In some implementations, in which shaft 160 remains coupled to interface 250zzp while assessing the valve's function, the shaft can influence the valve's function by supporting annulus 11 and/or leaflet 12. Although such support can in some cases be desirable, the valve's function while supported by shaft 160 can differ from the valve's function after the shaft is decoupled from interface 250zzp. In some such cases, the valve's function after decoupling shaft 160 from interface 250zzp can be less optimal than while supported by the shaft. In some implementations, it can be desirable to compensate for the shaft's support while assessing the valve's function, in order to more accurately predict the valve's function after decoupling the shaft from the interface.

In order to compensate for the shaft's support while assessing the valve's function, bloodflow through valve 10 can be imaged while interface 250zzp assumes the loose state (FIG. 84A). In some implementations, deflection of wing 220 and leaflet 12 while shaft 160 is coupled to interface 250zzp and the interface assumes the loose state can approximate, e.g., can be generally equal to, deflection of the wing and the leaflet while the interface is tightened into the tight state (FIG. 84B) and the shaft is disengaged from the interface (not shown).

In some implementations, and as shown, driver 170 is used to transition interface 250zzp from the loose state to the tight state by advancing anchor 30 through the interface 250zzp and further into tissue of annulus 11, such that anchor head 32 presses distally against the interface (FIG. 84B). In some implementations, and as shown in FIG. 84B, implant 200zzu becomes less deflectable along anchor axis a30 as interface 250 assumes the tight state.

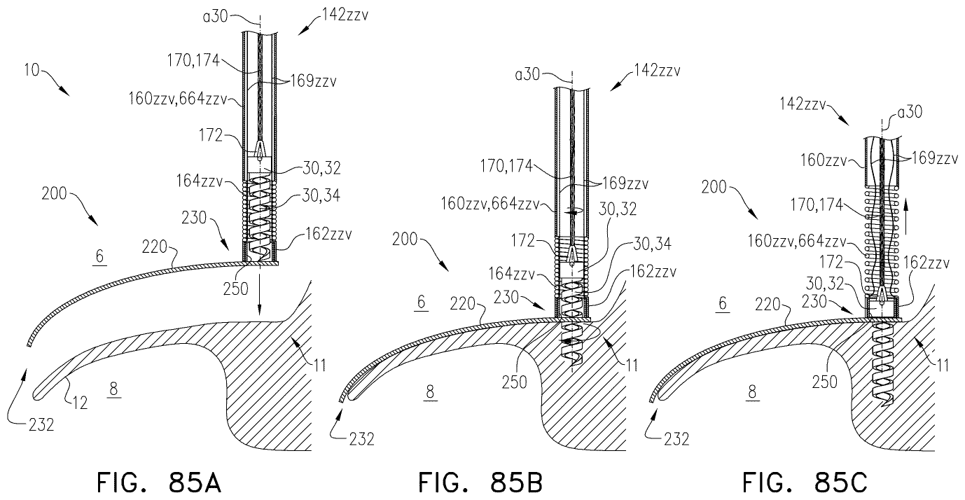

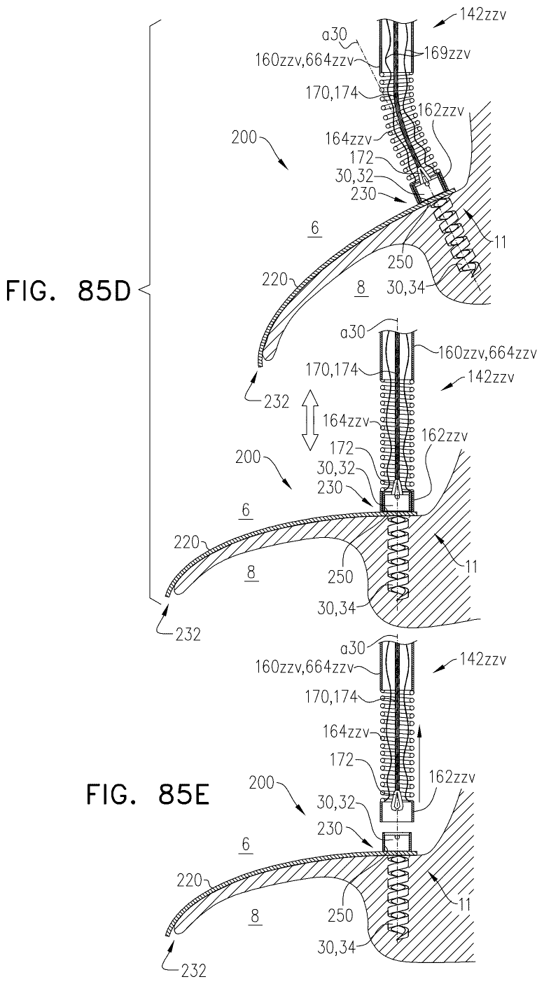

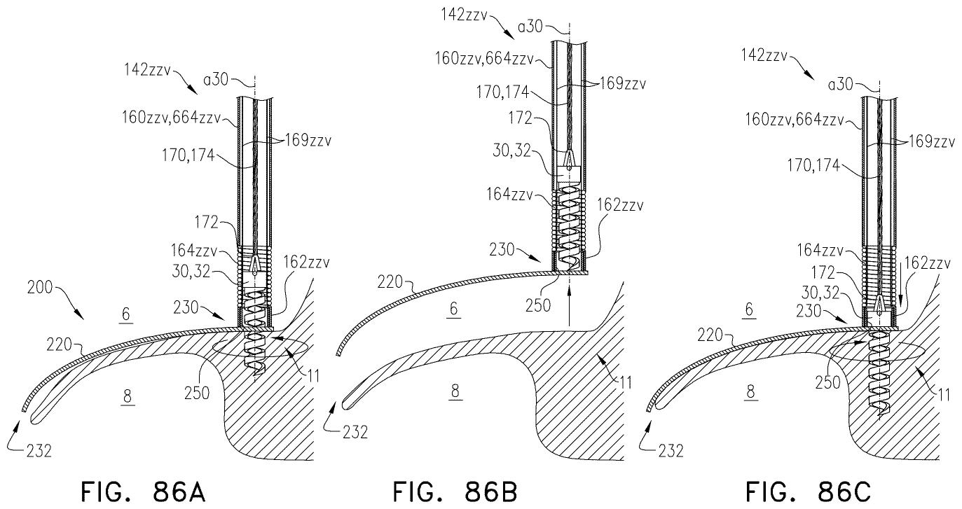

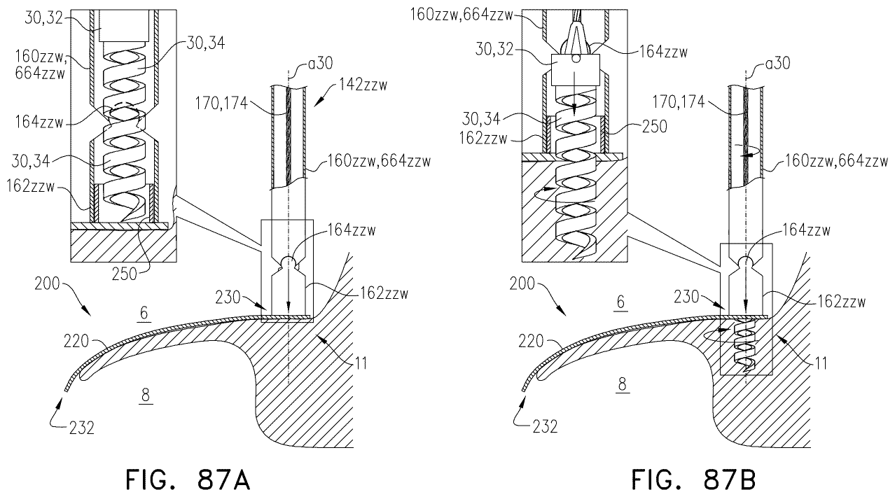

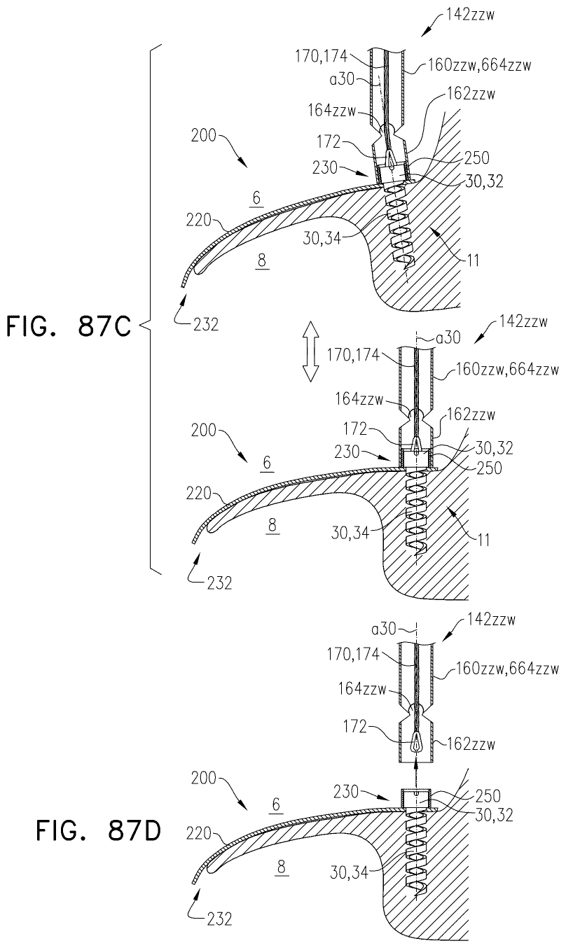

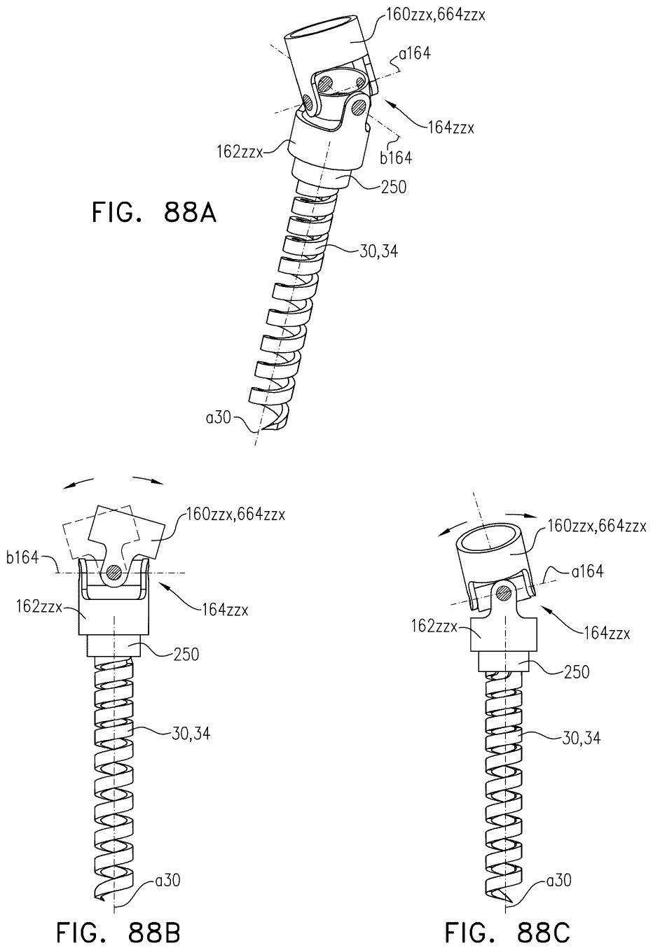

Reference is made to FIGS. 85A-E, 86A-C, 87A-D and 88A-C, which are schematic illustrations showing use of shafts 160zzv, 160zzw for implantation of implant 200 at annulus 11 of native valve 10, in accordance with some implementations of the invention. Shafts 160zzv, 160zzw can be considered to be variants of shaft 160, and can be similar, at least in their general purpose, e.g., deploying an implant out of a distal opening of a catheter. In contrast to shaft 160, shafts 160zzv, 160zzw are configured to be adjustably flexible. In some implementations, adjusting flexibility of shafts 160zzv, 160zzw can moderate deflectability of wing 220 while the shaft is engaged with interface 250 and the interface is anchored by anchor 30 to annulus 11.

FIGS. 85A-D, 86A-C and 87A-C show shaft 160zzv, 160zzw coupled to interface 250. In some implementations, and as shown, shaft 160zzv, 160zzw defines an adjustable coupling 164zzv, 164zzw between the shaft's proximal portion 664zzv, 664zzw and the shaft's distal end portion 162zzv, 162zzw. In some implementations, flexibility of coupling 164zzv, 164zzw is achieved without the coupling being materially wider than the shaft's proximal portion 664zzv, 664zzw (e.g., the coupling's outer diameter may be no more than 10 percent greater than the proximal portion's outer diameter). For example, the coupling may be no wider than the shaft's proximal portion.

FIGS. 85B-C and 87B-C each show respective coupling 164zzv, 164zzw transitioning from a rigid state (FIGS. 85B, 87B) to a flexible state (FIGS. 85C, 87C), thereby allowing greater deflection of wing 220 and/or leaflet 12 in response to the heart's cardiac cycle, while end portion 162zzv, 162zzw of shaft 160zzv, 160zzw is coupled to interface 250. FIGS. 85C, 87C show distal portion 142zzv, 142zzw of the delivery tool assuming an assessment state for assessing the valve's function (e.g., by visualizing bloodflow through the valve), in which: (i) coupling 164zzv, 164zzw remains in the flexible state, and (ii) end portion 162zzv, 162zzw of shaft 160zzv, 160zzw.

In some implementations, since the coupling's flexibility at least partially compensates for the shaft's support, function of valve 10 as distal portion 142zzv, 142zzw assumes the assessment state (FIG. 85D, 87C) approximates (e.g., be generally equal to) function of the valve after shaft 160zzv, 160zzw is disengaged from interface 250.

In some cases, if implant 200 is determined to positively affect function of valve 10, distal portion 142zzv, 142zzw of the delivery tool is released by disengaging driver 170 from anchor 30 and decoupling shaft 160zzv, 160zzw from interface 250, such that the delivery tool can be withdrawn from the subject (FIG. 85E, 87D).

In some implementations, and as shown in FIGS. 85A-E and 86A-C, coupling 164zzv comprises a spring. FIG. 85A shows distal portion 162zzv of shaft 160zzv being used to deliver implant 200 to annulus 11 of native valve 10, while the spring is tensioned in the rigid state. In some implementations, and as shown in FIG. 85B, driver 170 is used to drive anchor 30 along anchor axis a30, through interface 250 and into tissue of annulus 11, while the spring remains tensioned. In some implementations, and as shown in FIG. 85C, tension is released from the spring, e.g., by reducing tension on tethers 169zzv via controller 120, such that the coupling's spring transitions into the flexible state. FIG. 85D shows deflection of wing 220 and leaflet 12 while distal portion 142zzv of the delivery tool assumes the assessment state.

In some cases, if implant 200 is determined to suboptimally affect function of valve 10, rather than disengaging shaft 160zzv, 160zzw from interface 250, the user can choose to remove or relocate implant 200. In some implementations, coupling 164zzv is re-tensioned, e.g., by increasing tension on tethers 169zzv, prior to unscrewing anchor 30 from the site (FIG. 86A). In some implementations, after detaching implant 200 from the tissue (FIG. 86B), shaft 160zzv, 160zzw is used to redeploy implant 200, which is then re-anchored at an alternate tissue site (FIG. 86C).

In some implementations, and as shown in FIGS. 87A-D, coupling 164zzw comprises a hinge that articulatably couples proximal portion 664zzw of shaft 160zzw to distal end portion 162zzw of the shaft. In some implementations, while the hinge is in a rigid state, shaft 160zzv is used to deliver implant 200 to annulus 11 (FIG. 87A) and driver 170 is used to drive anchor 30 along anchor axis a30, through interface 250 and into tissue of the annulus (FIG. 87B).

In some implementations, and as shown in FIGS. 87B-C, movement of anchor 30 within coupling 164zzw (e.g., anchoring anchor 30 through interface 250 and into tissue of annulus 11) adjusts the coupling's flexibility. In some implementations, as long as anchor 30 is disposed within the coupling's hinge (insets of FIGS. 87A-B), the coupling remains in the rigid state in which the hinge has a limited range of articulation.

FIG. 87C shows anchor 30 having been driven past the hinge, such that coupling 164zzw assumes a flexible state in which the hinge is foldable across a wider range of articulation, such that wing 220 and leaflet 12 deflect while shaft 160 remains coupled to interface 250. In some implementations, and as shown, the coupling's hinge folds along an articulation-axis that is aligned with deflection of wing 220 and leaflet 12.

FIGS. 88A-C shows a coupling 164zzx, which is in many ways similar to coupling 164zzw, except that coupling 164zzx comprises a hinged joint, such as a universal joint. This joint couples proximal portion 664zzx of shaft 160zzx to distal end portion 162zzx of the shaft. In some implementations, and as shown, the joint facilitates articulation of proximal portion 664zzx with respect to distal end portion 162zzx along both a first articulation axis a164 (FIG. 88B) and a second articulation axis b164 (FIG. 88C). In some implementations, and as shown, first articulation-axis a164 is nonparallel to (e.g., orthogonal to) second articulation-axis b164.

In some implementations, and similarly to coupling 164zzw described hereinabove, movement of anchor 30 within coupling 164zzx (e.g., anchoring anchor 30 through interface 250 and into tissue of annulus 11) adjusts the coupling's flexibility. In some implementations, driving anchor 30 through interface 250 transitions coupling 164zzx from the rigid state to the flexible state, increasing the Cardan joint's range of articulation along articulation axes a164, b164.

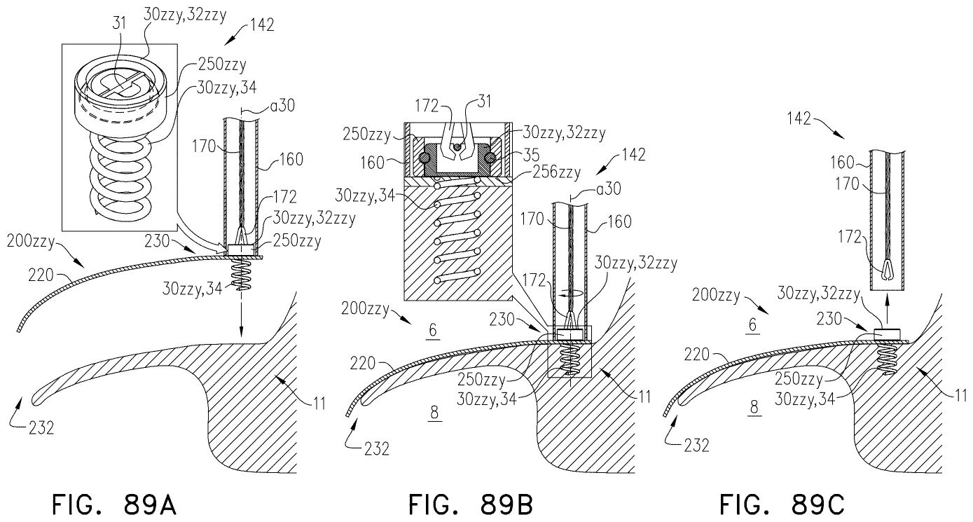

Reference is made to FIGS. 89A-C, which are schematic illustrations showing use of distal portion 142 of delivery tool 150 to implant an implant 200zzy using an anchor 30zzy, in accordance with some implementations of the invention. Implant 200zzy and anchor 30zzy can be respectively considered to be variants of implant 200 and anchor 30, and can be similar, at least in their general purpose, e.g., repairing the function of the native leaflet, to implant 200 and anchor 30 or any of the variants thereof disclosed herein.

As shown, implant 200zzy and anchor 30zzy are configured to inhibit passage of anchor head 32zzy past distal end 256zzy of interface 250zzy. In some implementations, anchor 30zzy is integral to implant 200zzy. Alternatively, anchor 30zzy can be provided separately from implant 200zzy, and inserted into place by the user.

In some implementations, and as shown, anchor head 32zzy is rotatably coupled to interface 250zzy in a manner that allows for rotation of anchor 30zzy with respect to (e.g., within) the interface, while the anchor head remains longitudinally fixed with respect to the interface. In some implementations, sheet 226zzy is shaped to define a hole at interface 250zzy, which can facilitate rotation of anchor head 32zzy within interface 250zzy.

In some implementations, and as shown, anchor head 32zzy and/or interface 250zzy are shaped to accommodate a ring 35 that keeps the anchor head longitudinally fixed with respect to interface 250zzy, while allowing for rotation of anchor 30zzy with respect to the interface. For example, and as shown, tissue-engaging element 34 can extend from implant 200 during deployment (FIG. 89A), such that screwing anchor 30zzy into tissue 3 sandwiches wing 220 between interface 250zzy and the tissue (FIG. 89B), after which the delivery tool is withdrawn (FIG. 89C).

Alternatively (not shown), rather than utilizing a ring to fit the anchor head to the interface, the anchor head can be shaped to fit around (e.g., to at least partially circumscribe) the interface. In some implementations, the anchor head has an inner diameter that is wider than both: helical tissue-engaging element 34, and the interface. In this way, the anchor head is prevented from advancing further distally from the interface as the tissue-engaging element is driven into the tissue.

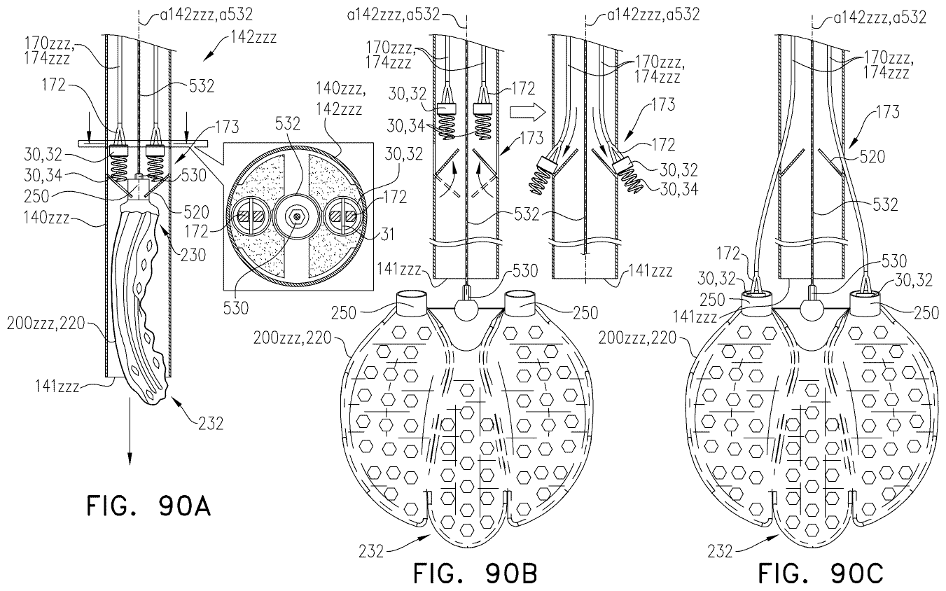

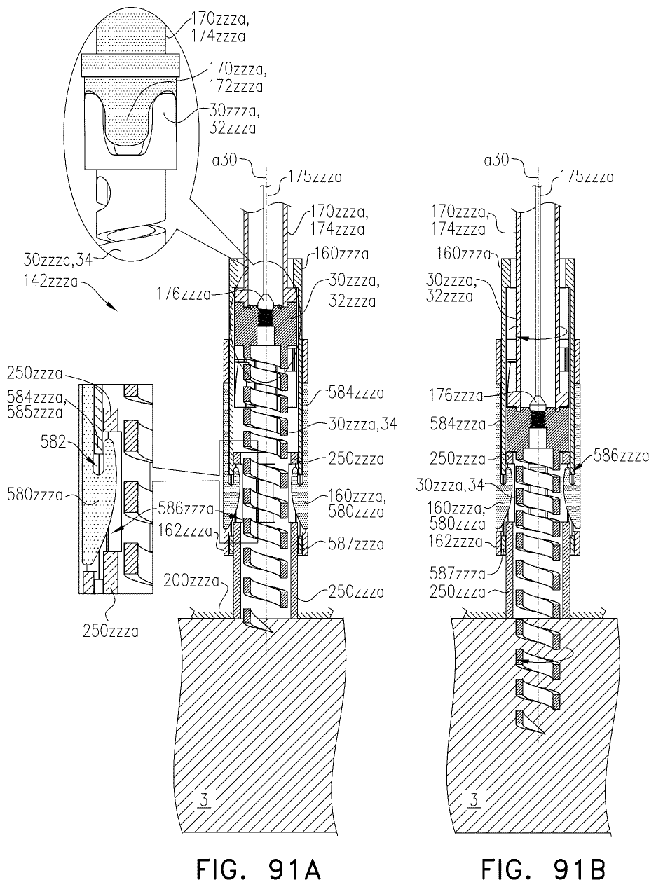

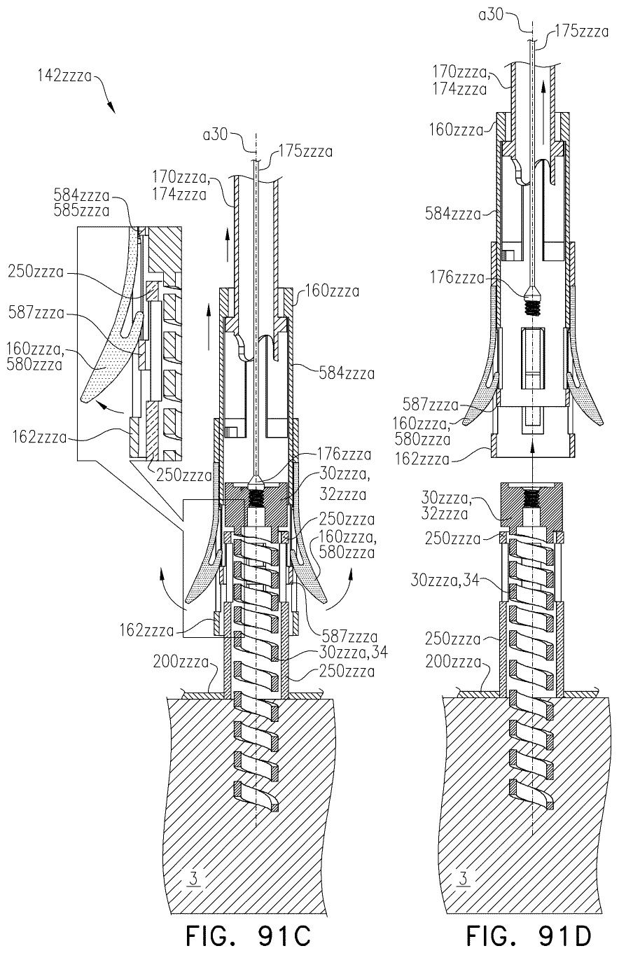

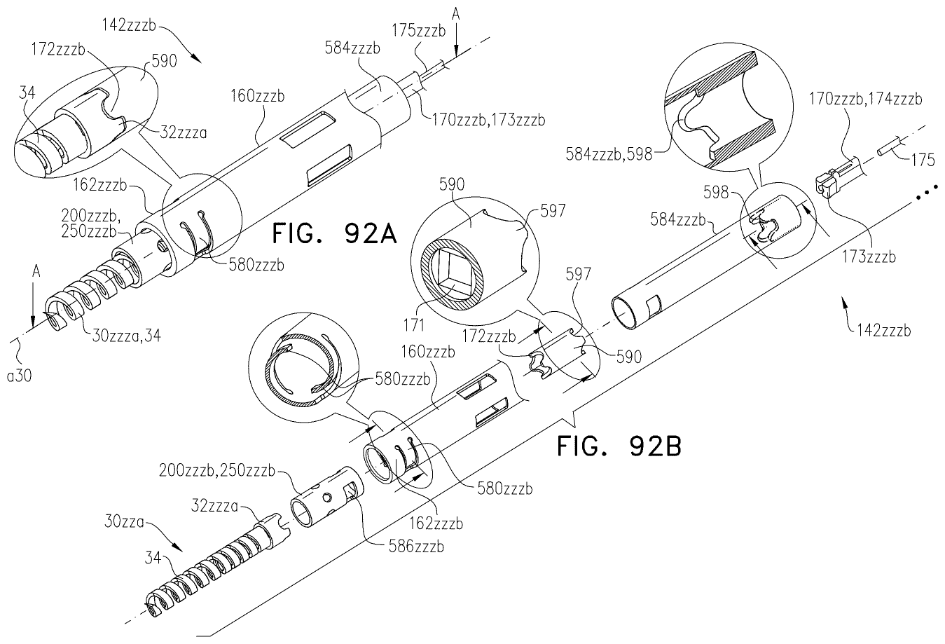

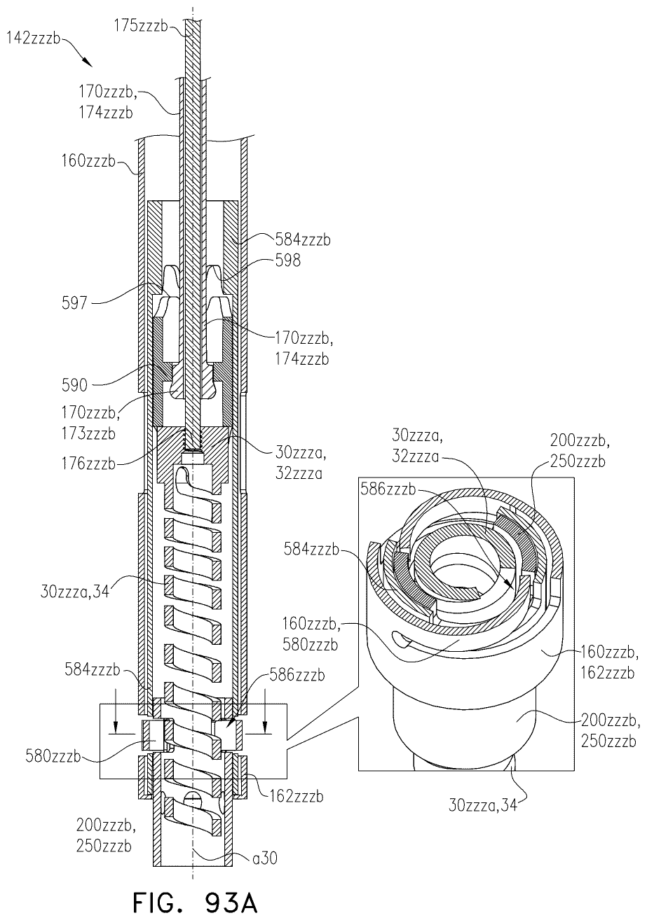

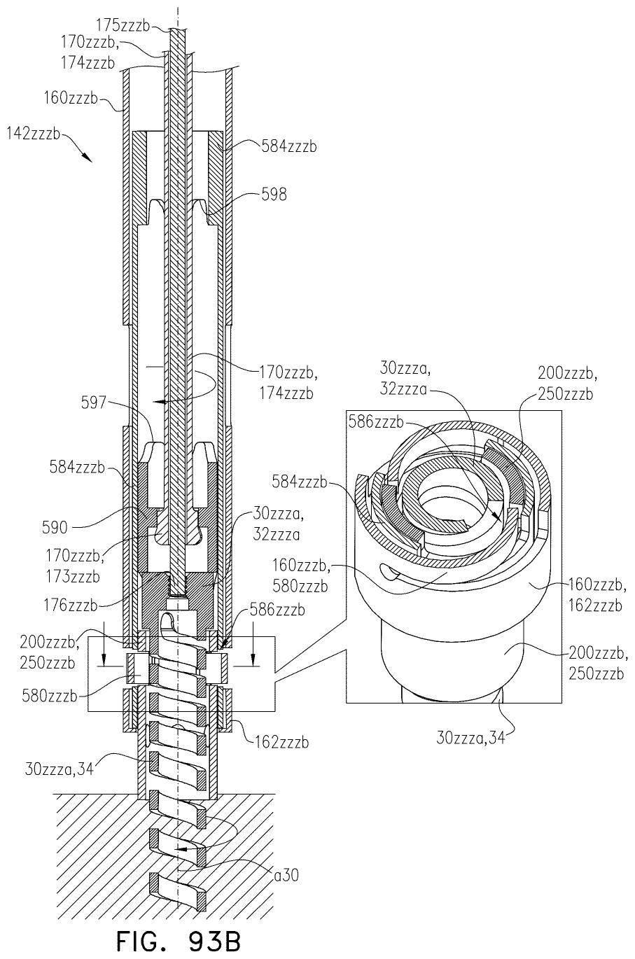

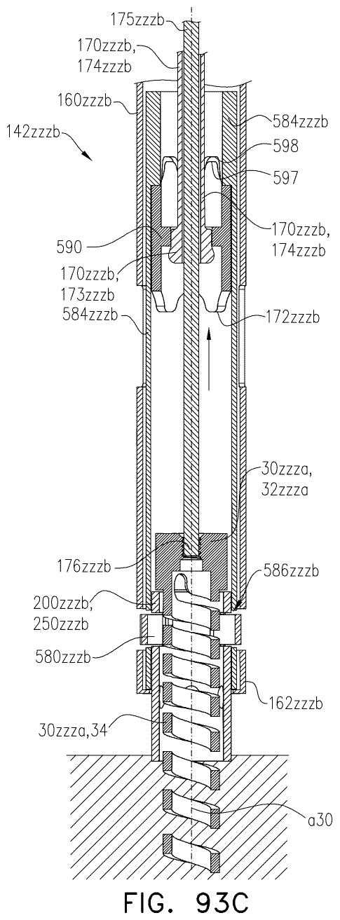

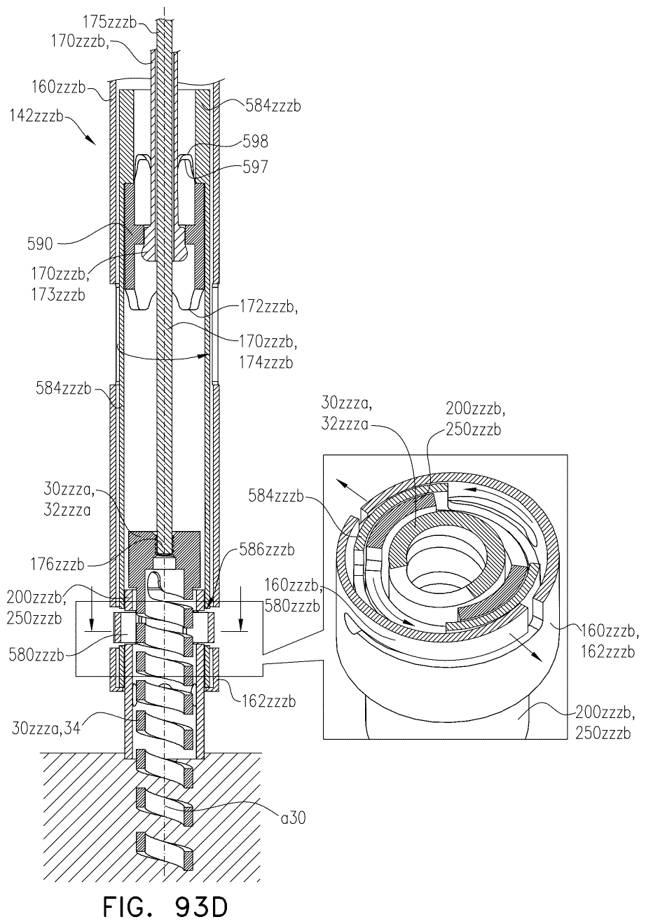

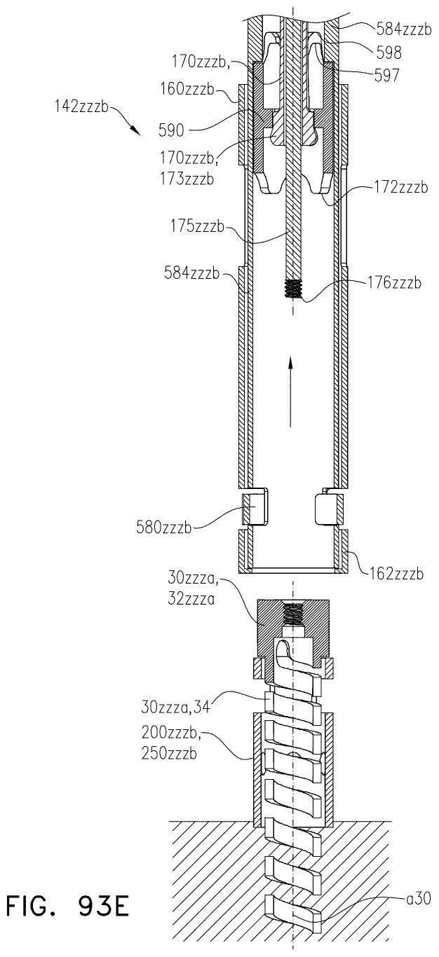

Reference is made to FIGS. 90A-C, which are schematic illustrations showing use of a distal portion 142zzz of a delivery tool to deploy an implant 200zzz from a delivery catheter 140zzz, in accordance with some implementations of the invention.

Implant 200zzz can be considered to be a variant of implant 200, and can be similar, at least in its general purpose, e.g., being anchored to a tissue of the heart so as to repair the function of the native leaflet, to implant 200 or any of the variants thereof disclosed herein, mutatis mutandis, except that implant 200zzz comprises a deployment interface 530 that is separate from interface (e.g., anchor receiver) 250.

In some implementations, and as shown, distal portion 142zzz of the delivery tool comprises a delivery catheter 140zzz that can be considered to be a variant of catheter 140, and can be similar, at least in its general purpose, e.g., to deliver implant 200 or any of the variants thereof disclosed herein to the native leaflet, mutatis mutandis, except that distal portion 142zzz of catheter 140zzz defines lateral openings 173, e.g., at which gates 520 are disposed.

In some implementations and as shown in FIG. 90B, implant 200zzz and anchors 30 are deployed from catheter 140zzz along different paths. In some implementations, catheter 140zzz has a distal opening 141zzz through which implant 200zzz is deployed distally, e.g., via deployment interface 530. In some implementations, anchors 30 exit catheter 140zzz laterally, e.g., via lateral openings 173 of catheter 140zzz (right frame of inset of FIG. 90B). in this way, implant 200zzz can be deployed along a longitudinal axis a142zzz of the catheter's distal portion 142zzz, whereas drivers 170zzz can deploy anchors 30 obliquely to axis a142zzz.

In some implementations, and as shown, distal portion 142zzz of the delivery tool comprises a rod 532 that is coupled to implant 200zzz via deployment interface 530, e.g., independently of drivers 170zzz and anchors 30. In some implementations, gates 520 are held closed by interface 530 and/or by rod 532 during delivery of implant 200zzz (FIG. 90zzz).

As shown, implant 200zzz is compressed within a distal portion of catheter 140zzz during delivery. In some implementations, and as shown, implant 200zzz is coupled to deployment rod 532 via deployment interface 530 during delivery of the implant. In some implementations, and as shown, drivers 170zzz and anchors 30 are disposed alongside each other within catheter 140zzz during delivery of implant 200zzz, such that each driver and anchor are disposed on either side of rod 532.

FIG. 90B shows implant 200zzz having been deployed from distal opening 141zzz of catheter 140zzz, e.g., by pushing rod 532 distally, such that wing 220 expands upon being released from the catheter. In some implementations, rod 532 and/or interface 530 facilitate expansion of implant 200zzz by comprising expansion elements 270e, 270f, 270g described hereinabove with reference to FIGS. 8A-B, 9A-B and 10A-B, mutatis mutandis.

In some implementations, and as shown in FIG. 90B, deployment of implant 200zzz via interface 530 and/or proximally retracting drivers 170zzz within catheter 140zzz release gates 520 from their closed position. In some implementations, gates 520 comprise a shape-memory material such that the gates open upon being released.

FIG. 90B shows use of drivers 170zzz to advance anchors 30 out of respective gates 520 (FIG. 90B), such that the anchors engage respective anchor receivers 250 (FIG. 90C). In some implementations, drivers 170zzz advance anchors 30 toward anchor receivers 250 along a path that diverges from the rod's longitudinal axis a532. In some implementations, anchors 30 are advanced divergently away from each other, e.g., in a direction that is generally oblique with respect to delivery axis, e.g., with respect to longitudinal axis a532.

In some implementations, In some implementations, a distal portion of the driver's driveshafts 174zzz comprise a shape-memory material that biases the driveshafts so as to steer the anchors toward the anchor receivers. In some implementations, an angle at which gates 520 are opened in relation to longitudinal axis a532 facilitates steering the anchors toward the anchor receivers.

After anchors 30 are disposed within anchor receivers 250 (FIG. 90C), implant 200zzz can be anchored to tissue of the heart by driving tissue-engaging elements 34 through the anchor receivers and into the tissue.

Reference is made to FIGS. 91A-D, 92A-B and 93A-E, which are schematic illustrations showing use of show a distal portion 142zzza, 142zzzb of a delivery tool to implant an implant 200zzza, 200zzzb, in accordance with some implementations of the invention. Implants 200zzza, 200zzzb can be considered to be variants of implant 200, and can be similar, at least in their general purpose, e.g., being anchored to a tissue of the heart so as to repair the function of the native leaflet, to implant 200 or any of the variants thereof disclosed herein, mutatis mutandis.

In some implementations, and as shown, distal portion 142zzza, 142zzzb comprises a shaft 160zzza, 160zzzb that defines a latch 580zzza, 580zzzb which reversibly engages the shaft to implant 200zzza, 200zzzb (e.g., an interface 250zzza, 250zzzb thereof). In some implementations, and as shown, sliding an insert 584zzza, 584zzzb between shaft 160zzza, 160zzzb and interface 250zzza, 250zzzb disengages the shaft from the interface, e.g., by displacing latch 580zzza, 580zzzb.

FIGS. 91A-D show distal portion 142zzza of the delivery tool that comprises shaft 160zzzb, which is reversibly engaged to interface 250zzzb. In some implementations, and as described hereinbelow, sliding insert 584zzza along anchor axis a30 disengages the shaft 160zzza from interface 250zzza by displacing latch 580zzza.

In some implementations, and as shown in the inset of FIG. 91A, interface 250zzza is shaped to define a window 586zzza, and latch 580zzza engages shaft 160zzza to the interface when the latch protrudes into the window. In some implementations, and as shown, insert 584zzza (e.g., a fastening portion 585zzza thereof) is disposed between the shaft 160zzza and interface 250zzza, e.g., within the latch's groove 582. As shown, latch 580zzza is held within window 586zzza while shaft 160zzza is used to position implant 200zzza against tissue 3 of the heart.

In some implementation, and as shown, insert 584zzza comprises an intervening tube (e.g., disposed coaxially between shaft 160zza and interface 250zza). Insert 584zza (e.g., the intervening tube) can be coupled to a driver 170zzza. The intervening tube can circumscribe a portion of the driver and/or anchor 30zzza.

In some implementations, and as shown, a rail 175zzza (e.g., a rail head 176zzza thereof) is connected to anchor head 32 while anchor 30 is screwed into the tissue, such that tensioning the rail keeps anchor head 32 fitted to drive head 172zzza while the anchor is screwed into the tissue.

As shown in FIGS. 91C-D, after tissue-engaging element 34 of anchor 30 is screwed through interface 250zzza and into tissue 3, shaft 160zzza is typically disengaged from interface 250zzza. In some implementations, insert 584zzza is slid, e.g., proximally along anchor axis a30, in order to disengage shaft from interface 250zzza. In some implementations, and as shown, removal of fastening portion 585zzza from within the latch's groove 582 disengages latch 580zzza from interface 250zzza. For example, latch 580zzza can comprise a shape-memory material that is biased such that removing insert 584zzza from groove 582 allows the latch to transition from a compressed shape to a relaxed shape in which the latch disengages from interface 250zzza.

In some implementations, and as shown, insert 584zzza comprises a disengaging portion 587zzza, and sliding the insert causes the disengaging portion to push latch 580zzza laterally outward and/or away from interface 250zzza.

In some implementations, and as shown in FIG. 91D, once implant 200zzza is disengaged from shaft 160zzza, rail 175zzza can be detached (e.g., rail head 176zzza can be unscrewed) from anchor head 32, such that shaft 160zzza and driver 170zzza are withdrawn from the subject.

Reference is again made to FIGS. 92A-B and 93A-E, which show distal portion 142zzzb of a delivery tool that comprises shaft 160zzzb which is reversibly engaged to interface 250zzzb. As described hereinbelow, rotating insert 584zzzb about anchor axis a30 disengages shaft 160zzzb from interface 250zzzb by displacing latch 580zzzb.

In some implementations, and as shown in an assembled view (FIG. 92A) and an exploded view (FIG. 92B) of distal portion 142zzzb, driver 170zzzb comprises a driveshaft 174zzzb that extends distally to wrench 590, such that the wrench is both slidable and rotatable with respect to shaft 160zzzb. In some implementations, driveshaft 174zzzb and wrench 590 fit around rail 175zzzb such that the driveshaft and the wrench are rotatable with respect to the rail and shaft 160zzzb, facilitating transfer of torque from the driveshaft to anchor 30zzzb.

In some implementations, and as shown in the upper inset of FIG. 92A, a distal portion of wrench 590 defines a drive head 172zzzb that is shaped to fit anchor head 32zzza. In such implementations, engagement between driveshaft 174zzb and wrench 590 facilitates transfer of torque from the driveshaft to the drive head. In the example shown, wrench 590 is shaped to define a driver receiver 171 that is reversibly engageable by a distal end portion 173zzzb of driveshaft 174zzzb. Alternatively, distal end portion 173zzzb of driveshaft 174zzzb can be fixedly coupled to wrench 590.

In some implementations, and as shown in FIG. 92B, a proximal portion 597 of wrench 590 is shaped to interface with intervening tube 584zzzb, e.g., with a key 598 defined by the intervening tube. In some implementations, wrench 590 is advanceable and retractable by sliding driveshaft 174zzzb. In this way, wrench 590 can apply torque either to anchor head 32zzza when the wrench is advanced in a distal position (FIG. 92A, 93A-B) or to intervening tube 584zzzb when the wrench is retracted to a proximal position (93C-E).

FIG. 93A shows a cross-sectional view of distal portion 142zzzb in a deployment state in which drive head 172zzzb is engaged to anchor head 32zzzb. In some implementations, and as shown, anchor 30zzza is disposed within shaft 160zzzb and/or interface 250zzzb during delivery. The inset of FIG. 93A shows shaft 160zzzb engaged to interface 250zzzb by latch 580zzzb that is defined by distal end portion 162zzzb of the shaft. In some implementations, and as shown, interface 250zzzb defines a window 586zzzb, and intervening tube 584zzzb is positioned to allow latch 580zzzb to enter the window, thereby engaging the interface to shaft 160zzzb.

FIG. 93B shows torque being applied via driveshaft 174zzzb to anchor 30zzza, which screws the anchor into tissue 3, while shaft 160zzzb remains engaged to interface 250zzzb via latch 580zzzb. In some implementations, rail 175zzzb is longitudinally slidable with respect to driveshaft 174zzzb, such that tension can be applied to the rail while screwing anchor 30zzza into the tissue. In some implementations, tensioning rail 175zzzb helps to keep drive head 172zzza fitted to anchor head 32zzza the while torque is applied to the anchor head, e.g., reducing a risk of the drive head slipping off of the anchor head.

FIGS. 93C-D show use of wrench 590 to disengage shaft 160zzzb from interface 250zzzb by sliding intervening tube 584zzzb between the shaft and the interface such that the shaft's latch 580zzzb leaves the interface's window 586zzzb. As shown in FIG. 93C, driveshaft 174zzzb is pulled proximally, such that the wrench's drive head 172zzzb disengages from anchor head 32zzzb and the wrench's proximal portion 597 interfaces with the intervening tube's key 598. In the next step (FIG. 93D), rotating tube 584zzzb pushes latch 580zzzb out of window 586zzzb, thereby disengaging shaft 160zzzb from interface 250zzzb (insert of FIG. 93D). At this stage, distal portion 142zzzb of the delivery tool can be withdrawn (FIG. 93E), such that interface 250zzzb remains anchored to tissue 3.

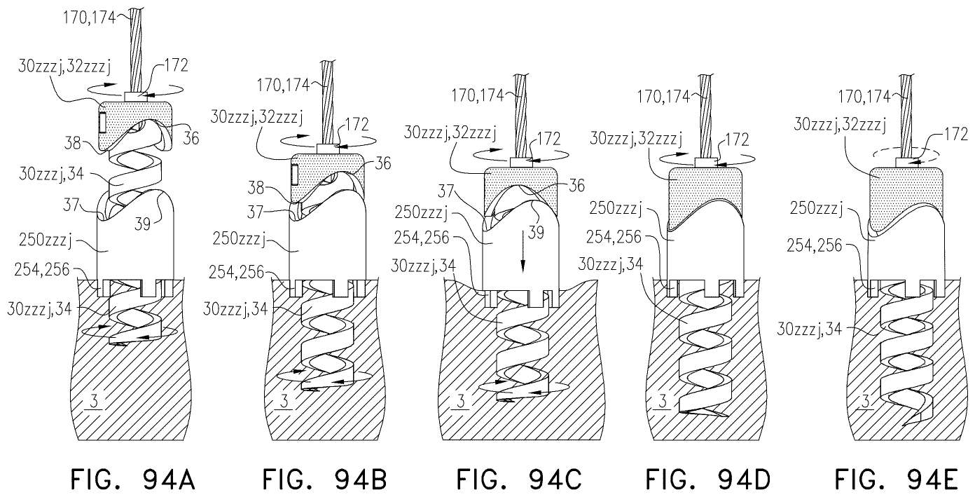

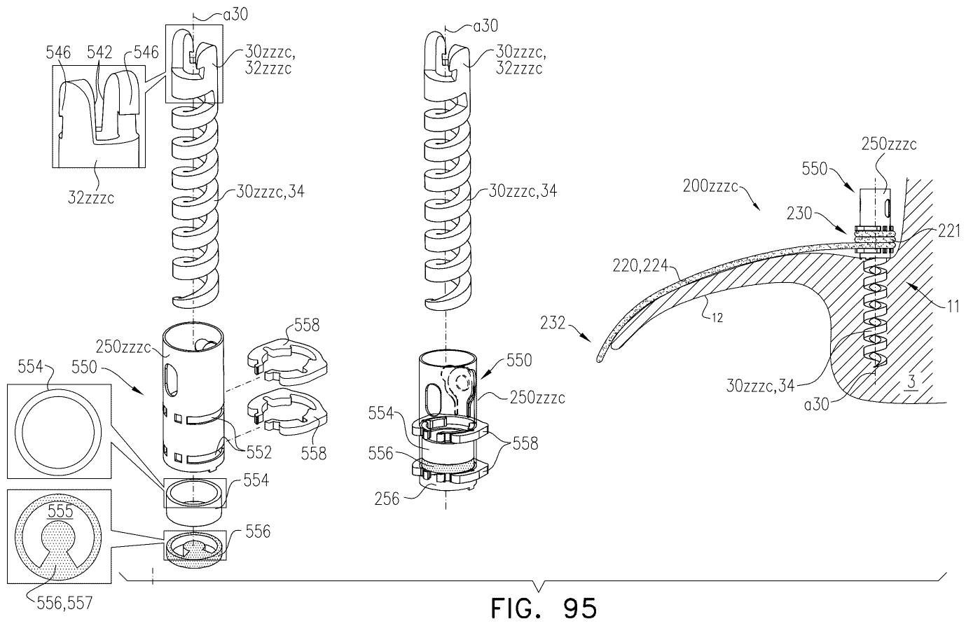

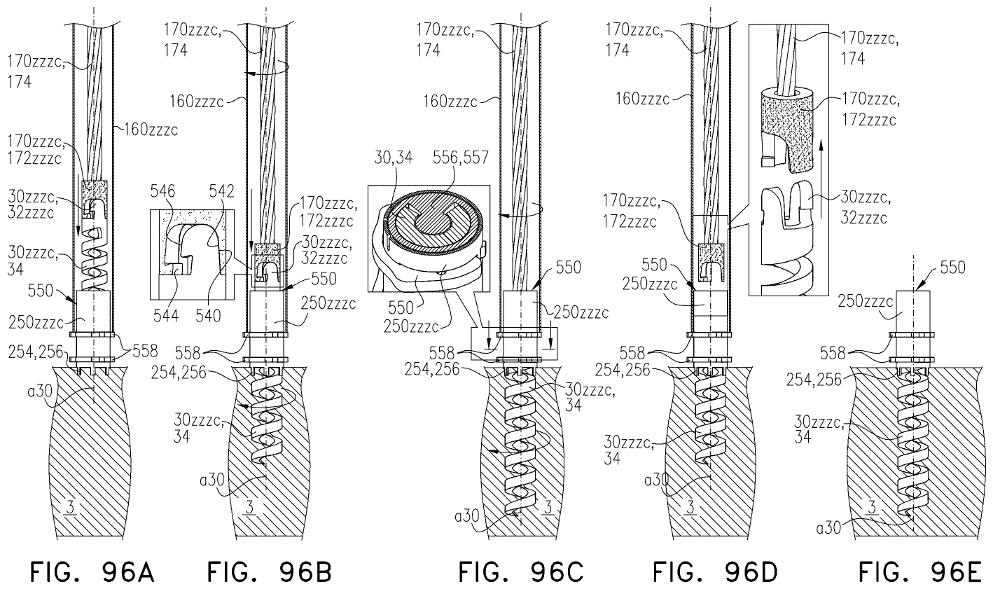

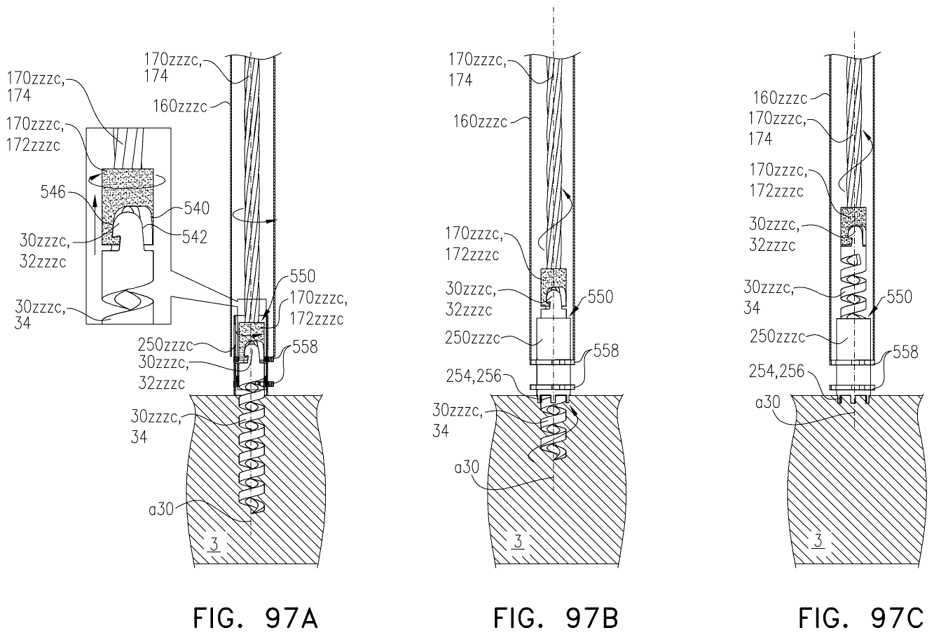

Reference is made to FIGS. 94A-E, which are schematic illustrations showing use of an anchor 30zzzj with an anchor receiver 250zzzj, in accordance with some implementations of the invention. Anchor 30zzzj and anchor receiver 250zzzj can be considered to be variants of anchor 30 and interface 250, and can be similar, at least in their general purpose, e.g., anchoring an implant to tissue of the heart, to anchor 30 and interface 250 or any of the variants thereof disclosed herein, mutatis mutandis. Anchor 30zzzj and anchor receiver 250zzzj are configured to supplement resistance that the tissue offers to torque that is applied to anchor head 32zzzj, via driveshaft 174 of driver 170, when anchoring the anchor to the anchor receiver.

In some implementations, and in contrast to the flat surface along which anchor head 32 and interface 250 are shown to interact in FIGS. 3B-E, anchor head 32zzzj and anchor receiver 250zzzj are each shaped to define peaks 38, 39 and troughs 36, 37 along which the anchor head interacts with the anchor receiver. In some implementations, and as shown in FIGS. 94A-E, anchor head 32zzzj and anchor receiver 250zzzj each define complementarily undulating contact surfaces, such that a portion of the torque that driver 170 transfers to anchor head 32zzzj is translated into a distal pushing force upon anchor receiver 250zzzj.

FIGS. 94A-C show drive head 172 being used to apply torque to anchor head 32zzzj in order to screw 30zzzj through anchor receiver 250zzzj and into tissue 3. Anchor receiver 250zzzj is typically part of a larger implant, such as implant 200 (not shown for clarity) or any of the variants thereof disclosed herein. FIG. 94A shows an initial stage of screwing anchor 30zzzj into anchor receiver 250zzzj, in which anchor head 32zzzj advances helically toward anchor receiver 250zzzj as tissue-engaging element 34 advances helically through tissue 3.

FIG. 94B-C shows anchor 30zzzj having further advanced with respect to anchor receiver 250zzzj. At this stage, during at least part of the anchor head's rotation, anchor head 32zzzj does not contact anchor receiver 250zzzj, and therefore anchor 30zzzj continues to advance helically toward the anchor receiver. However, during part of the anchor head's rotation, peaks 38 of the anchor head contact peaks 39 of the anchor receiver (FIG. 94B). As shown in FIG. 94C, at least a portion of the torque applied to anchor head 32zzzj pushes anchor receiver 250zzzj distally with respect to the anchor, thereby compressing tissue 3. In some implementations, resistance of tissue 3 to compression supplements resistance to the torque that is applied to anchor head 32zzzj, e.g., supplementing resistance that the tissue offers to helical advancement of tissue engaging element 34 therethrough, and/or to rotation of teeth 254 of the interface's distal end 256 within the tissue.

FIG. 94D shows anchor 30zzzj having advanced further with respect to anchor receiver 250zzzj, such that anchor head 32zzzj is fully seated within the anchor receiver, with peaks 38 of the anchor head seated against troughs 37 of the anchor receiver, and peaks 39 of the anchor receiver seated against troughs 36 of the anchor head. At this stage, application of additional torque to the anchor head (FIG. 94E) cannot necessarily result in rotation of the anchor, due to resistance that tissue 3 and anchor receiver 250zzzj present to the torque. That is, when anchor head 32zzzj is fully seated within anchor receiver 250zzzj, further rotation of anchor 30zzzj would require the anchor to move proximally away from anchor receiver 250zzzj, in order for peak 38 of anchor head 32zzzj to climb over peak 39 of anchor receiver. However, proximally elevating anchor head 32zzzj would entail also proximally elevating tissue-engaging element 34, together with tissue 3 within which element 34 is screwed. Tissue 3 would typically resist being pulled proximally upward, supplementing resistance to the torque that is applied to anchor head 32zzzj.

As a result, application of torque locks anchor 30zzzj and anchor receiver 250zzzj in place, which the user experiences as resistance to torque that is applied via driver 170 to the anchor. In some implementations, the resistance can indicate to the user to stop applying the torque.

The reader will note that FIGS. 94A-E relate to application of forward torque. In some implementations, torque in either rotational direction has a similar effect of locking anchor 30zzzj and anchor receiver 250zzzj in place. That is, when anchor head 32zzzj is fully seated within anchor receiver 250zzzv, application of reverse torque would similarly require the anchor to move proximally away from anchor receiver 250zzzj, in order for peak 38 of anchor head 32zzzj to climb over peak 39 of anchor receiver. The user would experience similar resistance to torque that is applied via driver 170 to the anchor as the torque locks anchor 30zzzj and anchor receiver 250zzzj in place, e.g., indicating to the user to stop applying the torque.

Reference is made to FIGS. 95, 96A-E and 97A-C, which are schematic illustrations showing use of an anchor 30zzzc with an interface 550 of an implant 200zzzc, in accordance with some implementations of the invention. Anchor 30zzzc and interface 550 can be considered to be variants of anchor 30 and interface 250 of implant 200, and can be similar, at least in their general purpose, e.g., anchoring implant 200zzzc to a tissue of the heart so as to repair the function of the heart, to anchor 30 and interface 250 or any of the variants thereof disclosed herein.

FIG. 95 shows an exploded view (left frame) and perspective views of anchor 30zzzc engaged to interface 550 (center frame) and implanted to tissue 3 of annulus 11 (right frame). In some implementations, and as shown, anchor 30zzzc defines an anchor head 32zzzc attached to a proximal end of tissue-engaging element 34, which extends distally from the anchor head along anchor axis a30. In some implementations, anchor head 32zzzc and tissue-engaging element 34 are cut from a unitary piece of stock tubing.

Anchor 30 is configured to anchor interface 550 to tissue 3 of the heart (e.g., tissue of annulus 11, as shown in FIG. 95) by helically advancing the anchor's tissue-engaging element 34 through the interface and into the tissue, in response to application of forward torque to anchor head 32zzzc. As described hereinbelow, interface 550 is configured to inhibit non-helical advancement of the anchor distally through the interface, thereby facilitating a snug fit between tissue 3 and distal end 256 of the interface when anchor 30zzzc is anchored to the tissue.

In some implementations, and as shown, interface 550 comprises a tubular anchor receiver 250zzzc defining a lumen, and a stopper 556 that is disposed within the anchor receiver's lumen. In some implementations, stopper 556 is shaped to define a window 555 shaped for the anchor's tissue-engaging element 34 to pass helically therethrough, and a wall 557 that is shaped to prevent the tissue-engaging element from being pushed through the stopper. In this way, tissue-engaging element 34 can be screwed through stopper 556, and helical advancement of anchor 30zzzc through interface 550 is halted when anchor head 32zzzc reaches the stopper.

In some implementations, and as shown, interface 550 further comprises a washer 554 that also fits within the anchor receiver's lumen, e.g., proximally of stopper 556. In some implementations, and as shown, washer 554 is shaped to facilitate passage of both tissue-engaging element 34 and anchor head 32zzzc therethrough as anchor 30zzzc is screwed through interface 550. By fitting between anchor head 32zzzc and anchor receiver 250zzzc, washer 554 can inhibit pivoting of interface 550 with respect to anchor axis a30, e.g., while wing 220 of implant 200zzzc deflects in response to the heart's cardiac cycle.

In some implementations, and as shown, interface 550 comprises a pair of clips 558 that snap-fit to notches 552 in anchor receiver 250zzzc so as to restrain washer 554 and stopper 556 in place as anchor 30zzzc is screwed through the interface.

In some implementations, and as shown in the inset of FIG. 95, anchor head 32zzzc is shaped to define a forward-torque face 542 (e.g., a smooth face that is closer to being perpendicular to the forward torque than to being parallel to the forward torque) and a reverse-torque face 546, e.g., shaped to define an anchor hook 547, as shown. As shown, anchor hook 547 faces forward (e.g., the rotational direction in which tissue-engaging element 34 can be helically advanced), and forward-torque face 542 faces an opposite, reverse rotational direction. In this way, forward torque can be applied to anchor head 32zzzc by pressing forward-torque face 542, and reverse torque can be applied by pressing reverse-torque face 546.

FIGS. 96A-E show use of a driver 170zzzc to screw anchor 30zzzc through interface 550 and into tissue 3 of the heart, thereby anchoring the interface to the tissue. In some implementations, and as shown, the driver's driveshaft 174 extends distally via shaft 160 to a drive head 172zzzc that is shaped to transfer torque to anchor head 32zzzc. As shown, drive head 172zzzc defines a smooth, forward-facing driver screw-in surface 540 (e.g., shaped complementarily to the anchor head's forward-torque face 542), and a reverse-facing driver hook 544 (e.g., shaped complementarily to the anchor head's anchor hook 547). In some implementations, drive head 172zzzc and anchor head 32zzzc are configured to be cut from a unitary piece of stock tubing.

In some implementations, and as described hereinbelow, driver head 172zzzc and anchor head 32zzzc not require a change in shape or conformation in order to: (i) apply forward torque to forward-torque face 542, (ii) pull the drive head away from the anchor head, or (iii) apply reverse torque to reverse-torque face 546.

FIG. 96A shows shaft 160 having been used to advance driver 170zzzc and anchor 30zzzc toward interface 550 and tissue 3. FIG. 96B-C shows tissue-engaging element 34 being screwed through interface 550 and into tissue 3. As shown in the inset of FIG. 96B, forward rotation of driver 170zzzc with respect to anchor head 32zzzc presses driver screw-in surface 540 against forward-torque face 542, while driver head 172zzzc presses the anchor head distally.

As shown in the inset of FIG. 96C, a proximal portion of tissue-engaging element 34 is disposed within the stopper's window 555. In some implementations, anchor 30zzzc is advanced through interface 550 until anchor head 32zzzc contacts the stopper's wall 557, which inhibits further advancement of the anchor into tissue. In this way, anchor 30zzzc is helically advanceable through interface 550, yet wall 557 inhibits non-helical advancement of the anchor, facilitating a snug fit of the interface to the tissue.

In some implementations, and as shown in FIG. 96D, after interface 550 is determined to be satisfactorily anchored to tissue 3, driver head 172zzzc is disengaged from anchor head 32zzzc (e.g., by tensioning driveshaft 174), and the driver is withdrawn from the subject together with shaft 160 (FIG. 96E).

In some cases, it can be desirable, after anchoring interface 550 to tissue 3, to remove anchor 30zzzc from the tissue, e.g., in order to re-anchor the interface to an alternate site of tissue, or to remove the implant from the subject. FIGS. 97A-C show use of driver 170zzzc to unscrew tissue-engaging element 34 from tissue 3 by applying reverse torque to anchor head 32zzzc. As shown in the inset of FIG. 97A, reverse rotation of drive head 172zzzc hooks driver hook 544 onto anchor hook 547, e.g., while sliding the drive head proximally with respect to the anchor head, which facilitates unscrewing anchor 30zzzc while tensioning driveshaft 174 by pulling the driveshaft proximally (FIG. 97B).

FIG. 97C shows tissue-engaging element 34 having been unscrewed from tissue. In some implementations, driver 170zzzc and shaft 160 are then removed from the subject, e.g., while the drive hook 544 remains hooked onto anchor head 32zzzc and driveshaft 174 remains tensioned.

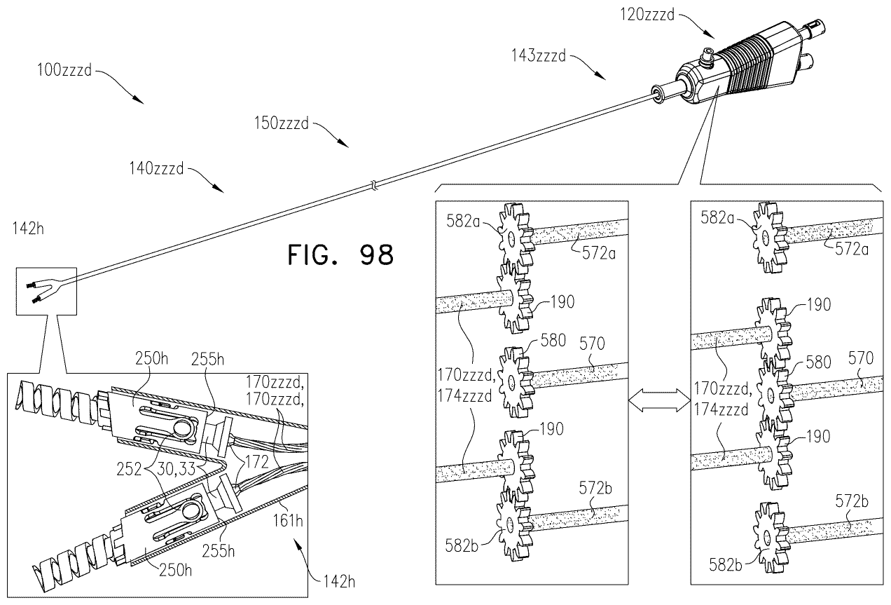

Reference is made to FIG. 98, which shows schematic illustrations of a system 100zzzd, in accordance with some implementations of the invention. System 100zzzd can be considered to be a variant of system 100, and can be similar, at least in its general purpose, e.g., driving anchor 30, to system 100 disclosed herein, except that system 100zzzd comprises a delivery tool 150zzzd having a distal portion 142zzzd comprising a pair of drivers 170zzzd for driving anchors 30. In this respect, distal portion 142zzzd is generally identical to distal portion 142h of delivery tool 150h described hereinabove, mutatis mutandis.

As shown, proximal portion 143zzzd of tool 150zzzd comprises a controller 120zzzd that is configured to operate drivers 170zzzd. In some implementations, controller 120zzzd is configured to operate drivers 170zzzd (a) simultaneously, or (b) individually. In some implementations, the operator can set the controller 120zzzd (e.g., via buttons, levers, dials, etc.) to operate drivers 170zzzd either simultaneously or individually, e.g., facilitating iterative transitioning between operating both of the drivers simultaneously or only one of the drivers at a given time.

For example, the inset on the right side of FIG. 98 schematically represents controller 120zzzd being set for operating drivers 170zzzd simultaneously (right frame) or individually (left frame). As shown, controller 120zzzd houses a main driveshaft 570, as well as auxiliary driveshafts 572a, 572b that are selectively activated via controller 120zzzd for providing torque to drivers 170zzzd. The right frame shows driveshafts 174zzzd of drivers 170zzzd having been shifted towards main driveshaft 570, e.g., such that respective driver gears 190 of the drivers interface with a main gear 580 of the main driveshaft, which simultaneously transfers torque to each driver via the interfaced gears.

In some cases, it can be preferable to drive each anchor 30 individually, e.g., sequentially. For such cases, the operator can set controller 120zzzd to operate each driver 170zzzd individually. For example, the left frame of the inset on the right side of FIG. 98 shows driveshafts 174zzzd having each been shifted away from main driveshaft 570 and towards a respective auxiliary driveshaft 572a, 572b, such that driver gears 190 each interface with a respective auxiliary gear 582a, 582b of the auxiliary driveshafts. In this way, torque can be selectively (e.g., sequentially) transmitted to drivers 170zzzd by operating auxiliary driveshafts 572a, 572b individually.

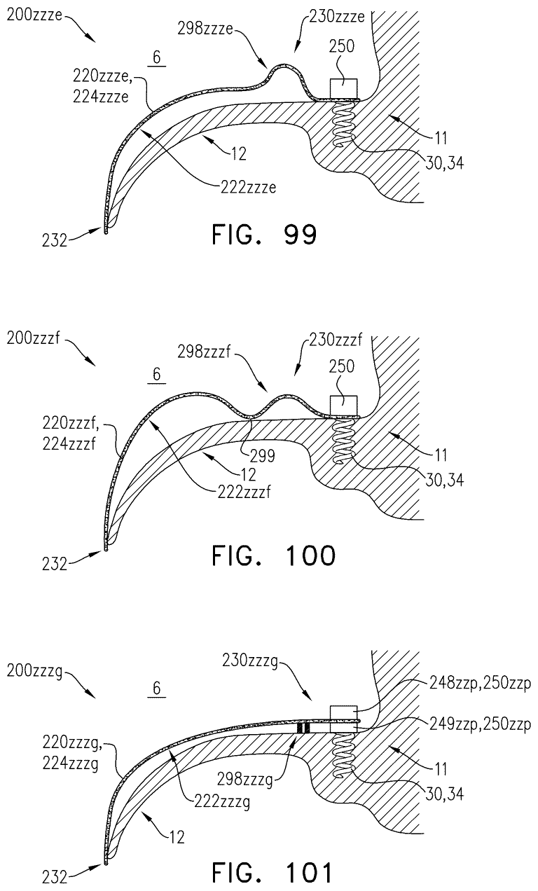

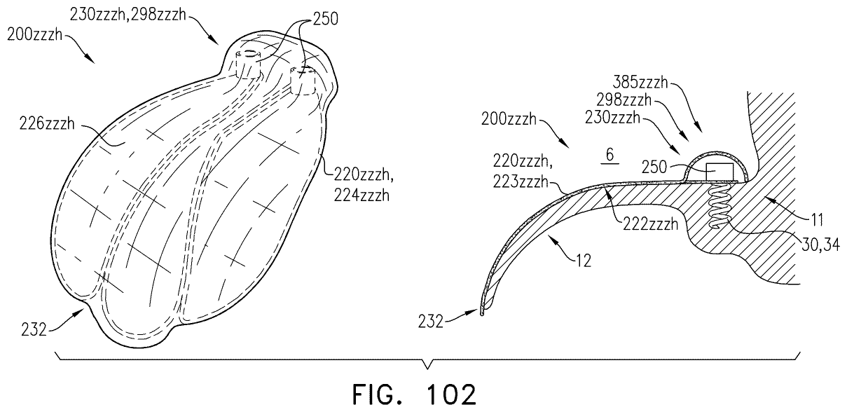

Reference is made to FIGS. 99-102, which are schematic illustrations showing implants 200zzze, 200zzzf, 200zzzg, 200zzzh, in accordance with some implementations of the invention. Implants 200zzze, 200zzzf, 200zzzg, 200zzzh can be considered to be variants of implant 200, and can be similar, at least in their general purpose, e.g., being anchored to a tissue of the heart so as to repair the function of the native leaflet, to implant 200 or any of the variants thereof disclosed herein, mutatis mutandis, except that implants 200zzze, 200zzzf, 200zzzg, 200zzzh are configured to influence ingrowth of tissue upon the implant following implantation in the heart. In some implementations, and as shown, anchor receiver 250 is anchored to annulus 11 and a wing 220zzze, 220zzzf, 220zzzg, 220zzzh extends over leaflet 12, towards an opposing leaflet (not shown).

In some implementations, implants 200zzze, 200zzzf, 200zzzg, 200zzzh are configured to facilitate ingrowth of tissue at anchor receiver 250, and to inhibit ingrowth of tissue at tip portion 232zzze, 232zzzf, 232zzzg, 232zzzh of wing 220zzze, 220zzzf, 220zzzg, 220zzzh. Whereas tissue ingrowth at anchor receiver 250 can support the anchoring of implant 200zzze, 200zzzf, 200zzzg, 200zzzh to annulus, inhibiting tissue ingrowth at tip portion 232zzze, 232zzzf, 232zzzg, 232zzzh of the wing can facilitate upstream and downstream deflection of the wing in response to the cardiac cycle.

In some implementations, implants 200zzze, 200zzzf, 200zzzg, 200zzzh define an obstacle 298e, 298f, 298g, 298h that is configured to inhibit the tissue ingrowth from progressing from anchor receiver 250 toward the wing's tip portion. In some implementations, wing 220zzze, 220zzzf, 220zzzg, 220zzzh is shaped to define the obstacle. For example, and as shown in FIGS. 99-100, root portion 230zzze, 230zzzf of wing 220zzze, 220zzzf (e.g., frame 224zzze, 224zzzf of the wing) can inhibit tissue ingrowth from progressing toward the wing's tip portion 232 by curving away from anchor receiver 250 in an upstream direction, thereby reducing contact between the root portion and leaflet 12. In some implementations, a portion of root portion 230zzze, 230zzzf that bends away from the leaflet comprises a flex element, e.g., as described hereinabove with reference to FIGS. 20-23.

In some implementations, and as shown in FIG. 99, frame 224zzze is shaped such that, while anchor receiver 250 is anchored to annulus, root portion 230zzze bends away from leaflet 12, whereas tip portion 232 of the wing curves in a downstream direction, toward, e.g., such that contact face 222zzze contacts leaflet 12 at the tip portion.

In some implementations, and as shown in FIG. 100, frame 224zzzf is shaped such that, while anchor receiver 250 is anchored to annulus, wing 220zzzf bends, as the wing extends away from the anchor receiver: (i) at root portion 230zzzf, away from leaflet 12 to form an obstacle 298zzzf to tissue ingrowth, (ii) back toward the leaflet to define a contact portion 299 of the wing, (iii) distally from the contact portion, again away from the leaflet, and (iv) at tip portion 232 the wing curves in a downstream direction, toward (e.g., contacting) the leaflet. In some such implementations, and as shown, contact face 222zzzf contacts leaflet 12 at tip portion 232 and/or at contact portion 299 as the leaflet deflects during the cardiac cycle.

In some implementations, and as shown in FIG. 101, obstacle 298zzzg comprises a stilt, attached to a contact face 222zzzg of root portion 230zzzg of wing 220zzzg. As shown, the stilt inhibits tissue ingrowth from progressing toward the wing's tip portion 232 by distancing the root portion's contact face 222zzzg from leaflet 12. In some implementations, and as shown, implant 200zzzg can comprise interface 250zzp described hereinabove with reference to FIGS. 79A-C, e.g., such that second collar 249zzo and stilt 298zzzg together distance root portion 230zzzg from leaflet 12, mutatis mutandis.

In some implementations, and as shown in FIG. 102, obstacle 298zzzh comprises a cage (e.g., formed by frame 224zzzh and/or sheet 226zzzh) on an opposing face 223zzzh of wing 220zzzh that is opposite a contact face 222zzzh of the wing. The cage comprises a barrier at root portion 230zzzh of the wing that blocks tissue ingrowth from progressing from anchor receiver 250 toward the wing's tip portion 232. In some implementations, the barrier comprises multiple layers, e.g., a bilayer. In some implementations, an interface-facing layer of the barrier comprises a material (e.g., a textured polyester or TPU) that promotes tissue growth thereupon, whereas an opposing layer of the barrier comprises a material (e.g., UHMWPE) that inhibits tissue growth thereupon.

In some implementations, and as shown, obstacle 298zzzh defines a backstop portion 385zzzh of wing 220zzzh that is shaped to press against tissue of the heart when anchor receiver 250 is anchored to annulus 11, e.g., similarly to backstop portions 385zzj of wing 220zzj described hereinabove with reference to FIGS. 72 and 73A-C, mutatis mutandis.

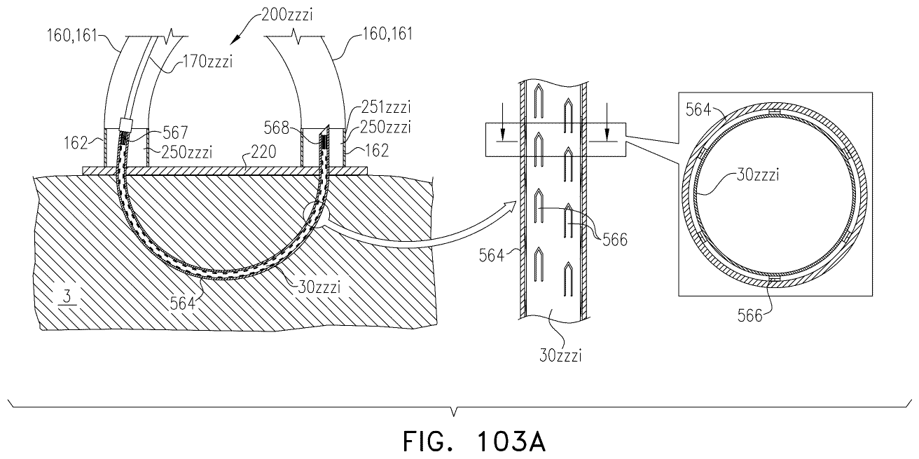

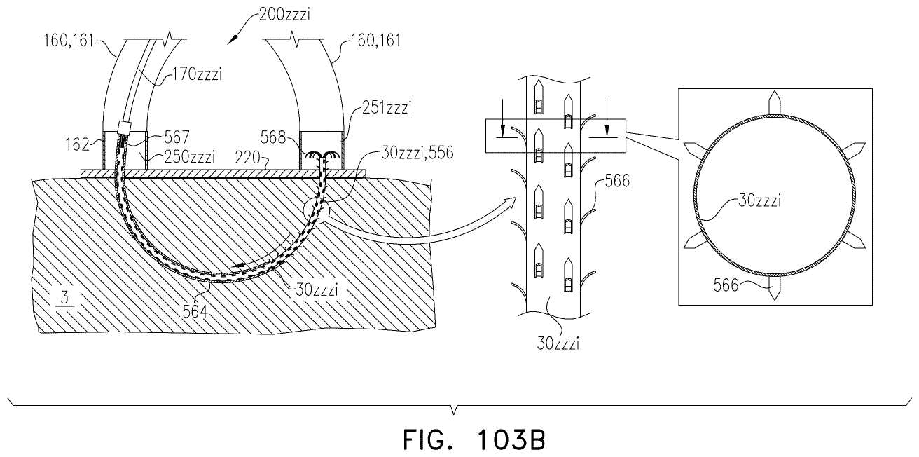

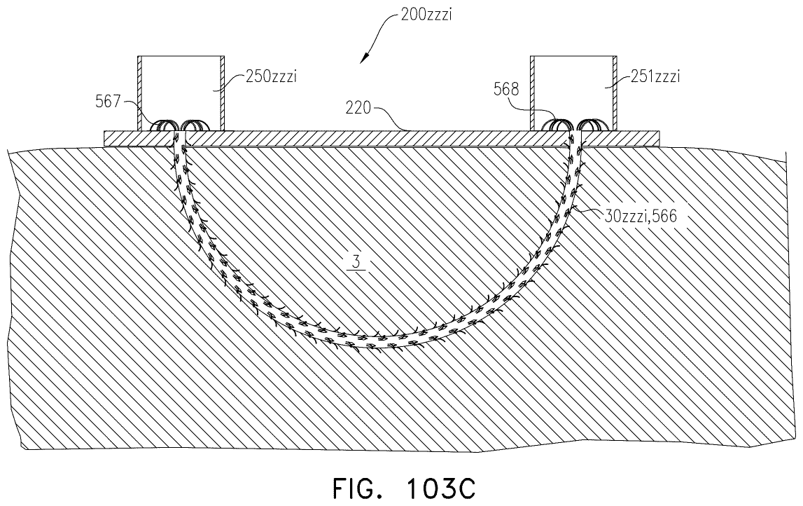

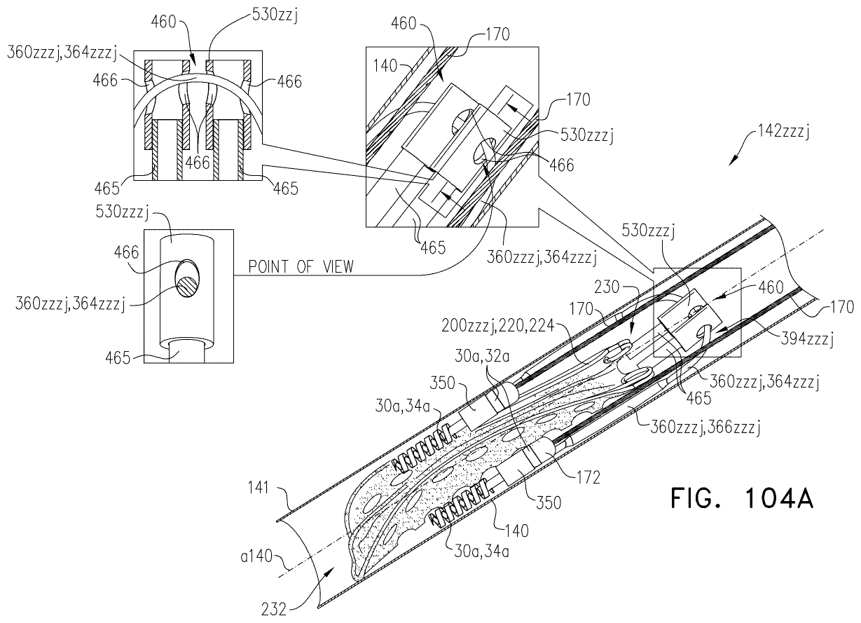

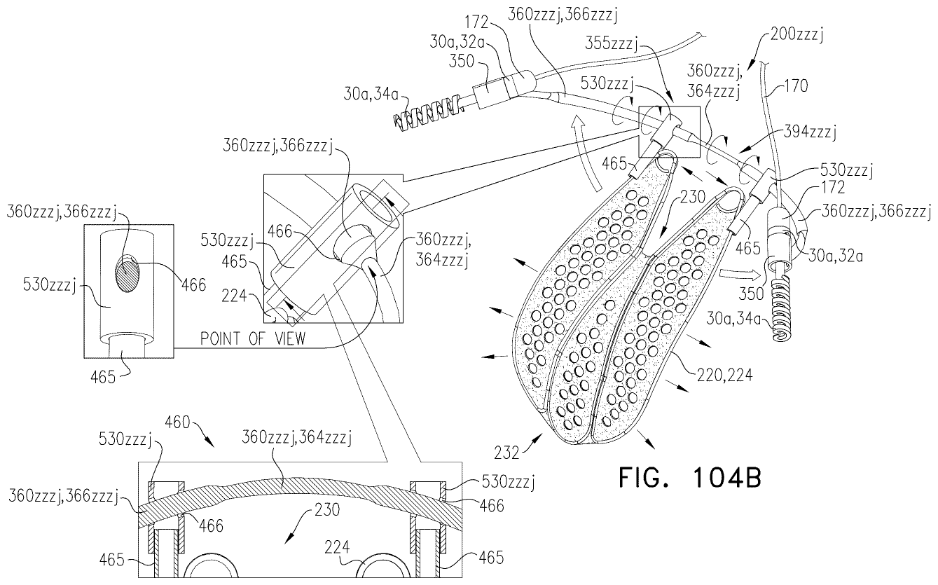

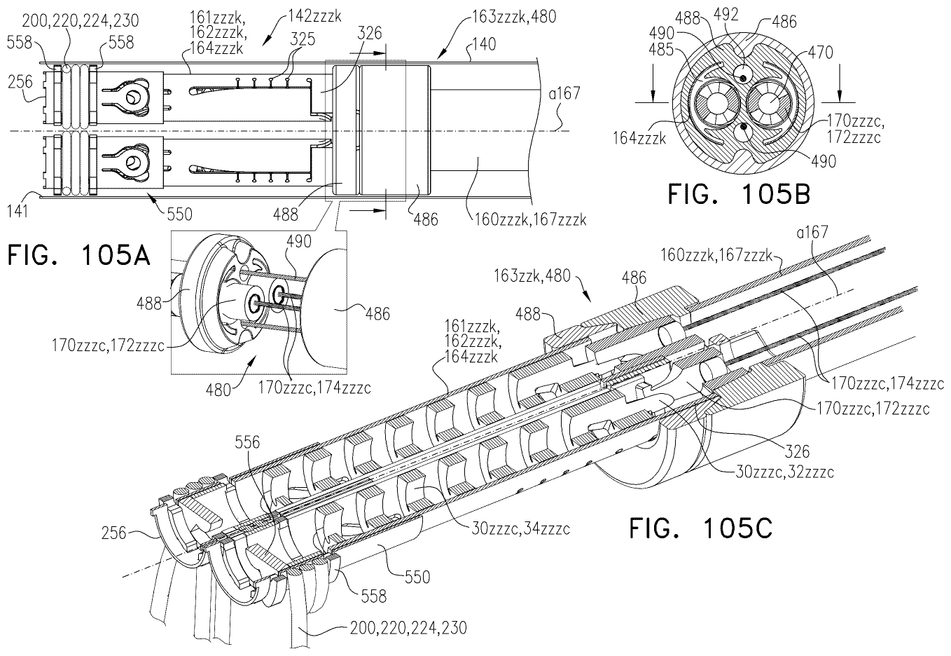

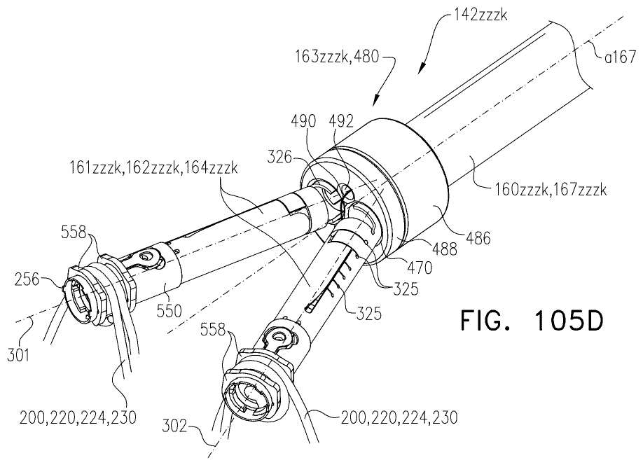

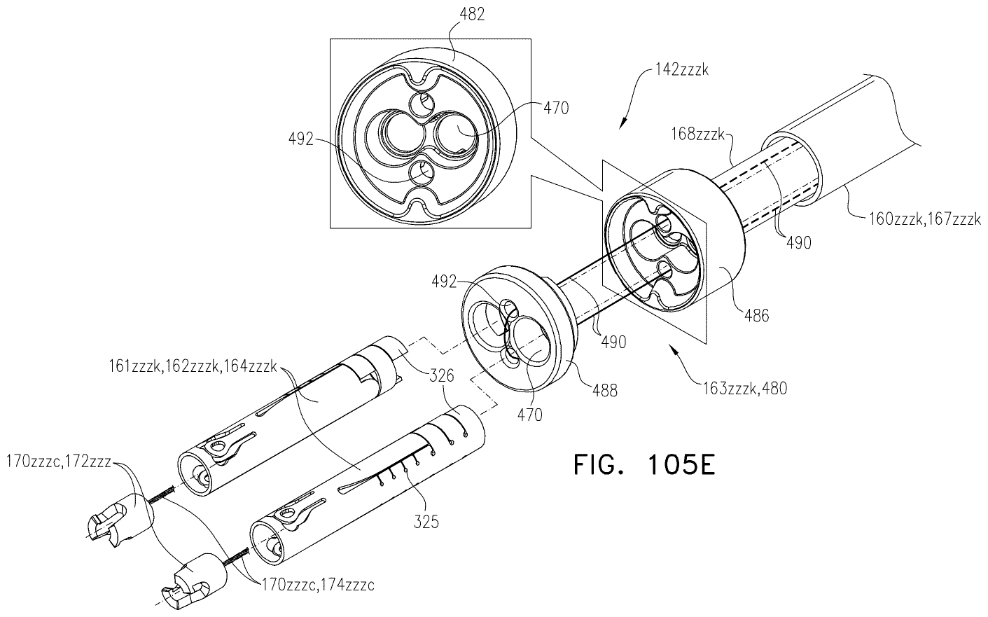

Reference is made to FIGS. 103A-C, which are schematic illustrations showing implantation of an implant 200zzzi, in accordance with some implementations of the invention. As shown, implant 200zzzi comprises wing 220 and interfaces 250zzzi. However, needle 564 and anchor 30zzzi can be used to anchor other implants to tissue of a subject, mutatis mutandis.

FIGS. 103A-B show use of shaft 160 that bifurcates into two branches 161 to deliver implant 200zzzi to tissue 3. However, needle 564 and/or anchor 30zzzi can be used to anchor implant 250zzzi to tissue while a shaft is engaged to only one interface 250zzzi, mutatis mutandis.

In some implementations, and as shown, distal end portions 162 of branches 161 each engage a respective interface 250 as implant 200zzzi is positioned at a surface of tissue 3. In some implementations, and as shown, while implant 200zzzi is in the position, a driver 170zzzi is used to advance an elongate anchor 30zzzi (e.g., housed within a flexible needle 564) through interface 250zzzi and the tissue's surface, along a curved path within the tissue such that a distal part 568 of the anchor exits the tissue and is received by anchor receiver 251zzzi.

In some implementations, anchor 30zzzi comprises a shape-memory material that the is restrained within needle 564 in a compressed state (FIG. 103A). As shown in FIG. 103B, retracting the needle releases anchor 30zzzi, allowing the anchor to expand to an expanded state. In some implementations, and as shown, release of anchor 30zzzi from needle 564 allows: barbs 566 to expand radially outward from anchor 30zzzi, and/or distal part 568 of the anchor to expand within anchor receiver 251zzzi, such that the anchor receiver engages the anchor's distal part.

FIG. 103C shows needle 564 and shaft 160 having been withdrawn, such that proximal part 567 of anchor 30zzzi expands within interface 250zzzi, which engages the anchor's proximal part. In this way, anchor 30zzzi anchors implant 200zzzi to tissue 3 following withdrawal of shaft 160.

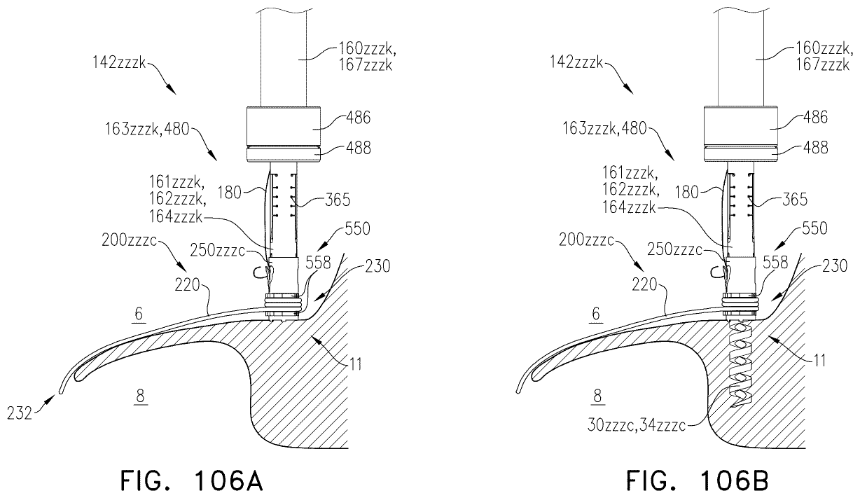

Reference is made to FIGS. 104A-B, which are schematic illustrations showing an example implant 200zzzj, in accordance with some implementations of the invention. Implant 200zzzj can be considered to be a variant of implant 200, and can be the same as or similar, at least in its general purpose, e.g., being anchored to a tissue of the heart so as to repair the function of the native leaflet, to implant 200 or any of the variants thereof disclosed herein. In some implementations, implant 200zzzj shares certain features with implants 200zk, 200zl 200zm, 200zn described hereinabove, for example, that implant 200zzzj comprises a pair of annular arms 360zzzj that each arcs away from root portion 230 of the wing to an anchor receiver 350.

In some implementations, annular arms 360zzzj are away from root portion 230 to define an annular support 355zzzj, e.g., similarly to annular support 355zk described hereinabove with reference to FIGS. 46A-B.

In some implementations, and as shown in FIGS. 104A-B, the delivery tool comprises a pair of drivers 170 for driving anchors 30a through anchor receivers 350 and into tissue of annulus 11, e.g., such that annular arms 360zzzj extend along an atrial surface of the annulus, as shown described hereinabove with reference to implant 200zk in FIG. 46B.

In accordance with some implementations, FIG. 104A shows implant 200zzzj in a compressed state in which the implant is housed within the distal portion of delivery catheter 140, similarly to as described hereinabove with reference to implant 200zn in FIG. 49B.

In some such implementations, implant 200zzzj comprises shape-memory material that biases the implant to expand into an expanded state (FIG. 104B) when the implant is advanced distally out of the catheter's distal end 141.

In some such implementations, implant 200zzzj is mechanically expandable and can be actuated to expand into an expanded state (FIG. 104B) when or after the implant is advanced distally out of the catheter's distal end 141.

In some implementations, and as shown, wing 220 (e.g., root portion 230 thereof) is connected to annular arms 360zzzj via a hinged coupling 460 that facilitates articulation of the wing (e.g., of root portion 230 thereof) with respect to the arms while implant 200zzzj is in the compressed state. In some such implementations, articulating wing 220 with respect to arms 360zzzj and/or interfaces 350 can facilitate positioning implant 200zzzj within catheter 140, e.g., such that the wing and the interfaces can both lie along a longitudinal axis a140 of catheter 140 (FIG. 104A).

In some implementations, and as shown, hinged coupling 460 comprises a pair of sleeves 530zzzj that are each connected to root portion 230 of wing 220 (e.g., via a respective connector 465) and slidably coupled to annular arms 360zzzj. In some such implementations, and as shown, annular arms 360zzzj are joined at a thin portion 364zzzj that passes through sleeves 530zzzj (e.g., through respective apertures 466 thereof) while the implant is in the compressed state. For example, and as shown in the upper left and lower left insets of FIG. 104A, apertures 466 can be larger (e.g., having an area at least one and a half times larger) than the cross-section of thin portion 364zzzj. The space between thin portion 364zzzj and apertures 466 reduces friction between the sleeves and the thin portion, facilitating articulation of wing 220 with respect to annular arms 360zzzj while the wing is in the compressed state. In some such implementations, and as shown in the lower left inset of FIG. 104A, aperture 466 may be shaped differently than the thin portion's cross-section. For example, and as shown, aperture 466 can have an oblong, (e.g., an oval) shape, whereas thin portion 364zzzf can have a circular profile, facilitating articulation of hinged coupling 460.

FIG. 104B shows wing 220zzzj having expanded into the expanded state upon or after being deployed from catheter 140.

In some implementations, wing 220zzzj (e.g., the wing's frame 224 and) comprises shape-memory material that biases the wing to expand into the expanded state upon release from catheter 140. In some implementations, shape-memory frame 224 applies sufficient expansion force to expand wing 220zzzj into the expanded state.

Alternatively or in addition, in some implementations, implant 200zzzj can comprise an expansion element that facilitates expansion (e.g., mechanical expansion, etc.) of the wing from the compressed state to the expanded state and/or resists compression of the wing toward the compressed state, e.g., similarly to expansion elements 270e, 270f, 270g of implants 200e, 200f, 200g described hereinabove with reference to FIGS. 8A-B, 9A-B and 10A-B, mutatis mutandis.

As shown in FIG. 104B, expansion of implant 200zzzj into the expanded state inhibits articulation of wing 220 with respect to annular arms 360zzzj by restraining hinged coupling 460. In some implementations, and as shown, expanding wing 220 restrains hinged coupling 460 by pushing sleeves 530zzzj (e.g., pushing the sleeves away from each other) from the arms' thin portion 364zzzj to a respective thick portion 466zzzj of the arms. In some such implementations, a tighter fit of thick portions 466zzz within apertures 466 restricts articulation of wing 220 with respect to atrial arms 360zzzj. For example, and as shown in the upper left inset of FIG. 104B, thick portions 366zzzj can have an oblong cross-section that fits the aperture's shape.

In some implementations, and as shown in the lower inset of FIG. 104B showing a cross-sectional view of hinged coupling 460 while wing 220zzzj is in the expanded state, apertures 466 on either side of sleeves 530zzzj are located and shaped to correspond to the arms' thick portions 366zzzj, e.g., by a friction fit between the apertures and the thick portions. In some such implementations, complementary shapes of apertures 466 and thick portions 366zzz are sufficient to restrain hinged coupling 460 and to keep wing 220zzzj from migrating longitudinally along arms 360zzzj, e.g., thereby keeping the wing centered between interfaces 350. Alternatively or in addition, thick portions 366zzzj can define widened stopper portions (not shown) that keep wing 220zzzj centered between interfaces 350.

In some implementations, annular arms 360zzzj are configured to extend along annulus 11 (e.g., to be seated at an atrial surface of the annulus) while interfaces 350 are secured to the annulus with anchors 30a, such that the restrained hinged coupling 460 inhibits articulation of wing 220zzzj with respect to arms 360zzzj and interfaces 350. In this way, arms 360zzzj can serve as an annular support 355zzzj that moderates deflection of wing 220zzzj, e.g., inhibits deflection of root portion 230 of wing 220 with respect to the annulus while tip portion 232 of the wing deflects during the cardiac cycle, similarly to annular support 355zk of implant 200zk (FIGS. 46A-B) described above. In some implementations, inhibiting upstream deflection of the wing's root portion by keeping root portion 230 of wing 220 in contact with the annulus inhibits upstream deflection of leaflet 12.

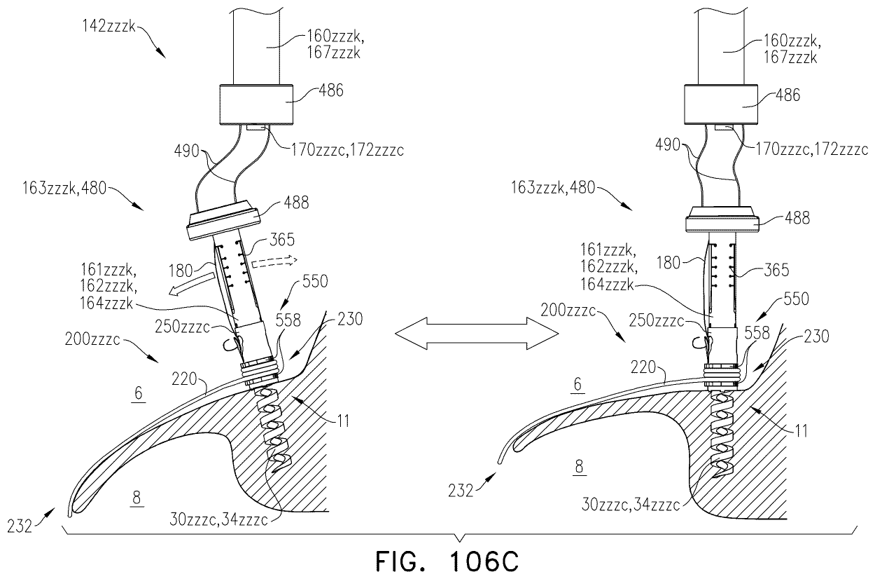

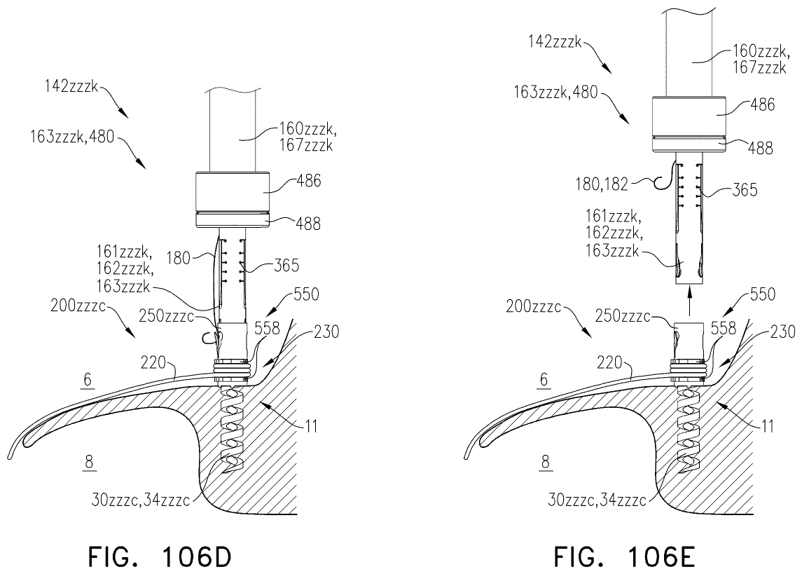

Reference is made to FIGS. 105A-E and 106A-E, which are schematic illustrations showing a distal portion 142zzzk of a system for delivering implant 200 to the heart, in accordance with some implementations of the invention. Distal portion 142zzzk can be similar, at least in its general purpose, e.g., anchoring implant 200 to a tissue of the heart so as to repair the function of the native leaflet, to distal portion 142 of system 100, or to any of the variants thereof disclosed herein. In some implementations, distal portion 142zzzk can be considered to be a variant of distal portion 142h, described hereinabove with reference to FIG. 11 as being used to screw a pair of anchors through respective anchor receivers 250h of implant 200h along nonparallel axes and into respective sites of the tissue.

In accordance with some implementations, FIGS. 105A, 105C, 105D show perspective and cross-sectional views of distal portion 142zzzk coupled to implant 200zzzc via a coupling 164zzzk at distal portion 163zzzk of shaft 160zzzk. As shown in FIGS. 106A-E, coupling 164zzzk is selectively secured to and released from the shaft's proximal portion 167zzzk.

In some implementations, and as shown, coupling 164zzzk comprises a lock 480 that reversibly locks the shaft's distal end portion 162zzzk to the shaft's proximal portion 167zzzk, e.g., such that lock 480 serves as a docking station at which distal end portion 162zzzk is reversibly coupled to proximal portion 167zzzk. In some implementations, unlocking lock 480 transitions coupling 164zzzk from a rigid state to a flexible state, as described hereinabove with reference to couplings 164zzv, 164zzw in FIGS. 85B and 87B.

In some implementations, the shaft's distal end portion 162zzzk bifurcates into branches 161zzzk comprising a shape-memory material that allows the branches to be compressed toward each other in order to fit within catheter 140 for delivery (e.g., arranging the branches along a proximal shaft axis a167, as shown in FIG. 105A), and that biases the branches to flex away from each other (FIG. 105D) upon release from the catheter, in order to facilitate screwing a pair of anchors 30zzzc into tissue along nonparallel axes 301, 302 (FIG. 105D), e.g., as described hereinabove with reference to FIG. 11. In some such implementations, and as shown, branches 161zzzk can comprise notches 325 that can open as the branches flex away from each other, e.g., orienting drivers 170 to drive anchors 30zzzc along nonparallel axes 301, 302 that are oblique to proximal shaft axis a167 (FIG. 105D).

In some implementations, and as shown, lock 480 comprises a proximal unit 486 fixedly coupled to the shaft's proximal portion 167zzzk, and a distal unit 488 fixedly coupled to coupling 164zzzk. In some such implementations, and as shown in FIG. 105D, distal unit 488 is coupled to shaft 160zzzk at the shaft's bifurcation into branches 161zzzk, which together define coupling 164zzzk. For example, proximal rings 326 of each branch 161zzzk fit into slots 485 defined by the lock's distal unit 488 (FIGS. 105B, 105D).

FIGS. 105A-D show lock 480 in a locked state, in which the lock's proximal unit 486 is mated to distal unit 488, thereby connecting the shaft's proximal portion 167zzzk to coupling 164zzzk. In some such implementations, and as shown in the cross-sectional view of the locked lock 480 in FIG. 105B, proximal unit 486 and distal unit 488 are complementarily shaped in a manner that inhibits rotation of the proximal and distal units with respect to each other.

The inset of FIG. 105A shows lock 480 in an unlocked state in which distal unit 488 is separated from proximal unit 486 (e.g., by intracardially reducing tension upon a tether 490). In some implementations, and as shown, tether 490 maintains connection between distal unit 484 and proximal unit 486 while the lock is unlocked. In some such implementations, and as shown in an exploded view of distal portion 142zzzk, tether is disposed within a tether lumen 492 defined by the lock's units 486, 484 (FIG. 105E). For example, loose attachment of the lock's proximal and distal units 486, 484 via tether 490 can allow for translation of distal unit 484 away from proximal unit 486 by releasing tension from tether 490, and for remating the distal and proximal units by tensioning the tether.

FIGS. 106A-E show use of distal portion 142zzzk to secure implant 200zzzc by advancing anchors 30zzzc through respective interfaces 250zzzc, similarly to as described hereinabove with reference to FIGS. 95, 96A-E, 97A-C. However, distal portion 142zzzk can be used to deliver and secure implant 200 or any of the variants thereof disclosed herein, mutatis mutandis. In some implementations, and as shown in FIG. 106A, implant 200zzzc is advanced to the valve's annulus 11 while lock 480 is locked. In some such implementations, and as shown, the wing's root portion 230 is positioned, via engagement of coupling 164zzzk with anchor receiver 250zzzc, in the position described hereinabove with reference to FIG. 3B.

As shown in FIG. 106B, while implant 200zzzk is held in position, driver 170zzzc is used secure implant 200zzzc in place by driving anchor 30zzzc into tissue of annulus 11, after which the implant's function is assessed (FIG. 106C). In accordance with some implementations, FIG. 106C shows lock 480 having been unlocked, (e.g., by intracardially reducing tension upon tether 490), such that distal unit 484 is loosely connected to proximal unit 486 via tether 490. In some such implementations, and as shown, coupling 164zzzk remains engaged to interface 250zzzc while lock 480 is unlocked.

In some implementations, and as shown, the loose connection between the proximal and distal units of lock 480 allow the distal unit to translate with respect to the proximal unit, thereby facilitating deflection of implant 200zzzc (e.g., tip portion 232 of wing 220) in response to the heart's cardiac cycle. In some such implementations, and as shown, distal unit 484 translates with respect to proximal unit 486 while coupling 164zzzk remains engaged to interface 550, such that the proximal unit and coupling 164zzzk deflect reciprocatingly together with implant 200zzzc in response to the cardiac cycle.

In some implementations, function of the valve is assessed (e.g., by monitoring and evaluating bloodflow therethrough) while the implant 200zzzc is secured to annulus 11 and lock 480 is unlocked. In some implementations, drive head 172zzzc is retracted proximally, together with the lock's proximal unit 486 while the lock is in the unlocked state (FIG. 106C), which can facilitate deflection of implant 200zzzc in response to the cardiac cycle while the valve's function is assessed.

In some implementations, drive head 172zzzc can remain engaged to anchors 30zzzc (not shown) during assessment of the valve's function.

In some implementations, if function of the valve and/or the implant is considered to be suitable, coupling 164zzzk can be disengaged from interface 550 (FIG. 106E), e.g., by retracting ripcord 180, as described hereinabove with reference to FIGS. 3D-E. In some such implementations, and as shown in FIG. 106D, lock 480 is relocked (e.g., by tensioning tether 490) prior to disengaging coupling 164zzzk from interface 550, after which distal portion 142zzzk is withdrawn from the heart (FIG. 106E).

In some implementations, coupling 164zzzk can be disengaged from interface 550 while lock 480 remains unlocked (not shown).

In some implementations, if the valve's function is found to be suboptimal, anchors 30zzzc can be retracted from annulus 11 and implant 200zzzc can either be repositioned or removed (not shown). In some implementations in which the implant is repositioned, function of the valve and/or the implant is reassessed after repositioning (e.g., by unlocking lock 480 after re-anchoring the implant), so as to allow implant 200zzzk and coupling 164zzzk to deflect reciprocatingly in response to the heart's cardiac cycle at the new position.

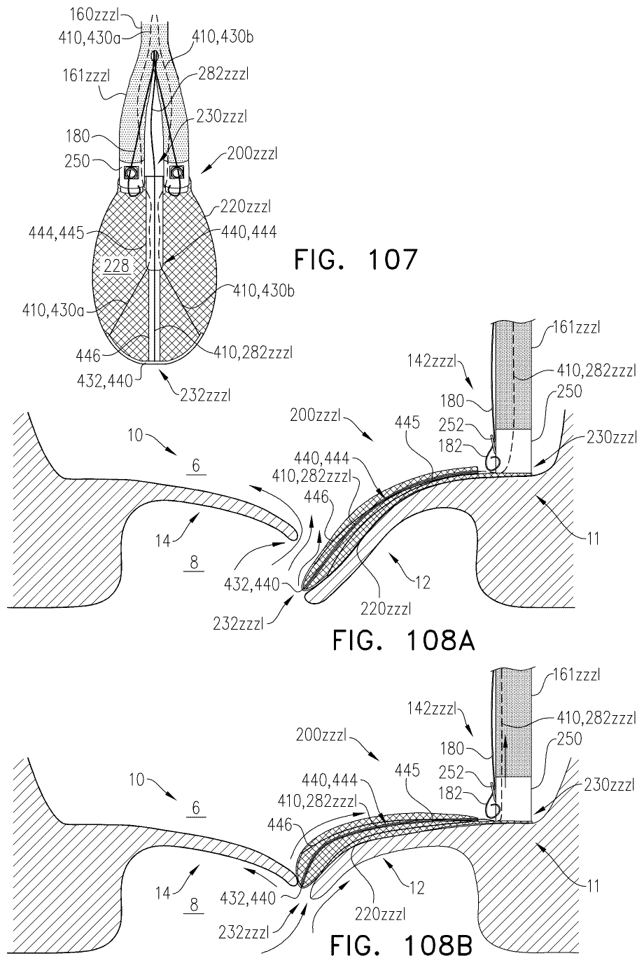

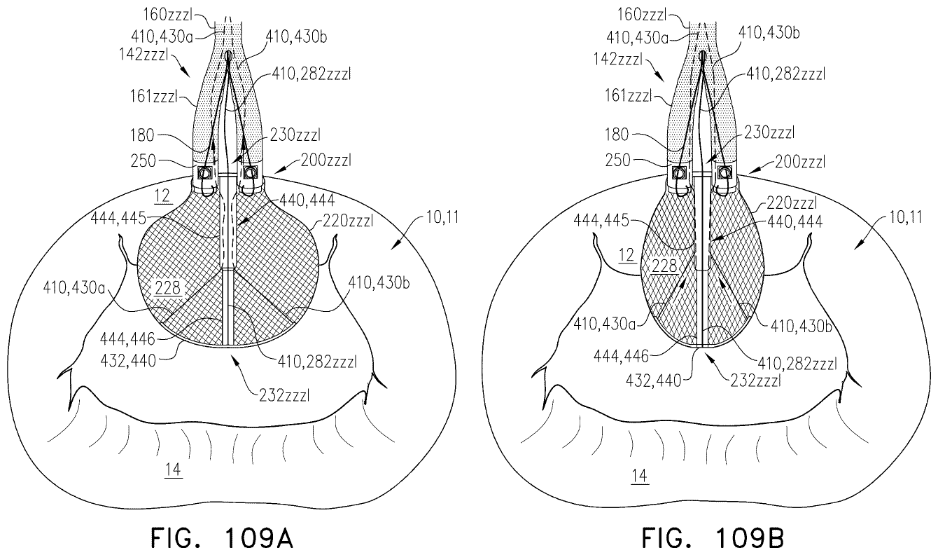

Reference is made to FIGS. 107, 108A-B and 109A-B, which are schematic illustrations showing use of an implant 200zzzl, in accordance with some implementations of the invention. Implant 200zzzl can be considered to be a variant of implant 200, and can be similar, at least in its general purpose, e.g., being anchored to a tissue of the heart so as to repair the function of the native leaflet, to implant 200 or any of the variants thereof disclosed herein, mutatis mutandis.

As described hereinbelow, in some implementations, implant 200zzzl comprises a beam 440 that is connected to the implant's wing 220zzzl, and a line 410 that is tensioned (e.g., intracardially tensioning a proximal portion of the line at controller 120 transfers tension to a distal portion of the line coupled to beam 440), thereby straining the beam and reshaping the wing. In some implementations, line 410 is independent of driver 170, such that the line can be tensioned to reshape wing 220zzzl before and/or after securing the wing to annulus 11.

As shown, beam 440 comprises a rod 444 and/or a plate 432 that is connected to the implant's wing 220zzzl, and line 410 comprises a tether 282zzzl that is connected to the rod, and/or cords 430a, 430b that are connected to the plate. FIGS. 107, 108A-B and 109A-B show implant 200zzzl comprising both rod 444 and tether 282zzzl, as well as plate 432 and cords 430a, 430b. In some implementations, rod 444 and plate 432 complement each other by facilitating reshaping of wing 220zzzl along more than one dimension. Alternatively, the implant may comprise either rod 444 and tether 282zzzl, or plate 432 and cords 430a, 430b.

In some implementations, rod 444 has a rigid portion 445 and a flexible portion 446, as shown in FIG. 107. In some such implementations, and as shown, rod 444 extends along a length of wing 220zzzl such that the rod's rigid portion 445 is disposed at the wing's root portion 230zzzl and the rod's flexible portion 446 is disposed at (e.g., connected to) the wing's tip portion 232zzzl.

In some implementations, straining rod 444 by tensioning tether 282zzzl can deflect the rod's flexible portion 446, and therefore the wing's tip portion 232zzzl as well. In some such implementations, tether 282zzzl is coupled to the rod's flexible portion 446, e.g., at the wing's tip portion 232zzzl. In this way, straining rod 444 by tensioning tether 282zzzl deflects the rod's flexible portion 446 to a greater degree than the rod's rigid portion 445, thereby causing the wing's tip portion 232zzzl to change shape to a greater degree than the wing's root portion 230zzzl. For example, straining rod 444 by tensioning tether 282zzzl can pull tip portion 232zzzl along the wing's normal plane. Although FIGS. 108A-B show tether 282zzzl being used to pull the wing's tip portion 232zzzl in the upstream direction, the tether may alternatively be configured to pull the tip portion in the downstream direction, mutatis mutandis.

FIG. 108A-B show use of tether 282zzzl to intracardially reshape wing 220zzzl while the wing's root portion 230zzzl is positioned at annulus 11, such that the wing extends along leaflet 12 to the wing's tip portion 232zzzl. FIGS. 108A-B show valve 10 during ventricular systole, and the arrows between the valve's leaflets 12, 14 represent retrograde bloodflow through a gap between the leaflets. In some implementations, and as shown in FIG. 108B, tensioning tether 282zzzl strains rod 444 (e.g., flexible portion 446 thereof), thereby reshaping wing 220zzzl so as to reduce retrograde bloodflow by closing the gap between leaflets 12, 14. In some such implementations, tensioning tether 282zzzl changes (e.g., increases, as shown) the wing's radius of curvature along a normal plane of the wing perpendicular to the wing's length.

Alternatively or in addition to rod 444, beam 440 comprises a rigid plate 432 that is connected to wing 220zzzl along the wing's tip portion 232zzzl. In some implementations, and as shown in FIGS. 107 and 109A-B, line 410 comprises cords 430a, 430b that are coupled to wing 220zzzl at plate 432, e.g., along a perimeter of tip portion 232zzzl. In some such implementations, and as shown, cords 430a, 430b extend distally from the delivery tool's proximal portion, through shaft 160zzzl and respective interfaces 250, to plate 432. In this way, tension applied to the proximal portion of cords 430a, 430b strains plate 432, thereby reshaping the wing's tip portion 232zzzl. As such, plate 432 can serve as an intermediary between cords 430a, 430b and tip portion 232zzzl, that both supports the tip portion and transfers tension to the tip portion. In some such implementations, rod 444 also serves as an intermediary between cords 430a, 430b and tip portion 232zzzl. For example, and as shown, cords 430a, 430b can pass through rod 444, e.g., rigid portion 445 thereof, which can modify a vector of force that the cords apply to plate 432 when the cords are tensioned.

FIGS. 109A-B show implant 200zzzl positioned such that root portion 230zzzl of wing 220zzzl is placed at annulus 11 (adjacent posterior leaflet 12), and the wing extends from the wing's root portion to the wing's tip portion 232zzzl, toward anterior leaflet 14. In some implementations, and as shown, cords 430a, 430b are attached to respective portions of plate 432. FIG. 109A shows cords 430a, 430b in a relaxed state, which allow wing 220zzzl to retain its native conformation.

In some implementations, tensioning cords 430a, 430b transfers tension via the cords to either end of plate 432, which decreases the plate's radius of curvature, thereby reducing the wing's width. In some such implementations, and as shown, wing 220zzzl comprises a mesh, e.g., a braided mesh 228 described hereinabove with reference to FIG. 19, mutatis mutandis. In some implementations in which wing 220zzzl comprises a braided mesh 228, tensioning cords 430a, 430b reshapes wing 220zzzl by reorienting the braided mesh's weave.

In some implementations, and as shown in FIG. 109B, wing 220zzzl is reshaped symmetrically by applying an equal amount of tension to each cord 430a, 430b.

In some implementations, it may be desirable to reshape wing 220zzzl into an asymmetric shape (not shown) and this can also or alternatively be done, e.g., by applying unequally tensioning cords 430a, 430b, or by tensioning only one of the cords.

Although FIGS. 108A-B and 109A-B show reshaping wing 220zzzl by tensioning either tether 228zzzl (FIGS. 108A-B) or cords 430a, 430b (FIGS. 109A-B), it may be desirable to change both the wing's radius of curvature along the wing's normal plane, and to change the wing's length or width, by tensioning both tether 228zzzl and cords 430a, 430b, e.g., simultaneously.

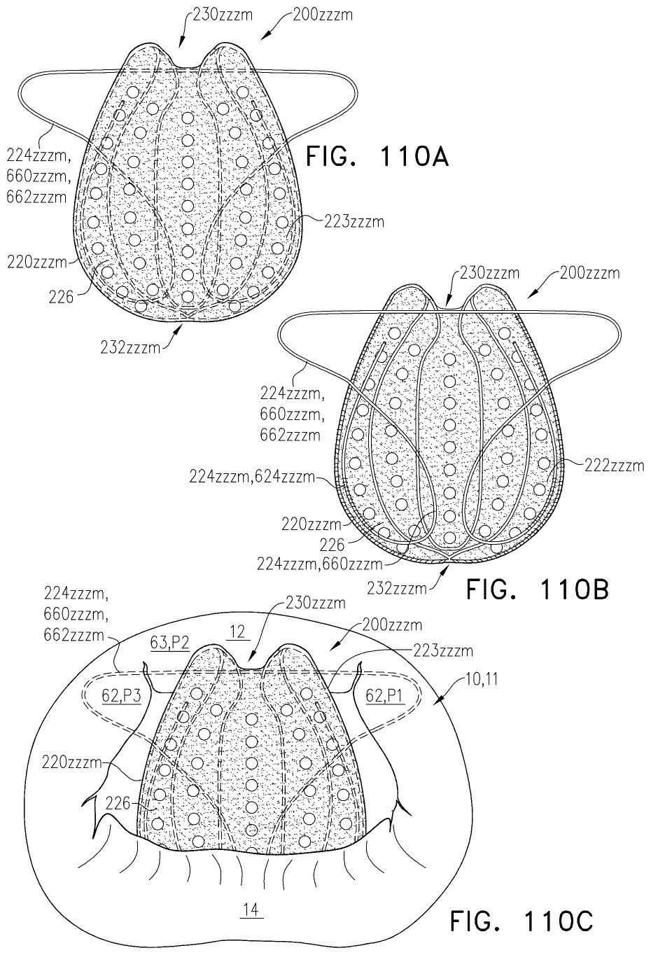

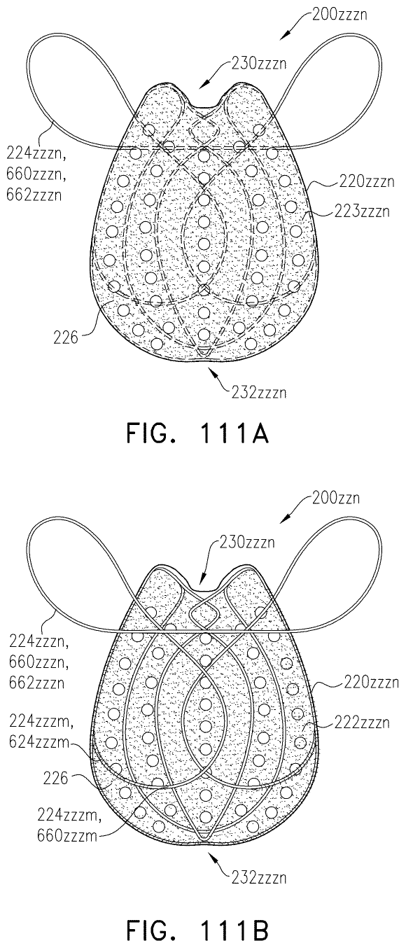

Reference is made to FIGS. 110A-C and 111A-B, which are schematic illustrations showing implants 200zzzm, 200zzzn, in accordance with some implementations of the invention. Implants 200zzzm, 200zzzn can be considered to be variants of implant 200, and can be similar, at least in their general purpose, e.g., being anchored to a tissue of the heart so as to repair the function of the native leaflet, to implant 200 or any of the variants thereof disclosed herein, mutatis mutandis, except that implants 200zzzm, 200zzzn each comprise a pair ventricular arms 660zzzm, 660zzzn that extend laterally from wing 220zzzm, 220zzzn. FIGS. 110B, 111B each show a respective contact face 222zzzm, 222zzzn of wing 220zzzm, 220zzzn, and FIGS. 110A, 111A show the respective wing's opposing face 223zzzm, 223zzzn.

In some implementations, and as shown in FIGS. 110B, 111B, arms 660zzzm, 660zzzn extend laterally away from the wing's tip portion 232zzzm, 232zzzn to define a lateral portion 662zzm, 662zzzn of each arm. In this way, arms 660zzzm, 660zzzn of each pair extend away from each other, to opposing lateral sides of implant 200zzzm, 200zzzn. In some such implementations, arms 660zzzm, 660zzzn are fastened (e.g., via sutures) to the wing's tip portion 232zzzm, 232zzzn. For example, and as shown in FIGS. 110B, 111B, the wing's frame 224zzzm, 224zzzn defines a support portion 664zzzm, 664zzzn that is fastened to wing 220zzzm, 220zzzn, and from which the frame extends to define arms 660zzzm, 660zzzn.

In some implementations, implant 200zzzm, 200zzzn is implanted at valve 10, e.g., using shaft 160 of delivery tool 150 as described hereinabove. In some such implementations, root portion 230zzzm, 230zzzn is placed along the valve's posterior leaflet 12 and/or annulus 11 and wing 220zzzm, 220zzzn extends from the root portion to tip portion 232zzzm, 232zzzn, toward anterior leaflet 14. For example, the implant (e.g., implant 200zzzm shown in FIG. 110C, and/or implant 200zzzn shown in FIGS. 111A-B) may not require anchors 30 or interfaces 250 to be secured in place. That is, opposing forces that the wing's root portion applies to the atrial side of valve 10, and that arms 660zzzm, 660zzzn apply to the valve's ventricular side (e.g., pinching the valve) can be sufficient to secure implant 200zzzm, 200zzzn in place. Alternatively, implant 200zzzm, 200zzzn can include interfaces 250 fastened to annulus 11 by driving anchors 30 therethrough, as described hereinabove.

FIG. 110C shows implant 200zzzm implanted at mitral valve 10, such that wing 220zzzm deflects as the heart transitions between systole and diastole. In view of similarity between implants 200zzzm, 200zzzn, FIG. 110C may be considered representative of certain aspects of both implants, e.g., the interaction between implants 200zzzm, 200zzzn and posterior leaflet 12 while the implant is implanted at valve 10. In some implementations, and as shown, implant 200zzzm, 200zzzn is implanted such that the arms' lateral portions 662zzzm, 662zzzn each press in an upstream direction against a respective lateral site 62 on a downstream side of leaflet 12. In some such implementations, the pressing force applied by arms 660zzzm, 660zzzn against lateral sites 62 (e.g., the posterior leaflet's p1 and p3 scallops) in turn presses contact face 222zzzm, 222zzzn against an upstream side of leaflet 12, at a medial site 63 (e.g., the posterior leaflet's p2 scallop) between the lateral sites, thereby pinching the leaflet between wing 220zzzm, 220zzzn and the arms' lateral portion 662zzzm, 662zzzn. In some such implementations, pressing contact face 222zzzm, 222zzzn against the upstream side at medial site 63 of leaflet 12 inhibits deflection of the root portion of wing 220zzzm, 220zzzn in the upstream direction, e.g., thereby defining the wing's deflection-limit, as described hereinabove.

In some implementations, when implant 200zzzm, 200zzzn is implanted as described hereinabove, the wing's tip portion 232zzzm, 232zzzn deflects in concert with the arms' lateral portions 662zzzm, 662zzzn and with tissue of lateral sites 62, responsively to the cardiac cycle. In some such implementations, tip portion 232zzzm, 232zzzn deflects at least to some degree independently of medial site 63, which be desirable in some cases in which the leaflet's p2 scallop does not function adequately, e.g., due to disease or injury. Implants 200zzzm, 200zzzn may therefore be particularly suitable for implantation at a heart valve having a diseased p2 scallop, alongside functional scallops p1 and p3.

As described hereinabove, implant 200zzzn is in many ways similar to implant 200zzzm. In some implementations, and as shown, lateral portions 662zzzn of the implant's arms 660zzzn are shaped to reach further away from the wing's tip portion 232zzzn than do lateral portions 662zzzm of implant 200zzzm. In some such implementations, the shape of arms 6660zzzn may improve fit of implant 200zzzn to valves 10 of certain geometry.

Various implementations of systems, devices, methods, etc. are disclosed herein, and any combination of their features, components, and options can be made unless specifically excluded. For example, various descriptions of the implants disclosed herein, can be used with any appropriate delivery tool, and/or implanted by any appropriate method, even if a specific combination is not explicitly described. Likewise, the different constructions and features of devices and systems can be mixed and matched, such as by combining any frame and limiter disclosed herein, even if not explicitly disclosed. In short, individual components of the disclosed systems can be combined unless mutually exclusive or physically impossible.

Furthermore, features of implants and/or delivery tools described herein can be combined and/or substituted with any of those disclosed in the following applications, each of which is incorporated herein by reference in its entirety for all purposes: International Patent Application No. PCT/US2021/039587 to Chau et al., filed Jun. 29, 2021, and titled “Systems and methods for heart valve leaflet repair”, which published as WO 2022/006087

International Patent Application No. PCT/US2022/052834 to Chau et al., filed Dec. 14, 2022, and titled “Systems and techniques for heart valve leaflet repair.”

For example, ring 252 of interface 250 for use with a ripcord 180; lateral flaps 260 of sheets 226a, 226b; ratcheting interface 250j; mesh 228 or frames 224k-224n that are stiffer at the root portion of the wing than at the tip portion of the wing; flex elements 280q-280z coupling the tip portion to the root portion; limiters 284y-284zi; and/or self-limiting wing 220zj can be applied to implant 200 and its variants, mutatis mutandis. Similarly, delivery tools described herein can be used, mutatis mutandis, with any of the implants disclosed in the above applications.

As another example, any of the implants described or shown herein can include a leg or extension that extends from the tip of the wing to an end portion of the leg, which can be the same as or similar to legs/extensions shown or described in International Patent Application No. PCT/US2022/052834 to Chau et al., titled “Systems and techniques for heart valve leaflet repair”, filed on Dec. 14, 2022 herewith.

In some implementations, when the implant is implanted, the leg or extension extends from the wing of the implant such that, upon implantation, the leg or extension protrudes into the chamber downstream of the valve being treated.

In some implementations, the leg or extension can be configured to bias the wing of the implant toward a particular position and/or orientation, and/or can be configured to inhibit the wing from prolapsing into the atrium upstream of the valve being treated.

In some implementations, the leg is configured to maintain contact between the wing and leaflet as the leaflet oscillates throughout multiple cardiac cycles.

Any of the various systems, assemblies, devices, components, apparatuses, etc. in this disclosure (including those in the examples below) can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.). Furthermore, the scope of the present disclosure includes, in some implementations, sterilizing one or more of any of the various systems, devices, apparatuses, etc. in this disclosure.

The techniques, methods, operations, steps, etc. described or suggested herein (including those in the examples below) or in the references incorporated herein can be performed on a living subject (e.g., human, other animal, etc.) or on a simulation, such as a cadaver, cadaver heart, simulator, imaginary person, etc. When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

Example Implementations (some non-limiting examples of the concepts herein are recited below):

Example 1. A system for use with a real or simulated valve of a real or simulated heart of a real or simulated subject, the valve having a first leaflet and an opposing leaflet, the heart having a chamber upstream of the valve, the system comprising: (i) an implant comprising: (a) a wing: having a contact face, and an opposing face opposite to the contact face, defining a tip portion, a root portion, and optionally a flex element that couples the tip portion to the root portion; and/or (b) an interface at the root portion; an anchor; and/or (ii) a delivery tool, comprising: (a) a catheter, transluminally advanceable to the chamber; and/or (b) a shaft disposed within the catheter, the shaft engaged with the interface.

Example 2. The system according to example 1, wherein: the anchor comprises: an anchor head, and a tissue-engaging element that extends from the anchor head; and/or an outer diameter of the anchor head is greater than an outer diameter of the tissue-engaging element.

Example 3. The system according to any one of examples 1-2, wherein the shaft is configured, via the engagement with the interface, to (i) deploy the implant out of the catheter, and (ii) position the implant in a position in which the interface is at a site upstream of the valve, the wing extends over the first leaflet toward the opposing leaflet, and the contact face faces the first leaflet.

Example 4. The system according to any one of examples 1-3, wherein the delivery tool further comprises a driver, engaged with the anchor and configured to secure the implant by using the anchor to anchor the interface to tissue of the heart.

Example 5. The system according to any one of examples 1-4, wherein at least one of the implant, the anchor, and the delivery tool is sterile.

Example 6. The system according to any one of examples 1-5, wherein the flex element protrudes from the contact face of the wing.

Example 7. The system according to any one of examples 1-5, wherein the flex element protrudes from the opposing face of the wing.

Example 8. The system according to any one of examples 1-7, wherein the flex element is a flexure.

Example 9. The system according to any one of examples 1-7, wherein the flex element is a hinge.

Example 10. The system according to any one of examples 1-7, wherein the flex element is a living hinge.

Example 11. The system according to any one of examples 1-5, wherein the flex element comprises a pair of interlocking loops, a first one of the loops defined by the root portion, and a second one of the loops defined by the tip portion.

Example 12. The system according to any one of examples 1-5, wherein the flex element comprises a plurality of coiled wires connecting the tip portion to the root portion.

Example 13. The system according to any one of examples 1-5, wherein the flex element comprises a plurality of rings connecting the tip portion to the root portion.

Example 14. The system according to any one of examples 1-5, wherein the flex element comprises a plurality of sutures connecting the tip portion to the root portion.

Example 15. The system according to any one of examples 1-5, wherein the flex element comprises a tube through which respective portions of the tip portion and the root portion extend alongside each other, such that the tip portion and root portion can articulate in relation to each other.

Example 16. The system according to any one of examples 1-15, wherein the root portion is stiffer than the tip portion.

Example 17. The system according to any one of examples 3-16, wherein the implant is configured such that, while the implant is secured in the position, flexing of the flex element facilitates deflection of the tip portion with respect to the root portion in response to a cardiac cycle of the heart.

Example 18. The system according to example 17, wherein the flex element is protrusive, and the implant is configured such that, while the implant is secured in the position, the flex element abuts a hinge-point between a leaflet of the valve and an annulus of the valve.

Example 19. The system according to any one of examples 3-18, wherein the flex element is protrusive so as to abut a hinge-point between a leaflet of the valve and an annulus of the valve and is positioned within the implant such that abutment of the flex element against the hinge-point positions the interface at the site.

Example 20. The system according to any one of examples 3-19, wherein the flex element is protrusive, and the shaft is configured to position the interface at the site by abutting the flex element against a hinge-point between a leaflet of the valve and an annulus of the valve.

Example 21. The system according to any one of examples 1-20, wherein: the wing comprises a frame, and a flexible sheet disposed over the frame, and the frame defines the flex element.

Example 22. The system according to example 21, wherein the flex element is a torsion spring.

Example 23. The system according to example 21, wherein the flex element is a hinge.

Example 24. The system according to example 23, wherein the flex element is a ball-and-socket hinge.

Example 25. A system for use with a real or simulated valve of a real or simulated heart of a real or simulated subject, the valve having a first leaflet and an opposing leaflet, the heart having a chamber upstream of the valve, the system comprising: (i) an anchor; an implant comprising: a first wing: extending from a first root portion of the first wing to a first tip portion of the first wing, and defining a contact face, and an opposing face opposite to the contact face, a second wing: extending, over the opposing face of the first wing, from a second root portion of the second wing to a second tip portion of the second wing, the first wing being deflectable toward and away from the second wing, and an interface coupled to the first root portion and to the second root portion; and/or (ii) a delivery tool, comprising: a catheter, transluminally advanceable to the chamber; a shaft disposed within the catheter, the shaft engaged to the interface.

Example 26. The system according to example 25, wherein the shaft is configured, via the engagement with the interface, to: deploy the implant out of the catheter, and position the implant in a position in which: the interface is at a site upstream of the valve, the first wing extends over the first leaflet toward the opposing leaflet, the contact face faces the first leaflet, and the second wing extends over the opposing face of the first wing.

Example 27. The system according to any one of examples 25-26, further comprising a driver, the driver engaged with the anchor and configured to secure the implant by using the anchor to anchor the interface to tissue of the heart.

Example 28. The system according to any one of examples 25-27, wherein at least one of the implant, the anchor, and the delivery tool is sterile.

Example 29. The system according to any one of examples 26-28, wherein the implant is configured such that, while the implant remains secured in the position, during ventricular diastole the first wing deflects farther into the chamber than does the second wing.

Example 30. The system according to example 29, wherein the implant further comprises a third wing, the third wing having a third root portion that is coupled to the interface, and extending, over the second wing, from the third root portion to a third tip portion of the third wing, the second wing being deflectable toward and away from the third wing.

Example 31. The system according to example 30, wherein the third wing is shorter than the second wing.

Example 32. The system according to any one of examples 30-31, wherein the second wing is more flexible than the third wing.

Example 33. The system according to example 32, wherein the first wing is more flexible than the second wing.

Example 34. The system according to example 32, wherein the implant is configured such that, while the implant remains secured in the position, during ventricular diastole the second wing deflects away from the third wing.

Example 35. The system according to example 34, wherein the implant is configured such that, while the implant remains secured in the position, during ventricular diastole the first wing deflects away from the second wing.

Example 36. The system according to example 35, wherein the implant is configured such that, while the implant remains secured in the position, during ventricular systole the first wing deflects into contact with the second wing.

Example 37. The system according to example 36, wherein the implant is configured such that, while the implant remains secured in the position, during ventricular systole the second wing deflects into contact with the third wing.

Example 38. The system according to any one of examples 29-37, wherein the first wing defines multiple holes therethrough.

Example 39. The system according to example 38, wherein the implant is configured such that, while the implant is secured in the position, during ventricular systole, the first wing deflects into contact with the second wing in a manner that obstructs bloodflow through the holes.

Example 40. The system according to example 39, wherein the second wing defines multiple holes therethrough, the holes of the first wing being positioned such that, while the first wing is in contact with the second wing, the holes of the first wing are offset with respect to the holes of the second wing.

Example 41. The system according to any one of examples 34-40, wherein the second wing is stiffer than the first wing.

Example 42. The system according to example 41, wherein the second wing is shorter than the first wing.

Example 43. The system according to example 26, wherein the implant further comprises a flexible pouch, the first and second wings being disposed within the pouch.

Example 44. The system according to example 43, wherein the pouch is configured to expand during ventricular diastole, and to contract during ventricular systole.

Example 45. The system according to example 43, wherein the pouch is coupled to the interface.

Example 46. The system according to example 43, wherein: on a first side of the pouch, the pouch defines multiple first-pouch-side holes that provide fluid communication between inside and outside of the pouch, and on a second side of the pouch, opposite the first side, the pouch defines multiple second-pouch-side holes that provide fluid communication between inside and outside of the pouch.

Example 47. The system according to example 46, wherein the pouch is configured such that, while the implant is secured in the position, when the first wing deflects toward the second wing during ventricular systole the first side of the pouch moves toward the second side of the pouch in a manner that inhibits bloodflow through the first-pouch-side holes and the second-pouch-side holes.

Example 48. The system according to example 43, wherein the first wing and the second wing are each stiffer than the pouch.

Example 49. The system according to example 46, wherein the first-pouch-side holes and the second-pouch-side holes are positioned such that, while the implant is secured in the position and the first wing deflects toward the second wing, the first-pouch-side holes are offset with respect to the second-pouch-side holes.

Example 50. An apparatus for use with a real or simulated valve of a real or simulated heart of a real or simulated subject, the valve having a first leaflet and an opposing leaflet, the heart having a first chamber upstream of the valve and a second chamber downstream of the valve, the apparatus comprising an implant, the implant comprising: (i) a wing, extending from a root portion of the wing to a tip portion of the wing; (ii) an interface, at the root portion, and configured to be anchored to a site in the first chamber such that the implant is secured in a position in which: the wing extends over the first leaflet toward the opposing leaflet, and responsively to a cardiac cycle of the heart, the wing deflects, in a reciprocating manner, in an upstream direction and in a downstream direction; and/or (iii) a limiter, configured to define a deflection-limit of the wing, and to inhibit deflection of the wing in the upstream direction beyond the deflection-limit by providing an opposing force upon the wing reaching the deflection-limit.

Example 51. The apparatus according to example 50, wherein: the limiter is intracardially adjustable in a manner that adjusts the deflection-limit of the wing, and the apparatus further comprises an anchor, the anchor configured to be driven into tissue of the first chamber, and wherein the deflection-limit is adjustable by adjustment of a depth of anchoring of the anchor within the tissue.

Example 52. The apparatus according to example 51, wherein the limiter is coupled to the interface, and the interface is configured to be anchored to the site by the anchor.

Example 53. The apparatus according to example 52, wherein the interface is configured to be anchored to the site by advancing the anchor through the interface and into tissue at the site, such that: while the implant is secured in the position, the limiter extends away from the interface and over the wing, and upon the wing reaching the deflection-limit, the wing contacts the limiter.

Example 54. The apparatus according to example 53, wherein the implant is configured such that the adjustment of the limiter by adjustment of the depth of anchoring comprises adjustment of an angle of the limiter with respect to the interface.

Example 55. The apparatus according to example 53, wherein a portion of the limiter is disposed between the interface and the wing, and the implant is configured such that the further advancement of the anchor sandwiches the portion of the limiter between the interface and the wing.

Example 56. The apparatus according to example 50, wherein the implant is sterile.

Example 57. The apparatus according to any one of examples 50-56, wherein the implant is configured such that, upon the wing reaching the deflection-limit, the wing contacts the limiter.

Example 58. The apparatus according to any one of examples 50-57, wherein the limiter comprises a tether, the tether being configured to become tensioned as the wing reaches the deflection-limit.

Example 59. The apparatus according to example 58, wherein the tether is configured to restrain the tip portion of the wing as the wing reaches the deflection-limit.

Example 60. The apparatus according to example 58, wherein the tether is connected to the tip portion of the wing.

Example 61. The apparatus according to example 58, wherein the tether is coupled to a portion of the wing such that shortening of the tether adjusts the deflection-limit.

Example 62. The apparatus according to example 61, further comprising a rotatable spool, wherein: a portion of the tether is wound around the spool, and the tether is configured to be shortenable by rotating the spool.

Example 63. The apparatus according to example 61, wherein the implant comprises a real or simulated tissue anchor, coupled to the tether, and configured to anchor the tether to tissue of the second chamber.

Example 64. The apparatus according to example 63, wherein: the tether defines a rail portion to which a proximal portion of the tether is slidably coupled; the tissue anchor is a first atraumatic tissue anchor, coupled to a first part of the rail portion, and configured to anchor the first part of the rail portion to trabeculae at a first site of the second chamber; and/or the implant further comprises a second atraumatic tissue anchor, coupled to a second part of the rail portion, and configured to anchor the second part of the rail portion to trabeculae at a second site of the second chamber.

Example 65. The apparatus according to example 61, wherein the tether is configured to be shortened by being slid with respect to the root portion of the wing.

Example 66. The apparatus according to example 65, wherein the tether is configured to be shortened by being slid through the interface.

Example 67. The apparatus according to any one of examples 50-58, wherein the limiter is coupled to the interface, such that the interface is deflectable toward and away from the limiter.

Example 68. The apparatus according to example 67, wherein the implant is configured such that, upon the wing reaching the deflection-limit, the interface contacts the limiter.

Example 69. The apparatus according to any one of examples 50-68, wherein the limiter is coupled to the interface, and extends, away from the interface and over the wing, such that the wing is deflectable toward and away from the limiter.

Example 70. The apparatus according to example 69, wherein the limiter is stiffer than the wing.

Example 71. The apparatus according to example 69, wherein the deflection-limit is defined by a relative position between the limiter and the wing.

Example 72. The apparatus according to example 69, wherein the limiter extends away from the interface and over a face of the wing, the wing is deflectable toward the limiter such that the wing contacts the limiter upon reaching the deflection-limit.

Example 73. The apparatus according to example 72, wherein the limiter is shaped such that the wing contacts the limiter at a contact-portion of the wing that is between the root portion of the wing and the tip portion of the wing.

Example 74. The apparatus according to example 72, wherein the limiter is shaped to define a cross-brace that, upon the wing reaching the deflection-limit, lies in contact with the wing, widthways across the wing.

Example 75. The apparatus according to example 69, wherein the wing is a first wing, and the limiter comprises a second wing.

Example 76. The apparatus according to example 75, wherein the second wing is shorter than the first wing.

Example 77. The apparatus according to example 75, wherein the second wing is narrower than the first wing.

Example 78. The apparatus according to example 69, wherein the limiter has a backstop portion that is shaped to press against tissue of the first chamber upon anchoring of the interface to the site.

Example 79. The apparatus according to example 78, wherein the interface serves as a fulcrum between the backstop portion and a part of the limiter that extends away from the interface and over the wing, such that the pressing the backstop portion against tissue of the first chamber adjusts the deflection-limit of the wing.

Example 80. The apparatus according to example 78, wherein the backstop portion is intracardially adjustable in a manner that adjusts pressing of the backstop portion against the tissue.

Example 81. The apparatus according to example 80, wherein the backstop portion is adjustable by inflation of the backstop portion.

Example 82. The apparatus according to example 81, further comprising a delivery tool, comprising a nozzle, and configured to inflate the backstop portion via the nozzle.

Example 83. The apparatus according to example 82, wherein the delivery tool: comprises a water-absorbing hydrogel that expands upon absorption of water, and is configured to inflate the backstop portion with the hydrogel.

Example 84. The apparatus according to example 78, wherein: the apparatus further comprises an anchor, and the backstop portion defines an anchor receiver that is configured to receive the anchor in a manner that anchors the anchor receiver to tissue of the first chamber.

Example 85. The apparatus according to example 78, wherein the backstop portion is wider than the wing.

Example 86. The apparatus according to example 78, wherein the backstop portion extends from the interface away from the wing.

Example 87. The apparatus according to example 78, wherein the limiter is shaped to define a cross-brace, along a width of the limiter, that is configured to press against tissue of the first chamber upon anchoring of the interface to the site.

Example 88. The apparatus according to example 69, wherein the limiter comprises a frame that comprises: a first portion that comprises or is formed from sheet metal, and a second portion, coupled to the first portion, that comprises or is formed from wire.

Example 89. The apparatus according to example 88, wherein the first portion is shaped to define a plurality of adjoining cells.

Example 90. The apparatus according to example 88, wherein the wire comprises a shape-memory alloy.

Example 91. The apparatus according to example 88, wherein the second portion is more flexible than the first portion.

Example 92. The apparatus according to any one of examples 50-91, wherein the implant is configured such that, as the wing approaches the deflection-limit, the interface approaches the limiter.

Example 93. The apparatus according to example 92, wherein the implant is configured such that, upon the wing reaching the deflection-limit, the interface contacts the limiter.

Example 94. The apparatus according to example 92, wherein the limiter is shaped to define a cradle such that, upon the wing reaching the deflection-limit, the interface becomes temporarily seated within the cradle.

Example 95. The apparatus according to example 92, wherein the implant comprises a spring configured to strain as the interface approaches the limiter.

Example 96. The apparatus according to example 92, wherein the implant comprises a spring configured to bias the interface away from the limiter.

Example 97. The apparatus according to example 92, wherein the limiter extends from the interface away from the wing.

Example 98. The apparatus according to example 92, wherein the limiter is disposed on an opposite side of the interface from the wing.

Example 99. The apparatus according to example 92, wherein the limiter is shaped such that anchoring of the interface to the site presses the limiter against tissue of the first chamber.

Example 100. An apparatus for use with a real or simulated valve of a real or simulated heart of a real or simulated subject, the valve having a first leaflet and an opposing leaflet, the heart having a chamber upstream of the valve, the apparatus comprising an implant, the implant comprising: (i) a wing, extending from a root portion of the wing to a tip portion of the wing; and/or (ii) an interface, at the root portion, and configured to be anchored to a site in the chamber such that the implant is in a position in which: the wing extends over the first leaflet toward the opposing leaflet, and responsively to a cardiac cycle of the heart, the wing deflects, in a reciprocating manner, in an upstream direction and in a downstream direction, the wing being configured to define a deflection-limit, and to become resistant to deflection in the upstream direction upon reaching the deflection-limit.

Example 101. The apparatus according to example 100, wherein the implant is sterile.

Example 102. The apparatus according to any one of examples 100-101, wherein the wing is configured to become resistant to deflection in the upstream direction by the tip portion of the wing contacting the root portion of the wing upon the wing reaching the deflection-limit.

Example 103. The apparatus according to any one of examples 100-102, wherein the wing comprises a hinge that articulatably couples the root portion of the wing to the tip portion of the wing.

Example 104. The apparatus according to example 103, wherein the hinge is configured with a range of motion that defines the deflection-limit of the wing.

Example 105. The apparatus according to any one of examples 100-104, wherein: the tip portion comprises at least a first part and a second part, the second part is deflectable with respect to the first part, and upon the wing reaching the deflection-limit, the second part contacts the first part.

Example 106. The apparatus according to example 105, wherein the first part is closer than the second part to the root portion.

Example 107. The apparatus according to example 105, wherein the first part is closer than the second part to the interface.

Example 108. The apparatus according to example 105, wherein: the tip portion further comprises a third part, the third part is deflectable with respect to the second part, and upon the wing reaching the deflection-limit, the third part contacts the second part.

Example 109. The apparatus according to example 108, wherein the second part is closer than the third part to the root portion.

Example 110. The apparatus according to example 108, wherein the second part is closer than the third part to the interface.

Example 111. The apparatus according to example 108, wherein at least one of the first part, the second part, and the third part has a different flexibility from at least another of the first part, the second part, and the third part.

Example 112. The apparatus according to any one of examples 100-111, wherein the wing comprises a flexible frame, the frame being more flexible to deflection in the downstream direction than to deflection in the upstream direction.

Example 113. The apparatus according to example 112, wherein the frame has a plurality of notches cut therein.

Example 114. The apparatus according to example 113, wherein the implant is configured such that deflection of the wing in the downstream direction causes the notches to widen, and deflection of the wing in the upstream direction causes the notches to narrow.

Example 115. The apparatus according to example 114, wherein the notches are notches of a first set of notches, the frame has a second set of notches cut therein, and the implant is configured such that: deflection of the frame in the downstream direction causes the first set of notches to widen and the second set of notches to narrow, and deflection of the frame in the upstream direction causes the first set of notches to narrow and the second set of notches to widen.

Example 116. The apparatus according to example 113, wherein the notches are on an upstream side of the frame.

Example 117. The apparatus according to example 116, wherein the notches are notches of a first set of notches, and the frame has a second set of notches cut therein, the second set of notches being on a downstream side of the frame.

Example 118. The apparatus according to example 117, wherein the frame is configured such that flexing of the frame in a first direction widens the notches of the first set and narrows the notches of the second set, and flexing of the frame in a second direction narrows the notches of the first set and widens the notches of the second set.

Example 119. A system for use with a real or simulated valve of a real or simulated heart of a real or simulated subject, the valve having a first leaflet and an opposing leaflet, the heart having a chamber upstream of the valve, the system comprising: (i) an implant comprising: (a) a wing: extending from a root portion of the wing to a tip portion of the wing, and defining a contact face, and an opposing face opposite to the contact face, (b) a first interface defining a first longitudinal axis and (c) a second interface defining a second longitudinal axis, each of the first and second interfaces: disposed at the root portion, and coupled to the wing such that the first longitudinal axis is nonparallel to the second longitudinal axis; (ii) a first anchor and a second anchor; and/or (iii) a delivery tool, comprising: a catheter, transluminally advanceable to the chamber; a first shaft and a second shaft disposed alongside each other within the catheter, each of the first and second shafts engaged with a corresponding one of the first and second interfaces.

Example 120. The system according to example 119, wherein the first and second shafts are each configured, via the engagement with the corresponding interfaces, to: deploy the implant out of the catheter, and position the implant in a position in which: the first interface is at a first site upstream of the valve, the second interface is at a second site upstream of the valve, and the wing extends over the first leaflet toward the opposing leaflet, such the contact face faces the first leaflet.

Example 121. The system according to claim 120, further comprising a first driver and a second driver, each driver engaged with a corresponding one of the first and second anchors, and configured to secure the implant by screwing: the first anchor along the first longitudinal axis to anchor the first interface to tissue at the first site, and the second anchor along the second longitudinal axis to anchor the second interface to tissue at the second site.

Example 122. The system according to any one of examples 119-121, wherein at least one of the implant, the first anchor, the second anchor and the delivery tool is sterile.

Example 123. The system according to any one of examples 119-122, wherein, for each of the first and second interfaces, the interface has a proximal end that is orthogonal to the longitudinal axis of the interface.

Example 124. The system according to any one of examples 119-123, wherein each of the first and second interfaces has a circular proximal end.

Example 125. The system according to any one of examples 119-124, wherein the root portion of the wing defines a plane that is oblique to both the first longitudinal axis and the second longitudinal axis.

Example 126. The system according to example 125, wherein, while the first and second shafts each engage the corresponding interfaces, each shaft is oblique to the plane defined by the root portion of the wing.

Example 127. The system according to example 125, wherein an angle between the first longitudinal axis and the plane defined by the root portion of the wing is equal to an angle between the second longitudinal axis and the plane defined by the root portion of the wing.

Example 128. The system according to example 125, wherein an angle between the first longitudinal axis and the plane defined by the root portion of the wing is unequal to an angle between the second longitudinal axis and the plane defined by the root portion of the wing.

Example 129. The system according to example 125, wherein: the implant defines a first angle between the first longitudinal axis and a region of the plane that is disposed between the first and second interfaces, and a second angle between the second longitudinal axis and the region of the plane, and the first angle is greater than the second angle.

Example 130. The system according to example 129, wherein the first angle and the second angle are both acute.

Example 131. The system according to example 129, wherein the first angle is obtuse.

Example 132. The system according to example 131, wherein the second angle is acute.

Example 133. The system according to example 119, wherein: the first interface comprises a first cylindrical tube extending along the first longitudinal axis, and the second interface comprises a second cylindrical tube extending along the second longitudinal axis.

Example 134. The system according to example 133, wherein each of the first and second cylindrical tubes has: a circular cross-section that is transverse to a respective longitudinal axis, and a non-circular, elliptical distal end.

Example 135. The system according to any one of examples 119-134, wherein each of the first and second interfaces has a distal end that is oblique to the longitudinal axis of the respective interface.

Example 136. The system according to example 135, wherein the distal end of each of the first and second interfaces is parallel with a plane defined by the root portion of the wing.

Example 137. The system according to example 136, wherein each of the first and second interfaces has a proximal end that is oblique with respect to the plane defined by the root portion.

Example 138. The system according to example 137, wherein: the anchor has an anchor head, from which a tissue-engaging element extends, and for each of the first and second anchors, the respective driver is configured to screw the anchor along the respective longitudinal axis until the anchor head abuts the proximal end of the respective interface.

Example 139. A system for use with a real or simulated tissue of a real or simulated subject, the system comprising: (i) an anchor; an (ii) implant, the implant comprising a ratcheting interface, the interface configured to be anchored to a site of the tissue; and/or (iii) a delivery tool, comprising: (a) a catheter, transluminally advanceable to the tissue, (c) a shaft disposed within the catheter, the shaft engaged to the interface, wherein the interface is configured to: inhibit non-helical advancement of the anchor distally through the interface, and facilitate non-helical withdrawal of the anchor proximally through the interface.

Example 140. The system according to example 139, wherein the shaft is configured, via the engagement with the interface, to: deploy the implant out of the catheter, and position the implant in a position in which the interface is at the site.

Example 141. The system according to any one of examples 139-140, further comprising a driver, the driver engaged with the anchor and configured to anchor the interface to the tissue by helically advancing the anchor distally through the interface and into the tissue.

Example 142. The system according to any one of examples 139-141, wherein at least one of the implant, the anchor and the delivery tool is sterile.

Example 143. The system according to any one of examples 139-142, wherein the interface comprises: a tubular anchor receiver defining a lumen, and a tab that protrudes into the lumen such that application of a non-helical distalward force to the anchor causes the anchor to abut the tab in a manner that inhibits the non-helical distal advancement.

Example 144. The system according to example 143, wherein the tab is configured to deflect outwardly in response to application of a non-helical proximal force to the anchor, facilitating the non-helical proximal withdrawal.

Example 145. The system according to example 143, wherein: the anchor comprises a helical tissue-engaging element, and the tab is configured such that application of the non-helical distal force to the anchor causes the helical tissue-engaging element to abut the tab in a manner that inhibits the non-helical distal advancement.

Example 146. The system according to example 145, wherein the helical tissue-engaging element is configured to helically slide over the tab during helical distal advancement of the anchor through the interface.

Example 147. The system according to example 145, wherein the tab is configured such that application of a non-helical proximal force to the anchor causes the helical tissue-engaging element to deflect the tab outwardly, facilitating the non-helical proximal withdrawal.

Example 148. The system according to example 145, wherein the tab is configured such that application of a non-helical proximal force to the anchor causes the helical tissue-engaging element to ratchet proximally past the tab and through the lumen, facilitating the non-helical proximal withdrawal.

Example 149. A system for use with a real or simulated valve of a real or simulated heart of a real or simulated subject, the valve having a first leaflet and an opposing leaflet, the heart having a chamber upstream of the valve, the system comprising: (i) an implant, comprising: (a) a wing: extending from a root portion of the wing to a tip portion of the wing, defining a contact face, and an opposing face opposite to the contact face, having a compressed state and an expanded state, and comprising a flexible frame that comprises a shape-memory material and biases the wing toward assuming the expanded state, and (b) an expansion element, coupled to the wing, and having: a compact state, and an extended state in which the expansion element resists compression of the wing toward the compressed state; and/or (ii) a delivery tool, comprising: (a) a catheter, transluminally advanceable to the chamber, the catheter housing the implant while the wing is in the compressed state and the expansion element is in the compact state; (b) a shaft disposed within the catheter, the shaft engaged to the implant, wherein the delivery tool is configured to: deploy the implant out of the catheter such that, within the chamber, the wing assumes the expanded state and the expansion element assumes the extended state, and position the implant in a position in which the wing extends over the first leaflet toward the opposing leaflet, and the contact face faces the first leaflet.

Example 150. The system according to example 149, wherein the implant is sterile.

Example 151. The system according to any one of examples 149-150, wherein the delivery tool is sterile.

Example 152. The system according to any one of examples 149-151, wherein the expansion element is configured to resist transition from the extended state toward the compact state.

Example 153. The system according to any one of examples 149-152, wherein the expansion element comprises a spring.

Example 154. The system according to any one of examples 149-153, wherein the expansion element comprises a plurality of subunits, and the expansion element is configured such that extending the expansion element into the extended state causes the subunits to fit together.

Example 155. The system according to any one of examples 149-154, wherein the expansion element comprises a plurality of subunits, configured to lock together upon the expansion element assuming the extended state.

Example 156. The system according to any one of examples 149-155, wherein the expansion element is straighter in the extended state than in the compact state.

Example 157. The system according to example 156, wherein the expansion element comprises a hinge, and the expansion element is configured such that straightening the hinge straightens the expansion element.

Example 158. The system according to any one of examples 149-157, wherein the delivery tool further comprises an extension actuator, the extension actuator configured to transluminally extend the expansion element from the compact state to the extended state.

Example 159. The system according to example 158, wherein the implant is configured such that extension of the expansion element from the compact state to the extended state applies an expansion force to the wing, the expansion force facilitating expansion of the wing from the compressed state to the expanded state.

Example 160. The system according to example 158, wherein: the implant comprises a pair of interfaces at the root portion of the wing, the shaft bifurcates at a distal portion of the shaft into two branches, each of the branches being engaged with a corresponding one of the interfaces, and the expansion element is configured to push the interfaces away from each other as the expansion element extends toward its extended state.

Example 161. The system according to example 160, wherein the expansion element is coupled to the wing via the pair of interfaces.

Example 162. The system according to example 160, wherein the extension actuator is disposed between the branches.

Example 163. A system for use with a real or simulated valve of a real or simulated heart of a real or simulated subject, the valve having a first leaflet and an opposing leaflet, the heart having a chamber upstream of the valve, the system comprising: (i) an implant comprising: (a) a wing: extending from a root portion of the wing to a tip portion of the wing, comprising: a frame that provides mechanical support to the wing, and a flexible sheet covering the frame, and extending beyond the frame to define lateral flaps, and (b) an interface at the root portion; (ii) an anchor; and/or (iii) a delivery tool, comprising: a catheter, transluminally advanceable to the chamber; a shaft disposed within the catheter, the shaft engaged with the interface.

Example 164. The system according to example 163, wherein the shaft configured, via the engagement with the interface, to: deploy the implant out of the catheter, and position the implant in a position in which the interface is at a site upstream of the valve, the wing extends over the first leaflet toward the opposing leaflet, and the lateral flaps extend over the first leaflet toward respective commissures of the valve.

Example 165. The system according to any one of examples 163-164, further comprising a driver, engaged with the anchor and configured to secure the implant by using the anchor to anchor the interface to tissue of the heart.

Example 166. The system according to any one of examples 163-165, wherein at least one of the implant, the anchor and the delivery tool is sterile.

Example 167. The system according to any one of examples 163-166, wherein the wing is more flexible at the lateral flaps than at a medial region in which the frame is disposed.

Example 168. The system according to any one of examples 163-167, wherein the flexible sheet has a shape that resembles that of a manta ray.

Example 169. The system according to any one of examples 163-168, wherein each of the lateral flaps defines a lateral extremity between a root portion of the lateral flap and a tip portion of the lateral flap.

Example 170. The system according to example 169, wherein the lateral extremity is angular.

Example 171. An apparatus for use with a real or simulated valve of a real or simulated heart of a real or simulated subject, the valve having a first leaflet and an opposing leaflet, the heart having a chamber upstream of the valve, the apparatus comprising an implant, the implant comprising: (i) a wing: extending from a root portion of the wing to a tip portion of the wing, the root portion being stiffer than the tip portion, and defining a contact face, and an opposing face opposite to the contact face; and/or (ii) an interface at the root portion; wherein the implant is configured to be implanted in a position in which: the interface is at a site upstream of the valve, the wing extends over the first leaflet toward the opposing leaflet, and the contact face faces the first leaflet.

Example 172. The apparatus according to example 171, wherein the implant is sterile.

Example 173. The apparatus according to any one of examples 171-172, wherein the wing comprises a flexible frame that provides mechanical support to the root portion of the wing.

Example 174. The apparatus according to any one of examples 171-173, wherein the tip portion of the wing comprises a flexible sheet.

Example 175. The apparatus according to example 174, wherein flexible sheet comprises a polymer.

Example 176. The apparatus according to example 171, wherein the wing comprises a flexible frame that provides mechanical support to the wing.

Example 177. The apparatus according to example 176, wherein the frame defines less open space at the root portion of the wing than at the tip portion of the wing.

Example 178. The apparatus according to example 176, wherein members of the frame are thicker at the root portion of the wing than at the tip portion of the wing.

Example 179. The apparatus according to example 176, wherein members of the frame are spaced more closely to each other at the root portion of the wing than at the tip portion of the wing.

Example 180. The apparatus according to example 176, wherein the frame comprises a wire frame, and the wire frame comprises thicker wires at the root portion of the wing than at the tip portion of the wing.

Example 181. The apparatus according to example 176, wherein: the frame at the root portion of the wing comprises a first material, the frame at the tip portion of the wing comprises a second material, and the first material is stiffer than the second material.

Example 182. The apparatus according to example 176, wherein the frame comprises a wire frame, and the wire frame is more densely populated with wires at the root portion of the wing than at the tip portion of the wing.

Example 183. The apparatus according to example 182, wherein the wire frame comprises thicker wires at the root portion of the wing than at the tip portion of the wing.

Example 184. The apparatus according to any one of examples 171-172, wherein the wing comprises a wire mesh.

Example 185. The apparatus according to example 184, wherein the wing further comprises a flexible frame over which the wire mesh is disposed.

Example 186. The apparatus according to example 184, wherein the wire mesh has a weave that is more densely woven at the root portion than at the tip portion.

Example 187. The apparatus according to example 184, wherein the wire mesh comprises thicker wire at the root portion than at the tip portion.

Example 188. The apparatus according to any one of examples 171-187, wherein the wing defines a flex element, the flex element coupling the tip portion of the wing to the root portion of the wing.

Example 189. The apparatus according to example 188, wherein the implant is configured such that, while the implant is secured in the position, flexing of the flex element facilitates deflection of the tip portion with respect to the root portion in response to a cardiac cycle of the heart.

Example 190. A method for use with a real or simulated valve of a real or simulated heart of a real or simulated subject, the valve having an annulus, a first leaflet, and an opposing leaflet, the heart having a chamber upstream of the valve, the method comprising: (i) within a catheter, advancing to the chamber: (a) a shaft, and (b) an implant that includes: an interface, engaged with a distal end of the shaft, a flexible wing coupled to the interface; and/or (ii) using the shaft, deploying the implant out of the catheter and into the chamber; (iii) using the shaft, positioning the implant in a position in which the interface is at a site on the annulus and the wing extends over the first leaflet toward the opposing leaflet; (iv) anchoring the interface at the site; (v) subsequently, releasing the distal end of the shaft from the interface by pulling on a ripcord; and/or (vi) subsequently, withdrawing the catheter and the shaft from the subject.

Example 191. The method according to example 190, further comprising sterilizing the implant.

Example 192. The method according to any one of examples 190-191, further comprising sterilizing the catheter and the shaft.

Example 193. A system for use at a real or simulated tissue of a real or simulated subject, the system comprising: (i) a first anchor and a second anchor; (ii) an implant comprising a first interface and a second interface; and/or (iii) a delivery tool extending from a proximal portion to a distal portion, the delivery tool comprising: (a) a catheter, transluminally advanceable to the tissue, (b) a shaft, extending distally through the catheter, a distal part of the shaft bifurcating into a first branch and a second branch, each branch disposed alongside each other within the catheter, each of the first and second branches engaged with a corresponding one of the first and second interfaces, (c) a driver: extending or extendable distally through the shaft, a drive head of the first driver engaged or engageable with at least one of the first anchor and the second anchor, and being configured to anchor at least one of the first interface and the second interface to the tissue by driving the anchor distally through the first interface or second interface and into the tissue.

Example 194. The system according to example 193, wherein the driver is a first driver extending or extendable distally through the shaft and into the first branch where a drive head of the first driver is engaged or engageable with the first anchor, the first driver being configured to anchor the first interface to the tissue by driving the anchor distally through the first interface and into the tissue, and wherein the system further comprises a second driver: extending or extendable distally through the shaft alongside the first driver, and into the second branch where a drive head of the second driver is engaged or engageable with the second anchor, and configured to anchor the second interface to the tissue by driving the anchor distally through the second interface and into the tissue.

Example 195. The system according to any one of examples 193-194, wherein the implant is sterile.

Example 196. The system according to any one of examples 193-195, wherein the first anchor and the second anchor are sterile.

Example 197. The system according to any one of examples 193-196, wherein the delivery tool is sterile.

Example 198. The system according to any one of examples 193-197, wherein: the first branch has a first width, the second branch has a second width, and a portion of the shaft, proximal from the first and second branches, is narrower than the sum of the first and second widths.

Example 199. The system according to any one of examples 193-198, wherein: the catheter defines a first lumen, and a second lumen alongside the first lumen, and each of the first and second drivers extend, from the proximal portion to the distal portion, within a respective one of the lumens.

Example 200. The system according to example 194, wherein the delivery tool further comprises a controller configured to operate the first driver and the second driver.

Example 201. The system according to example 200, wherein the controller is transitionable between: a first setting in which the controller operates the first and second driver simultaneously, and a second setting in which the controller operates only one of the first and second drivers at a given time.

Example 202. A method for use with a real or simulated valve of a real or simulated heart of a real or simulated subject, the valve having a first leaflet and an opposing leaflet, the heart having a first chamber upstream of the valve and a second chamber downstream of the valve, the method comprising: (i) within a catheter, advancing to the first chamber: (a) a shaft, and (b) an implant that includes: an interface, engaged with a distal end of the shaft, and a flexible wing coupled to the interface; (iii) using the shaft: (a) deploying the implant out of the catheter and into the first chamber, and (b) anchoring the implant in a position in which: the interface is at a site in the first chamber, the wing extends over the first leaflet toward the opposing leaflet, and responsively to a cardiac cycle of the heart, the wing deflects, in a reciprocating manner, in an upstream direction and in a downstream direction; and/or (iv) subsequently, intracardially adjusting a deflection-range of the wing.

Example 203. The method according to example 202, further comprising sterilizing the implant, the shaft and the catheter.

Example 204. The method according to any one of examples 202-203, wherein: the site is at an annulus of the valve, and anchoring the implant in the position comprises anchoring the interface to the annulus of the valve.

Example 205. The method according to example 204, wherein: the interface is coupled to a root portion of the wing; and/or anchoring the interface to the annulus comprises anchoring the interface to the annulus such that the root portion is disposed at the annulus and the wing extends, from the root portion, over the first leaflet toward the opposing leaflet.

Example 206. The method according to any one of examples 202-204, wherein: the implant further includes a limiter that defines a deflection-limit of the wing during the cardiac cycle of the heart by inhibiting deflection of the wing in the upstream direction beyond the deflection-limit, and adjusting the deflection-range of the wing comprises intracardially adjusting the deflection-limit of the wing by adjusting the limiter.

Example 207. The method according to example 206, wherein: anchoring the implant in the position comprises driving an anchor into tissue at the site, and adjusting the limiter comprises adjusting the limiter by applying torque to the anchor.

Example 208. The method according to example 206, wherein: the limiter includes a tether, coupled to the wing; and/or adjusting the deflection-range of the wing comprises adjusting the deflection-limit of the wing by intracardially adjusting tension on the tether.

Example 209. The method according to example 208, further comprising anchoring the tether to tissue of the second chamber prior to adjusting the tension.

Example 210. The method according to example 208, wherein: a portion of the tether is wound around a rotatable spool; and/or adjusting tension on the tether comprises, using an extracorporeal controller, adjusting tension on the tether via the catheter by rotating the spool.

Example 211. The method according to example 208, wherein intracardially adjusting tension on the tether comprises intracardially sliding the tether with respect to the wing.

Example 212. The method according to example 208, wherein: a first portion of the tether is coupled to the wing, and adjusting tension on the tether comprises passing a second portion of the tether, in the upstream direction, through a root portion of the wing.

Example 213. The method according to example 212, wherein adjusting tension on the tether comprises passing the second portion of the tether, in the upstream direction, through the interface.

Example 214. The method according to example 206, wherein: anchoring the implant in the position comprises anchoring the interface to tissue at the site by driving an anchor into the tissue, the anchor having an anchor head, and a tissue-engaging element that extends from the anchor head to define an anchor axis of the anchor, and adjusting the limiter comprises deflecting the limiter with respect to the anchor axis.

Example 215. The method according to example 214, wherein deflecting the limiter comprises changing a curvature of the limiter.

Example 216. The method according to example 214, wherein deflecting the limiter comprises bringing the limiter into greater contact with the wing.

Example 217. The method according to example 216, wherein deflecting the limiter comprises deflecting the limiter such that a portion of the limiter contacts the wing upon the wing reaching the deflection-limit.

Example 218. The method according to example 217, wherein deflecting the limiter comprises deflecting the limiter such that the portion of the limiter does not contact the wing during ventricular diastole of the cardiac cycle.

Example 219. The method according to example 206, wherein: anchoring the implant in the position comprises driving an anchor into tissue at the site, and adjusting the limiter comprises adjusting the limiter by driving the anchor deeper into the tissue at the site.

Example 220. The method according to example 219, wherein: the anchor has an anchor head and a tissue-engaging element, the tissue-engaging element extending from the anchor head to define an anchor axis of the anchor, and adjusting the limiter comprises deflecting the limiter with respect to the anchor axis.

Example 221. The method according to example 206, wherein: the limiter defines a backstop portion, and adjusting the limiter comprises pressing the backstop portion against tissue of the first chamber.

Example 222. The method according to example 221, wherein: the backstop portion defines a spring, and pressing the backstop portion against the tissue of the first chamber comprises tensioning the spring.

Example 223. The method according to example 221, wherein: the backstop portion is an inflatable backstop portion, and pressing the backstop portion against the tissue of the first chamber comprises pressing the backstop portion against the tissue by inflating the backstop portion.

Example 224. The method according to any one of examples 202-204, wherein: the implant includes a tether, coupled to the wing; and/or intracardially adjusting the deflection-range of the wing comprises, intracardially adjusting the deflection-range of the wing by adjusting tension on the tether.

Example 225. The method according to example 224, wherein: the tether is coupled to a tip portion of the wing, and adjusting tension on the tether comprises adjusting deflectability of the tip portion of the wing.

Example 226. The method according to example 224, wherein: the tether is slidably coupled to a root portion of the wing, and adjusting tension on the tether comprises adjusting deflectability of the root portion of the wing by sliding the tether through a sleeve at the root portion of the wing.

Example 227. The method according to example 224, wherein: the tether is slidably coupled to a root portion of the wing, and the method further comprises anchoring the tether to tissue of the first chamber.

Example 228. The method according to example 224, wherein: the tether is coupled to the wing, and the method further comprises anchoring the tether to tissue of the second chamber.

Example 229. The method according to example 228, wherein: the tether defines a rail portion to which a proximal portion of the tether is slidably coupled; and/or the step of anchoring comprises: anchoring a first part of the rail portion to trabeculae at a first site of the second chamber, and anchoring a second part of the rail portion to trabeculae at a second site of the second chamber.

Example 230. The method according to example 224, wherein: a first portion of the tether is coupled to the wing, and adjusting tension on the tether comprises passing a second portion of the tether, in the upstream direction, through a root portion of the wing.

Example 231. The method according to example 230, wherein adjusting tension on the tether comprises passing the second portion of the tether, in the upstream direction, through the interface.

Example 232. The method according to example 224, wherein adjusting the deflection-range of the wing by adjusting tension on the tether comprises pivoting the wing with respect to the interface by adjusting tension on the tether.

Example 233. The method according to example 232, wherein: anchoring the implant in the position comprises anchoring the interface to the site by driving, into tissue at the site, an anchor that defines: an anchor head, and a tissue-engaging element extending from the anchor head along an anchor axis, and pivoting the wing with respect to the interface by adjusting tension on the tether comprises pivoting the wing with respect to the anchor axis by adjusting tension on the tether.

Example 234. The method according to any one of examples 202-204, wherein: the interface is an adjustable interface, and adjusting the deflection-range of the wing comprises intracardially adjusting the deflection-range by adjusting the interface.

Example 235. The method according to example 234, wherein: the adjustable interface defines a seat, anchoring the implant comprises seating the seat against tissue at the site in the first chamber, and adjusting the interface comprises adjusting an angle between a root portion of the wing and the seat of the interface.

Example 236. The method according to example 235, wherein: the step of anchoring comprises, using an anchor, anchoring the implant in the position; the anchor defines an anchor head and a tissue-engaging element, the tissue-engaging element extending from the anchor head along an anchor axis; and/or adjusting the interface comprises adjusting an angle between the root portion of the wing and the anchor axis.

Example 237. The method according to example 235, wherein: the adjustable interface includes an adjustment mechanism, and adjusting the angle between the root portion of the wing and the seat of the interface comprises adjusting the angle between the root portion of the wing and the seat of the interface by actuating the adjustment mechanism.

Example 238. The method according to example 237, wherein: the adjustable interface includes a base to which the root portion of the wing is fixedly coupled, and adjusting the angle between the root portion of the wing and the seat of the interface comprises adjusting the angle between the base and the seat of the interface, by actuating the adjustment mechanism.

Example 239. The method according to example 238, wherein: the adjustment mechanism includes a lead screw, and actuating the adjustment mechanism comprises rotating the lead screw.

Example 240. The method according to example 239, wherein: the step of anchoring comprises, using an anchor, anchoring the implant in the position; the anchor defines an anchor head and a tissue-engaging element, the tissue-engaging element extending from the anchor head along an anchor axis; and/or screwing the lead screw comprises screwing the lead screw along a lead screw axis that is offset with respect to the anchor axis.

Example 241. The method according to example 239, wherein: the step of anchoring comprises, using an anchor, anchoring the implant in the position; the anchor defines an anchor head and a tissue-engaging element, the tissue-engaging element extending from the anchor head along an anchor axis; and/or screwing the lead screw comprises screwing the lead screw along a lead screw axis that is colinear with the anchor axis.

Example 242. A method for use with a real or simulated valve of a simulated heart of a real or simulated subject, the valve having a first leaflet and an opposing leaflet, the heart having a first chamber upstream of the valve and a second chamber downstream of the valve, the method comprising: (i) within a catheter, advancing to the first chamber: (a) a shaft, and (b) an implant that includes: an interface, engaged with a distal end of the shaft, and a flexible wing coupled to the interface; (iii) using the shaft: (a) deploying the implant out of the catheter and into the first chamber, and (b) anchoring the implant in a position in which: the interface is at a site in the first chamber, the wing extends over the first leaflet toward the opposing leaflet, and responsively to a cardiac cycle of the heart, the wing deflects, in a reciprocating manner, in an upstream direction and in a downstream direction; and/or (iv) subsequently, intracardially adjusting a deflection-range of the wing.

Example 243. A method for use with a real or simulated valve of a real or simulated heart of a real or simulated subject, the valve having an annulus, a first leaflet, and an opposing leaflet, the heart having a chamber upstream of the valve, the method comprising: (i) advancing an implant to the chamber, the implant including: (a) a wing, extending from a root portion of the wing to a tip portion of the wing, (b) an interface at the root portion of the wing, and (c) a shape-memory member, coupled to the wing; (ii) positioning the implant in a position in which: (a) the interface is at a site in the chamber, and (b) the wing extends, from the site, over the first leaflet toward the opposing leaflet; (iii) anchoring the interface to the site; and/or (iv) while the implant remains in the position, inducing the shape-memory member to chronically change a size of the wing by temporarily heating the shape-memory member.

Example 244. The method according to example 243, further comprising sterilizing the implant.

Example 245. The method according to any one of examples 243-244, wherein the step of inducing comprises, while the implant remains in the position, inducing the shape-memory member to chronically change a width of the wing by temporarily heating the shape-memory member.

Example 246. The method according to any one of examples 243-245, wherein the step of inducing comprises, while the implant remains in the position, inducing the shape-memory member to chronically change a length of the wing by temporarily heating the shape-memory member.

Example 247. The method according to example 246, wherein: a first end of the shape-memory member is coupled to the root portion of the wing, a second end of the shape-memory member is coupled to the tip portion of the wing, and the step of inducing comprises, while the implant remains in the position, inducing the shape-memory member to chronically change a distance between the first end of the shape-memory member and the second end of the shape-memory member by temporarily heating the shape-memory member.

Example 248. The method according to any one of examples 243-247, wherein the step of heating comprises temporarily heating the shape-memory member by applying electrical power to the shape-memory member.

Example 249. The method according to example 248, wherein applying the electrical power to the shape-memory member comprises wirelessly applying the electrical power to the shape-memory member.

Example 250. The method according to example 248, wherein applying the electrical power to the shape-memory member comprises applying the electrical power to the shape-memory member via a catheter.

Example 251. The method according to any one of examples 243-250, wherein: the wing includes a locking mechanism that configures the wing to chronically remain at the changed size, and the method further comprises temporarily unlocking the locking mechanism.

Example 252. The method according to example 251, wherein: the shape-memory member is a first shape-memory member, the locking mechanism comprises a second shape-memory member, and temporarily unlocking the locking mechanism comprises temporarily heating the second shape-memory member.

Example 253. A method for use with a real or simulated valve of a simulated heart of a real or simulated subject, the valve having an annulus, a first leaflet, and an opposing leaflet, the heart having a chamber upstream of the valve, the method comprising: (i) advancing an implant to the chamber, the implant including: (a) a wing, extending from a root portion of the wing to a tip portion of the wing, (b) an interface at the root portion of the wing, and (c) a shape-memory member, coupled to the wing; (ii) positioning the implant in a position in which: (a) the interface is at a site in the chamber, and (b) the wing extends, from the site, over the first leaflet toward the opposing leaflet; (iii) anchoring the interface to the site; and/or (iv) while the implant remains in the position, inducing the shape-memory member to chronically change a size of the wing by temporarily heating the shape-memory member.

Example 254. A system for use with a real or simulated valve of a real or simulated heart of a real or simulated subject, the valve having an annulus, a first leaflet and an opposing leaflet, the heart having first chamber upstream of the valve and a second chamber downstream of the valve, the system comprising an implant, the implant comprising: (i) a flexible wing, the wing: extending from a root portion of the wing to a tip portion of the wing; and/or (ii) a limb or extension coupled to the wing, (iii) wherein: (a) the root portion of the wing is configured to be placed against a site on the annulus, adjacent a root of the first leaflet, in a manner that supports the wing extending, from the root portion of the wing, over the first leaflet toward the opposing leaflet, and (b) the limb/extension is shaped such that, when the root portion of the wing is placed against the site, the limb/extension extends away from the wing to contact tissue of the heart adjacent a root of the opposing leaflet, in a manner that moderates deflection of the wing with respect to the site in an upstream direction.

Example 255. The system according to example 254, wherein the implant is sterile.

Example 256. The system according to any one of examples 254-255, wherein the implant is configured such that when the root portion of the wing is placed against the site, and the limb/extension extends away from the wing to contact tissue of the heart, the tip portion of the wing deflects with respect to the root portion of the wing, reciprocatingly in the upstream direction and in a downstream direction, responsively to a cardiac cycle of the heart.

Example 257. The system according to any one of examples 254-256, wherein the limb/extension is shaped such that, when the root portion of the wing is placed against the site, the limb/extension extends away from the root portion of the wing to contact tissue of the heart in a manner that moderates deflection of the wing with respect to the site in the upstream direction.

Example 258. The system according to any one of examples 254-257, wherein the limb/extension is shaped such that, when the root portion of the wing is placed against the site, the limb/extension extends away from the tip portion of the wing to contact tissue of the heart in a manner that moderates deflection of the wing with respect to the site in the upstream direction.

Example 259. The system according to any one of examples 254-258, wherein the limb/extension is shaped such that, when the root portion of the wing is placed against the site, the limb/extension extends away from the wing to contact tissue of the annulus in a manner that moderates deflection of the wing with respect to the site in the upstream direction.

Example 260. The system according to example 259, wherein the limb/extension defines an arm that is shaped such that, when the root portion of the wing is placed against the site, the arm is disposed against an atrial surface of the annulus in a manner that moderates deflection of the wing with respect to the site in the upstream direction.

Example 261. The system according to example 260, wherein the implant further comprises an interface at the root portion of the wing, the interface configured to be secured to the site on the annulus by driving an anchor into tissue at the site.

Example 262. The system according to example 260, wherein the arm comprises an anchor receiver, the anchor receiver configured to be secured to the atrial surface of the annulus, when the root portion of the wing is placed against the site, by driving an anchor through the anchor receiver and into tissue at the atrial surface of the annulus.

Example 263. The system according to example 262, wherein the arm is shaped such that, when the root portion of the wing is placed against the site, the anchor receiver is disposed adjacent a commissure of the valve.

Example 264. The system according to example 262, wherein the arm is shaped such that, when the root portion of the wing is placed against the site, the anchor receiver is disposed adjacent a root portion of the first leaflet.

Example 265. The system according to example 262, wherein the arm is shaped such that, when the root portion of the wing is placed against the site, the anchor receiver is disposed adjacent a root portion of the opposing leaflet.

Example 266. The system according to any one of examples 254-258, wherein the limb/extension defines a leg that is shaped such that, when the root portion of the wing is placed against the site, the leg contacts tissue of the ventricle in a manner that moderates deflection of the wing with respect to the site in the upstream direction.

Example 267. The system according to example 266, wherein the implant further comprises an interface at the root portion of the wing, the interface configured to be secured to the site on the annulus by driving an anchor into tissue at the site.

Example 268. The system according to example 266, wherein leg that is shaped such that, when the root portion of the wing is placed against the site, the leg contacts tissue of an underside of the valve in a manner that moderates deflection of the wing with respect to the site in the upstream direction.

Example 269. The system according to example 268, wherein the leg is shaped such that, when the root portion of the wing is placed against the site, the leg is disposed adjacent a commissure of the valve.

Example 270. The system according to example 268, wherein leg is shaped such that, when the root portion of the wing is placed against the site, the leg is disposed in a subannular groove of the valve.

Example 271. The system according to example 266, wherein the implant further comprises an atrial support, the atrial support coupled to the wing and configured such that, when the root portion of the wing is placed against the site, the atrial support presses against an atrial surface of the annulus in a manner that presses the leg against the tissue of the ventricle.

Example 272. The system according to example 271, wherein the atrial support is shaped to circumscribe the atrial surface of the annulus.

Example 273. The system according to example 271, wherein the atrial support is defined by a pair of arms that extend, from the root portion, in opposite directions around the atrial surface of the annulus.

Example 274. The system according to example 266, wherein: the ventricle is a left ventricle, the valve is a mitral valve, the first leaflet is a posterior leaflet of the mitral valve, the opposing leaflet is an anterior leaflet of the mitral valve; and/or the leg is shaped such that, when the root portion of the wing is placed against the site, the leg contacts tissue of the left ventricle behind the anterior leaflet.

Example 275. The system according to example 274, wherein the leg is shaped such that, when the root portion of the wing is placed against the site, the leg contacts a fibrous trigone of the left ventricle.

Example 276. The system according to example 254, wherein: the wing has a compressed state, and is biased to expand into an expanded state; and/or the limb/extension comprises an annular support, coupled to the root portion of the wing and configured such that: in the compressed state of the wing, the wing has a hinged coupling to the annular support that facilitates articulation, at the hinged coupling, of the wing with respect to the annular support, and expansion of the wing toward the expanded state inhibits the articulation by restraining the hinged coupling.

Example 277. The system according to example 276, wherein: the implant further comprises an interface at the root portion of the wing; the wing defines a contact face, and an opposing face opposite to the contact face; the system further comprises: an anchor, and a delivery tool, comprising: a catheter, transluminally advanceable to the atrium with the implant housed in the catheter while the wing is in the compressed state, and a driver, configured to: deploy the implant out of the catheter such that, within the first chamber, the wing assumes the expanded state, position the implant in a position in which: the wing extends over the first leaflet toward the opposing leaflet, and the contact face faces the first leaflet, and while the implant is positioned in the position and the wing is in the expanded state, secure the interface to the annulus by driving the anchor through the interface and into tissue of the annulus.

Example 278. The system according to example 277, wherein the annular support is shaped such that, while the implant is secured to the annulus and the wing is in the expanded state, the annular support is disposed against an atrial surface of the annulus such that, the restrained hinged coupling inhibits deflection of the root portion of the wing with respect to the annulus.

Example 279. The system according to example 278, wherein: the interface is a first interface; the annular support comprises a first annular arm that extends away from the hinged coupling to the first interface; and/or the annular support further comprises a second annular arm that: is coupled to the hinged coupling, and extends away from the hinged coupling to a second interface.

Example 280. The system according to example 279, wherein the first annular arm is joined to the second annular arm, at the hinged coupling.

Example 281. The system according to example 276, wherein the implant is configured such that: the hinged coupling comprises a sleeve defining an aperture, while the wing is in the compressed state, a thin portion of the annular support is disposed within the aperture, and expansion of the wing toward the expanded state slides the sleeve from the thin portion to a thick portion of the annular support, the thick portion having a cross-section that is dimensioned to restrain the hinged coupling by fitting the aperture.

Example 282. The system according to example 281, wherein the thick portion of the annular support has an oblong cross-section that is dimensioned to restrain the hinged coupling by fitting the aperture.

Example 283. The system according to example 281, wherein: the sleeve is a first sleeve defining a first aperture, the hinged coupling further comprises a second sleeve defining a second aperture, the annular support comprises a pair of annular arms, each annular arm having: a thin portion at which the annular arms are joined, and a thick portion that extends away from the thin portion; wherein expansion of the wing toward the expanded state slides each sleeve from the thin portion to the thick portion of a respective annular arm, thereby restraining the hinged coupling.

Example 284. A system and/or apparatus for use with a real or simulated valve of a real or simulated heart of a real or simulated subject, the valve having a first leaflet and an opposing leaflet, the heart having a chamber upstream of the valve, the system/apparatus comprising an implant, the implant comprising: (i) a wing, extending from a root portion of the wing to a tip portion of the wing; (ii) an interface at the root portion of the wing, the interface configured to be anchored to a site in the chamber such that the implant is secured in a position in which: (a) the wing extends over the first leaflet toward the opposing leaflet, and (b) responsively to a cardiac cycle of the heart, the wing deflects, in a reciprocating manner, in an upstream direction and in a downstream direction; and/or (iii) an adjustment member that is adjustable in a manner that adjusts a deflection-range of the wing.

Example 285. The system/apparatus according to example 284, wherein the implant is sterile.

Example 286. The system/apparatus according to any one of examples 284-285, wherein the adjustment member is an adjustment mechanism that is actuatable by application of torque.

Example 287. The system/apparatus according to any one of examples 284-286, wherein: the adjustment member comprises an adjustable limiter that defines a deflection-limit of the wing during the cardiac cycle of the heart by inhibiting deflection of the wing in the upstream direction beyond the deflection-limit, and the limiter is adjustable in a manner that intracardially adjusts the deflection-limit of the wing.

Example 288. The system/apparatus according to example 287, wherein the system/apparatus further comprises: a catheter, and an extracorporeal controller, the controller configured to adjust the limiter via the catheter.

Example 289. The system/apparatus according to example 287, wherein the limiter: defines a backstop portion, and is adjustable by pressing the backstop portion against tissue of the chamber.

Example 290. The system/apparatus according to example 289, wherein: the backstop portion defines a spring, and the limiter is configured such that tensioning the spring presses the backstop portion against the tissue of the chamber.

Example 291. The system/apparatus according to example 289, wherein: the backstop portion is an inflatable backstop portion, and the limiter is configured such that inflating the backstop portion presses the backstop portion against the tissue of the chamber.

Example 292. The system/apparatus according to example 287, wherein the limiter is configured such that such that a portion of the limiter contacts the wing upon the wing reaching the deflection-limit.

Example 293. The system/apparatus according to example 292, wherein the limiter is configured such that such that the portion of the limiter does not contact the wing during ventricular diastole of the cardiac cycle.

Example 294. The system/apparatus according to example 287, further comprising: an anchor; and/or a driver, engaged with the anchor, and configured to: secure the implant in the position by using the anchor to anchor the interface to the site by driving the anchor into tissue at the site, and adjust the limiter by driving the anchor deeper into the tissue at the site.

Example 295. The system/apparatus according to example 294, wherein the driver is configured to secure the implant in the position, and to anchor the interface to the site, by driving the anchor into tissue at the site.

Example 296. The system/apparatus according to example 295, wherein: the anchor comprises an anchor head, and a tissue-engaging element that extends from the anchor head to define an anchor axis of the anchor, and the limiter is configured such that driving the anchor deeper into the tissue at the site adjusts the limiter by deflecting the limiter with respect to the anchor axis.

Example 297. The system/apparatus according to example 295, wherein the driver is configured to drive the anchor deeper into the tissue at the site by applying torque to the anchor.

Example 298. The system/apparatus according to example 294, wherein the limiter is configured such that driving the anchor deeper into the tissue at the site adjusts the limiter by bringing the limiter into greater contact with the wing.

Example 299. The system/apparatus according to example 298, wherein the limiter is configured such that driving the anchor deeper into the tissue at the site adjusts the limiter by changing a curvature of the limiter.

Example 300. The system/apparatus according to example 287, wherein: the limiter comprises a tether, the tether coupled to the wing, and the limiter is configured such that intracardially adjusting tension on the tether adjusts the deflection-limit of the wing.

Example 301. The system/apparatus according to example 300, wherein the limiter is configured such that intracardially adjusting tension on the tether, by intracardially sliding the tether with respect to the wing, adjusts the deflection-limit of the wing.

Example 302. The system/apparatus according to example 300, wherein: a portion of the tether is wound around a rotatable spool, and the limiter is configured such that intracardially adjusting tension on the tether, by rotating the spool, adjusts the deflection-limit of the wing.

Example 303. The system/apparatus according to example 300, wherein: a first portion of the tether is coupled to the wing, and the tether is configured such that passing a second portion of the tether, through the root portion of the wing in the upstream direction, adjusts tension on the tether.

Example 304. The system/apparatus according to example 303, wherein the tether is configured such that passing the second portion of the tether, through the interface in the upstream direction, adjusts tension on the tether.

Example 305. The system/apparatus according to any one of examples 284-286, wherein: the adjustment member comprises a tether, and the implant is configured such that adjusting tension on the tether adjusts the deflection-range of the wing.

Example 306. The system/apparatus according to example 305, wherein the system/apparatus further comprises: a catheter, and an extracorporeal controller, the controller configured to adjust the tension on the tether via the catheter.

Example 307. The system/apparatus according to example 305, wherein: the tether is coupled to the tip portion of the wing, and the implant is configured such that adjusting tension on the tether adjusts deflectability of the tip portion of the wing.

Example 308. The system/apparatus according to example 305, wherein: a first portion of the tether is coupled to the wing, and the implant is configured such that passing a second portion of the tether through the root portion of the wing adjusts the deflection-range of the wing.

Example 309. The system/apparatus according to example 308, wherein the implant is configured such that passing a second portion of the tether through the interface adjusts the deflection-range of the wing.

Example 310. The system/apparatus according to example 305, wherein the implant is configured such that adjusting tension on the tether adjusts the deflection-range of the wing by pivoting the wing with respect to the interface.

Example 311. The system/apparatus according to example 310, wherein: the system/apparatus further comprises an anchor, the anchor having an anchor head and a tissue-engaging element that extends from the anchor head to define an anchor axis, and the implant is configured such that adjusting tension on the tether adjusts the deflection-range of the wing by pivoting the wing with respect to the anchor axis.

Example 312. The system/apparatus according to any one of examples 284-286, wherein the adjustment member is defined by the interface, which is an adjustable interface comprising an adjustment mechanism that is adjustable in a manner that adjusts the deflection-range of the wing.

Example 313. The system/apparatus according to example 312, wherein the system/apparatus further comprises: a catheter, and an extracorporeal controller, the controller configured to adjust the adjustment mechanism via the catheter.

Example 314. The system/apparatus according to example 312, wherein the adjustment mechanism: defines a seat, configured to be seated against tissue at the site, and is configured to adjust the deflection-range of the wing by adjusting an angle between the root portion of the wing and the seat.

Example 315. The system/apparatus according to example 314, wherein: the system/apparatus further comprises an anchor having an anchor head and a tissue-engaging element that extends from the anchor head to define an anchor axis, and the adjustment mechanism is configured to adjust the deflection-range of the wing by adjusting an angle between the root portion of the wing and the anchor axis.

Example 316. The system/apparatus according to example 314, further comprising: a catheter, and an extracorporeal controller, the controller configured to adjust the adjustment mechanism via the catheter.

Example 317. The system/apparatus according to example 316, wherein the adjustment mechanism: comprises a base to which the root portion of the wing is fixedly coupled, and is configured to adjust the deflection-range of the wing by adjusting the angle between the base and the seat.

Example 318. The system/apparatus according to example 317, wherein the adjustment mechanism: comprises a lead screw, and is configured such that rotation of the lead screw adjusts the angle between the base and the seat.

Example 319. The system/apparatus according to example 318, further comprising an anchor, having an anchor head and a tissue-engaging element that extends from the anchor head to define an anchor axis of the anchor, wherein the lead screw defines a lead screw axis that is offset with respect to the anchor axis.

Example 320. The system/apparatus according to example 318, further comprising an anchor, having an anchor head and a tissue-engaging element that extends from the anchor head to define an anchor axis of the anchor, wherein the lead screw defines a lead screw axis that is colinear with the anchor axis.

Example 321. A system and/or an apparatus for use with a real or simulated valve of a real or simulated heart of a real or simulated subject, the valve having an annulus, a first leaflet and an opposing leaflet, the heart having a chamber upstream of the valve, the system/apparatus comprising an implant, the implant comprising: (i) a flexible wing, extending from a root portion of the wing to a tip portion of the wing; (ii) a pair of arms: coupled to the wing, arcing divergently away from the wing, each of the arms: (a) having an anchor point, (b) configured to be anchored to the annulus such that: the arm arcs, from the anchor point, along the annulus to the wing, and the wing is secured in a position in which: (1) the root portion is disposed against the annulus, (2) the wing extends, from the root portion, over the first leaflet toward the opposing leaflet, and (3) responsively to a cardiac cycle of the heart, the wing deflects, in a reciprocating manner, in an upstream direction and in a downstream direction.

Example 322. The system/apparatus according to example 321, wherein the implant is sterile.

Example 323. The system/apparatus according to any one of examples 321-322, wherein each of the pair of arms are away from each other, along a face of the wing.

Example 324. The system/apparatus according to any one of examples 321-323, wherein each of the pair of arms are away from each other, and away from a face of the wing.

Example 325. The system/apparatus according to any one of examples 321-324, wherein each of the pair of arms is: coupled to the root portion of the wing, and arcs divergently away from the root portion of the wing.

Example 326. The system/apparatus according to any one of examples 321-325, further comprising, for each of the arms, a respective pair of anchors, each of the anchors having an anchor head and a tissue-engaging element that extends from the anchor head to define an anchor axis of the anchor that is generally perpendicular to a portion of the respective arm.

Example 327. The system/apparatus according to any one of examples 321-326, wherein each arm is articulatably coupled by a hinge to the wing such that, while the wing is secured in the position by the anchor point of each arm being anchored to the annulus, the arms articulate with respect to the wing in response to reciprocating deflection of the wing.

Example 328. The system/apparatus according to example 327, wherein each arm is articulatably coupled to the wing such that, while the wing is secured in the position by the anchor point of each arm being anchored to the annulus, an angle defined by the pair of arms becomes more acute as the wing deflects in the upstream direction.

Example 329. The system/apparatus according to any one of examples 321-325, wherein the implant further comprises an interface at the root portion of the wing, the interface configured to secure the root portion to the annulus by the interface being anchored to the annulus.

Example 330. The system/apparatus according to example 329, further comprising a plurality of anchors, each anchor: defining an anchor head and a tissue-engaging portion extending from the anchor head along an anchor axis, wherein: the plurality of anchors comprises: a root anchor configured to anchor the interface to the annulus by being driven into tissue of the annulus along a root anchor axis, and a pair of arm anchors, each arm anchor configured to anchor, to the annulus, the anchor point of a respective one of the arms by being driven into tissue of the annulus along an arm anchor axis; and/or while the wing is secured in the position by the plurality of anchors, upstream deflection of the wing is closer to being parallel to the root anchor axis than to either of the arm anchor axes.

Example 331. The system/apparatus according to example 330, wherein while the wing is secured in the position by the plurality of anchors, upstream deflection of the wing is in a direction that is generally parallel to the root anchor axis.

Example 332. The system/apparatus according to any one of examples 329-331, wherein the implant further comprises a limiter, the limiter: coupled to the wing, and configured to inhibit deflection of the wing.

Example 333. The system/apparatus according to example 332, wherein the limiter is configured to define a deflection-limit of the wing during the cardiac cycle of the heart by inhibiting deflection of the wing in the upstream direction beyond the deflection-limit.

Example 334. The system/apparatus according to example 332, wherein the limiter defines a backstop portion that is shaped to press against tissue of the chamber upon anchoring of the interface to the annulus.

Example 335. A system and/or an apparatus for use with a real or simulated valve of a real or simulated heart of a real or simulated subject, the valve having an annulus, a first leaflet and an opposing leaflet opposing the first leaflet, the heart having a first chamber upstream of the valve and a second chamber downstream of the valve, the system/apparatus comprising an implant, (i) the implant comprising: (a) a flexible wing, extending from a root portion of the wing to a tip portion of the wing; and/or (b) a leg, extending from the wing to an end portion of the leg; (ii) wherein the implant is configured to be secured in a position in which: (a) the root portion is disposed against a site at an atrial surface of the annulus, (b) the wing extends, from the root portion, over the first leaflet toward the opposing leaflet, and (b) the leg extends away from the wing to press against an underside of the valve.

Example 336. The system/apparatus according to example 335, wherein the implant is sterile.

Example 337. The system/apparatus according to any one of examples 335-336, wherein: the implant further comprises an interface at the root portion of the wing; and/or the system/apparatus further comprises: an anchor, and a delivery tool, comprising: a catheter, transluminally advanceable to the first chamber, and configured to house the implant, a shaft, housing the anchor, engaged with the interface, and configured, via the engagement with the interface, to, while the anchor remains within the shaft: deploy the implant out of the catheter such that, within the first chamber, the wing extends away from the interface, and position the implant in a position in which: the interface is at the site, the wing extends over the first leaflet toward the opposing leaflet, and the leg extends, from the tip portion, away from the wing and toward a real or simulated tissue of the second chamber, and a driver, engaged with the anchor, and configured to secure the implant in the position by using the anchor to anchor the interface to the annulus.

Example 338. The system/apparatus according to any one of examples 335-336, wherein the implant is configured such that when the root portion of the wing is disposed against the site, and the leg extends away from the wing to press against the underside of the valve, the tip portion of the wing deflects with respect to the root portion of the wing, reciprocatingly in an upstream direction and in a downstream direction, responsively to a cardiac cycle of the heart.

Example 339. The system/apparatus according to example 338, wherein the leg is shaped such that, when the root portion of the wing is disposed against the site, the leg extends away from the root portion of the wing to press against the underside of the valve in a manner that moderates deflection of the wing with respect to the site in the upstream direction.

Example 340. The system/apparatus according to any one of examples 338-339, wherein the leg is shaped such that, when the root portion of the wing is disposed against the site, the leg extends away from the tip portion of the wing to contact tissue of the heart in a manner that moderates deflection of the wing with respect to the site in the upstream direction.

Example 341. The system/apparatus according to any one of examples 338-340, wherein the leg is shaped such that, when the root portion of the wing is disposed against the site, the leg presses against the underside of the valve in a manner that moderates deflection of the wing with respect to the site in the upstream direction.

Example 342. The system/apparatus according to example 341, wherein the implant further comprises an interface at the root portion of the wing, the interface configured to be secured to the site by driving an anchor into tissue at the site.

Example 343. The system/apparatus according to example 341, wherein the leg is shaped such that, when the root portion of the wing is placed against the site, the leg presses against tissue adjacent a commissure of the valve.

Example 344. The system/apparatus according to example 341, wherein leg is shaped such that, when the root portion of the wing is placed against the site, the leg presses against tissue at a subannular groove of the valve.

Example 345. The system/apparatus according to example 341, wherein the implant further comprises an atrial support, the atrial support coupled to the wing and configured such that, when the root portion of the wing is placed against the site, the atrial support presses against the atrial surface of the annulus in a manner that presses the leg against tissue of the second chamber.

Example 346. The system/apparatus according to example 345, wherein the atrial support is shaped to circumscribe the atrial surface of the annulus.

Example 347. The system/apparatus according to example 345, wherein the atrial support is defined by a pair of arms that extend, from the root portion, in opposite directions around the atrial surface of the annulus.

Example 348. The system/apparatus according to example 341, wherein: the second chamber is a left ventricle, the valve is a mitral valve, the first leaflet is a posterior leaflet of the mitral valve, the opposing leaflet is an anterior leaflet of the mitral valve; and/or the leg is shaped such that, when the root portion of the wing is placed against the site, the leg presses against tissue of the left ventricle behind the anterior leaflet.

Example 349. The system/apparatus according to example 348, wherein the leg is shaped such that, when the root portion of the wing is placed against the site, the leg presses against a fibrous trigone of the left ventricle.

Example 350. A system and/or an apparatus for use with a real or simulated valve of a real or simulated heart of a real or simulated subject, the valve having a first leaflet and an opposing leaflet, the heart having a chamber upstream of the valve, the system/apparatus comprising an implant, (i) the implant comprising: (a) a flexible wing, the wing: extending from a root portion of the wing to a tip portion of the wing; and/or a (b) limiter, coupled to the wing; (ii) wherein the implant is configured to be anchored to a site in the chamber such that the implant is secured in a position in which: (a) the wing extends over the first leaflet toward the opposing leaflet, (b) responsively to a cardiac cycle of the heart, the wing deflects, in a reciprocating manner, in an upstream direction and in a downstream direction, and (c) the limiter inhibits deflection of the wing in the upstream direction beyond the deflection-limit by providing an opposing force upon the wing reaching the deflection-limit.

Example 351. The system/apparatus according to example 350, wherein the implant is sterile.

Example 352. The system/apparatus according to any one of examples 350-351, wherein the wing comprises a frame that is laser-cut from a piece of sheet metal.

Example 353. The system/apparatus according to example 352, wherein the frame is shaped to define a buttress at the root portion of the wing, such that the root portion of the wing is stiffer than the tip portion of the wing.

Example 354. The system/apparatus according to any one of examples 350-353, wherein the implant further comprises a pair of arced arms, each arm coupled to the wing at a first portion of the arm, and shaped such that, when the implant is secured in the position, a second portion of the arm contacts tissue of the chamber.

Example 355. The system/apparatus according to example 354, wherein the pair of arms arc symmetrically away from the wing.

Example 356. The system/apparatus according to example 354, wherein the pair of arms are asymmetrically away from the wing.

Example 357. The system/apparatus according to example 354, wherein each arm is coupled to the wing in a manner that allows the arm to pivot with respect to the wing.

Example 358. The system/apparatus according to example 357, wherein the pair of arms are asymmetrically away from the wing such that, when the arms pivot toward each other, the arms become nested with respect to each other.

Example 359. The system/apparatus according to example 354, wherein each arm defines an anchor receiver configured to be anchored to the site by advancing an anchor through the anchor receiver and into tissue of the chamber.

Example 360. The system/apparatus according to example 359, wherein each arm is shaped such that, when the implant is secured in the position, each anchor receiver is disposed adjacent a respective commissure of the valve.

Example 361. The system/apparatus according to example 359, wherein, when the implant is configured such that, when the implant is secured in the position by advancing an anchor through the anchor receiver and into tissue of the chamber, the root portion of the wing maintains contact with the site as the wing deflects in response to the cardiac cycle.

Example 362. The system/apparatus according to example 361, wherein the implant is configured such that, when the implant is secured in the position by advancing an anchor through the anchor receiver and into tissue of the chamber, an angle defined by the arms becomes more acute as the wing deflects in the upstream direction.

Example 363. The system/apparatus according to any one of examples 350-362, wherein the limiter defines a backstop portion that is shaped to press against tissue of the chamber upon the wing reaching the deflection-limit.

Example 364. The system/apparatus according to example 363, wherein the backstop portion is shaped to define an anchor receiver, and the implant is configured to be secured to the site by advancing an anchor through the anchor receiver and into tissue at the site.

Example 365. The system/apparatus according to example 363, wherein the limiter further defines a plurality of ribs that extend from the backstop portion and along the wing, from the root portion of the wing toward the tip portion of the wing.

Example 366. The system/apparatus according to example 365, wherein, while the implant is secured in the position, the limiter inhibits deflection of the wing in the upstream direction beyond the deflection-limit by the ribs providing the opposing force upon the wing reaching the deflection-limit.

Example 367. The system/apparatus according to example 363, wherein the limiter defines a pair of arms, each arm arcing away from the backstop portion.

Example 368. The system/apparatus according to example 367, wherein each arm defines an anchor receiver configured to be anchored to the site by advancing an anchor through the anchor receiver and into tissue of the chamber.

Example 369. The system/apparatus according to example 368, wherein each arm is shaped such that, when the implant is secured in the position, each anchor receiver is disposed adjacent to a respective commissure of the valve.

Example 370. A system for use with a real or simulated valve of a real or simulated heart of a real or simulated subject, the valve having a first leaflet and an opposing leaflet, the heart having a chamber upstream of the valve, the system comprising: (i) an anchor; (ii) an implant, comprising: (a) a wing, extending from a root portion of the wing to a tip portion of the wing; (b) an anchor receiver at the root portion of the wing, and configured to be anchored to a site in the chamber by the anchor extending through the anchor receiver and into tissue at the site; and/or (iii) a delivery tool, comprising: (a) a catheter, transluminally advanceable to the chamber; (b) a shaft disposed within the catheter, the shaft engaged with the implant, and configured, via the engagement with the implant, to: (1) deploy the implant out of the catheter, and (2) position the implant in a position in which the anchor receiver is at the site, and the wing extends over the first leaflet toward the opposing leaflet; and (c) an adjustment rod, reversibly coupled to the wing such that, while the anchor extends through the anchor receiver and into the tissue at the site, axial movement of the adjustment rod adjusts a position of the wing by sliding the anchor receiver with respect to the tissue and the anchor.

Example 371. The system according to example 370, wherein the anchor, the implant and the delivery tool are sterile.

Example 372. A system for use with a real or simulated valve of a real or simulated heart of a real or simulated subject, the valve having a first leaflet and an opposing leaflet, the heart having a chamber upstream of the valve, the system comprising: (i) an anchor, having: (a) an anchor head having a diameter, and (b) a tissue-engaging element that extends from the anchor head; and/or (ii) an implant, (a) comprising an anchor receiver defining an oblong opening delimited by a rim, (b) wherein the implant is configured to be anchored to a site in the heart by the anchor head being seated against the rim of the opening while the tissue-engaging element extends through the opening and into tissue at the site, the opening having: a first dimension that is smaller than the diameter, and a second dimension, transverse to the first dimension, that is greater than the diameter.

Example 373. The system according to example 372, wherein the anchor and the implant are sterile.

Example 374. The system according to any one of examples 372-373, further comprising a delivery tool comprising: (i) a catheter, transluminally advanceable to the chamber; a shaft disposed within the catheter, the shaft engaged with the implant, and configured, via the engagement with the implant, to: (a) deploy the implant out of the catheter, and (b) position the implant in a position in which the implant is at the site, and the implant extends over the first leaflet toward the opposing leaflet; and/or (ii) an adjustment rod, reversibly coupled to the implant such that, while the tissue-engaging element extends through the opening and into tissue at the site, axial movement of the adjustment rod adjusts a position of the implant by sliding the implant with respect to the tissue and the anchor.

Example 375. The system according to any one of examples 372-374, wherein the second dimension is oriented along a length of the implant.

Example 376. The system according to any one of examples 372-375, wherein the implant is configured such that: when the tissue-engaging element extends through the opening, to a first depth of tissue at the site, the implant is slidable along the second dimension, relative to the tissue and the anchor, and when the tissue-engaging element extends through the opening, to a second, greater depth of tissue at the site: the anchor head is seated against the rim, and the implant ceases to be slidable with respect to the anchor.

Example 377. A system for use with a real or simulated heart of a real or simulated subject, the system comprising: (i) an anchor, having: an anchor head, and a tissue-engaging element that extends from the anchor head; and/or (ii) an implant, comprising: (a) an implant body, an interface having a diameter, and (b) an anchor receiver defining an oblong opening delimited by a rim, the opening having: a first dimension that is smaller than the diameter, and a second dimension, transverse to the first dimension, that is greater than the diameter; wherein the anchor receiver is configured to be anchored to a site in the heart by the tissue-engaging element of the anchor extending through the interface and the anchor receiver, into tissue at the site.

Example 378. The system according to example 377, wherein the anchor and the implant are sterile.

Example 379. The system according to any one of examples 377-378, wherein the implant is configured to be anchored to the site by the anchor head seating the interface against the rim of the opening while the tissue-engaging element extends through the interface and the opening, and into tissue at the site.

Example 380. The system according to any one of examples 377-379, further comprising a delivery tool, comprising: a catheter, transluminally advanceable to the heart; a shaft disposed within the catheter, the shaft engaged with the interface, and configured, via the engagement with the interface, to: deploy the implant out of the catheter, and position the implant in a position in which the anchor receiver is at the site; and/or an adjustment rod, reversibly coupled to the implant such that, while the tissue-engaging element extends through the interface and into the tissue at the site, axial movement of the adjustment rod adjusts the position of the implant by sliding the implant body and the anchor receiver with respect to the tissue and the anchor.

Example 381. The system according to example 380, wherein the adjustment rod is reversibly coupled to the implant such that, while the tissue-engaging element extends through the interface and into the tissue at the site, axial movement of the adjustment rod adjusts the position of the implant by sliding the implant body and the anchor receiver with respect to the interface, the tissue and the anchor.

Example 382. The system according to any one of examples 377-381, wherein: the interface comprises: a collar having the diameter, and a neck that is narrower than the collar; and/or the implant is configured to be anchored to the site by the anchor head seating the collar against the rim of the opening while the anchor receiver circumscribes the neck of the interface.

Example 383. The system according to example 382, wherein: the collar is a first collar, the interface further comprises a second collar, and the implant is configured to be anchored to the site by sandwiching the anchor receiver between the first collar and the second collar.

Example 384. A method for use with a real or simulated heart of a real or simulated subject, the method comprising: (i) advancing to the heart: (a) an anchor, the anchor comprising: an anchor head, and a tissue-engaging element that extends from the anchor head, and (b) an implant, the implant comprising an anchor receiver defining an oblong opening delimited by a rim, the oblong opening having a major axis; (ii) advancing the tissue-engaging element of the anchor through the anchor receiver and into tissue at a site of the heart to a first tissue-depth; (iii) while the tissue-engaging element remains within the tissue, sliding the implant along the major axis with respect to the anchor, and/or (iv) subsequently, locking the implant to the anchor such that the implant ceases to be slidable with respect to the anchor.

Example 385. The method according to example 384, further comprising sterilizing the anchor and the implant.

Example 386. The method according to any one of examples 384-385, wherein the step of locking comprises: advancing the tissue-engaging element of the anchor further through the anchor receiver and into tissue at the site, to a second tissue-depth, and seating the anchor head against the rim.

Example 387. A method for use with a simulated heart of a real or simulated subject, the method comprising: (i) advancing to the heart: (a) an anchor, the anchor comprising: an anchor head, and a tissue-engaging element that extends from the anchor head, and (b) an implant, the implant comprising an anchor receiver defining an oblong opening delimited by a rim, the oblong opening having a major axis; (ii) advancing the tissue-engaging element of the anchor through the anchor receiver and into tissue at a site of the heart to a first tissue-depth; (iii) while the tissue-engaging element remains within the tissue, sliding the implant along the major axis with respect to the anchor, and/or (iv) subsequently, locking the implant to the anchor such that the implant ceases to be slidable with respect to the anchor.

Example 388. A method for use with a real or simulated heart of a real or simulated subject, the method comprising: (i) advancing to the heart: (a) an anchor, the anchor comprising: an anchor head, and a tissue-engaging element that extends from the anchor head, and (b) an implant, the implant comprising: (1) an implant body, an (2) interface having a diameter, and (3) an anchor receiver defining an oblong opening delimited by a rim, the oblong opening having a major axis; and/or (ii) anchoring the implant to a site in the heart by: advancing the tissue-engaging element of the anchor, through the interface and the anchor receiver, and into tissue at the site to a first tissue-depth, (iii) subsequently: sliding the implant body, with respect to the interface, along the major axis, and locking implant body to the interface by: advancing the tissue-engaging element of the anchor, further through the interface and the anchor receiver, and into tissue at the site, to a second tissue-depth, and using the anchor head, seating the interface against the rim.

Example 389. The method according to example 388, further comprising sterilizing the anchor and the implant.

Example 390. The method according to any one of examples 388-389, wherein: the interface comprises a first collar and a second collar, and the step of seating the interface comprises, using the anchor head, sandwiching the anchor receiver between the first collar and the second collar.

Example 391. A method for use with a simulated heart of a real or simulated subject, the method comprising: (i) advancing to the heart: (a) an anchor, the anchor comprising: an anchor head, and a tissue-engaging element that extends from the anchor head, and (b) an implant, the implant comprising: an implant body, an interface having a diameter, and an anchor receiver defining an oblong opening delimited by a rim, the oblong opening having a major axis; and/or (ii) anchoring the implant to a site in the heart by: (a) advancing the tissue-engaging element of the anchor, through the interface and the anchor receiver, and into tissue at the site to a first tissue-depth, (b) subsequently: sliding the implant body, with respect to the interface, along the major axis, and (c) locking implant body to the interface by: advancing the tissue-engaging element of the anchor, further through the interface and the anchor receiver, and into tissue at the site, to a second tissue-depth, and using the anchor head, seating the interface against the rim.

Example 392. A system for use with a real or simulated valve of a real or simulated heart of a real or simulated subject, the valve having a first leaflet and an opposing leaflet, the heart having a chamber upstream of the valve, the system comprising: (i) an implant, the implant comprising: (a) a wing, the wing extending from a root portion of the wing to a tip portion of the wing, and (b) an interface at the root portion of the wing, the interface configured to be anchored to a site in the chamber, and (c) a bulking element coupled to the wing; and/or (ii) a delivery tool, comprising: (a) a catheter, transluminally advanceable to the chamber; (b) a shaft disposed within the catheter, the shaft engaged with the interface, and configured, via the engagement with the interface, to: (1) deploy the implant out of the catheter, and (2) position the implant in a position in which the interface is at the site, the wing extends over the first leaflet, and the tip portion is disposed between the first leaflet and the opposing leaflet, and (c) an actuator, operatively coupled to the bulking element such that actuation of the actuator changes a bulkiness of at least a portion of the implant.

Example 393. The system according to example 392, wherein the implant and the delivery tool are sterile.

Example 394. The system according to any one of examples 392-393, wherein the actuator is extracorporeally controllable to transition the bulking element from a delivery state to an actuated state.

Example 395. The system according to any one of examples 392-394, wherein: the bulking element comprises a braided structure, the braided structure having a delivery state and an actuated state, and by transitioning from the delivery state to the actuated state, the braided structure becomes shorter and wider.

Example 396. A method for use with a real or simulated valve of a real or simulated heart of a real or simulated subject, the valve having a first leaflet and an opposing leaflet, the heart having a first chamber upstream of the valve and a second chamber downstream of the valve, the method comprising: (i) within a catheter, advancing to the first chamber: (a) a shaft, and (b) an implant that includes: (1) an interface, engaged with a distal end of the shaft, (2) a flexible wing coupled to the interface, the wing extending from a root portion of the wing to a tip portion of the wing, and (3) a bulking element coupled to the wing; (ii) using the shaft: (a) deploying the implant out of the catheter and into the first chamber, and (b) anchoring the implant in a position in which: the interface is at a site in the first chamber, the wing extends over the first leaflet toward the opposing leaflet, and responsively to a cardiac cycle of the heart, the wing deflects, in a reciprocating manner, in an upstream direction and in a downstream direction; and/or using an actuator, actuating the bulking element in a manner that changes a bulkiness of at least a portion of the implant.

Example 397. The method according to example 396, further comprising sterilizing the catheter, the shaft and the implant.

Example 398. A method for use with a real or simulated valve of a simulated heart of a real or simulated subject, the valve having a first leaflet and an opposing leaflet, the heart having a first chamber upstream of the valve and a second chamber downstream of the valve, the method comprising: (i) within a catheter, advancing to the first chamber: (a) a shaft, and (b) an implant that includes: (1) an interface, engaged with a distal end of the shaft, (2) a flexible wing coupled to the interface, the wing extending from a root portion of the wing to a tip portion of the wing, and (3) a bulking element fixedly coupled to the tip portion of the wing; (ii) using the shaft: deploying the implant out of the catheter and into the first chamber, and/or (iii) anchoring the implant in a position in which: the interface is at a site in the first chamber, the wing extends over the first leaflet toward the opposing leaflet, and responsively to a cardiac cycle of the heart, the wing deflects, in a reciprocating manner, in an upstream direction and in a downstream direction; and/or using an actuator, actuating the bulking element in a manner that changes a bulkiness of the tip portion.

Example 399. A system for use with a real or simulated heart of a real or simulated subject, the system comprising an implant, the implant: configured to be transluminally implanted in the heart, and comprising: (i) a wing, extending from a root portion of the wing to a tip portion of the wing, and (ii) shape-memory member, coupled to the wing, and configured: to be intracardially heated to a temperature greater than 40 degrees C., and such that temporary heating of the shape-memory member to the temperature resizes the wing to a size, wherein the wing is configured to retain the size after cessation of the temporary heating.

Example 400. The system according to example 399, wherein the implant is sterile.

Example 401. The system according to any one of examples 399-400, wherein the wing is configured such that temporary heating of the shape-memory member to the temperature resizes the wing by changing a shape of the shape-memory member.

Example 402. The system according to any one of examples 399-401, wherein the implant comprises a power source that is configured to heat the shape-memory member.

Example 403. The system according to any one of examples 399-402, wherein the implant further comprises an antenna that is configured to wirelessly receive power that heats the shape-memory member.

Example 404. A system for use with a real or simulated valve of a real or simulated heart of a real or simulated subject, the valve having a first leaflet and an opposing leaflet, the heart having a chamber upstream of the valve, the system comprising: (i) an implant, the implant: comprising: (a) a wing, extending from a root portion of the wing to a tip portion of the wing, (b) an interface at the root portion of the wing, and/or (c) a shape-memory member, coupled to the wing, and configured such that temporarily heating the shape-memory member chronically changes a size of the wing; an anchor; (ii) a delivery tool, comprising: (a) a catheter, transluminally advanceable to the chamber; a shaft disposed within the catheter, engaged with the interface and configured, via the engagement, to: deploy the implant out of the catheter, and position the implant in a position in which the interface is at a site upstream of the valve, and the wing extends over the first leaflet toward the opposing leaflet; and/or (b) a driver, engaged with the anchor and configured to secure the implant in the position by using the anchor to anchor the interface to tissue of the heart at the site, the delivery tool being electrically connected to the shape-memory member and configured, via the electrical connection, to electrically heat the shape-memory member.

Example 405. The system according to example 404, wherein the implant, the anchor and the delivery tool are sterile.

Example 406. The system according to any one of examples 404-405, wherein the implant is configured such that temporarily heating the shape-memory member causes a change in shape of the shape-memory member that chronically changes the size of the wing.

Example 407. The system according to any one of examples 404-405, wherein the implant is configured such that heating the shape-memory member causes a change in shape of the shape-memory member.

Example 408. The system according to example 407, wherein the implant comprises a lock, the lock configured to transition between: a locked state in which the change in shape of the shape-memory member does not change a size of the wing, and an unlocked state in which the change in shape of the shape-memory member changes the size of the wing.

Example 409. A system for use with a real or simulated heart of a real or simulated subject, the system comprising: (i) an implant, comprising an interface; (ii) an anchor, comprising: an anchor head, and a tissue-engaging element that extends from the anchor head; and/or (iii) a delivery tool, comprising: (a) a catheter, transluminally advanceable to the heart, (b) a shaft disposed within the catheter, a distal end portion of the shaft engaged with the interface and configured, via the engagement, to: (1) deploy the implant out of the catheter, and (2) position the implant such that the interface is disposed at a site in the heart, and (c) a driver, engaged with the anchor and configured to secure the implant at the site by using the anchor to anchor the interface to tissue of the heart at the site, wherein a distal segment of the shaft: has (i) a rigid state, and (ii) a flexible state in which the distal segment is more flexible than when the distal segment assumes the rigid state, and is transitionable between the rigid state and the flexible state while the distal end portion remains connected to the interface.

Example 410. The system according to example 409, wherein at least one of the implant, the anchor, and the delivery tool is sterile.

Example 411. The system according to any one of examples 409-410, wherein: the shaft defines a shaft-lumen, the driver is slidably advanceable through the shaft-lumen such that a drive head of the driver is engaged with the anchor head, and the driver comprises a driveshaft that is more flexible than the distal segment of the shaft while the distal segment assumes the rigid state.

Example 412. The system according to any one of examples 409-411, wherein the distal segment has an outer diameter that is no more than 10 percent greater than an outer diameter of a proximal portion of the shaft.

Example 413. The system according to any one of examples 409-412, wherein: the distal segment of the shaft comprises a spring, and the distal segment is transitionable between the rigid state and the flexible state by altering tension on the spring.

Example 414. The system according to any one of examples 409-413, wherein: the distal segment of the shaft comprises a tether, and the distal segment is transitionable between the rigid state and the flexible state by altering tension on the tether.

Example 415. The system according to any one of examples 409-414, wherein the distal segment of the shaft is transitionable between the rigid state and the flexible state while the driver remains engaged with the anchor.

Example 416. The system according to example 415, wherein the distal segment of the shaft is transitionable between the rigid state and the flexible state while the interface is anchored to tissue of the heart at the site.

Example 417. The system according to any one of examples 409-416, wherein: the distal segment of the shaft comprises a hinge, and the distal segment is transitionable between the rigid state and the flexible state by regulating articulation of the hinge.

Example 418. The system according to example 417, configured such that transitioning the distal segment from the rigid state to the flexible state increases an articulation-range of the hinge along an articulation axis.

Example 419. The system according to example 418, wherein: the hinge is a first hinge and the articulation axis is a first articulation axis, the distal segment further comprises a second hinge configured to articulate along a second articulation axis that is nonparallel to the first articulation axis, and the distal segment is configured such that transitioning the distal segment from the rigid state to the flexible state increases a second articulation-range of the hinge along the second articulation axis.

Example 420. The system according to example 419, wherein the second articulation axis is orthogonal to the first articulation axis.

Example 421. The system according to example 417, wherein the distal segment is transitionable between the rigid state and the flexible state by moving the anchor longitudinally through the distal segment.

Example 422. The system according to example 421, wherein the distal segment is transitionable from the rigid state to the flexible state by anchoring the interface to tissue of the heart at the site.

Example 423. A method for use with a real or simulated valve of a real or simulated heart of a real or simulated subject, the method comprising: (i) using a catheter, advancing an implant including an interface to the heart; (ii) using a shaft, a distal end portion of the shaft coupled to the interface, deploying the implant out of a distal opening of the catheter; (iii) using a driver that is engaged to an anchor, anchoring the interface to tissue at a site of the heart by driving an anchor into the tissue; and/or (iv) subsequently to the step of deploying: while the distal end portion of the shaft remains coupled to the interface, transitioning a distal segment of the shaft from a rigid state to a flexible state in which the distal segment is more flexible than when the distal segment assumes the rigid state, and subsequently: disengaging the driver from the anchor; and/or (v) decoupling the distal end portion of the shaft from the interface.

Example 424. The method according to example 423, wherein the step of transitioning is subsequent to the step of anchoring.

Example 425. The method according to example 423, wherein: the distal segment of the shaft comprises a docking station at which the distal end portion of the shaft is reversibly coupled to a proximal portion of the shaft, and the step of transitioning comprises transitioning the distal segment of the shaft from the rigid state to the flexible state by decoupling the distal end portion of the shaft from the proximal portion of the shaft.

Example 426. The method according to example 425, wherein: the shaft includes a tether, and the step of transitioning comprises transitioning the distal segment of the shaft from the rigid state to the flexible state by decoupling the distal end portion of the shaft from the proximal portion of the shaft by reducing tension on the tether.

Example 427. The method according to example 426, wherein the method further comprises, prior to the step of disengaging, recoupling the distal end portion of the shaft to the proximal portion of the shaft by increasing tension on the tether.

Example 428. The method according to example 423, further comprising sterilizing the implant, the shaft and the catheter.

Example 429. The method according to any one of examples 423-428, wherein: the shaft includes a tether, and the step of transitioning comprises transitioning the distal segment from the rigid state to the flexible state by adjusting tension on the tether.

Example 430. The method according to any one of examples 423-429, wherein: the distal segment of the shaft includes a spring, and the step of transitioning comprises transitioning the distal segment from the rigid state to the flexible state by adjusting tension on the spring.

Example 431. The method according to any one of examples 423-430, further comprising, using the shaft, prior to the step of anchoring, positioning the interface at the site prior to anchoring the interface to the tissue.

Example 432. The method according to example 431, wherein the step of positioning comprises positioning the interface at the site while the distal segment of the shaft assumes the rigid state.

Example 433. The method according to any one of examples 423-432, further comprising, subsequently to the step of anchoring: re-transitioning the distal segment of the shaft from the flexible state to the rigid state, and withdrawing the shaft and the driver from the subject.

Example 434. The method according to example 433, further comprising, prior to the step of re-transitioning, assessing function of the valve.

Example 435. The method according to example 434, wherein the step of assessing is prior to the step of disengaging.

Example 436. The method according to example 434, wherein the step of assessing is prior to the step of decoupling.

Example 437. The method according to example 434, wherein: the site is a first site; and/or the method further comprises, responsively to the step of assessing: using the driver, removing the anchor from the tissue at the first site, using the shaft, redeploying the implant to a second site of the heart, and using the driver, re-anchoring the interface to tissue at the second site of the heart by driving the anchor into the tissue at the second site.

Example 438. The method according to example 437, wherein the step of re-transitioning is prior to the step of re-anchoring.

Example 439. The method according to any one of examples 423-438, wherein the step of transitioning comprises transitioning the distal segment from the rigid state to the flexible state by driving the anchor through the interface and into the tissue.

Example 440. The method according to example 439, wherein: the distal segment includes a hinge, and the step of transitioning comprises increasing a range of articulation of the hinge along an articulation-axis.

Example 441. The method according to example 440, wherein the hinge is a first hinge having a first range of articulation along a first articulation-axis, the distal segment further includes a second hinge having a second range of articulation along a second articulation-axis, and the step of transitioning further comprises increasing the second range of articulation of the second hinge.

Example 442. A method for use with a real or simulated valve of a simulated heart of a real or simulated subject, the method comprising: (i) using a catheter, advancing an implant including an interface to the heart; (ii) using a shaft, a distal end portion of the shaft coupled to the interface, deploying the implant out of a distal opening of the catheter; (iii) using a driver that is engaged to an anchor, anchoring the interface to tissue at a site of the heart by driving an anchor into the tissue; and/or (iv) subsequently to the step of deploying: while the distal end portion of the shaft remains coupled to the interface, transitioning a distal segment of the shaft from a rigid state to a flexible state in which the distal segment is more flexible than when the distal segment assumes the rigid state, and subsequently: disengaging the driver from the anchor; and/or (v) decoupling the distal end portion of the shaft from the interface.

Example 443. The method according to example 442, wherein the step of transitioning is subsequent to the step of anchoring.

Example 444. The method according to example 442, wherein: the distal segment of the shaft comprises a docking station at which the distal end portion of the shaft is reversibly coupled to a proximal portion of the shaft, and the step of transitioning comprises transitioning the distal segment of the shaft from the rigid state to the flexible state by decoupling the distal end portion of the shaft from the proximal portion of the shaft.

Example 445. The method according to example 444, wherein: the shaft includes a tether, and the step of transitioning comprises transitioning the distal segment of the shaft from the rigid state to the flexible state by decoupling the distal end portion of the shaft from the proximal portion of the shaft by reducing tension on the tether.

Example 446. The method according to example 445, wherein the method further comprises, prior to the step of disengaging, recoupling the distal end portion of the shaft to the proximal portion of the shaft by increasing tension on the tether.

Example 447. A system for use with a real or simulated valve of a real or simulated heart of a real or simulated subject, the valve having a first leaflet and an opposing leaflet, the heart having a chamber upstream of the valve, the system comprising: (i) an implant, the implant comprising: (a) a wing, extending from a root portion of the wing to a tip portion of the wing, and (b) an interface at the root portion of the wing; (ii) an anchor, rotatably coupled and axially fixed to the interface, the anchor comprising: an anchor head, and a tissue-engaging element extending from the anchor head to define an anchor axis of the anchor; and/or (iii) a delivery tool, comprising: (a) a catheter, transluminally advanceable to the chamber; (b) a shaft disposed within the catheter, engaged with the interface and configured, via the engagement, to: (1) deploy the implant out of the catheter, and (2) position the implant in a position in which the interface is at a site upstream of the valve, and the wing extends over the first leaflet toward the opposing leaflet; (c) a driver, engaged with the anchor and configured to secure the implant in the position by using the anchor to anchor the interface to tissue of the heart at the site.

Example 448. The system according to example 447, wherein at least one of the implant, the anchor and the delivery tool is sterile.

Example 449. A system for use with a real or simulated heart of a real or simulated subject, the system comprising: (i) an implant, the implant comprising: an interface, an anchor receiver, and a wing, coupled to the interface and to the anchor receiver; (ii) an anchor; and/or (iii) a delivery tool, comprising: (a) a catheter that is transluminally advanceable to the heart, the catheter defining a distal opening and a lateral opening, (b) a shaft disposed within the catheter, engaged with the interface and configured, via the engagement, to: deploy the implant out of the distal opening of the catheter, and position the implant such that the anchor receiver is disposed at a site of the heart, and (c) a driver, engaged with the anchor and configured to secure the implant to the heart by: advancing the anchor out of the lateral opening of the catheter and toward the anchor receiver, and driving the anchor through the anchor receiver and into tissue of the heart at the site.

Example 450. The system according to example 449, wherein at least one of the implant, the anchor, and the delivery tool is sterile.

Example 451. The system according to any one of examples 449-450, wherein: the implant has a compressed state and an expanded state, and comprises: a flexible frame that comprises a shape-memory material and biases the implant toward assuming the expanded state, and an expansion element, having: a compact state, and an extended state in which the expansion element resists compression of the implant toward the compressed state, wherein: the catheter is configured to house the implant while the implant is in the compressed state and the expansion element is in the compact state, and the shaft is configured, via the engagement to deploy the implant out of the distal opening of the catheter such that, within the heart, the implant assumes the expanded state and the expansion element assumes the extended state.

Example 452. The system according to example 451, wherein the implant is configured such that extension of the expansion element from the compact state to the extended state applies an expansion force to the implant, the expansion force facilitating expansion of the implant from the compressed state to the expanded state.

Example 453. The system according to example 451, wherein the expansion element is configured to resist transition from the extended state toward the compact state.

Example 454. The system according to example 451, wherein the expansion element comprises a spring.

Example 455. The system according to example 453, wherein the expansion element comprises a plurality of subunits, and the expansion element is configured such that extending the expansion element into the extended state causes the subunits to fit together.

Example 456. The system according to example 455, wherein the expansion element comprises a plurality of subunits, configured to lock together upon the expansion element assuming the extended state.

Example 457. The system according to example 455, wherein the expansion element is straighter in the extended state than in the compact state.

Example 458. The system according to example 457, wherein the expansion element comprises a hinge, and the expansion element is configured such that straightening the hinge straightens the expansion element.

Example 459. The system according to example 458, wherein the delivery tool further comprises an extension actuator, the extension actuator configured to transluminally extend the expansion element from the compact state to the extended state.

Example 460. The system according to any one of examples 449-459, wherein: the anchor is a first anchor, and the system further comprises a second anchor; the anchor receiver is a first anchor receiver, and the implant further comprises a second anchor receiver; the driver is a first driver, and the delivery tool further comprises a second driver; the lateral opening is a first lateral opening, and the catheter further defines a second lateral opening opposite the first lateral opening; and/or the first and second drivers are each engaged with a respective anchor and configured to secure the implant to the heart by: advancing out of one of the lateral openings and toward one of the anchor receivers, and driving one of the first and second anchors through one of the anchor receivers and into tissue of the heart at the site.

Example 461. The system according to example 460, wherein the first and second drivers are configured to diverge away from each other as the first and second drivers advance out of one of the lateral openings and toward the anchor receivers.

Example 462. The system according to any one of examples 449-459, wherein: the catheter further comprises a gate at the lateral opening, the gate comprising a shape-memory material; and/or the catheter is configured to transition between: a delivery state in which the gate is closed, and a deployment state in which the gate is open.

Example 463. The system according to example 462, configured such that deploying the implant out of the distal opening facilitates transitioning the catheter from the delivery state to the deployment state.

Example 464. The system according to example 462, wherein the catheter is configured such that while the catheter is in the deployment state, the open gate guides the driver and the anchor out of the lateral opening of the catheter and toward the anchor receiver.

Example 465. A method for use with a real or simulated heart of a real or simulated subject, the method comprising: (i) advancing to the heart an implant compressed within a catheter, the implant including: an interface, an anchor receiver, and a wing, coupled to the interface and to the anchor receiver; (ii) using a shaft coupled to the interface, deploying the implant out of a distal opening of the catheter such that the wing expands within the heart; (iii) using a driver: advancing an anchor out of a lateral opening of the catheter and to the anchor receiver, and anchoring the implant to tissue of the heart by driving a tissue-engaging element of the anchor through the anchor receiver and into the tissue.

Example 466. The method according to example 465, further comprising sterilizing the implant and the catheter.

Example 467. The method according to any one of examples 465-466, wherein: the step of deploying comprises deploying the implant out of a distal opening of the catheter in a direction that is generally parallel to a longitudinal axis of a distal portion of the shaft, and the step of advancing comprises advancing the anchor out of the lateral opening of the catheter in a direction that is oblique with respect to the longitudinal axis of the distal portion of the shaft.

Example 468. The method according to any one of examples 465-467, wherein: the driver is a first driver; the lateral opening is a first lateral opening of the catheter; the anchor receiver is a first anchor receiver, and the implant further includes a second anchor receiver; the step of advancing comprises, using the first driver and a second driver: advancing a first anchor out of the first lateral opening of the catheter and to the first anchor receiver, and advancing a second anchor out of a second lateral opening of the catheter and to the second anchor receiver; and/or the step of anchoring comprises anchoring the implant to tissue of the heart by driving each anchor through a respective anchor receiver and into tissue of the heart.

Example 469. The method according to example 468, wherein the step of advancing comprises advancing the first driver and the second driver divergently away from each other.

Example 470. The method according to any one of examples 465-469, wherein: the catheter further comprises a shape-memory gate at the lateral opening; and/or the method further comprises transitioning the catheter from a delivery state in which the gate is closed, to a deployment state in which the gate is open.

Example 471. The method according to example 470, wherein the step of transitioning comprises transitioning the catheter from the delivery state to the deployment state by deploying the implant out of the distal opening of the catheter.

Example 472. The method according to example 470, wherein the step of transitioning comprises transitioning the catheter from the delivery state to the deployment state by proximally retracting the driver within the catheter.

Example 473. A method for use with a simulated heart of a real or simulated subject, the method comprising: (i) advancing to the heart an implant compressed within a catheter, the implant including: an interface, an anchor receiver, and a wing, coupled to the interface and to the anchor receiver; (ii) using a shaft coupled to the interface, deploying the implant out of a distal opening of the catheter such that the wing expands within the heart; (iii) using a driver: advancing an anchor out of a lateral opening of the catheter and to the anchor receiver, and anchoring the implant to tissue of the heart by driving a tissue-engaging element of the anchor through the anchor receiver and into the tissue.

Example 474. A system for use with a real or simulated heart of a real or simulated subject, the system comprising: (i) an implant, the implant comprising an interface; (ii) an anchor; (iii) a catheter that is transluminally advanceable to the heart; (iv) a latch; (v) a shaft: disposed within the catheter, reversibly engaged to the interface via the latch, and configured, via the engagement, to: deploy the implant out of the catheter, and position the implant in a position in which the interface is disposed at a site of the heart; (vi) a driver, disposed within the catheter, and configured to anchor the implant in the position by driving the anchor into tissue at the site; and/or (vii) an insert, disposed between the shaft and the interface, and slidable in a manner that disengages the shaft from the interface by displacing the latch.

Example 475. The system according to example 474, wherein at least one of the implant, the anchor, the catheter and the shaft is sterile.

Example 476. The system according to any one of examples 474-475, wherein the shaft is shaped to define the latch.

Example 477. The system according to any one of examples 474-476, wherein the driver is shaped to define the insert.

Example 478. The system according to any one of examples 474-477, wherein the driver is configured to anchor the implant in the position by driving the anchor through the interface and into tissue at the site.

Example 479. The system according to any one of examples 474-478, configured such that the driver extends distally, from outside the subject, within the catheter and through the shaft.

Example 480. The system according to any one of examples 474-479, wherein: the interface is shaped to define a window; and/or the system is configured such that: while the shaft is engaged to the interface, the latch is disposed within the window, and the insert is slidable in a manner that disengages the shaft from the interface by displacing the latch from within the window.

Example 481. The system according to any one of examples 474-480, wherein: the anchor defines an anchor head and a helical tissue-engaging element that extends away from the anchor head along an anchor axis, and the system is configured such that the insert is rotatable with respect to the latch about the anchor axis in a manner that disengages the shaft from the interface by displacing the latch.

Example 482. The system according to any one of examples 474-480, wherein: the anchor defines an anchor head and a helical tissue-engaging element that extends away from the anchor head along an anchor axis, and the system is configured such that the insert is slidable with respect to the latch along the anchor axis in a manner that disengages the shaft from the interface by displacing the latch.

Example 483. The system according to any one of examples 474-482, wherein: the latch comprises a shape-memory material having a compressed shape and a relaxed shape, and the insert is slidable in a manner that disengages the shaft from the interface by causing the latch to: transition between the compressed shape and the relaxed shape, and disengage from the interface.

Example 484. The system according to any one of examples 474-484, wherein the insert comprises an intervening tube disposed between the shaft and the interface.

Example 485. The system according to example 484, wherein the intervening tube is slidable with respect to the shaft and the interface.

Example 486. The system according to example 484, wherein the intervening tube circumscribes a portion of the driver.

Example 487. The system according to example 484, wherein the intervening tube circumscribes a portion of the anchor.

Example 488. The system according to example 484, wherein the intervening tube circumscribes a portion of the interface.

Example 489. A method for use at a real or simulated heart of a real or simulated subject, the method comprising: (i) advancing to the heart: (a) an anchor, an implant defining an interface, (b) a catheter housing the implant, (c) a latch, (d) a shaft reversibly engaged to the interface via the latch, and (e) an insert disposed between the shaft and the interface; (ii) deploying the implant out of the catheter; (iii) using the shaft, positioning the implant in a position in which the interface is disposed at a site of the heart; (iv) using a driver, securing the implant in the position by driving a portion of the anchor through the interface and into tissue at the site; and/or (v) subsequently, disengaging the shaft from the implant by sliding the insert between the shaft and the interface.

Example 490. The method according to example 489, further comprising sterilizing the implant, the anchor, the shaft and the catheter.

Example 491. The method according to any one of examples 489-490, wherein the step of disengaging comprises displacing the latch from the interface.

Example 492. The method according to example 491, wherein: the interface is shaped to define a window; and/or the step of disengaging comprises displacing the latch from within the window.

Example 493. The method according to example 491, wherein: the latch comprises a shape-memory material having a compressed shape and a relaxed shape, and the step of disengaging comprises displacing the latch from the interface by sliding the insert between the shaft and the interface in a manner that allows the latch to transition between the compressed shape and the relaxed shape.

Example 494. The method according to example 491, wherein step of disengaging comprises displacing the latch from the interface by sliding the insert between the shaft and the interface in a manner that expands the latch laterally with respect to the interface.

Example 495. The method according to example 491, wherein: the anchor defines: an anchor head, and an anchor axis along which a helical tissue-engaging element extends from the anchor head; and/or the step of disengaging comprises displacing the latch from the interface by sliding the insert with respect to the latch along the anchor axis.

Example 496. The method according to example 491, wherein: the anchor defines: an anchor head, and an anchor axis along which a helical tissue-engaging element extends from the anchor head; and/or the step of disengaging comprises displacing the latch from the interface by rotating the insert with respect to the latch about the anchor axis.

Example 497. The method according to example 496, wherein: (i) the anchor is a first anchor, the interface is a first interface, and the implant further includes a second interface that defines a longitudinal axis along which the second interface is configured to receive a second anchor, the latch is a first latch, the shaft is a first branch of the shaft, and the insert is a first insert; and/or (ii) the method further comprises advancing to the heart: (a) the second anchor, a second latch, a second branch of the shaft reversibly engaged to the second interface via the second latch, and a second insert disposed between the second branch of the shaft and the second interface; and/or (iii) the step of disengaging comprises disengaging the shaft from the implant by: rotating the first insert with respect to the first latch in a first direction about the longitudinal axis of the first interface, and rotating the second insert with respect to the second latch in a second, opposite direction about the longitudinal axis of the second interface.

Example 498. A method for use at a simulated heart of a real or simulated subject, the method comprising: (i) advancing to the heart: an anchor, an implant defining an interface, a catheter housing the implant, a latch, a shaft reversibly engaged to the interface via the latch, and an insert disposed between the shaft and the interface; (ii) deploying the implant out of the catheter; (iii) using the shaft, positioning the implant in a position in which the interface is disposed at a site of the heart; (iv) using a driver, securing the implant in the position by driving a portion of the anchor through the interface and into tissue at the site; and/or (v) subsequently, disengaging the shaft from the implant by sliding the insert between the shaft and the interface.

Example 499. A system for use with a real or simulated valve of a real or simulated heart of a real or simulated subject, the valve having a first leaflet and an opposing leaflet, the heart having a chamber upstream of the valve, the system comprising: (i) an anchor; and/or (ii) an implant, the implant comprising: (a) a wing, extending from a root portion of the wing to a tip portion of the wing, and (b) an anchor receiver: configured to promote tissue ingrowth thereon, and coupled to the root portion of the wing such that anchoring of the anchor receiver to an annulus of the valve positions the wing such that: (1) the wing extends over the first leaflet toward the opposing leaflet, and (2) responsively to a cardiac cycle of the heart, the wing deflects, in a reciprocating manner, in an upstream direction and in a downstream direction, the implant defining an obstacle that is configured to inhibit the tissue ingrowth from progressing from the anchor receiver toward the tip portion of the wing.

Example 500. The system according to example 499, wherein at least one of the implant and the anchor is sterile.

Example 501. The system according to any one of examples 499-500, wherein: the wing defines a contact face, and an opposing face opposite to the contact face, and the obstacle comprises a cage, the cage: disposed on the opposing face, at the root portion of the wing, and comprising a barrier configured to mechanically inhibit the tissue ingrowth from progressing from the anchor receiver toward the tip portion of the wing.

Example 502. The system according to example 501, wherein: the implant has an interface at the root portion of the wing; and/or the barrier is a bilayer barrier comprising: an interface-facing layer of the barrier comprising a material that promotes tissue growth thereupon, and an opposing layer comprising material that inhibits tissue growth thereupon.

Example 503. The system according to example 501, wherein the cage defines a backstop portion that is shaped to press against tissue of the chamber upon anchoring of the anchor receiver to the annulus.

Example 504. The system according to any one of examples 499-503, wherein the obstacle is configured to inhibit the tissue ingrowth from progressing from the anchor receiver toward the tip portion of the wing by reducing contact between the root portion of the wing and the first leaflet.

Example 505. The system according to example 504, wherein the wing is shaped to define the obstacle such that, while the anchor receiver is anchored to the annulus: the root portion of the wing curves in the upstream direction, away from the first leaflet, and the tip portion of the wing curves in the downstream direction, toward the first leaflet.

Example 506. The system according to any one of examples 499-505, wherein: the wing defines a contact face, and an opposing face opposite to the contact face, and the obstacle comprises a stilt attached to the contact face at the root portion of the wing, the stilt configured such that while the wing extends over the first leaflet toward the opposing leaflet, the stilt inhibits contact between the root portion of the wing and the first leaflet.

Example 507. A system for use at a site in a real or simulated heart of a real or simulated subject, the system comprising: (i) an implant, comprising an anchor receiver; and/or (ii) an anchor, comprising: an anchor head, and a helical tissue-engaging element that extends distally from the anchor head along an anchor axis, and configured to be screwed through the anchor receiver and into tissue by rotation of the anchor head in a rotational direction at least until the anchor head reaches the anchor receiver, wherein, the anchor head and the anchor receiver are shaped such that, upon the anchor head reaching the anchor receiver, further rotation of the anchor head in the rotational direction pushes the anchor receiver distally with respect to the anchor.

Example 508. The system according to example 507, wherein at least one of the implant and the anchor is sterile.

Example 509. The system according to any one of examples 507-508, further comprising a delivery tool comprising: a catheter, transluminally advanceable to the heart; a shaft disposed within the catheter, a distal end portion of the shaft engaged with the implant and configured, via the engagement, to: deploy the implant out of the catheter, and position the implant such that the implant is disposed at the site; and/or a driver configured to secure the implant at the site by using the anchor to anchor the anchor receiver to tissue of the heart at the site.

Example 510. The system according to any one of examples 507-509, wherein the anchor head and the anchor receiver are shaped such that, upon the anchor head reaching the anchor receiver, further rotation of the anchor head in the rotational direction pushes the anchor head proximally away from the anchor receiver.

Example 511. The system according to example 510, wherein the anchor head and the anchor receiver are each shaped to define complementarily undulating surfaces along which the anchor head interfaces with the anchor receiver.

Example 512. A method for use at a real or simulated heart of a real or simulated subject, the method comprising: (i) advancing to the heart: (a) an anchor including an anchor head and a tissue-engaging portion extending from the anchor head, and (b) an implant compressed within a catheter, the implant including an anchor receiver; (ii) using a shaft coupled to the anchor receiver, deploying the implant out of a distal opening of the catheter such that the anchor receiver is disposed at a site of the heart; (iii) using a driver, screwing the anchor through the anchor receiver and into tissue at the site by rotating the anchor head at least until the anchor head reaches the anchor receiver; and/or (iv) after the anchor head reaches the anchor receiver, pushing the anchor receiver into tissue at the site by further rotating the anchor head.

Example 513. The method according to example 512, further comprising sterilizing the shaft, the anchor and the implant.

Example 514. A method for use at a simulated heart of a real or simulated subject, the method comprising: (i) advancing to the heart: (a) an anchor including an anchor head and a tissue-engaging portion extending from the anchor head, and (b) an implant compressed within a catheter, the implant including an anchor receiver; (ii) using a shaft coupled to the anchor receiver, deploying the implant out of a distal opening of the catheter such that the anchor receiver is disposed at a site of the heart; (iii) using a driver, screwing the anchor through the anchor receiver and into tissue at the site by rotating the anchor head at least until the anchor head reaches the anchor receiver; and/or (iv) after the anchor head reaches the anchor receiver, pushing the anchor receiver into tissue at the site by further rotating the anchor head.

Example 515. A system for use at a real or simulated tissue site of a real or simulated subject, (i) the system comprising: an implant comprising (a) a frame and (b) an interface, the interface comprising: a first collar, configured to interface with an anchor head, a second collar, and a neck portion, connecting the first collar to the second collar, and extending through a loop portion of the frame; (2) wherein: the interface has: a loose state in which the loop portion is loosely coupled to the interface, and a tight state in which the loop portion is sandwiched between the first collar and the second collar.

Example 516. The system according to example 515, wherein the implant is sterile.

Example 517. The system according to any one of examples 515-516, further comprising an anchor, wherein the interface is configured to transition from the loose state to the tight state by advancing the anchor distally through the interface.

Example 518. The system according to example 517, wherein: the anchor comprises the anchor head and a tissue-engaging portion extending away from the anchor head along an anchor axis, and the system is configured such that while the interface is in the loose state, at least a portion of the tissue-engaging portion protrudes distally through the interface.

Example 519. The system according to example 517, wherein: the anchor comprises the anchor head and a tissue-engaging portion extending away from the anchor head along an anchor axis, and the interface is configured to transition from the loose state to the tight state by advancing the tissue-engaging portion distally through the interface such that: the anchor head presses distally against the interface, and the tissue-engaging portion passes distally through the interface.

Example 520. The system according to example 519, configured such that a deflectability of the implant along the anchor axis is reduced as the interface transitions from the loose state to the tight state.

Example 521. The system according to example 517, further comprising a delivery tool comprising: a catheter, transluminally advanceable to the tissue site; a shaft disposed within the catheter, a distal end portion of the shaft engaged with the interface and configured, via the engagement, to: deploy the implant out of the catheter, and position the implant such that the interface is disposed at the tissue site; and/or a driver configured to: secure the implant at the tissue site, and transition the interface from the loose state to the tight state by advancing the anchor distally through the interface.

Example 522. The system according to example 521, configured such that while the interface is in the loose state: the drive head is engaged with the anchor head, and the shaft is engaged with the interface.

Example 523. The system according to example 522, wherein the interface is configured to transition from the loose state to the tight state while: the drive head remains engaged with the anchor head, and the shaft remains engaged with the interface.

Example 524. The system according to example 522, configured such that while the interface is in the loose state, the implant is deflectable with respect to the interface along an assessment deflection-range that is generally equal to a deployment deflection-range along which the implant is deflectable while the system assumes a deployment state in which: the interface is in the tight state, and the shaft is disengaged from the interface.

Example 525. A method for use at a real or simulated tissue of a real or simulated subject, the method comprising: (i) using a catheter, advancing to a site of the tissue: (a) an anchor defining an anchor head and a tissue-engaging portion extending away from the anchor head along an anchor axis, and (b) an implant including (1) a frame and (2) an interface, the interface including: a first collar, configured to interface with the anchor head, a second collar, and a neck portion, connecting the first collar to the second collar, and extending through a loop portion of the frame; and/or (ii) using a driver, transitioning the interface from: a loose state in which the loop portion is loosely coupled to the interface, to a tight state in which the loop portion is sandwiched between the first collar and the second collar.

Example 526. The method according to example 525, further comprising sterilizing the anchor and the implant.

Example 527. The method according to any one of examples 525-526, wherein: the step of transitioning comprises transitioning the interface from the loose state to the tight state by advancing the anchor through the interface and into tissue at the site.

Example 528. The method according to example 527, wherein the step of transitioning comprises transitioning the interface from the loose state to the tight state such that the anchor head presses distally against the interface.

Example 529. The method according to example 527, wherein the step of transitioning comprises reducing a deflectability of the implant along the anchor axis.

Example 530. A method for use at a simulated tissue of a real or simulated subject, the method comprising: (i) using a catheter, advancing to a site of the tissue: (a) an anchor defining an anchor head and a tissue-engaging portion extending away from the anchor head along an anchor axis, and (b) an implant including a (1) frame and (2) an interface, the interface including: a first collar, configured to interface with the anchor head, a second collar, and a neck portion, connecting the first collar to the second collar, and extending through a loop portion of the frame; and/or (ii) using a driver, transitioning the interface from: (a) a loose state in which the loop portion is loosely coupled to the interface, to (b) a tight state in which the loop portion is sandwiched between the first collar and the second collar.

Example 531. A system for use with a real or simulated valve of a real or simulated heart of a real or simulated subject, the valve having a first leaflet and an opposing leaflet, the heart having a chamber upstream of the valve, the system comprising: (i) an anchor defining an anchor axis; (ii) an implant, comprising: (a) a wing; (b) an interface at a root portion of the wing; and/or (iii) a delivery tool, (a) comprising: (1) a catheter, transluminally advanceable to the heart, (2) a shaft disposed within the catheter, (3) a coupling, attached to a distal end of the shaft, the shaft being configured, via engagement of the coupling with the interface, to: deploy the implant out of the catheter, and position the implant in a position in which: the wing extends over the first leaflet toward the opposing leaflet, and responsively to a cardiac cycle of the heart, the wing deflects, in a reciprocating manner, in an upstream direction and in a downstream direction; a driver, configured to secure the implant in the position by driving the anchor into tissue of the heart; (b) wherein the delivery tool is configured to transition the system between: a first state, in which the coupling is engaged with the interface, and a second state, in which the coupling is engaged with the interface, and the wing has greater deflectability with respect to the anchor axis than in the first state, and a deployed state, in which the coupling is disengaged from the interface.

Example 532. The system according to example 531, wherein at least one of the implant, the anchor, and the delivery tool is sterile.

Example 533. The system according to example 531, wherein: the shaft has a proximal portion, proximal from the coupling and the distal end of the shaft, while the system is in the first state, the distal end of the shaft is secured to the proximal portion of the shaft, and the delivery tool is configured to transition the system into the second state by releasing the distal end of the shaft from the proximal portion of the shaft.

Example 534. The system according to example 533, wherein the delivery tool is configured such that while the delivery tool is in the deployed state, the distal end of the shaft is secured to the proximal portion of the shaft.

Example 535. The system according to example 533, wherein: the shaft further comprises a tether, and the delivery tool is configured to transition between the first state and the second state by adjusting tension on the tether.

Example 536. The system according to example 535, wherein the distal end of the shaft is reversibly coupled to the proximal portion of the shaft, such that: increasing tension on the tether secures the distal end of the shaft to the proximal portion of the shaft, and reducing tension on the tether decouples the distal end of the shaft from the proximal portion of the shaft.

Example 537. The system according to any one of examples 531-532, wherein the deflectability of the wing along the anchor axis while the system is in the second state is generally equal to the deflectability of the wing along the anchor axis while the system is in the deployed state.

Example 538. The system according to any one of examples 531-537, wherein the coupling has an outer diameter that is no more than 10 percent greater than an outer diameter of a proximal portion of the shaft.

Example 539. The system according to any one of examples 531-538, wherein: the coupling comprises a spring, and the system is transitionable between the first state and the second state by altering tension on the spring.

Example 540. The system according to any one of examples 531-539, wherein: the coupling comprises a tether, and the system is transitionable between the first state and the second state by altering tension on the tether.

Example 541. The system according to any one of examples 531-540, wherein the system is transitionable between the first state and the second state while the driver is engaged with the anchor.

Example 542. The system according to example 541, wherein the system is transitionable between the first state and the second state while the interface is anchored to tissue of the heart.

Example 543. The system according to any one of examples 531-542, wherein: the coupling comprises a hinge, and the system is transitionable between the first state and the second state by regulating articulation of the hinge.

Example 544. The system according to example 543, configured such that transitioning the system from the second state to the first state increases an articulation-range of the hinge along an articulation axis.

Example 545. The system according to example 544, wherein: the hinge is a first hinge and the articulation axis is a first articulation axis, the coupling further comprises a second hinge configured to articulate along a second articulation axis that is nonparallel to the first articulation axis, and the coupling is configured such that transitioning the system from the second state to the first state increases a second articulation-range of the hinge along the second articulation axis.

Example 546. The system according to example 545, wherein the second articulation axis is orthogonal to the first articulation axis.

Example 547. The system according to example 543, wherein the system is transitionable between the first state and the second state by moving the anchor longitudinally through the coupling.

Example 548. The system according to example 547, wherein the system is transitionable from the second state to the first state by anchoring the interface to tissue of the heart.

Example 549. The system according to any one of examples 531-538, wherein: the interface comprises: a first collar, a second collar, and a neck portion, connecting the first collar to the second collar, to which a loop portion of the wing is coupled; and/or the system is configured such that: while the system is in the first state, the loop portion is sandwiched between the first collar and the second collar, and while the system is in the second state, the loop portion is loosely coupled to the interface.

Example 550. The system according to example 549, wherein the system is configured to be transitioned from the second state to the first state by advancing the anchor distally through the interface.

Example 551. The system according to example 550, wherein: the anchor comprises an anchor head and a tissue-engaging portion extending away from the anchor head, and the system is configured to transition from the second state to the first state by advancing the tissue-engaging portion distally through the interface such that the anchor head presses distally against the interface.

Example 552. The system according to example 550, configured such that while the interface is in the second state, at least a portion of the anchor protrudes distally through the interface.

Example 553. A system for use with a real or simulated tissue of a real or simulated subject, the system comprising: (i) an anchor defining an anchor head and a helical tissue-engaging element extending distally from the anchor head along an anchor axis; and/or (ii) an implant, the implant comprising an interface (a) configured to be anchored to a site of the tissue by advancing the tissue-engaging element helically through the interface and into the tissue; and/or (b) wherein the interface comprises: (1) a tubular anchor receiver defining a lumen, and (2) a stopper disposed within the lumen, the stopper defining: a window dimensioned to facilitate helical advancement of the tissue-engaging element therethrough, until the anchor head meets the stopper, and a wall configured to inhibit non-helical advancement of the anchor distally through the interface.

Example 554. The system according to example 553, wherein at least one of the anchor and the implant is sterile.

Example 555. Apparatus for use with a cardiovascular system of a real or simulated subject, the apparatus comprising: (i) an anchor, comprising: (a) a helical tissue-engaging element defining an anchor axis of the anchor, having a distal point, and (b) configured to be: (1) screwed into tissue of the cardiovascular system by rotation of the anchor in a first rotational direction about the anchor axis, and (2) unscrewed from the tissue by rotation of the anchor in a second rotational direction about the anchor axis, the second rotational direction being opposite to the first rotational direction, and (b) an anchor head, attached to a proximal end of the tissue-engaging element, and shaped to define: a smooth forward-torque face, facing in the second rotational direction, and an anchor hook facing in the first rotational direction around the anchor axis; and/or (ii) a driver, (a) comprising: (1) a driveshaft, and (2) a drive head that defines: a smooth driver screw-in surface, facing in the first rotational direction, and a driver hook, facing in the second rotational direction, and shaped complementarily to the anchor hook, (b) wherein the driver is configured: (1) to screw the helical tissue-engaging element into the tissue by applying torque, in the first rotational direction, to the anchor head by pressing the driver screw-in surface against the forward-torque face while pressing the anchor head distally, and (2) to unscrew the helical tissue-engaging element from the tissue by applying torque, in the second rotational direction, to the anchor head by hooking the driver hook into the anchor hook and pressing the driver hook against the anchor hook while pulling the anchor hook proximally.

Example 556. The apparatus according to example 555, wherein the anchor is sterile.

Example 557. The apparatus according to any one of examples 555-556, wherein the anchor head and the tissue-engaging element are configured to be cut from a unitary piece of stock tubing.

Example 558. The apparatus according to any one of examples 555-557, wherein the anchor head and the drive head are configured to be cut from a unitary piece of stock tubing.

Example 559. A system for use with a real or simulated tissue of a real or simulated subject, the system comprising: (i) an anchor, having: (a) a helical tissue-engaging element that has a distal tip, and (b) an anchor head, at a proximal end of the tissue-engaging element, the anchor head being shaped to define: a forward-torque face, and a reverse-torque face; and/or (ii) a delivery tool, comprising: (a) a catheter, transluminally advanceable to the tissue, and (b) a driver, comprising (1) a driveshaft, and (2) a drive head at a distal end of the driveshaft, the drive head being shaped such that: (I) while the driveshaft is under compression, rotation of the drive head in a forward rotational direction screws the tissue-engaging element into the tissue by applying forward torque to the forward-torque face; (II) rotation of the drive head in a reverse rotational direction: (A) hooks the drive head onto the anchor head in a manner that facilitates tensioning of the driveshaft while the drive head remains in contact with the anchor head, and (B) unscrews the tissue-engaging element from the tissue by applying reverse torque to the reverse-torque face while the drive head remains hooked onto the anchor head and the driveshaft is under tension; and/or (III) tensioning the driveshaft while the drive head is not hooked onto the anchor head pulls the drive head away from the anchor head.

Example 560. The system according to example 559, wherein at least one of the anchor and the catheter is sterile.

Example 561. The system according to any one of examples 559-560, wherein the reverse-torque face of the anchor head is configured to hook the drive head in a manner that maintains contact between the anchor head and the drive head while the driveshaft is under tension.

Example 562. The system according to any one of examples 559-561, wherein the forward-torque face of the anchor head defines a smooth face that is closer to being perpendicular to the forward torque than to being parallel to the forward torque.

Example 563. The system according to any one of examples 559-562, wherein the driver is configured to (i) screw the tissue-engaging element into the tissue, (ii) hook the drive head onto the anchor head, and (iii) unscrew the tissue-engaging element from the tissue, and (iv) disengage from the anchor head, without any change of conformation of the anchor head.

Example 564. The system according to any one of examples 559-562, wherein the driver is configured to (i) screw the tissue-engaging element into the tissue, (ii) hook the drive head onto the anchor head, and (iii) unscrew the tissue-engaging element from the tissue, and (iv) disengage from the anchor head, without any change of conformation of the drive head.

Example 565. A method for use at a real or simulated tissue of a real or simulated subject, the method comprising: (i) using a driver that includes a driveshaft and a drive head at a distal end of the driveshaft, advancing to the tissue an anchor that has: (a) an anchor head defining: a forward-torque face, and a reverse-torque face, and (b) a helical tissue-engaging element extending from the anchor head; (ii) anchoring the anchor into the tissue by, while the drive head is engaged with the anchor head, driving the helical tissue-engaging element into the tissue by using the driver to apply forward torque to the forward-torque face; and/or (iii) disengaging the drive head from the anchor head; (iv) wherein the step of disengaging does not comprise changing a shape or a conformation of: the drive head or the anchor head.

Example 566. The method according to example 565, further comprising sterilizing the anchor.

Example 567. The method according to any one of examples 565-566, further comprising subsequently unscrewing the tissue-engaging element from the tissue by: hooking the drive head onto the anchor head, and applying reverse torque to the reverse-torque face while pulling the anchor proximally using the drive head hooked onto the anchor head.

Example 568. The method according to any one of examples 565-567, wherein the step of hooking comprises hooking the drive head onto the anchor head by rotating the driver in a reverse rotational direction.

Example 569. The method according to example 568, wherein the step of hooking comprises hooking the drive head onto the anchor head by sliding the drive head proximally with respect to the anchor head.

Example 570. A method for use at a simulated tissue of a real or simulated subject, the method comprising: (i) using a driver that includes a driveshaft and a drive head at a distal end of the driveshaft, advancing to the tissue an anchor that has: (a) an anchor head defining: a forward-torque face, and a reverse-torque face, and (b) a helical tissue-engaging element extending from the anchor head; (ii) anchoring the anchor into the tissue by, while the drive head is engaged with the anchor head, driving the helical tissue-engaging element into the tissue by using the driver to apply forward torque to the forward-torque face; and/or (iii) disengaging the drive head from the anchor head; (iv) wherein the step of disengaging does not comprise changing a shape or a conformation of: the drive head or the anchor head.

Example 571. A system for use at a real or simulated tissue of a real or simulated subject, the system comprising: (i) an implant comprising an interface and an anchor receiver; (ii) an elongate anchor; and/or (iii) a delivery tool extending from a proximal portion to a distal portion, the delivery tool: (a) comprising: (1) a catheter housing the implant, the catheter transluminally advanceable to the tissue, (2) a shaft, extending distally through the catheter, the shaft configured to: deploy the implant out of the catheter, and position the implant such that the interface and the anchor receiver are disposed against a surface of the tissue, and (b) being configured to anchor the implant to the tissue by driving the anchor through the interface and a surface of the tissue, along a curved path within the tissue, such that a distal part of the anchor exits the tissue and is received by the anchor receiver.

Example 572. The system according to example 571, wherein at least one of the implant, the anchor, and the delivery tool is sterile.

Example 573. The system according to any one of examples 571-572, wherein a distal part of the shaft extends distally through the catheter, the distal part of the shaft bifurcating into a first branch and a second branch, each branch: disposed alongside each other within the catheter, the first branch is engaged with the interface, and the second branch is engaged with the anchor receiver.

Example 574. The system according to any one of examples 571-573, wherein the delivery tool further comprises a flexible needle housing the anchor, the needle being deliverable, via the shaft, through the interface and the surface of the tissue, and along the curved path within the tissue to the anchor receiver.

Example 575. The system according to example 574, wherein: the anchor comprises a shape-memory material, the needle is: configured to restrain the anchor in a compressed state, and retractable with respect to the anchor, such that retracting the needle releases the anchor from the compressed state to an expanded state.

Example 576. A method for use with tissue of a real or simulated heart of a real or simulated subject, the method comprising: (i) transluminally advancing to the heart, within a catheter: (a) an implant including an interface and an anchor receiver, and (b) a shaft coupled to the interface; (ii) using the shaft, deploying the implant out of a distal opening of the catheter such that the interface and the anchor receiver are disposed against a surface of the tissue; and/or (iii) anchoring the implant to the tissue by driving an anchor through the interface and a surface of the tissue, along a curved path within the tissue, such that a distal part of the anchor exits the tissue and is received by the anchor receiver.

Example 577. The method according to example 576, further comprising sterilizing the implant, the shaft and the catheter.

Example 578. The method according to any one of examples 576-577, wherein: the anchor includes a shape-memory material; the step of driving the anchor comprises driving the anchor within a flexible needle, through the interface and a surface of the tissue, along a curved path within the tissue, such that a distal part of the anchor exits the tissue and is received by the anchor receiver; and/or the method further comprises, subsequently to the step of anchoring, retracting the needle proximally from the anchor such that the anchor transitions from a compressed state to an expanded state.

Example 579. The method according to any one of examples 576-578, wherein: a distal part of the shaft bifurcates into a first branch coupled to the interface, and a second branch coupled to the anchor receiver; and/or the step of advancing comprises transluminally advancing the first branch and the second branch of the distal part of the shaft alongside each other within the catheter.

Example 580. A method for use with simulated tissue of a real or simulated heart of a real or simulated subject, the method comprising: (i) transluminally advancing to the heart, within a catheter: (a) an implant including an interface and an anchor receiver, and (b) a shaft coupled to the interface; (ii) using the shaft, deploying the implant out of a distal opening of the catheter such that the interface and the anchor receiver are disposed against a surface of the tissue; and/or (iii) anchoring the implant to the tissue by driving an anchor through the interface and a surface of the tissue, along a curved path within the tissue, such that a distal part of the anchor exits the tissue and is received by the anchor receiver.

Example 581. The method according to example 580, further comprising sterilizing the implant, the shaft and the catheter.

Example 582. Apparatus for use with a real or simulated valve of a real or simulated heart of a real or simulated subject, the valve defining an annulus and having a first leaflet and an opposing leaflet, the heart having a chamber upstream of the valve, and the apparatus comprising an implant (i) that comprises: (a) a wing: extending from a root portion of the wing to a tip portion of the wing, having a compressed state, and biased to expand into an expanded state; and/or (b) an interface, connected to the root portion of the wing, configured to be anchored to tissue of the annulus such that the wing extends over the first leaflet toward the opposing leaflet, (ii) wherein the implant is configured such that: while the wing is in the compressed state, the implant has a hinged coupling between the root portion and the interface that facilitates articulation, at the hinged coupling, of the root portion with respect to the interface, and expansion of the wing into the expanded state inhibits the articulation by restraining the hinged coupling.

Example 583. The apparatus according to example 582, wherein the implant is sterile.

Example 584. A system for use with a real or simulated valve of a real or simulated heart of a real or simulated subject, the valve defining an annulus and having a first leaflet and an opposing leaflet, the heart having a chamber upstream of the valve, the system comprising: (i) an implant, comprising: (a) a wing that extending from a root portion of the wing to a tip portion of the wing, and (b) an interface; an (c) anchor; and/or (ii) a delivery tool, comprising: (a) a catheter, transluminally advanceable to the chamber, and configured to house the implant while the wing is in a compressed state, and (b) a shaft, engaged with the interface, and configured, via the engagement with the interface, to deploy the implant out of the catheter and into the chamber, wherein: the wing is biased to expand into an expanded state upon being deployed, and the implant is configured such that: while the wing is in the compressed state, the root portion has a hinged coupling to the interface that facilitates articulation, at the hinged coupling, of the root portion with respect to the interface, and expansion of the wing into the expanded state inhibits the articulation by restraining the hinged coupling.

Example 585. The system according to example 584, wherein at least one of the implant, the anchor, and the delivery tool is sterile.

Example 586. A system for use with a real or simulated valve of a real or simulated heart of a real or simulated subject, the valve defining an annulus and having a first leaflet and an opposing leaflet, the heart having a chamber upstream of the valve, the system comprising (i) an implant that comprises: (a) a wing: extending from a root portion of the wing to a tip portion of the wing, having a compressed state, and biased to expand into an expanded state; and/or (b) an annular support, connected to the root portion of the wing, and configured such that: in the compressed state of the wing, the wing has a hinged coupling to the annular support that facilitates articulation, at the hinged coupling, of the wing with respect to the annular support, and expansion of the wing toward the expanded state inhibits the articulation by restraining the hinged coupling.

Example 587. The system according to example 586, wherein the implant is sterile.

Example 588. The system according to any one of examples 586-587, wherein: (i) the implant defines an interface; (ii) the wing defines a contact face, and an opposing face opposite to the contact face; and/or (iii) the system further comprises: (a) an anchor, and (b) a delivery tool, comprising: (1) a catheter, transluminally advanceable to the chamber with the implant housed in the catheter while the wing is in the compressed state, and (2) a driver, configured to: deploy the implant out of the catheter such that, within the chamber, the wing assumes the expanded state, position the implant in a position in which: the wing extends over the first leaflet toward the opposing leaflet, and the contact face faces the first leaflet, and while the implant is positioned in the position and the wing is in the expanded state, secure the interface to the annulus by driving the anchor through the interface and into tissue of the annulus.

Example 589. The system according to any one of examples 586-588, wherein the annular support is shaped such that, while the implant is secured to the annulus and the wing is in the expanded state, the annular support is disposed against an atrial surface of the annulus such that, the restrained hinged coupling inhibits deflection of the root portion of the wing with respect to the annulus.

Example 590. The system according to any one of examples 588-589, wherein: the interface is a first interface; the annular support comprises a first annular arm that extends away from the hinged coupling to the first interface; and/or the annular support further comprises a second annular arm that: is coupled to the hinged coupling, and extends away from the hinged coupling to a second interface.

Example 591. The system according to example 590, wherein the first annular arm is joined to the second annular arm, at the hinged coupling.

Example 592. The system according to any one of examples 586-589, wherein the implant is configured such that: the hinged coupling comprises a sleeve defining an aperture, while the wing is in the compressed state, a thin portion of the annular support is disposed within the aperture, and expansion of the wing toward the expanded state slides the sleeve from the thin portion to a thick portion of the annular support, the thick portion having a cross-section that is dimensioned to restrain the hinged coupling by fitting the aperture.

Example 593. The system according to example 592, wherein the thick portion of the annular support has an oblong cross-section that is dimensioned to restrain the hinged coupling by fitting the aperture.

Example 594. The system according to example 592, wherein: the sleeve is a first sleeve defining a first aperture, the hinged coupling further comprises a second sleeve defining a second aperture, the annular support comprises a pair of annular arms, each annular arm having: a thin portion at which the annular arms are joined, and a thick portion that extends away from the thin portion; wherein expansion of the wing toward the expanded state slides each sleeve from the thin portion to the thick portion of a respective annular arm, thereby restraining the hinged coupling.

Example 595. The system according to example 588, wherein the annular support comprises an expansion element having: a compact state, and an extended state in which the expansion element resists compression of the wing toward the compressed state.

Example 596. The system according to example 595, wherein the annular support is configured such that extension of the expansion element from the compact state to the extended state applies an expansion force to the wing, the expansion force facilitating expansion of the wing from the compressed state to the expanded state.

Example 597. The system according to example 595, wherein the expansion element is configured to resist transition from the extended state toward the compact state.

Example 598. The system according to example 597, wherein the expansion element comprises a spring.

Example 599. The system according to example 597, wherein the expansion element comprises a plurality of subunits, and the expansion element is configured such that extending the expansion element into the extended state causes the subunits to fit together.

Example 600. The system according to example 599, wherein the plurality of subunits is configured to lock together upon the expansion element assuming the extended state.

Example 601. The system according to example 599, wherein the expansion element is straighter in the extended state than in the compact state.

Example 602. The system according to example 601, wherein the expansion element comprises a hinge, and the expansion element is configured such that straightening the hinge straightens the expansion element.

Example 603. The system according to example 602, wherein the delivery tool further comprises an extension actuator, the extension actuator configured to transluminally extend the expansion element from the compact state to the extended state.

Example 604. A system for use with a real or simulated valve of a real or simulated heart of a real or simulated subject, the valve having a first leaflet and an opposing leaflet, the heart having a chamber upstream of the valve, the system comprising: (i) an implant (a) transitionable between a compressed state and an expanded state, (b) the implant comprising: (1) a wing: extending from a root portion of the wing to a tip portion of the wing, and defining a contact face, and an opposing face opposite to the contact face, and (2) a pair of arms, each arm: fastened to the tip portion of the wing, and extending away from the wing and the other arm of the pair to define a lateral portion of the arm that is disposed laterally from the wing; and/or (ii) a delivery tool: comprising a catheter, transluminally advanceable to the chamber, the catheter housing the implant while the implant is in the compressed state, and configured to deploy the implant out of the catheter and position the implant in a position in which: the implant is in its expanded state, the wing extends, from the root portion, over the first leaflet toward the opposing leaflet, the lateral portion of each arm presses, at a respective lateral site lateral from the wing, in an upstream direction against a downstream side of the first leaflet in a manner that presses the contact face against an upstream side of the first leaflet between the lateral sites.

Example 605. The system according to example 604, wherein at least one of the implant and the delivery tool is sterile.

Example 606. The system according to any one of examples 604-605, wherein: the catheter is configured to house the implant while the implant is in the compressed state, and the implant comprises a flexible frame biased to expand the implant into the expanded state upon deployment from the catheter.

Example 607. The system according to any one of examples 604-606, wherein the delivery tool further comprises a shaft, reversibly engageable to the implant and configured to: deploy the implant from the catheter, position the implant in the position, and while the implant is in the position, release the implant.

Example 608. The system according to example 607, wherein the implant is configured to maintain the position upon the shaft releasing the implant, while the implant is in the position, by pinching the first leaflet between the wing and the lateral portion of each arm.

Example 609. The system according to any one of examples 604-608, configured such that, while the implant is positioned in the position: the lateral portion of each arm presses, at the respective lateral site lateral from the wing, in the upstream direction against a downstream side of the first leaflet in a manner that presses the contact face of the root portion of the wing against an upstream side of the first leaflet between the lateral sites, and the tip portion of the wing deflects in concert with the lateral portion of each arm and with tissue of the lateral sites, responsively to a cardiac cycle of the heart, in a reciprocating manner, in the upstream direction and in a downstream direction.

Example 610. The system according to example 609, configured such that, while the implant is positioned in the position, the pressing of the contact face of the root portion of the wing, against the upstream side of the first leaflet between the lateral sites, inhibits deflection of the root portion in the upstream direction.

Example 611. The system according to example 610, configured such that, while the implant is positioned in the position, the pressing of the contact face of the root portion of the wing, against the upstream side of the first leaflet between the lateral sites defines a deflection-limit of the wing during the cardiac cycle by inhibiting deflection of the wing in the upstream direction beyond the deflection-limit.

Example 612. A system for use with a real or simulated heart of a real or simulated subject, the system comprising: (i) an anchor; (ii) an implant, comprising an interface; and/or (ii) a delivery tool, (a) comprising: (1) a driver, engaged with the anchor, and (2) a shaft comprising: (I) a coupling engageable to the interface, and (II) a lock at a distal portion of the shaft, the lock: comprising a first unit and a second unit, and having a locked state in which the first unit is mated with the second unit, and having an unlocked state in which the first unit is separated and translatable away from the second unit; (b) wherein the delivery tool is configured to: (1) while the lock is locked: via engagement of the coupling with the interface, position the implant within the heart, and use the driver to secure the implant to tissue of the heart by driving the anchor into the tissue, (2) reversibly and repeatedly transition the lock between the locked state and the unlocked state, and/or (3) while the implant remains secured to the tissue, disengage the coupling from the interface.

Example 613. The system according to example 612, wherein at least one of the implant, the anchor, and the delivery tool is sterile.

Example 614. The system according to any one of examples 612-613, wherein the coupling is configured to remain engaged to the interface while the lock transitions between the locked state and the unlocked state.

Example 615. The system according to any one of examples 612-613, wherein the delivery tool is configured to disengage the coupling from the interface while the lock is locked.

Example 616. The system according to any one of examples 612-613, wherein the delivery tool is configured to disengage the coupling from the interface while the lock is unlocked.

Example 617. The system according to any one of examples 612-616, wherein the distal portion of the shaft, at which the lock is disposed, is disposed proximally of the coupling.

Example 618. The system according to example 617, wherein a distal end portion of the shaft defines the coupling.

Example 619. The system according to any one of examples 612-614, configured such that transitioning the lock from the locked state to the unlocked state, while the coupling remains engaged to the interface and the implant remains secured to the tissue, facilitates deflection of the implant responsively to a cardiac cycle of the heart.

Example 620. The system according to example 619, wherein: the heart has: a real or simulated valve having a first leaflet and an opposing leaflet, and a chamber upstream of the valve; the implant comprises a wing defining a contact face and an opposing face opposite to the contact face; the shaft is configured, via engagement of the coupling to the interface, to position the implant in a position in which the wing extends over the first leaflet toward the opposing leaflet, and the system is configured such that transitioning the lock from the locked state to the unlocked state, while the implant remains in the position, the coupling remains engaged to the interface and the implant remains secured to the tissue, facilitates deflection of the wing, in an upstream direction and in a downstream direction, responsively to the cardiac cycle.

Example 621. The system according to example 620, wherein: the interface of the implant is disposed at a root portion of the wing, and the wing extends, away from the interface to a tip portion of the wing, over the first leaflet toward the opposing leaflet, the delivery tool is configured to use the driver to secure the root portion of the wing to tissue of the chamber by driving the anchor through the interface and into the tissue of the chamber, and the delivery tool is configured to transition the lock from the locked state to the unlocked state while the root portion of the wing remains secured to the tissue of the chamber, in a manner that facilitates deflection of the tip portion of the wing, in the upstream direction and in the downstream direction, responsively to the cardiac cycle.

Example 622. The system according to any one of examples 612-621, wherein: the system comprises a pair of anchors; the interface is a first interface, and the implant further comprises a second interface; the coupling comprises: a first branch engageable to the first interface along a first-branch axis, and a second branch engageable to the second interface, along a second-branch axis that is nonparallel to the first-branch axis; and/or the delivery tool comprises a pair of drivers, each driver configured to drive one of the anchors along a respective axis, through a respective interface and into the tissue.

Example 623. The system according to example 622, wherein a distal end portion of the shaft: defines the coupling, and bifurcates into the first branch and the second branch, distally of the lock.

Example 624. The system according to any one of examples 622-623, configured such that: while the lock is in the locked state, the coupling is secured to a proximal portion of the shaft; while the lock is in the unlocked state, the coupling is released from the proximal portion of the shaft; and/or the delivery tool comprises a catheter, configured to house the implant and the shaft such that the first branch of the coupling and the second branch of the coupling are oriented along a proximal shaft axis of the proximal portion of the shaft.

Example 625. The system according to example 624, wherein the coupling comprises a shape-memory material that biases the first branch and the second branch to flex away from each other upon release from the catheter.

Example 626. The system according to example 624, wherein the first branch and the second branch are configured to flex away from each other upon release from the catheter such that the first-branch axis and the second-branch axis are each oblique to the proximal shaft axis.

Example 627. The system according to any one of examples 612-626, wherein the delivery tool: comprises a tether, and is configured such that intracardially adjusting tension on the tether transitions the lock between the locked state and the unlocked state.

Example 628. The system according to example 627, wherein the delivery tool is configured such that while the lock is in the unlocked state: the first unit is separated from the second unit, and the tether connects the first unit to the second unit.

Example 629. A method for use at a real or simulated tissue of a real or simulated heart of a real or simulated subject, the method comprising: (i) using a catheter, transluminally advancing to the heart of the subject: (a) an implant, including an interface, and (b) a shaft engaged to the interface, the shaft including a lock at a distal portion of the shaft, the lock having a first unit and a second unit; (ii) while the lock is locked such that the first unit is mated with the second unit: (a) deploying the implant out of the catheter, and (b) using a driver engaged with an anchor, securing the interface to the tissue by driving the anchor through the interface and into the tissue; and/or (iii) subsequently: unlocking the lock such that the first unit is separated from the second unit, disengaging the coupling from the interface, and withdrawing the shaft from the subject.

Example 630. The method according to example 629, further comprising sterilizing the implant, the anchor, the shaft and the catheter.

Example 631. The method according to any one of examples 629-630, wherein the step of disengaging comprises disengaging the coupling from the interface while the lock remains unlocked.

Example 632. The method according to any one of examples 629-630, further comprising, prior to the step of disengaging, relocking the lock such that the first unit is mated with the second unit.

Example 633. The method according to example 632, wherein the step of disengaging comprises disengaging the coupling from the interface while the lock remains locked.

Example 634. The method according to any one of examples 629-633, wherein: the shaft includes a tether, adjustably coupled to the lock, and the step of unlocking comprises unlocking the lock by intracardially reducing tension upon the tether.

Example 635. The method according to example 634, further comprising, prior to the step of disengaging, relocking the lock, such that the first unit is mated with the second unit, by increasing tension on the tether.

Example 636. The method according to any one of examples 629-635, further comprising, prior to the step of disengaging, and while the lock remains unlocked, assessing function of the implant.

Example 637. The method according to example 636, wherein the step of assessing comprises assessing function of the implant while the driver remains engaged with the anchor.

Example 638. The method according to example 636, further comprising, prior to the step of assessing, disengaging the driver from the anchor.

Example 639. The method according to example 636, wherein the step of assessing comprises assessing deflection of the implant responsively to a cardiac cycle of the heart.

Example 640. The method according to example 639, wherein: the heart has: a real or simulated valve having a first leaflet and an opposing leaflet, and a chamber upstream of the valve; and/or the implant includes a wing defining a contact face and an opposing face opposite to the contact face; the step of securing comprises securing the interface to tissue of the chamber by driving the anchor through the interface and into the tissue of the chamber; the step of unlocking comprises facilitating deflection of the wing, in an upstream direction and in a downstream direction, responsively to the cardiac cycle; and/or the step of assessing comprises assessing deflection of the wing in the upstream direction and in the downstream direction, responsively to the cardiac cycle.

Example 641. The method according to example 640, wherein: the interface is disposed at a root portion of the wing; the step of securing comprises securing the root portion of the wing to the tissue of the chamber by driving the anchor through the interface and into the tissue of the chamber, such that the wing extends, away from the interface to a tip portion of the wing, over the first leaflet toward the opposing leaflet; the step of unlocking comprises unlocking the lock while the root portion of the wing remains secured to the tissue of the chamber, thereby facilitating deflection of the tip portion of the wing, in the upstream direction and in the downstream direction, responsively to the cardiac cycle; and/or the step of assessing comprises assessing deflection of the tip portion of the wing in the upstream direction and in the downstream direction, responsively to the cardiac cycle.

Example 642. The method according to example 636, wherein the step of disengaging and the step of withdrawing comprise, responsively to the step of assessing: disengaging the coupling from the interface, and withdrawing the shaft from the subject.

Example 643. The method according to example 642, further comprising, prior to the step of disengaging, relocking the lock such that the first unit is mated with the second unit.

Example 644. The method according to example 642, further comprising, prior to the step of disengaging and responsively to the step of assessing: using the driver, removing the anchor from the tissue; using the shaft, repositioning the implant; and/or repeating the step of securing and the step of assessing.

Example 645. A method for use at a simulated tissue of a real or simulated heart of a real or simulated subject, the method comprising: (i) using a catheter, transluminally advancing to the heart of the subject: (a) an implant, including an interface, and (b) a shaft engaged to the interface, the shaft including a lock at a distal portion of the shaft, the lock having a first unit and a second unit; (ii) while the lock is locked such that the first unit is mated with the second unit: (a) deploying the implant out of the catheter, and (b) using a driver engaged with an anchor, securing the interface to the tissue by driving the anchor through the interface and into the tissue; and/or (iii) subsequently: unlocking the lock such that the first unit is separated from the second unit, disengaging the coupling from the interface, and withdrawing the shaft from the subject.

Example 646. A system for use with a real or simulated valve of a real or simulated heart of a real or simulated subject, the valve having a first leaflet and an opposing leaflet, the heart having a chamber upstream of the valve, and the system comprising: (i) an implant, comprising: (a) an interface, (b) a flexible wing, coupled to the interface, and having a contact face and an opposing face opposite the contact face, (c) a beam, connected to the wing along a part of the wing, and (d) a line, coupled to the beam such that tensioning the line strains the beam; (ii) an anchor; and/or (iii) a delivery tool, comprising: (a) a catheter, transluminally advanceable to the chamber, and configured to house the implant, (b) a shaft, engaged with the interface, and configured, via the engagement with the interface, to: (1) deploy the implant out of the catheter such that, within the chamber, the wing extends away from the interface, and (2) position the implant in a position in which the interface is at a site in the heart, the wing extends over the first leaflet toward the opposing leaflet, and the contact face faces the first leaflet, and (c) a driver, engaged with the anchor, and configured to secure the implant in the position by using the anchor to anchor the interface to tissue of the heart, wherein the delivery tool is configured to intracardially reshape the wing by straining the beam by tensioning the line.

Example 647. The system according to example 646, wherein at least one of the implant, the anchor, and the delivery tool is sterile.

Example 648. The system according to any one of examples 646-647, wherein: the line extends from a proximal portion of the line at a proximal portion of the delivery tool to a distal portion of the line coupled to the beam, and the proximal portion of the delivery tool is configured to intracardially reshape the wing by straining the beam by tensioning the proximal portion of the line.

Example 649. The system according to example 648, wherein the line extends from the proximal portion of the line, through the interface and to the distal portion of the line.

Example 650. The system according to any one of examples 646-649, wherein the wing defines a root portion at which the interface is disposed, and a tip portion along which the beam is connected to the wing.

Example 651. The system according to example 650, wherein the beam is connected to the wing along a portion of a perimeter of the tip portion of the wing.

Example 652. The system according to example 650, wherein the line extends from a proximal portion of the line at a proximal portion of the delivery tool to the beam at the tip portion of the wing.

Example 653. The system according to example 652, wherein the proximal portion of the delivery tool is configured to intracardially reshape the tip portion of the wing by straining the beam by tensioning the proximal portion of the line.

Example 654. The system according to example 650, wherein the delivery tool is configured to intracardially change a radius of curvature of the wing along a normal plane of the wing extending from the root portion to the tip portion by tensioning the line.

Example 655. The system according to example 654, wherein the delivery tool is configured to intracardially increase the radius of curvature of the wing along the normal plane of the wing extending from the root portion to the tip portion by tensioning the line.

Example 656. The system according to example 654, wherein the delivery tool is configured to intracardially decrease the radius of curvature of the wing along the normal plane of the wing extending from the root portion to the tip portion by tensioning the line.

Example 657. The system according to example 651, wherein: the beam is a rigid beam, and the beam is connected to the wing along the portion of the perimeter of the tip portion of the wing such that the tip portion has a greater stiffness, along the normal plane of the wing extending from the root portion to the tip portion, than the root portion.

Example 658. The system according to example 654, wherein a distal portion of the line is generally parallel to the normal plane of the wing extending from the root portion to the tip portion.

Example 659. The system according to any one of examples 646-658, wherein the delivery tool is configured to intracardially reshape the wing by reshaping the beam by tensioning the line.

Example 660. The system according to example 659, wherein: the wing comprises a braided mesh, and the delivery tool is configured to intracardially reshape the wing by reorienting a weave of the braided mesh by reshaping the beam by tensioning the line.

Example 661. The system according to example 659, wherein: the line is a first line connected to a first portion of the beam, the implant further comprises a second line, connected to a second portion of the beam, and tensioning the first line and the second line reshapes the wing by changing a distance between the first portion of the beam and the second portion of the beam.

Example 662. The system according to example 659, wherein the delivery tool is configured to intracardially change a width of the wing by changing a radius of curvature of the beam by tensioning the line.

Example 663. The system according to example 662, wherein the delivery tool is configured to intracardially reduce the width of the wing by decreasing the radius of curvature of the beam by tensioning the line.

Example 664. The system according to any of the above examples, wherein at least one of the implant and the delivery tool is sterilized.

Example 665. A system for use with a real or simulated heart of a real or simulated subject, the system comprising: (i) an implant, the implant comprising: an interface, and a wing coupled to the interface; (ii) at least one anchor; and/or (iii) a delivery tool, comprising: (a) a catheter that is transluminally advanceable to the heart, the catheter defining a distal opening, (b) a shaft disposed or disposable within the catheter, the shaft engaged or engageable with the implant and configured, via the engagement, to: deploy the implant out of the distal opening of the catheter, and position the implant such that the interface is disposed at a site of the heart, and (c) a driver engaged or engageable with the anchor and configured to secure the implant to the heart by: advancing the anchor out of the catheter, and driving the anchor through the interface and into tissue of the heart at the site.

Example 666. The system according to example 665, wherein at least one of the implant, the anchor, and the delivery tool is sterile.

Example 667. The system according to any one of examples 665-666, wherein: the implant has a compressed state and an expanded state, and comprises: a flexible frame biased toward causing the implant to assume the expanded state.

Example 667. The system according to any one of examples 665-667, further comprising an expansion element, having: a compact state, and an extended state in which the expansion element resists compression of the implant toward a compressed state, wherein: the catheter is configured to house the implant while the implant is in the compressed state and the expansion element is in the compact state, and the shaft is configured, via the engagement to deploy the implant out of the distal opening of the catheter such that, within the heart, the implant assumes an expanded state and the expansion element assumes the extended state.

Example 668. The system according to example 667, wherein the implant is configured such that extension of the expansion element from the compact state to the extended state applies an expansion force to the implant, the expansion force facilitating expansion of the implant from the compressed state to the expanded state.

Example 669. The system according to any one of examples 667-668, wherein the expansion element is configured to resist transition from the extended state toward the compact state.

Example 670. The system according to any one of examples 667-669, wherein the expansion element comprises a spring.

Example 671. The system according to any one of examples 667-670, wherein the expansion element comprises a plurality of subunits, and the expansion element is configured such that extending the expansion element into the extended state causes the subunits to fit together.

Example 672. The system according to any one of examples 667-671, wherein the expansion element comprises a plurality of subunits, configured to lock together upon the expansion element assuming the extended state.

Example 673. The system according to any one of examples 667-672, wherein the expansion element is straighter in the extended state than in the compact state.

Example 674. The system according to any one of examples 667-673, wherein the expansion element comprises a hinge, and the expansion element is configured such that straightening the hinge straightens the expansion element.

Example 675. The system according to any one of examples 667-673, wherein the delivery tool further comprises an extension actuator, the extension actuator configured to transluminally extend the expansion element from the compact state to the extended state.

Example 676. The system according to any one of examples 665-675, wherein: the anchor is a first anchor, and the system further comprises a second anchor; and wherein the interface comprises a first anchor receiver, and the implant further comprises a second anchor receiver; the driver is a first driver, and the delivery tool further comprises a second driver; and/or the first and second drivers are each engaged or engageable with a respective anchor and configured to secure the implant to the heart by driving one of the first and second anchors through one of the first and second anchor receivers and into tissue of the heart at the site.

Example 677. The system according to example 676, wherein the first and second drivers are configured to diverge away from each other as the first and second drivers advance out of one of the lateral openings and toward the anchor receivers.

Example 678. The system according to any one of examples 665-677, wherein: the catheter further comprises a gate at a lateral opening of the catheter, the gate comprising a shape-memory material; and/or the catheter is configured to transition between: a delivery state in which the gate is closed, and a deployment state in which the gate is open.

Example 679. The system according to example 678, configured such that deploying the implant out of the distal opening facilitates transitioning the catheter from the delivery state to the deployment state.

Example 680. The system according to example 678, wherein the catheter is configured such that while the catheter is in the deployment state, the open gate guides the driver and the anchor out of the lateral opening of the catheter and toward the interface.

Example 681. The system according to any one of examples 665-680, wherein the wing has a contact face, an opposing face opposite to the contact face, a tip portion, a root portion.

Example 682. The system according to example 681, wherein the wing includes a flex element that couples the tip portion to the root portion.

Example 683. The system according to example 682, wherein the flex element protrudes from at least one of (a) the contact face of the wing and (b) the opposing face of the wing.

Example 684. The system according to any one of examples 682-683, wherein the flex element is or comprises at least one of (a) a flexure, (b) a hinge, and (c) a living hinge.

Example 685. The system according to any one of examples 682-683, wherein the flex element comprises a pair of interlocking loops, a first one of the loops defined by the root portion, and a second one of the loops defined by the tip portion.

Example 686. The system according to any one of examples 682-683, wherein the flex element comprises at least one of (a) a plurality of coiled wires connecting the tip portion to the root portion, (b) a plurality of rings connecting the tip portion to the root portion, and (c) a plurality of sutures connecting the tip portion to the root portion.

Example 687. The system according to any one of examples 682-683, wherein the flex element comprises a tube through which respective portions of the tip portion and the root portion extend alongside each other, such that the tip portion and root portion can articulate in relation to each other.

Example 688. The system according to any one of examples 682-683, wherein the root portion is stiffer than the tip portion.

Example 689. The system according to any one of examples 682-688, wherein the implant is configured such that, after the anchor is driven into the tissue at the site, flexing of the flex element facilitates deflection of the tip portion with respect to the root portion in response to a cardiac cycle of the heart.

Example 690. The system according to example 689, wherein the flex element is protrusive, and the implant is configured such that, after the anchor is driven into the tissue at the site, the flex element abuts a hinge-point between a leaflet of a valve of the heart and an annulus of the valve.

Example 691. The system according to any one of examples 682-690, wherein the flex element is protrusive so as to abut a hinge-point between a leaflet of a valve of the heart and an annulus of the valve and is positioned within the implant such that abutment of the flex element against the hinge-point positions an interface of the implant at the site.

Example 692. The system according to any one of examples 682-691, wherein the wing comprises a frame, and a flexible sheet disposed over the frame, and the frame defines the flex element.

Example 693. The system according to example 692, wherein the flex element comprises at least one of a torsion spring, a hinge, and a ball-and-socket hinge.

Example 694. The system according to any one of examples 665-692, wherein the wing is a first wing, and wherein the implant further comprises a second wing: extending, over the opposing face of the first wing, from a second root portion of the second wing to a second tip portion of the second wing, the first wing being deflectable toward and away from the second wing, and an interface coupled to the first root portion and to the second root portion.

Example 695. The system according to example 694, wherein the implant is configured such that, while the implant remains secured in the position, during ventricular diastole the first wing deflects farther into the chamber than does the second wing.

Example 696. The system according to example 695, wherein the implant further comprises a third wing, the third wing having a third root portion that is coupled to the interface, and extending, over the second wing, from the third root portion to a third tip portion of the third wing, the second wing being deflectable toward and away from the third wing.

Example 697. The system according to example 695, wherein the third wing is at least one of (a) shorter than the second wing, and (b) less flexible than the second wing and/or the first wing.

Example 698. The system according to any one of examples 696-697, wherein the implant is configured such that, after the anchor is driven into the tissue at the site, during ventricular diastole one or both of the following occurs: (a) the second wing deflects away from the third wing, and (b) the first wing deflects away from the second wing.

Example 699. The system according to any one of examples 694-698, wherein the first wing defines multiple holes therethrough.

Example 700. The system according to example 699, wherein the implant is configured such that, after the anchor is driven into the tissue at the site, during ventricular systole, the first wing deflects into contact with the second wing in a manner that obstructs blood flow through the holes.

Example 701. The system according to example 700, wherein the second wing defines multiple holes therethrough, the holes of the first wing being positioned such that, while the first wing is in contact with the second wing, the holes of the first wing are offset with respect to the holes of the second wing.

Example 702. The system according to any one of examples 694-701, wherein the implant further comprises a flexible pouch, the first and second wings being disposed within the pouch.

Example 703. The system according to any one of examples 665-702, wherein the implant further comprises a limiter configured to (a) define a deflection-limit of the wing, and (b) to inhibit deflection of the wing in the upstream direction beyond the deflection-limit by providing an opposing force upon the wing reaching the deflection-limit.

Example 704. The system according to example 703, wherein: the limiter is intracardially adjustable in a manner that adjusts the deflection-limit of the wing, and wherein the deflection-limit is adjustable by adjustment of a depth of anchoring of the anchor within the tissue.

Example 705. The system according to example 704, wherein the limiter is coupled to an interface, and the interface is configured to be anchored to the site by the anchor.

Example 706. The system according to example 705, wherein the interface is configured to be anchored to the site by advancing the anchor through the interface and into the tissue at the site, such that: while the implant is secured in the position, the limiter extends away from the interface and over the wing, and upon the wing reaching the deflection-limit, the wing contacts the limiter.

Example 707. The system according to example 706, wherein the implant is configured such that the adjustment of the limiter by adjustment of the depth of anchoring comprises adjustment of an angle of the limiter with respect to the interface.

Example 708. The system according to any one of examples 703-707, wherein the limiter comprises a tether, the tether being configured to become tensioned as the wing reaches the deflection-limit.

Example 709. The system according to example 708, wherein the tether is coupled to a portion of the wing such that shortening of the tether adjusts the deflection-limit.

Example 710. The system according to example 709, further comprising a rotatable spool, wherein: a portion of the tether is wound around the spool, and the tether is configured to be shortenable by rotating the spool.

Example 711. The system according to example 709, wherein the implant comprises a tissue anchor, coupled to the tether, and configured to anchor the tether to tissue of a second chamber of the heart.

Example 712. The system according to example 711, wherein: the tether defines a rail portion to which a proximal portion of the tether is slidably coupled; the tissue anchor is a first atraumatic tissue anchor, coupled to a first part of the rail portion, and configured to anchor the first part of the rail portion to trabeculae at a first site of the second chamber; and/or the implant further comprises a second atraumatic tissue anchor, coupled to a second part of the rail portion, and configured to anchor the second part of the rail portion to trabeculae at a second site of the second chamber.

Example 713. The system according to example 709, wherein the tether is configured to be shortened by being slid with respect to the root portion of the wing.

Example 714. The system according to any one of examples 705-713, wherein the limiter is coupled to the interface, and extends, away from the interface and over the wing, such that the wing is deflectable toward and away from the limiter.

Example 715. The system according to example 714, wherein the limiter has a backstop portion that is shaped to press against tissue of a first chamber upon anchoring of the interface to the site.

Example 716. The system according to example 715, wherein the interface serves as a fulcrum between the backstop portion and a part of the limiter that extends away from the interface and over the wing, such that the pressing the backstop portion against tissue of the first chamber adjusts the deflection-limit of the wing.

Example 717. The system according to example 715, wherein the backstop portion is intracardially adjustable in a manner that adjusts pressing of the backstop portion against the tissue.

Example 718. The system according to any one of examples 665-717, wherein the wing is configured to define a deflection-limit, and to become resistant to deflection in the upstream direction upon reaching the deflection-limit.

Example 719. The system according to any one of examples 665-718, wherein the implant comprises (a) a first interface defining a first longitudinal axis and (b) a second interface defining a second longitudinal axis, each of the first and second interfaces: disposed at a root portion of the wing, and coupled to the wing such that the first longitudinal axis is nonparallel to the second longitudinal axis.

Example 720. The system according to example 719, the shaft is a first shaft, and wherein the delivery tool comprises a second shaft disposed or disposable alongside the first shaft within the catheter, each of the first and second shafts engaged or engageable with a corresponding one of the first and second interfaces.

Example 721. The system according to example 720, wherein the first and second shafts are each configured, via the engagement with the corresponding interfaces, to: deploy the implant out of the catheter, and position the implant in a position in which: the first interface is at a first site upstream of a native valve of the heart, the second interface is at a second site upstream of the native valve, and the wing extends over a first leaflet of the native valve toward an opposing leaflet of the native valve, such that a contact face of the wing faces the first leaflet.

Example 722. The system according to any one of examples 720-721, further comprising a first driver and a second driver, each driver engaged with a corresponding one of first and second anchors of the implant, and configured to secure the implant by screwing: the first anchor along the first longitudinal axis to anchor the first interface to tissue at the first site, and the second anchor along the second longitudinal axis to anchor the second interface to tissue at the second site.

Example 723. The system according to example 722, wherein (a) the first driver extends or is extendable distally into the first shaft where a first drive head of the first driver is engaged or engageable with the first anchor, and being configured to anchor the first interface to the tissue by driving the first anchor distally through the first interface and into the tissue, and (b) the second driver extends or is extendable distally into the second branch where a second drive head of the second driver is engaged or engageable with the second anchor, and configured to anchor the second interface to the tissue by driving the second anchor distally through the second interface and into the tissue.

Example 724. The system according to any one of examples 665-723, wherein the implant comprises an interface configured to inhibit non-helical advancement of the anchor distally through the interface, and facilitate non-helical withdrawal of the anchor proximally through the interface.

Example 725. The system according to any one of examples 665-724, wherein the implant comprises a flexible sheet covering a frame of the wing, and extending beyond the frame to define lateral flaps.

Example 726. The system according to any one of examples 665-725, wherein the wing has a root portion and a tip portions, the root portion being stiffer than the tip portion.

Example 727. The system according to any one of examples 665-726, further comprising a ripcord configured such that pulling the ripcord releases a distal end of the shaft from the implant.

Example 728. The system according to any one of examples 665-727, wherein the implant is configured such that a deflection-range of the wing can be intracardially adjusted.

Example 729. The system according to any one of examples 665-728, wherein a shape-memory member is coupled to the wing, the shape-memory member configured such that a size of the wing can by chronically changed by temporarily heating the shape-memory member.

Example 730. The system according to any one of examples 665-729, wherein the implant comprises a limb or extension coupled to the wing and shaped such that, when the wing is anchored to the tissue at the site, the limb/extension extends away from the wing to contact tissue of the heart adjacent a root of a leaflet of a valve of the heart, in a manner that moderates deflection of the wing with respect to the site in an upstream direction.

Example 731. The system according to any one of examples 665-730, wherein the implant comprises an adjustment member that is adjustable to adjust a deflection-range of the wing.

Example 732. The system according to any one of examples 665-731, wherein the implant comprises a pair of arms coupled to the wing, wherein the pair of arms arc divergently away from the wing.

Example 733. The system according to example 732, wherein each arm of the pair of arms: (a) has an anchor point, and (b) is configured to be anchored to the annulus such that: the arm arcs, from the anchor point, along an annulus of a valve of the heart to the wing.

Example 734. The system according to any one of examples 665-733, wherein the implant comprises a leg extending from the wing to an end portion of the leg, the leg configured to extend away from the wing to press against an underside of a valve of the heart when the anchor is anchored to the tissue at the site.

Example 735. The system according to any one of examples 665-734, wherein the implant comprises an adjustment rod reversibly coupled to the wing such that, when the anchor is anchored into the tissue at the site, axial movement of the adjustment rod adjusts a position of the wing by sliding the interface with respect to the tissue and the anchor.

Example 736. The system according to any one of examples 665-735, wherein the interface defines an oblong opening delimited by a rim, (b) wherein the implant is configured to be anchored to the site in the heart by an anchor head of the anchor being seated against the rim of the opening while a tissue-engaging element of the anchor extends through the opening and into tissue at the site.

Example 737. The system according to example 736, wherein the opening has: a first dimension that is smaller than a diameter of the anchor head, and a second dimension transverse to the first dimension that is greater than the diameter.

Example 738. The system according to any one of examples 665-737, wherein the implant is configured such that, after the anchor is anchored to the tissue at the site, the implant can slide relative to the anchor, and subsequently, the implant can be locked relative to the anchor such that the implant ceases to be slidable with relative to the anchor.

Example 739. The system according to any one of examples 665-738, wherein the implant comprises a bulking element coupled to or integral with the wing.

Example 740. The system according to example 739, further comprising an actuator operatively coupled to the bulking element such that actuation of the actuator changes a bulkiness of at least a portion of the implant.

Example 741. The system according to any one of examples 665-740, wherein the implant comprises a shape-memory member coupled to the wing, the shape-memory member configured to be intracardially heated to a temperature greater than 40 degrees C., and such that temporary heating of the shape-memory member to the temperature resizes the wing to a size, wherein the wing is configured to retain the size after cessation of the temporary heating.

Example 742. The system according to example 741, wherein the delivery tool is electrically connected or connectable to the shape-memory member and configured, via the electrical connection, to electrically heat the shape-memory member.

Example 743. The system according to any one of examples 665-742, wherein the wing comprises a flexible state and a rigid state, wherein a segment of the wing is more flexible in the flexible state than in the rigid state, and wherein the wing is configured such that it is transitionable between the rigid state and the flexible state.

Example 744. The system according to any one of examples 665-743, further comprising an insert disposed between the shaft and an interface of the implant, wherein the insert is slidable in a manner that disengages the shaft from the interface.

Example 745. The system according to example 744, further comprising a latch, wherein the insert is slidable in a manner that disengages the shaft from the interface by displacing the latch.

Example 746. The system according to any one of examples 665-745, wherein the interface is configured to promote tissue ingrowth thereon.

Example 747. The system according to example 746, wherein the implant defines an obstacle that is configured to inhibit the tissue ingrowth from progressing from the interface toward a tip portion of the wing.

Example 748. The system according to any one of examples 665-747, wherein an anchor head of the anchor and the interface are shaped such that, upon the anchor head reaching the interface, further rotation of the anchor head in the rotational direction pushes the interface distally with respect to the anchor.

Example 749. The system according to any one of examples 665-748, wherein the implant comprises an interface comprising: (a) a first collar configured to interface with an anchor head of the anchor, (b) a second collar, and (c) a neck portion connecting the first collar to the second collar and extending through a portion of the wing.

Example 750. The system according to example 749, wherein the interface has: (a) a loose state in which the portion of the wing is loosely coupled to the interface, and (b) a tight state in which the portion of the wing is sandwiched between the first collar and the second collar.

Example 751. The system according to any one of examples 749-750, wherein the interface is configured such that a driver can transition the interface from the loose state to the tight state.

Example 752. The system according to any one of examples 749-751, wherein the portion of the wing is a loop portion.

Example 753. The system according to any one of examples 665-752, wherein the delivery tool is configured to transition the system between: (a) a first state, in which a coupling is engaged with an interface of the implant, and (b) a second state, in which the coupling is engaged with the interface, and the wing has greater deflectability with respect to an axis of the anchor than in the first state, and (c) a deployed state, in which the coupling is disengaged from the interface.

Example 754. The system according to any one of examples 665-753, wherein the interface comprises a tubular anchor receiver defining a lumen, and a stopper is disposed within the lumen.

Example 755. The system according to example 754, wherein the stopper defines: a window dimensioned to facilitate helical advancement of a helical tissue-engaging element of the anchor therethrough, until an anchor head of the anchor meets the stopper, and a wall configured to inhibit non-helical advancement of the anchor distally through the interface.

Example 756. The system according to any one of examples 665-755, wherein an anchor head of the anchor is attached to a proximal end of a helical tissue-engaging element of the anchor, wherein the anchor head is shaped to define: an anchor hook facing in a first rotational direction around an axis of the anchor, and a smooth forward-torque face, facing in second rotational direction.

Example 757. The system according to example 756, wherein the delivery tool comprises a driver comprising: (1) a driveshaft, and (2) a drive head that defines: a smooth driver screw-in surface, facing in the first rotational direction, and a driver hook, facing in the second rotational direction, and shaped complementarily to the anchor hook.

Example 758. The system according to example 757, wherein the driver is configured: (a) to screw the helical tissue-engaging element into the tissue by applying torque, in the first rotational direction, to the anchor head by pressing the driver screw-in surface against the forward-torque face while pressing the anchor head distally, and (b) to unscrew the helical tissue-engaging element from the tissue by applying torque, in the second rotational direction, to the anchor head by hooking the driver hook into the anchor hook and pressing the driver hook against the anchor hook while pulling the anchor hook proximally.

Example 759. The system according to any one of examples 665-755, wherein the anchor is configured such that the anchor can be driven through an interface of the implant and the tissue, along a curved path within the tissue, such that a distal part of the anchor exits the tissue and is received by the interface.

Example 760. The system according to any one of examples 665-759, wherein the implant is configured such that: while the wing is in a compressed state, the implant has a hinged coupling between a root portion of the wing and the interface, wherein the hinged coupling facilitates articulation, at the hinged coupling, of the root portion with respect to the interface, and wherein expansion of the wing into an expanded state inhibits the articulation by restraining the hinged coupling.

Example 761. The system according to any one of examples 665-759, wherein the implant comprises an annular support, connected to a root portion of the wing, and configured such that: in a compressed state of the wing, the wing has a hinged coupling to the annular support that facilitates articulation, at the hinged coupling, of the wing with respect to the annular support, and wherein expansion of the wing toward an expanded state inhibits the articulation by restraining the hinged coupling.

Example 762. The system according to any one of examples 665-761, further comprising a lock at a distal portion of the shaft, wherein the lock comprises a first unit and a second unit, and has a locked state in which the first unit is mated with the second unit, and an unlocked state in which the first unit is separated and translatable away from the second unit.

Example 763. The system according to example 762, wherein the delivery tool is configured to: (1) while the lock is locked: via engagement of a coupling with an interface of the implant, position the implant within the heart, and a driver can be used to secure the implant to the tissue at the site of the heart by driving the anchor into the tissue, (2) reversibly and repeatedly transition the lock between the locked state and the unlocked state, and/or (3) while the implant remains secured to the tissue, disengage the coupling from the interface.

Example 764. The system according to any one of examples 665-763, wherein the implant comprises a beam coupled to the wing along a part of the wing, and a line coupled to the beam such that tensioning the line strains the beam, wherein the delivery tool is configured to intracardially reshape the wing by straining the beam by tensioning the line.

Although the operations of some of the disclosed methods are described in a particular, sequential order for convenient presentation, it should be understood that this manner of description encompasses rearrangement, unless a particular ordering is required by specific language set forth below. For example, operations described sequentially can in some cases be rearranged or performed concurrently. Moreover, for the sake of simplicity, the attached figures may not show the various ways in which the disclosed systems, apparatuses, devices, methods, etc. can be used in conjunction with other systems, apparatuses, devices, methods, etc.

The present invention is not limited to the examples that have been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

What is claimed is:

1. A system for use with a tissue of a subject, the system comprising:
an anchor, having:
a helical tissue-engaging element that has a distal tip, and
an anchor head, at a proximal end of the tissue-engaging element, the anchor head being shaped to define: a forward-torque face, and a reverse-torque face;
a driver, configured to transluminally advance the anchor to the tissue, and comprising:
a driveshaft, and
a drive head at a distal end of the driveshaft, the drive head being shaped to engage the anchor head in a manner in which:
while the driveshaft presses the drive head distally into engagement with the anchor head rotation of the drive head in a forward rotational direction screws the tissue-engaging element into the tissue by applying forward torque to the forward-torque face, and
tensioning the driveshaft while applying the forward torque to the forward-torque face pulls the drive head away from the anchor head,
rotation of the drive head in a reverse rotational direction hooks the drive head onto the anchor head, and
while the drive head remains hooked onto the anchor head, the driver is configured to unscrew the tissue-engaging element from the tissue by:
rotating the drive head in a reverse direction by applying reverse torque to the reverse-torque face, and
pulling the anchor proximally by tensioning of the driveshaft.

2. The system according to claim 1, wherein the reverse-torque face of the anchor head is configured to hook the drive head, by rotation of the drive head in the reverse rotational direction, in a manner that maintains contact between the anchor head and the drive head while the driveshaft is under tension.

3. The system according to claim 1, wherein the forward-torque face of the anchor head defines a smooth face that is closer to being perpendicular to the forward torque than to being parallel to the forward torque.

4. The system according to claim 1, wherein the driver is configured to, without any change of conformation of the drive head:
screw the tissue-engaging element into the tissue,
hook the drive head onto the anchor head, and
unscrew the tissue-engaging element from the tissue, and
disengage from the anchor head.

5. The system according to claim 1, wherein the driver is configured to, without any change of conformation of the anchor head:
screw the tissue-engaging element into the tissue,
hook the drive head onto the anchor head, and
unscrew the tissue-engaging element from the tissue, and
disengage from the anchor head.

6. The system according to claim 1, wherein:
the anchor head comprises an anchor hook that defines the reverse-torque face;
the drive head defines:
a smooth driver screw-in surface, facing in the forward rotational direction; and
a driver hook, facing in the reverse rotational direction, and shaped complementarily to the anchor hook; and
the driver is configured to:
screw the helical tissue-engaging element into the tissue by applying torque, in the forward rotational direction, to the anchor head by pressing the driver screw-in surface against the forward-torque face while pressing the anchor head distally, and
unscrew the helical tissue-engaging element from the tissue by applying torque, in the reverse rotational direction, to the anchor head by hooking the driver hook into the anchor hook and pressing the driver hook against the anchor hook while pulling the anchor hook proximally.

7. A method for use at a tissue of a subject, the method comprising:
using a driver that includes a driveshaft and a drive head at a distal end of the driveshaft, advancing to the tissue an anchor that has:
an anchor head defining:
a forward-torque face, and
a reverse-torque face, and
a helical tissue-engaging element extending from the anchor head;
driving the helical tissue-engaging element into the tissue by, while the drive head is engaged with the anchor head, applying forward torque to the forward-torque face by rotating the drive head in a forward rotational direction; and
disengaging the drive head from the anchor head by pulling the drive head away from the anchor head;
wherein the step of disengaging does not comprise changing a shape or a conformation of: the drive head or the anchor head.

8. The method according to claim 7, wherein disengaging comprises, while applying the forward torque to the forward-torque face, disengaging the drive head from the anchor head by pulling the drive head away from the anchor head.

9. The method according to claim 7, further comprising hooking the drive head onto the anchor head by rotating the drive head in a reverse rotational direction.

10. The method according to claim 9, wherein hooking the drive head onto the anchor head further comprises sliding the drive head proximally with respect to the anchor head.

11. The method according to claim 7, further comprising, prior to the step of disengaging, unscrewing the tissue-engaging element from the tissue by:
hooking the drive head onto the anchor head, and while the drive head remains hooked onto the anchor head:
applying reverse torque to the reverse-torque face by rotating the drive head in a reverse rotational direction, and
pulling the anchor proximally by tensioning of the driveshaft.

12. The method according to claim 11, further comprising, subsequently to unscrewing, removing the anchor from the subject by sliding the driver proximally while the drive head remains hooked onto the anchor head.

13. The method according to claim 11, wherein unscrewing comprises unscrewing the tissue-engaging element from the tissue, without changing a conformation of the drive head.

14. The method according to claim 11, wherein unscrewing comprises unscrewing the tissue-engaging element from the tissue, without changing a conformation of the anchor head.

15. A system for use with a tissue of a subject, the system comprising:
an anchor, having:
a helical tissue-engaging element that has a distal tip, and
an anchor head, at a proximal end of the tissue-engaging element, the anchor head being shaped to define: a forward-torque face, and a reverse-torque face;
a driver, configured to transluminally advance the anchor to the tissue, and comprising:
a driveshaft, and
a drive head at a distal end of the driveshaft, the drive head being shaped to engage the anchor head in a manner in which:
applying forward torque to the drive head screws the tissue-engaging element into the tissue by rotationally pressing a screw-in surface of the drive head against the forward-torque face,
applying reverse torque to the drive head hooks the drive head onto the anchor head and unscrews the tissue-engaging element from the tissue by rotationally pressing a screw-out surface of the drive head against the reverse-torque face,
pulling the driveshaft proximally while the screw-in surface is pressed rotationally against the forward-torque face disengages the drive head from the anchor head, and
pulling the driveshaft proximally while the drive head remains hooked onto the anchor head pulls the anchor proximally.

16. The system according to claim 15, wherein the reverse-torque face of the anchor head is configured to hook the drive head, by rotation of the drive head in the reverse rotational direction, in a manner that maintains contact between the anchor head and the drive head while the driveshaft is under tension.

17. The system according to claim 15, wherein the forward-torque face of the anchor head defines a smooth face that is closer to being perpendicular to the forward torque than to being parallel to the forward torque.

18. The system according to claim 15, wherein the driver is configured to, without any change of conformation of the drive head:
screw the tissue-engaging element into the tissue,
hook the drive head onto the anchor head, and
unscrew the tissue-engaging element from the tissue, and
disengage from the anchor head.

19. The system according to claim 15, wherein the driver is configured to, without any change of conformation of the anchor head:
screw the tissue-engaging element into the tissue,
hook the drive head onto the anchor head, and
unscrew the tissue-engaging element from the tissue, and
disengage from the anchor head.

20. The system according to claim 15, wherein:
the anchor head comprises an anchor hook that defines the reverse-torque face;
the drive head defines:
a smooth driver screw-in surface, facing in the forward rotational direction; and
a driver hook, facing in the reverse rotational direction, and shaped complementarily to the anchor hook; and
the driver is configured to:
screw the helical tissue-engaging element into the tissue by applying torque, in the forward rotational direction, to the anchor head by pressing the driver screw-in surface against the forward-torque face while pressing the anchor head distally, and
unscrew the helical tissue-engaging element from the tissue by applying torque, in the reverse rotational direction, to the anchor head by hooking the driver hook into the anchor hook and pressing the driver hook against the anchor hook while pulling the anchor hook proximally.
